(19) United States
(12) Patent Application Publication
(10) Pub. No.: US 2024/0189423 A1
(43) Pub. Date: Jun. 13, 2024

(54) BISPECIFIC BINDING AGENT-LIGAND FUSIONS FOR THE DEGRADATION OF TARGET PROTEINS

(71) Applicant: The Regents of the University of California, Oakland, CA (US)

(72) Inventors: James A. WELLS, San Francisco, CA (US); Katarina PANCE, San Francisco, CA (US); Josef A. GRAMESPACHER, San Francisco, CA (US); Kaan Kumru, San Francisco, CA (US)

(21) Appl. No.: 18/553,447

(22) PCT Filed: Mar. 30, 2022
(86) PCT No.: PCT/US22/22672

§ 371 Date: Sep. 29, 2023

Related U.S. Application Data
(60) Provisional application No. 63/168,554, filed on Mar. 31, 2021.

(51) Int. Cl. A61K 39/00; A61K 47/68; A61P 35/00; C07K 14/475; C07K 14/48; C07K 14/49; C07K 14/495; C07K 14/50; C07K 14/505; C07K 14/51; C07K 14/52; C07K 14/525; C07K 14/53; C07K 14/54; C07K 14/55; C07K 14/56; C07K 14/565; C07K 14/57; C07K 14/575; C07K 14/61; C07K 14/65; C07K 16/28; C12N 5/0781

(52) U.S. Cl. CPC A61K 39/4644; A61K 39/4612; A61K 39/4633; A61K 47/6851; A61P 35/00; C07K 14/475; C07K 14/48; C07K 14/49; C07K 14/495; C07K 14/50; C07K 14/505; C07K 14/51; C07K 14/521; C07K 14/525; C07K 14/53; C07K 14/5403; C07K 14/5406; C07K 14/5409; C07K 14/5412; C07K 14/5418; C07K 14/5425; C07K 14/5431; C07K 14/5434; C07K 14/5437; C07K 14/55; C07K 14/56; C07K 14/565; C07K 14/57; C07K 14/575; C07K 14/61; C07K 14/65; C07K 16/2803; C07K 16/2887; C12N 5/0635; C07K 2317/31; C07K 2319/30; C12N 2510/00

(57) ABSTRACT

The present disclosure relates to targeted degradation platform technology. For example, the present disclosure relates to bispecific binding agents for degrading endogenous proteins, whether membrane-associated or soluble, using the lysosome pathway. The disclosure also provides methods useful for producing such agents, nucleic acids encoding same, host cells genetically modified with the nucleic acids, as well as methods for modulating an activity of a cell and/or for the treatment of various disorders.

### CROSS-REFERENCE TO RELATED APPLICATIONS

This application claims priority to U.S. Provisional Patent Application No. 63/168,554, filed Mar. 31, 2021, the disclosure of which is incorporated by reference herein in its entirety, including any drawings.

### STATEMENT REGARDING FEDERALLY-SPONSORED RESEARCH AND DEVELOPMENT

This invention was made with government support under grant nos. R35 GM122451 and R01 CA248323 awarded by The National Institutes of Health. The government has certain rights in the invention.

### FIELD

The present disclosure relates to targeted degradation platform technology. For example, the present disclosure relates to bispecific binding agents for degrading endogenous proteins, whether membrane-associated or soluble, using the lysosome pathway. The disclosure also provides methods useful for producing such agents, nucleic acids encoding same, host cells genetically modified with the nucleic acids, as well as methods for modulating an activity of a cell and/or for the treatment of various disorders.

### BACKGROUND

The small molecule targeted degradation field has demonstrated that, in many cases, degradation of a target protein is more efficacious than inhibition. However, all of the E3 ligases targeted by small molecule degraders reside within cells, thereby limiting the intracellular mechanisms of action by small molecule degraders. As such, few examples exist of cell surface or extracellular proteins being targeted for degradation. Recently, lysosome targeting chimeras (LYTACs) consisting of antibody-glycan conjugates demonstrated successful degradation of cell surface and extracellular proteins via recruitment of mannose-6-phosphate receptor, which shuttles the target protein to the lysosome for degradation. However, the non-recombinant nature of these antibody-glycan conjugates and multi-step glycan synthesis make them difficult to express and manufacture on a large scale.

The disclosure provided herein overcomes the limitations of both small molecule degraders and LYTACs due to the ability to target cell surface proteins and the ease of the recombinant one-step production of our bispecific binding agent-ligand fusions. Further, the disclosure provided herein can improve the clinical efficacy of already approved antagonistic and inhibitory antibodies. In addition, the disclosure provided herein utilizes a mechanism of action independent of ubiquitin transfer and is capable of degrading soluble extracellular proteins or proteins with small intracellular domains that do not contain accessible lysine residue.

### BRIEF SUMMARY

The present disclosure demonstrates the development of a new targeted degradation platform technology, termed cytokine receptor targeting chimeras (KineTACs), which comprise of fully recombinant bispecific binding agents that utilize CXCL12-mediated internalization of its cognate receptors to target various therapeutically relevant cell surface proteins for lysosomal degradation.

Provided herein, among others, includes a bispecific binding agent comprising: a first binding domain comprising a cytokine selected from the group consisting of CXCL12, CCL1, CCL2, CCL3, CCL3L1, CCL4, CC4L1, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, CCL28, CXCL11, CXCL1, CXCL2, CXCL3, CXCL4, CXCL5, CXCL6, CXCL7, CXCL8, CXCL9, CXCL10, CXCL13, CXCL14, CXCL16, CXCL17, CX3CL1, XCL1, XCL2, vMIPII, vCXC1 that specifically binds to at least one endogenous cell surface receptor, and a second binding domain that specifically binds to a target protein. In some embodiments, the endogenous cell surface receptor is membrane associated. In some embodiments, the binding of the first binding domain to the at least one endogenous cell surface receptor results in the internalization of the target protein bound to the bispecific binding agent.

Provided herein, among others, includes a bispecific binding agent comprising: a first binding domain that specifically binds to at least one endogenous cell surface receptor, and a second binding domain that specifically binds to a target protein. In some embodiments, the endogenous cell surface receptor is membrane associated. In some embodiments, the binding of the first binding domain to the at least one endogenous cell surface receptor results in the internalization of the target protein bound to the bispecific binding agent.

In some embodiments, the first binding domain specifically binds to one endogenous cell surface receptor. In some embodiments, the first binding domain specifically binds to no more than two endogenous cell surface receptors. In some embodiments, the at least one endogenous cell surface receptor comprises targeting receptors and recycling receptors. In some embodiments, the at least one endogenous cell surface receptor comprises single-pass and multi-pass membrane proteins. In certain embodiments, the at least one endogenous cell surface receptor comprises at least one cytokine receptor. In other embodiments, the at least one cytokine receptor comprises at least one chemokine receptor. In some specific embodiments, the at least one chemokine receptors are selected from the group consisting of CXCR1, CXCR2, CXCR3, CXCR4, CXCR5, CXCR6, CXCR7 (or ACKR3), XCR1, XCR2, CCR1, CCR2, CCR3, CCR4, CCR5, CCR6, CCR7, CCR8, CCR9, CCR10, CCR11, CX3CR1, ACKR1, ACKR2, ACKR4, and ACKR5.

In one embodiment, the at least one chemokine receptors are selected from the group consisting of CCR1, CCR2, CCR3, CCR4, CCR5, CCR6, CCR7, CCR8, CCR9, CCR10, and CCR11.

In one embodiment, the at least one chemokine receptors are selected from the group consisting of CXCR7, CXCR4, CXCR3, CXCR1, CXCR2, CXCR5, CXCR6, CX3CR1, XCR1, and XCR2.

In one embodiment, the at least one chemokine receptors are selected from the group consisting of ACKR1, ACKR2, CXCR7, and ACKR4.

In one embodiment, the at least one cytokine receptor comprises at least one interleukin receptor. In one embodiment, the at least one interleukin receptors are selected from the group consisting of CD25, IL2RB, IL2RG, IL3RA, IL4R, IL13RA1, IL13RA2, IL5RA, IL6R, IL7R, IL8R, IL9R, IL10RA, IL10RB, IL11RA, IL12RB1, IL12RB2, IL15RA, CD4, IL17RA, IL17RC, IL17RB, IL17RE, IL27RA, IL18R1, and IL20RA.

In one embodiment, the at least one cytokine receptor comprises at least one interferon receptor. In one embodiment, the at least one interferon receptors are selected from the group consisting of IFNAR1, IFNAR2, IFNGR1, and IFNGR2.

In one embodiment, the at least one cytokine receptor comprises at least one prolactin receptor. In one embodiment, the at least one prolactin receptors are selected from the group consisting of EPOR, GHR, PRLR, CSF3R, LEPR, and CSF1R.

In one embodiment, the at least one cytokine receptor comprises at least one TNF receptor. In one embodiment, the at least one TNF receptors are selected from the group consisting of TNFR1, TNFR2, DR4, DR5, DCR1, DCR2, DR3, LTBR, BAFFR, TACI, OPG, RANK, CD40, EDAR, DCR3, FAS, and CD27.

In one embodiment, the at least one endogenous cell surface receptor comprises at least one growth factor receptor. In one embodiment, the at least one growth factor receptors are selected from the group consisting of FGFR2B, VEGFR2, PDGFRA, PDGFRB, NGFR, TRKC, TRKB, M6PR, and IGF1R.

In certain embodiments, the binding of the first binding domain to the at least one endogenous cell surface receptor results in the degradation of the target protein bound to the bispecific binding agent.

In some embodiments, the first binding domain comprises a cytokine, a chemokine, a growth factor or an isoform or a derivative capable of binding thereof. In some embodiments, the chemokine comprises a CXC chemokine, CCL chemokine, viral chemokine, or an isoform or a derivative capable of binding thereof. In certain embodiments, the chemokine is selected from the group consisting of CCL1, CCL2, CCL3, CCL3L1, CCL4, CC4L1, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, and CCL28. In one embodiment, the chemokine is selected from the group consisting of CXCL12, CXCL11, CXCL1, CXCL2, CXCL3, CXCL4, CXCL5, CXCL6, CXCL7, CXCL8, CXCL9, CXCL10, CXCL13, CXCL14, CXCL16, CXCL17, CX3CL1, XCL1, and XCL2. In one embodiment, the chemokine is selected from the group consisting of vMIPII, U83, and vCXC1.

In one embodiment, the cytokine is selected from the group consisting of interleukins, interferons, prolactins, tumor necrosis factors, and TGF-betas.

In one embodiment, the cytokine is an interleukin. In one embodiment, the interleukin is selected from the group consisting of IL2, IL3, IL4, IL5, IL6, IL7, IL8, IL9, IL10, IL11, IL12A, IL12B, IL13, IL15, IL16, IL17A, IL17B, IL17C, IL17F, IL18, IL19, IL20, IL21, IL22, IL24, IL25, IL26, IL27, IL28A, IL28B, IL29, IL31, IL32, IL33, IL34, IL36A, IL36B, IL36G, IL36RA, IL37, IL38, IL1A, IL1B, and IL1RN.

In one embodiment, the cytokine is an interferon. In one embodiment, the interferon is selected from the group consisting of IFNA, IFNA1, IFNA2, IFNA4, IFNA5, IFNA6, IFNA7, IFNA8, IFNA10, IFNA14, IFNA16, IFNA17, IFNA21, IFNB, and IFNG.

In one embodiment, the cytokine is a prolactin. In one embodiment, the prolactin is selected from the group consisting of EPO, GH1, GH2, PRL, CSF3, LEP, and CSF1.

In one embodiment, the cytokine is a tumor necrosis factor. In one embodiment, the prolactin is selected from the group consisting of TNFA, TNFB, TRAIL, TL1, BAFF, APRIL, RANKL, CD40LG, EDA, FASLG, and CD70.

In one embodiment, the cytokine is a TGF-beta. In one embodiment, the TGF-beta is selected from the group consisting of TGFB1, TGFB2, TGFB3, GDF15, GDF2, BMP10, INHA, and BMP3.

In one embodiment, the first binding domain comprises a growth factor. In one embodiment, the growth factor is selected from the group consisting of FGF1, FGF2, FGF3, FGF4, FGF5, FGF19, FGF21, FGF23, KGF, VEGF, PDGFA, PDGFB, NGF, NTF3, NTF4, BDNF, IGF1, and IGF2.

In some embodiments, the target protein comprises a soluble target protein and a membrane-associated target protein. In some embodiments, the target protein is a membrane-associated target protein, and wherein the second binding domain binds to an extracellular epitope of a membrane-associated target protein. In some embodiments, the target cell comprises a neoplastic cell. In some exemplary embodiments, the target cell is a cancer cell selected from the group consisting of breast cancer, B cell lymphoma, pancreatic cancer, Hodgkin's lymphoma, ovarian cancer, prostate cancer, mesothelioma, lung cancer, non-Hodgkin's B-cell (B-NHL), melanoma, chronic lymphocytic leukemia, acute lymphocytic leukemia, neuroblastoma, glioma, glioblastoma, bladder cancer, and colorectal cancer. In other embodiments, the target cell comprises an immune cell.

In some embodiments, the target protein is an immune checkpoint protein. In some embodiments, the target protein comprises a cancer antigen. In certain embodiments, the cancer antigen comprises HER2, EGFR, CDCP1, CD38, IGF-1R, MMP14, and TROP2.

In some embodiments, the target protein comprises an immunomodulatory protein. In certain embodiments, the immunomodulatory protein comprises PD-L1, PD-1, CTLA-4, B7-H3, B7-H4, LAG3, NKG2D, TIM-3, VISTA, CD39, CD73 (NT5E), A2AR, SIGLEC7, and SIGLEC15. In some embodiments, the target protein comprises a B cell antigen. In some exemplary embodiments, the B cell antigen comprises CD19 and CD20.

In some embodiments, the target protein comprises a soluble target protein. In some embodiments, the soluble target protein comprises an inflammatory cytokine, a growth factor (GF), a toxic enzyme, a target associated with metabolic diseases, a neuronal aggregate, or an autoantibody. In certain embodiments, the inflammatory cytokine comprises lymphotoxin, interleukin-1 (IL-1), IL-2, IL-5, IL-6, IL-12, IL-13, IL-17, IL-18, IL-23, tumor necrosis factor alpha (TNF-α), interferon gamma (IFNγ), and granulocyte-macrophage colony stimulating factor (GM-CSF). In certain embodiments, the growth factor comprises EGF, FGF, NGF, PDGF, VEGF, IGF, GMCSF, GCSF, TGF, RANK-L, erythropieitn, TPO, BMP, HGF, GDF, neurotrophins, MSF, SGF, GDF, and an isoform thereof. In certain embodiments, the toxic enzyme comprises a protein arginine deiminase 1 (PAD1), PAD2, PAD3, PAD4, and PAD6, leucocidin, hemolysin, coagulase, treptokinase, hyaluronidase. In certain embodiments, the toxic enzyme comprises PAD2 or PAD4. In some embodiments, the neuronal aggregate comprises A3, TTR, α-synuclein, TAO, and prion. In certain embodiments, the autoantibody comprises IgA, IgE, IgG, IgM and IgD.

In some embodiments, the first binding domain and the second binding domain are each independently selected from the group consisting of natural ligands or a fragment, derivative, or small molecule mimetic thereof, IgG, half antibodies, single-domain antibodies, nanobodies, Fabs, monospecific Fab2, Fc, scFv, minibodies, IgNAR, V-NAR, hcIgG, VHH domain, camelid antibodies, and peptibodies.

In some embodiments, the first binding domain and the second binding domain together form a bispecific antibody, a bispecific diabody, a bispecific Fab2, a bispecific camelid antibody, or a bispecific peptibody scFv-Fc, a bispecific IgG, a knob and hole bispecific IgG, a Fc-Fab, and a knob and hole bispecific Fc-Fab, a cytokine-IgG fusion, a cytokine-Fab fusion, and a cytokine-Fc-scFv fusion. In some embodiments, the first binding domain comprises an Fc-fusion, and the second binding domain comprises an Fc-Fab.

In some embodiments, the bispecific binding agent provided herein comprises one or more sequences selected from Table 1.

Also provided herein incudes a nucleic acid that encodes the bispecific binding agent of the present disclosure. In some embodiments, the nucleic acid is operably connected to a promoter.

Further provided herein incudes an engineered cell capable of protein expression comprising the nucleic acid of the present disclosure. In some embodiments, the engineered cell comprises a B cell, a B memory cell, or a plasma cell.

Another aspect of the present disclosure relates to a method for making a bispecific binding agent provided herein. In some embodiments, the method comprises: i) providing a cell capable of protein synthesis, comprising the nucleic acid disclosed herein and ii) inducing expression of the bispecific binding agent.

The present disclosure further provides a vector which comprises the nucleic acid described herein. In some embodiments, the vector further comprises a promoter, wherein the promoter is operably linked to the nucleic acid.

Another aspect of the present disclosure provides an immunoconjugate comprising: i) a bispecific binding agent of any one of the preceding claims, ii) a small molecule, and iii) a linker.

The present disclosure also provides a pharmaceutical composition. In some embodiments, the pharmaceutical composition comprises the bispecific binding agent, the nucleic acid, the vector, the engineered cell, or the immunoconjugate described herein, and a pharmaceutically acceptable excipient.

In another aspect, the present disclosure provides a method of treating a disorder in a subject. In some embodiments, the method comprising administering to a subject in need thereof, a therapeutically effective amount of the bispecific binding agent, the nucleic acid, the vector, the engineered cell, the immunoconjugate, or the pharmaceutical composition provided herein.

In some embodiments, the disorder comprises a neoplastic disorder, an inflammatory disease, a metabolic disorder, an endocrine disorder, and a neurological disorder. In certain embodiments, the neoplastic disorder comprises breast cancer, B cell lymphoma, pancreatic cancer, Hodgkin's lymphoma, ovarian cancer, prostate cancer, mesothelioma, lung cancer, non-Hodgkin's B-cell (B-NHL), melanoma, chronic lymphocytic leukemia, acute lymphocytic leukemia, neuroblastoma, glioma, glioblastoma, bladder cancer, and colorectal cancer. In certain embodiments, the inflammatory disease comprises inflammatory intestinal disease, rheumatoid arthritis, lupus, Crohn's disease, and ulcerative colitis. In certain embodiments, the metabolic disorder comprises diabetes, Gaucher disease, Hunter syndrome, Krabbe disease, maple syrup urine disease, metachromatic leukodystrophy, mitochondrial encephalopathy, lactic acidosis, stroke-like episodes (MELAS), Niemann-Pick, phenylketonuria (PKU), porphyria, Tay-Sachs disease, and Wilson's disease. In certain embodiments, the neurological disorder comprises Parkinson's disease, Alzheimer's disease, and multiple sclerosis.

### BRIEF DESCRIPTION OF THE DRAWINGS

FIGS. 1A-1B: 1A) CXCL12-mediated receptor internalization could be used for targeted degradation. A) Schematic of CXCL12 signaling via CXCR4 and CXCR7. 1B) Schematic of KineTAC concept, in which a bispecific binding agent could simultaneously bind CXCR4/CXCR7 and a protein of interest, causing the lysosomal degradation of the target protein.

FIGS. 2A-2F: KineTACs target cell surface protein PD-L1 for degradation. 2A) Schematic of CXCL12-Tecentriq KineTAC construct. 2B) Multipoint BLI measurement of CXCL12-Tec shows high affinity to PD-L1 Fc fusion. 2C) Flow cytometry showing CXCL12-Isotype binding on MDA-MB-231 cells endogenously expressing CXCR4 and CXCR7. 2D) Dose escalation experiment showing PD-L1 degradation in MDA-MB-231 cells after 24 hr treatment with CXCL12-Tec. 2E) PD-L1 levels after 24 hr treatment of MDA-MB-231 cells with 100 nM of Tecentriq controls and CXCL12-Tec. 2F) PD-L1 levels after 24 hr treatment with MDA-MB-231 cells with 100 nM CXCL12-Tec or 100 nM CXCL12-Isotype+100 nM Tecentriq Fab. Data are representative of at least two independent biological replicates. Densitometry was used to calculate protein levels and normalized to PBS control.

FIGS. 3A-3F: KineTAC platform is generalizable to targeting other therapeutically relevant cell surface proteins. Dose escalation showing HER2 degradation in 3A) MCF-7, 3B) MDA-MB-175VII, or 3C) SK-BR-3 cells after 24 hr treatment with CXCL12-Tras or 100 nM Trastuzumab Fab. 3D) Summary of HER2 degradation in MCF-7, MDA-MB-175VII, and SK-BR-3 cell lines. Error bars represent the standard deviation of two biological replicates. 3E) Dose escalation showing EGFR degradation in HeLa cells after 24 hr treatment with CXCL12-Ctx or 100 nM Ctx-Isotype. 3F) Dose escalation showing CDCP1 degradation in HeLa cells after 24 hr treatment with CXCL12-4A06 or 100 nM 4A06 Fab. Data are representative of at least two independent biological replicates. Densitometry was used to calculate protein levels and normalized to PBS control.

FIGS. 4A-4D: CXCL12-Tec causes degradation of PD-L1 in MDA-MB-231 in a lysosomal- and time-dependent manner. 4A) 1 hr pre-treatment of MDA-MB-231 cells with either 100 nM bafilomycin or 5 μM MG-132 followed by 24 hr treatment with 100 nM CXCL12-Tec indicates that CXCL12-Tec degrades PD-L1 in a lysosome-dependent manner. 4B) Time course experiment showing degradation of PD-L1 at various time points after treatment with 100 nM CXCL12-Tec. 4C) Data from time course experiment plotting levels of PD-L1 relative to control versus time (hr). Error bars represent the standard deviation of three biological replicates. 4D) Levels of cell surface and whole cell PD-L1 after 24 hr treatment of MDA-MB-231 cells with 100 nM CXCL12-Tec or Tecentriq Fab shows marginal differences between cell surface and whole cell PD-L1 levels. Data are representative of at least two independent biological replicates.

FIGS. 5A-5C: KineTAC-mediated degradation is not only dependent on CXCR4 internalization. 5A) Schematic of proposed mechanisms of KineTAC-mediated degradation of target proteins. 5B) siRNA knockdown of CXCR4 in HeLa cells after 48 hr transfection is dependent on CXCR4-targeting siRNA pool. C) EGFR levels are unchanged after siRNA knockdown of CXCR4 for 48 hrs followed by 24 hr treatment with 100 nM CXCL12-Ctx in HeLa cells. Data are representative of at least two independent biological replicates. Densitometry was used to calculate protein levels and normalized to PBS control.

FIGS. 6A-6D: KineTACs do not induce major cellular perturbations. Fold change in the abundance of 6A) surface enriched or 6B) whole cell MDA-MB-231 proteins detected using quantitative proteomics analysis after 48 hr treatment with 100 nM CXCL12-Tec. Data are the mean of two biological replicates and two technical replicates. 6C) Washout experiment showing that PD-L1 levels do not recover up to 48 hr post-treatment with 100 nM CXCL12-Tee in MDA-MB-231 cells. 6D) Washout experiment showing that EGFR levels recover at 24 hr post-treatment with 100 nM CXCL12-Ctx in HeLa cells. Data are representative of at least two independent biological replicates. Densitometry was used to calculate protein levels and normalized to PBS control.

FIGS. 7A-7E: Requirements for efficient KineTAC-mediated degradation of PD-L1. 7A) PD-L1 levels after treatment with agonistic and antagonistic 100 nM CXCL12-Tec variants for 24 hr in MDA-MB-231 cells. 7B) PD-L1 levels after treatment with 100 nM CXCL12-Tec wild-type or alanine mutants for 24 hr in MDA-MB-231 cells. 7C) Correlation of PD-L1 levels as calculated by densitometry and either K_{D}, Kₒₙ, or K_{off}. Wild-type Tecentriq is indicated in red. Error bars represent the standard deviation of three biological replicates. 7D) PD-L1 levels after treatment with 100 nM CXCL12-Tec or pH-sensitive binder CXCL12-BMS936559 for 24 hr in MDA-MB-231 cells. 7E) PD-L1 levels after treatment with 100 nM aglycosylated or glycosylated CXCL12-Tec for 24 hr in MDA-MB-231 cells. Data are representative of at least two independent biological replicates. Densitometry was used to calculate protein levels and normalized to PBS control.

FIGS. 8A-8B: KineTAC diabody construct does not induce significant degradation of PD-L1 as compared to bispecific. 8A) Multipoint BLI measurement of CXCL12-Tec diabody shows high affinity to PD-L1 Fc fusion. 8B) PD-L1 levels after treatment with 100 nM CXCL12-Tec bispecific or diabody for 24 hr in MDA-MB-231 cells shows that the bispecific construct is superior in degrading PD-L1. Data are representative of at least two independent biological replicates. Densitometry was used to calculate protein levels and normalized to PBS control.

FIGS. 9A-9B: HER2-targeting KineTAC reduces in vitro cell viability of breast cancer cell lines. In vitro potency of CXCL12-Tras in two HER2-positive breast cancer cell lines, 9A) MDA-MB-175VII and 9B) SK-BR-3, as compared to Trastuzumab Fab or IgG. Three biological data points are shown.

FIGS. 10A-10C: KineTACs target cell surface protein PD-L1 for degradation. FIG. 10A is flow cytometry showing degradation of surface PD-L1 on MDA-MB-231 cells after 24 hr treatment with 100 nM CXCL12-Atz, but not after addition of controls. FIG. 10B shows levels of cell surface and whole cell PD-L1 after 24 hr treatment of MDA-MB-231 cells with 100 nM CXCL12-Atz or atezolizumab Fab shows marginal differences between cell surface and whole cell PD-L1 levels. FIG. 10C is a representative western blot showing PD-L1 levels after 24 hr treatment of MDA-MB-231 cells with high concentrations (50-500 nM) of CXCL12-Atz shows that no “hook effect” is observed in this concentration range. Data are representative of at least three independent biological replicates. Densitometry was used to calculate protein levels and normalized to PBS control.

FIG. 11: Correlation of HER2 levels to CXCR7/HER2 transcript level ratio as calculated by densitometry after treatment with 100 nM CXCL12-Tras for 24 hrs in MCF7, MDA-MB-175VII, and SK-BR-3 cells. Error bars represent the standard deviation of at least two biological replicates.

FIGS. 12A-12F: FIG. 12A shows a summary of EGFR degradation in various EGFR-expressing cell lines following 24 hr treatment with CXCL12-Ctx. FIG. 12B shows dose escalation showing EGFR degradation in FIG. 12B, MDA-MB-231, FIG. 12C, A431, and FIG. 12D, NCI-H292 cells following treatment with CXCL12-Ctx or 100 nM Cetuximab isotype. FIG. 12E shows EGFR levels after treatment with 100 nM CXCL12-Ctx or Ctx isotype control for 24 hrs in non-small cell lung cancer cell lines A549, NCI-H358, and HCC827. FIG. 12F shows correlation of EGFR levels to CXCR7/EGFR transcript level ratio as calculated by densitometry after treatment with 100 nM CXCL12-Ctx for 24 hrs in HeLa, A431, NCI-H292, MDA-MB-231, A549, NCI-H358, and HCC827 cells. Error bars represent the standard deviation of at least two biological replicates.

FIG. 13: Dose escalation showing TROP2 degradation in MCF7 cells following treatment with CXCL12-Sacituzumab or sacituzumab isotype. Data are representative of at least two independent biological replicates. Densitometry was used to calculate protein levels and normalized to PBS control. P-values were determined by unpaired two-tailed t-tests.

FIGS. 14A-14C: KineTACs mediate degradation of PD-1 on primary human CD8+ T cells. Representative flow cytometry showing presence of CD8+ T cell activation markers, FIG. 14A, PD-1 or FIG. 14B, CD25, following 4 day incubation with activation cocktail (IL-2, IL-15, anti-CD3, anti-CD28). FIG. 14C shows representative flow demonstrating cell surface PD-1 degradation on activated primary human CD8+ T cells following 24 hr treatment with 100 nM CXCL12-Nivo or nivolumab isotype.

FIGS. 15A-15F: FIG. 15A shows PD-L1 in MDA-MB-231 or EGFR in HeLa cells are degraded after 24 hr treatment with 100 nM CXCL11-Atz or CXCL11-Ctx, respectively, at levels similar to CXCL12 KineTACs. CXCL11 and vMIPII are alternative CXCR7-targeting KineTACs that degrade PD-L1 and EGFR. FIG. 15B shows a representative western blot showing PD-L1 degradation in MDA-MB-231 cells following 24 hr treatment with various doses of CXCL11-Atz or 100 nM atezolizumab Fab. FIG. 15C shows a comparison of dose response of PD-L1 degradation in MDA-MB-231 cells following 24 hr treatment with CXCL11- or CXCL12-Atz. FIG. 15D shows a representative western blot showing EGFR degradation in HeLa cells following 24 hr treatment with various doses of CXCL11-Ctx or 100 nM cetuximab isotype. FIG. 15E shows a comparison of dose response of EGFR degradation in HeLa cells following 24 hr treatment with CXCL11- or CXCL12-Ctx. FIG. 15F shows PD-L1 in MDA-MB-231 cells is degraded after 24 hr treatment with 100 nM vMIPII-Atz at levels similar to CXCL12-Atz. Data are representative of at least three independent biological replicates. P-values were determined by unpaired two-tailed t-tests. Densitometry was used to calculate protein levels and normalized to PBS control.

FIGS. 16A-16B: KineTACs mediate target degradation in a highly selective, lysosomal-, time-, and CXCR7-dependent manner. Fold change in abundance of FIG. 16A, surface enriched or FIG. 16B, whole cell HeLa proteins detected using quantitative proteomics analysis after 48 hr treatment with 100 nM CXCL12-Ctx reveals highly selective EGFR degradation. Data are the mean of two biological replicates and two technical replicates. Proteins showing a >2-fold change from PBS control with a significance of P<0.01 were considered significantly changed.

FIGS. 17A-17B: KineTACs are highly selective and functionally active in vitro. FIG. 17A shows a comparison between KineTAC and LYTAC whole cell quantitative proteomics experiments in HeLa cells shows large overlap in total proteins identified. FIG. 17B shows 23 of 25 proteins that were significantly up- or down-regulated in the LYTAC dataset were identified in the KineTAC whole cell dataset.

FIG. 18: Quantification of CXCL12-Tras plasma levels in male nude mice injected intravenously at 5, 10, or 15 mg/kg. The data show values of three different mice. Densitometry was used to calculate protein levels and normalized to whole protein levels. P-values were determined by unpaired two-tailed t-tests.

FIGS. 19A-19F: CXCL12-Atz is cross-reactive to mouse cell lines and stable in vivo. Flow cytometry showing that human CXCL12 is cross reactive and binds to the surface of FIG. 19B, MC38 and FIG. 19C, CT26 mouse cell lines. FIG. 19D shows a dose escalation showing mouse PD-L1 degradation in MC38 and CT26 cells following 24 hr treatment with CXCL12-Atz. Representative western blot of dose escalation showing mouse PD-L1 degradation in FIG. 19E, MC38 or FIG. 19F, CT26 mouse cells following 24 hr treatment with CXCL12-Atz and mouse IFNg. FIG. 19A shows a representative western blot showing plasma levels of CXCL12-Tras in male nude mice injected intravenously with 5, 10, or 15 mg/kg. Data are representative of three independent biological replicates or mice. Densitometry was used to calculate protein levels and normalized to PBS control.

FIGS. 20A-20H: KineTACs enable intracellular uptake of soluble extracellular proteins. FIG. 20 is a schematic of KineTAC concept for targeting extracellular proteins for lysosomal degradation. FIG. 20B shows representative flow cytometry showing shift in median fluorescence intensity in HeLa cells treated for 24 hr with 50 nM CXCL12-Beva and 25 nM VEGF-647 compared to VEGF alone. FIG. 20C is a summary of flow cytometry demonstrating shift in median fluorescence intensity in HeLa cells following 24 hr treatment with 50 nM CXCL12-Beva or isotype controls and 25 nM VEGF-647 compared to VEGF alone. FIG. 20D shows a comparison of HeLa cells lifted with versene (normal lift) or 0.25% trypsin-EDTA (trypsin lift) following 24 hr treatment with 50 nM CXCL12-Beva or isotype controls and 25 nM VEGF-647. No significant change in median fluorescence intensity suggests that fluorescence shift represents accumulation of intracellular VEGF-647. FIG. 20E is a summary of flow cytometry demonstrating decrease in median fluorescence intensity in HeLa cells following pre-treatment with 100 nM bafilomycin and 24 hr treatment with 50 nM CXCL12-Beva and 25 nM VEGF-647, as compared to no pretreatment with bafilomycin. FIG. 20F is a time course experiment showing increase in VEGF-647 uptake overtime in HeLa cells treated with 50 nM CXCL12-Beva and 25 nM VEGF-647. FIG. 20G shows HeLa cells treated for 24 hr with varying ratios of CXCL12-Beva to VEGF, at constant 25 nM VEGF-647, demonstrate that increasing the KineTAC:VEGF ratio increases VEGF uptake. FIG. 20H shows a panel of cell lines for VEGF-647 uptake experiments, demonstrating increased VEGF-647 uptake for CXCL12-Beva treated cells compared to bevacizumab isotype or VEGF alone. Median fluorescence intensity (MFI) was measured using live cell flow cytometry. Data are representative of at least three independent biological replicates and error bars show standard deviation between replicates. P-values were determined by unpaired two-tailed t-tests. Fold changes are reported relative to incubation with soluble ligand alone.

FIGS. 21A-21C: KineTACs mediate uptake of extracellular VEGF. FIG. 21A shows representative flow cytometry showing levels of VEGF-647 cell surface labeling after incubation with HeLa cells for 1 hr at 4° C. and normal versene lift. FIG. 21B shows representative flow cytometry showing reduction of VEGF-647 cell surface labeling after incubation with HeLa cells for 1 hr at 4° C. and lift with 0.25% trypsin-EDTA (trypsin lift). FIG. 21C shows correlation of VEGF uptake as calculated by flow cytometry to CXCR7 transcript levels. Error bars represent the standard deviation of three biological replicates. Linear regression analysis using GraphPad Prism was used to calculate the coefficient of determination (R²) to determine correlation. Data are representative of two biological replicates.

FIGS. 22A-22C: KineTACs mediate the uptake of extracellular TNFa. FIG. 22A shows representative flow cytometry showing shift in median fluorescence intensity in HeLa cells treated for 24 hr with 50 nM CXCL12-Ada and 25 nM TNFa-647 compared to TNFa alone. FIG. 22B shows a summary of flow cytometry demonstrating shift in median fluorescence intensity in HeLa cells following 24 hr treatment with 50 nM CXCL12-Ada or adalimumab isotype, and 25 nM TNFa-647 compared to TNFa alone. FIG. 22C shows HeLa cells treated for 24 hr with varying ratios of CXCL12-Ada to TNFa, at constant 25 nM TNFa-647, demonstrate that increasing the KineTAC:TNFa ratio increases TNFa uptake. P-values were determined by unpaired two-tailed t-tests. Fold changes are reported relative to incubation with soluble ligand alone.

FIGS. 23A-23B: IL2-bearing KineTACs can co-opt CD25 to degrade cell surface PD-1. FIG. 23A show a schematic of the KineTAC concept for targeting cell surface proteins for lysosomal degradation via IL2-mediated endocytosis. FIG. 23B shows a summary of flow cytometry data demonstrating degradation of cell surface PD-1 on activated primary human CD8+ T cells. Data are representative of at least three independent biological replicates. Data are representative of at least three biological replicates. P-values were determined by unpaired two-tailed t-tests.

FIGS. 24A-24B: Requirements for efficient KineTAC-mediated degradation of PD-L1. FIG. 24A shows PD-L1 levels after treatment with CXCL12-Atz wild-type or CXCL12 N-terminal variants (100 nM) for 24 hr in MDA-MB-231 cells. FIG. 24B shows correlation of PD-L1 levels to CXCR7 IC50 (nM) of CXCL12 variants as calculated by densitometry after treatment with 100 nM CXCL12-Atz variants for 24 hrs in MDA-MB-231 cells. Wild-type CXCL12 is indicated in red. Error bars represent the standard deviation of three biological replicates.

FIGS. 25A-25B: FIG. 25A shows HER2 levels after treatment with 100 nM CXCL12-Tras or CXCL12-Ptz in MCF7 cells demonstrate that different epitope binders affect degradation of HER2. FIG. 25B shows EGFR levels after treatment with 100 nM of CXCL12-Ctx, Depa, Nimo, Matu, Neci, or Pani in HeLa cells demonstrate that there is dependence on EGFR binding epitope for degradation efficiency. Data are representative of at least three independent biological replicates. Densitometry was used to calculate protein levels and normalized to PBS control. P-values were determined by unpaired two-tailed t-tests. Linear regression analysis using GraphPad Prism was used to calculate the coefficient of determination (R²) to determine correlation.

FIGS. 26A-26B: FIG. 26D shows a schematic of EGFR extracellular domains (I-IV) with locations of anti-EGFR antibody epitopes (left) and crystal structure of domain III (PDB: 5SX4) with the epitopes of anti-EGFR binders highlighted in their respective colors. FIG. 26B shows correlation of EGFR levels to K_{D} as calculated by densitometry after treatment with 100 nM CXCL12-Depa, Nimo, Pani, Neci, Matu, or Ctx for 24 hrs in HeLa cells. Error bars represent the standard deviation of three biological replicates.

FIGS. 27A-27B: FIG. 27A is a schematic of CXCL12-Atz IgG fusion construct where CXCL12 chemokine is fused to the N-terminus of the heavy chain (HC) or light chain (LC) of atezolizumab IgG via an Avi tag linker. FIG. 27B shows PD-L1 levels after treatment with 100 nM CXCL12-Atz bispecific or IgG fusion for 24 hr in MDA-MB-231 cells shows that the bispecific construct is superior in degrading PD-L1. Data are representative of at least two independent biological replicates. Densitometry was used to calculate protein levels and normalized to PBS control.

FIGS. 28A-28C: KineTACs are highly selective and functionally active in vitro. FIG. 28A shows in vitro potency of CXCL12-Tras in HER2-expressing breast cancer cell line MDA-MB-175VII demonstrates superior cell killing compared to CXCL12 isotype, which is functionally inactive. FIG. 28B shows in vitro potency of CXCL12-Tras in MDA-MB-175VII cells demonstrates superior cell killing compared to trastuzumab Fab alone. FIG. 28E shows in vitro potency of CXCL12-Ctx in EGFR-expressing non-small cell lung cancer cell line NCI-H358 demonstrates superior cell killing compared to cetuximab IgG. Data are representative of at least two biological replicates. P-values were determined by unpaired two-tailed t-tests.

FIG. 29: Other chemokines, cytokines, and growth factors are useful KineTACs. Fold change in median fluorescence intensity in THP-1 cells after 50 nM VEGF-647 and 25 nM KineTAC treatment is shown indicating likely uptake of VEGF triggered by KineTACs consisting of bevacizumab hole Fc and the indicated cytokine knob Fc.

FIG. 30: Further evidence that other chemokines, cytokines, and growth factors are useful KineTACs. Fold change in median fluorescence intensity in THP-1 cells after 50 nM VEGF-pHrodoRed and 25 nM KineTAC treatment is shown indicating likely uptake of VEGF triggered by KineTACs consisting of bevacizumab hole Fc and the indicated cytokine knob Fc. Error bars represent standard deviation of 2 biological replicates.

FIG. 31: Confocal microscopy images of HeLa cells treated for 24 hr with 100 nM CXCL12-Ctx shows near complete removal of EGFR from the cell surface.

### DETAILED DESCRIPTION

The present disclosure provides, among others, fully recombinant bispecific binding agents comprising a first binding domain and a second binding domain for targeted degradation of a target protein, whether soluble or membrane-associated. As used herein, the targeted degradation can be mediated by the lysosome pathway. The first binding domain can specifically bind to at least one endogenous cell surface receptor. In some embodiments, the binding of the first binding domain to the at least one endogenous cell surface receptor results in the internalization of the endogenous cell surface receptor and the bispecific binding agent. In certain embodiments, the endogenous cell surface receptor is membrane associated. Further, the second binding domain can specifically bind to a target protein. The bispecific binding agents of the present disclosure are useful as a targeted degradation platform. The first and second binding domains can be altered and combined for specific purposes.

Targeted protein degradation has emerged over the past two decades as a potential rival to traditional therapeutic modalities for a variety of human diseases. Traditional inhibitors, such as small molecules and biologics, operate through occupancy-driven pharmacology. This paradigm requires high binding potency and frequent dosing to maintain a prolonged therapeutic effect. Furthermore, non-enzymatic protein functions, such as scaffolding functions of kinases, are difficult to block using inhibitors due to lack of ligandable binding areas. Degrader technologies, on the other hand, operate via event-driven pharmacology, enabling one degrader molecule to catalytically degrade multiple target protein molecules. Small molecule degraders, such as PROteolysis TArgeting Chimeras (PROTACs), are heterobifunctional molecules comprised of a ligand to an E3 ubiquitin ligase chemically linked to a protein of interest ligand. Simultaneous binding to both the E3 ligase and target protein enables the transfer of ubiquitin onto the target protein and its subsequent degradation by the proteasome. Small molecule degraders have demonstrated success in degrading over 60 protein targets, providing greater therapeutic benefit compared to the parent inhibitor, overcoming classical resistance mechanisms, and targeting “undruggable” proteins. Furthermore, two PROTACs are currently being tried in phase I clinical trials to test their efficacy and safety as therapeutic agents.

Due to their intracellular mechanism of action, small molecule PROTACs are limited to targeting proteins with cytosolic domains with ligandable surfaces. As such, very few examples exist for PROTACs degrading membrane proteins. Given the vast number of cell surface and extracellular disease-related proteins, there is a critical need to develop degraders capable of targeting this portion of the proteome. Two recent platforms have expanded targeted protein degradation to this important class. One in particular, termed antibody-based PROTACs (AbTACs), utilizes bispecific IgGs to hijack cell surface E3 ligase RNF43 to degrade checkpoint inhibitor protein programmed death-ligand 1 (PD-L1) via the lysosome. The second, termed lysosome-targeting chimeras (LYTACs), utilizes IgG-glycan bioconjugates to co-opt lysosome shuttling receptors, such as mannose-6-phosphate receptor (M6PR) and asialoglycoprotein receptor (ASGPR), to degrade both cell surface and soluble extracellular targets. However, LYTAC production requires complex chemical synthesis and in vitro bioconjugation, thereby limiting the modularity of this platform.

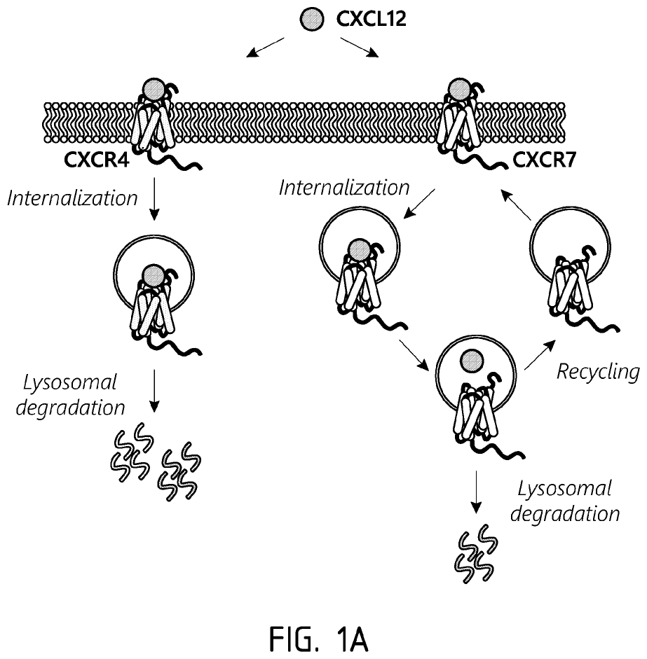

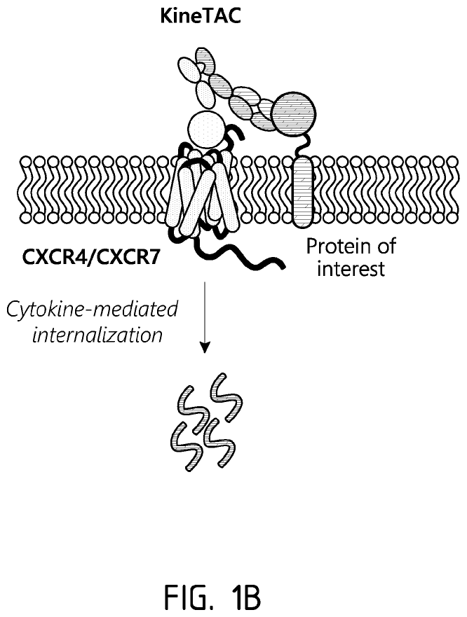

Cytokines and growth factors are each a diverse class of soluble extracellular proteins. Upon binding to their cognate receptors on the surface of cells, cytokines and growth factors trigger downstream signaling, leading to internalization of the cytokine-receptor complex. The present disclosure demonstrates that cytokine-mediated and growth factor-mediated internalization could be co-opted for targeted degradation applications. For instance, chemokine CXCL12 is known to specifically bind to two chemokine receptors, CXCR4 and CXCR7, and subsequently internalize via two distinct mechanisms (FIG. 1A). Binding to CXCR4 agonizes the receptor, leading to its internalization and shuttling to the lysosome for degradation. CXCR7, however, is constitutively internalized and recycled back to the cell surface, independent of ligand binding. Therefore, binding to CXCR7 causes the internalization of CXCL12 without subsequent degradation of CXCR7 or downstream signaling. In some non-limiting exemplary embodiments, the present disclosure demonstrates the development of a new targeted degradation platform technology, termed cytokine receptor targeting chimeras (KineTACs), which comprise of fully recombinant bispecific binding agents that utilize CXCL12-mediated internalization of its cognate receptors to target various therapeutically relevant cell surface proteins for lysosomal degradation (FIG. 1B).

Among others, the present disclosure demonstrates that these fully recombinant bispecific binding agents (e.g., the KineTACs) can efficiently degrade the target proteins, and that the degradation is dependent on the bispecific KineTAC scaffold and occurs in a dose-dependent manner. Further, the target degradation mediated by the bispecific binding agents of the present disclosure does not induce unwanted, off-target proteome-wide changes. Further, the present disclosure demonstrates that the levels of degradation of the target protein are dependent on the binding affinity of the antibody arm to the target protein. Additionally, the present disclosure shows that the stability and pharmacokinetic properties of KineTACs can be improved, e.g., by glycosylation, for in vivo use without major disruption to degradation efficiency. The present disclosure also shows that some bispecific IgGs can be more effective than a diabody construct. Furthermore, the present disclosure demonstrates that KineTAC-mediated degradation can cause functional consequences in reducing cancer cell viability in vitro, and that significant degradation of the HER2 is not needed to induce major reductions in cell viability. The present disclosure further demonstrates that the bispecific binding agents provided herein (e.g., the KineTACs) are generalizable to multiple targets in multiple cell types, and therefore could be expanded to targeting various protein targets for degradation.

### Definition

The singular form “a”, “an”, and “the” include plural references unless the context clearly dictates otherwise. For example, the term “a cell” includes one or more cells, including mixtures thereof “A and/or B” is used herein to include all of the following alternatives: “A”, “B”, “A or B”, and “A and B.”

The terms “administration” and “administering”, as used interchangeably herein, refer to the delivery of a composition or formulation by an administration route including, but not limited to, intravenous, intra-arterial, intracerebral, intrathecal, intramuscular, intraperitoneal, subcutaneous, intramuscular, and combinations thereof. The term includes, but is not limited to, administration by a medical professional and self-administration.

The terms “host cell” and “recombinant cell” are used interchangeably herein. It is understood that such terms, as well as “cell culture”, “cell line”, refer not only to the particular subject cell or cell line but also to the progeny or potential progeny of such a cell or cell line, without regard to the number of transfers. It should be understood that not all progeny are exactly identical to the parental cell. This is because certain modifications may occur in succeeding generations due to either mutation (e.g., deliberate or inadvertent mutations) or environmental influences, such progeny may not, in fact, be identical to the parent cell, but are still included within the scope of the term as used herein, so long as the progeny retain the same functionality as that of the original cell or cell line.

The term “operably linked”, as used herein, denotes a physical or functional linkage between two or more elements, e.g., polypeptide sequences or polynucleotide sequences, which permits them to operate in their intended fashion.

The term “heterologous”, refers to nucleic acid sequences or amino acid sequences operably linked or otherwise joined to one another in a nucleic acid construct or chimeric polypeptide that are not operably linked or are not contiguous to each other in nature.

The term “percent identity,” as used herein in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See, e.g., the NCBI web site at ncbi.nlm.nih.gov/BLAST. This definition also refers to, or may be applied to, the complement of a test sequence. This definition also includes sequences that have deletions and/or additions, as well as those that have substitutions. Sequence identity typically is calculated over a region that is at least about 20 amino acids or nucleotides in length, or over a region that is 10-100 amino acids or nucleotides in length, or over the entire length of a given sequence. Sequence identity can be calculated using published techniques and widely available computer programs, such as the GCS program package (Devereux et al, Nucleic Acids Res (1984) 12:387), BLASTP, BLASTN, FASTA (Atschul et al., J Mol Biol (1990) 215:403). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof

The term “treatment” used in reference to a disease or condition means that at least an amelioration of the symptoms associated with the condition afflicting an individual is achieved, where amelioration is used in a broad sense to refer to at least a reduction in the magnitude of a parameter, e.g., a symptom, associated with the condition being treated. Treatment also includes situations where the pathological condition, or at least symptoms associated therewith, are completely inhibited, e.g., prevented from happening, or eliminated entirely such that the host no longer suffers from the condition, or at least the symptoms that characterize the condition. Thus, treatment includes: (i) prevention (i.e., reducing the risk of development of clinical symptoms, including causing the clinical symptoms not to develop, e.g., preventing disease progression), and (ii) inhibition (i.e., arresting the development or further development of clinical symptoms, e.g., mitigating or completely inhibiting an active disease).

As used herein, and unless otherwise specified, a “therapeutically effective amount” of an agent is an amount sufficient to provide a therapeutic benefit in the treatment or management of the cancer, or to delay or minimize one or more symptoms associated with the cancer. A therapeutically effective amount of a compound means an amount of therapeutic agent, alone or in combination with other therapeutic agents, which provides a therapeutic benefit in the treatment or management of the cancer. The term “therapeutically effective amount” can encompass an amount that improves overall therapy, reduces or avoids symptoms or causes of the cancer, or enhances the therapeutic efficacy of another therapeutic agent. An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 2010); Lloyd, The Art, Science and Technology of Pharmaceutical Compounding (2016); Pickar, Dosage Calculations (2012); and Remington: The Science and Practice of Pharmacy, 22nd Edition, 2012, Gennaro, Ed., Lippincott, Williams & Wilkins).

As used herein, a “subject” or an “individual” includes animals, such as human (e.g., human individuals) and non-human animals. In some embodiments, a “subject” or “individual” can be a patient under the care of a physician. Thus, the subject can be a human patient or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, and non-mammals, such as non-human primates, sheep, dogs, cows, chickens, amphibians, reptiles, and the like.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

All ranges disclosed herein also encompass any and all possible sub-ranges and combinations of sub-ranges thereof. Any listed range can be recognized as sufficiently describing and enabling the same range being broken down into at least equal halves, thirds, quarters, fifths, tenths, and so forth. As a non-limiting example, each range discussed herein can be readily broken down into a lower third, middle third and upper third, and so forth. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

Although features of the disclosures may be described in the context of a single embodiment, the features may also be provided separately or in any suitable combination. Conversely, although the disclosures may be described herein in the context of separate embodiments for clarity, the disclosures may also be implemented in a single embodiment. Any published patent applications and any other published references, documents, manuscripts, and scientific literature cited herein are incorporated herein by reference for any purpose. In the case of conflict, the present specification, including definitions, will control. In addition, the materials, methods, and examples are illustrative only and not intended to be limiting.

### Compositions of the Disclosure

The present disclosure provides, among others, fully recombinant bispecific binding agents comprising a first binding domain and a second binding domain. The first binding domain can specifically bind to at least one endogenous cell surface receptor. In some embodiments, the binding of the first binding domain to the at least one endogenous cell surface receptor results in the internalization of the endogenous cell surface receptor and the bispecific binding agent. In other embodiments, the endogenous cell surface receptor can be internalized on its own, and thus results in the internalization of the target protein, which is described in greater detail below, due to simultaneous binding of bispecific binding agent to the endogenous cell surface receptor and target protein. In certain embodiments, the endogenous cell surface receptor is membrane associated. Further, the second binding domain can specifically bind to a target protein. In some non-limiting exemplary embodiments, the present disclosure demonstrates the development of a new targeted degradation platform technology, termed cytokine receptor targeting chimeras (KineTACs), which includes fully recombinant bispecific binding agents that utilize endogenous cell surface receptor-mediated internalization (e.g., through the binding of CXCL12 and its receptors) to target various therapeutically relevant proteins for lysosomal degradation (FIG. 1B).

The disclosure also provides, among others, nucleic acids that encode the bispecific binding agents, cells comprising the nucleic acid, immunoconjugates of the bispecific binding agents, and pharmaceutical compositions comprising the bispecific binding agents. The disclosure also provides methods of treatment using bispecific binding agents or immunoconjugates, nucleic acids encoding bispecific binding agents or pharmaceutical compositions comprising the bispecific binding agents, immunoconjugates, and/or nucleic acids encoding the bispecific binding agents. The disclosure also provides compositions and methods useful for producing such agents, nucleic acids encoding same, host cells genetically modified with the nucleic acids, as well as methods for modulating an activity of a cell and/or for the treatment of various diseases such as cancers.

In the following detailed description, reference is made to the accompanying drawings, which form a part hereof. In the drawings, similar symbols generally identify similar components, unless context dictates otherwise. The illustrative alternatives described in the detailed description, drawings, and claims are not meant to be limiting. Other alternatives may be used and other changes may be made without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of this application.

### Bispecific Binding Agents

The bispecific binding agents provided herein comprise a first binding domain and a second binding domain. The first binding domain can specifically bind to at least one endogenous cell surface receptor. In some embodiments, the binding of the first binding domain to the at least one endogenous cell surface receptor results in the internalization of the endogenous cell surface receptor and the bispecific binding agent. In other embodiments, the endogenous cell surface receptor can be internalized on its own, and pull in the target protein, which is described in greater detail below, due to simultaneous binding of bispecific binding agent to the endogenous cell surface receptor and target protein. In certain embodiments, the endogenous cell surface receptor is membrane associated. Further, the second binding domain can specifically bind to a target protein.

The first binding domain of the bispecific binding agents provided herein can be a cytokine (e.g., a chemokine), or an isoform or a derivative capable of binding thereof. A functional derivative of a cytokine can be any agent that possesses the binding activity of a cytokine. For example, in some embodiments, the first binding domain of the bispecific binding agents can be an antagonistic variant of a cytokine which does not have a functional effect but binds to the endogenous cell surface receptors of the cytokine. In this instance, the antagonistic variant of a cytokine is a functional derivative of the cytokine. In other embodiments, the first binding domain of the bispecific binding agents can be a binding agent (e.g., an antibody or a fragment thereof, a peptide, or a small molecule) that binds to the endogenous cell surface receptors of a cytokine.

A functional derivative of a cytokine can be any agent that maintains binding affinity and/or selectivity of the cytokine to a cytokine receptor. The functional derivative may or may not have the same activity as the native cytokine. For example, a functional derivative of a cytokine may share the binding affinity of the cytokine to the cytokine receptor but the functional derivative may lack the agonistic or antagonistic activity of the cytokine. In certain embodiments, a functional derivative of a cytokine shares the binding affinity of the cytokine to the cytokine receptor and the functional derivative maintains similar agonistic or antagonistic activity relative to the cytokine.

In some embodiments, the first binding domain is a binding agent, e.g., an antibody or a fragment thereof, a peptide, a small molecule, that binds to the same epitope on a cytokine receptor as a chemokine selected from CCL1, CCL2, CCL3, CCL3L1, CCL4, CC4L1, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, and CCL28. In some embodiments, the first binding domain binds to the same epitope of a cytokine receptor as a chemokine selected from CXCL12, CXCL11, CXCL1, CXCL2, CXCL3, CXCL4, CXCL5, CXCL6, CXCL7, CXCL8, CXCL9, CXCL10, CXCL13, CXCL14, CXCL16, CXCL17, CX3CL1, XCL1, and XCL2. In some embodiments, the first binding domain binds the same epitope on the cytokine receptor as CXCL12. In certain embodiments, the first binding domain binds CXCR7.

Cytokines are a diverse class of soluble extracellular proteins and can include interleukins, chemokines, interferons, tumor necrosis factors, prolactins, transforming growth factor betas, and lymphokines. Upon binding to their cognate receptors on the surface of cells, cytokines trigger downstream signaling, which is in many cases coupled to internalization of the cytokine-receptor complex. Cytokines exert their action through high-affinity receptors on the cell surface that are linked to pathways of cellular activation, survival, proliferation and differentiation. Cross-linking of receptor subunits on the outside of the cell membrane can lead to abutting of kinases associated with the intracellular receptor tails. This intracellular association of signaling molecules results in phosphorylation of tyrosine residues in the receptor tail and binding of further signaling molecules that have phospho-tyrosine-binding domains. In some instances, different activated receptor cytoplasmic domains can bind a common signaling molecule or family of signaling molecules. Hapel A J and Stanley R E. Cytokines, Receptors and Signaling Pathways Involved in Macrophage and Dendritic Cell Development. In: Madame Curie Bioscience Database. Austin (TX): Landes Bioscience; 2000-2013. Thus, the triggering event (such as the binding of a cytokine to its receptor) required to trigger a cytokine mediated signaling can be much lower than that is required for a non-cytokine receptor. The present disclosure demonstrates that cytokine-mediated internalization could be co-opted for targeted degradation applications. Cytokine is used as its common meaning in the field and refers to a broad category of peptides important in cell signaling. Some non-limiting examples of cytokines include chemokines, interferons, interleukins, prolactins, transforming growth factor betas, lymphokines, and tumor necrosis factors.

Chemokines, or chemotactic cytokines, are small chemoattractant secreted molecules regulating cell positioning and cell recruitment into tissues, playing a pivotal role in embryogenesis, tissue development and immune response. Approximately 50 chemokines and 20 chemokine receptors have been discovered so far. Chemokines and their receptors have been reported to play important roles in immune cell migration and inflammation, as well as in tumor initiation, promotion, and progression. Marcuzzi E, et al. Chemokines and Chemokine Receptors: Orchestrating Tumor Metastasization. Int J Mol Sci. 2018 Dec. 27; 20(1):96. Chemokines can be widely divided into two major groups based on their prominent functions: inflammatory and homeostatic chemokines. Among inflammatory chemokines, which are induced by inflammation, some non-limiting examples include CXCL1, CXCL2, CXCL3, CXCL5, CXCL7, CXCL8, CXCL9, CXCL10, CXCL11, and CXCL14. On the other hand, homeostatic chemokines such as, without being limited to, CCL14, CCL19, CCL20, CCL21, CCL25, CCL27, CXCL12 and CXCL13 are constitutively expressed and are involved in homeostatic leukocyte trafficking. In some embodiments, the chemokine comprises a CXC chemokine, or an isoform or a derivative capable of binding thereof. In certain non-limiting exemplary embodiments, the chemokine can be CXCL12, CCL1, CCL2, CCL3, CCL3L1, CCL4, CC4L1, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, CCL28, CXCL11, CXCL1, CXCL2, CXCL3, CXCL4, CXCL5, CXCL6, CXCL7, CXCL8, CXCL9, CXCL10, CXCL13, CXCL14, CXCL16, CXCL17, CX3CL1, XCL1, XCL2, vMIPII, U83, and vCXC1. In some embodiments, the chemokine includes CCL1, CCL2, CCL3, CCL3L1, CCL4, CC4L1, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, and CCL28. In some embodiments, the chemokine includes CXCL12, CXCL11, CXCL1, CXCL2, CXCL3, CXCL4, CXCL5, CXCL6, CXCL7, CXCL8, CXCL9, CXCL10, CXCL13, CXCL14, CXCL16, CXCL17, CX3CL1, XCL1, XCL2. In some embodiments the chemokine includes vMIPII, U83, or vCXC1.

Interleukins are cytokines that play essential roles in the activation and differentiation of immune cells, as well as proliferation, maturation, migration, and adhesion. They also have pro-inflammatory and anti-inflammatory properties. The primary function of interleukins is to modulate growth, differentiation, and activation during inflammatory and immune responses. In certain non-limiting exemplary embodiments, the interleukin can be IL2, IL3, IL4, IL5, IL6, IL7, IL8, IL9, IL10, IL11, IL12A, IL12B, IL13, IL15, IL16, IL17A, IL17B, IL17C, IL17F, IL18, IL19, IL20, IL21, IL22, IL24, IL25, IL26, IL27, IL28A, IL28B, IL29, IL31, IL32, IL33, IL34, IL36A, IL36B, IL36G, IL36RA, IL37, IL38, IL1A, IL1B, IL1RN.

Interferons belong to the large class of proteins known as cytokines and are made and released by host cells in response to the presence of several viruses. More than twenty distinct IFN genes and proteins have been identified in animals, including humans. They are typically divided among three classes: Type I IFN, Type II IFN, and Type III IFN. In certain non-limiting exemplary embodiments, the interferon can be IFNA, IFNA1, IFNA2, IFNA4, IFNA5, IFNA6, IFNA7, IFNA8, IFNA10, IFNA14, IFNA16, IFNA17, IFNA21, IFNB, and IFNG.

Prolactins are both hormones and cytokines. Prolactins exert cytokine effects via interference with immune system modulation, mainly inhibiting the negative selection of autoreactive B lymphocytes. In certain non-limiting exemplary embodiments, the prolactin can be EPO, GH1, GH2, PRL, CSF3, LEP, and CSF1.

Tumor necrosis factors are a multifunctional cytokines that play important roles in diverse cellular events such as cell survival, proliferation, differentiation, and death. In certain non-limiting exemplary embodiments, the tumor necrosis factos can be TNFA, TNFB, TRAIL, TL1, BAFF, APRIL, RANKL, CD40LG, EDA, FASLG, CD70.

Transforming growth factor betas are multifunctional cytokine belonging to the transforming growth factor superfamily that includes three different mammalian isoforms (TGFB1, TGFB2, TGFB3) and many other signaling proteins. Among its key functions is regulation of inflammatory processes and playing a role in stem cell differentiation as well as T-cell regulation and differentiation. In certain non-limiting exemplary embodiments, the TGF-beta can be TGFB1, TGFB2, TGFB3, GDF15, GDF2, BMP10, INHA, and BMP3.

The first binding domain of the bispecific binding agents provided herein can also be a growth factor, or an isoform or a derivative capable of binding thereof. A functional derivative of a growth factor can be any agent that possesses the binding activity of a growth factor. For example, in some embodiments, the first binding domain of the bispecific binding agents can be an antagonistic variant of a growth factor which does not have a functional effect but binds to the endogenous cell surface receptors of the growth factor. In this instance, the antagonistic variant of a growth factor is a functional derivative of the growth factor. In other embodiments, the first binding domain of the bispecific binding agents can be a binding agent (e.g., an antibody or a fragment thereof, a peptide, or a small molecule) that binds to the endogenous cell surface receptors of a growth factor. Some non-limiting examples of growth factors include FGF1, FGF2, FGF3, FGF4, FGF5, FGF19, FGF21, FGF23, KGF, VEGF, PDGFA, PDGFB, NGF, NTF3, NTF4, BDNF, IGF1, and IGF2.

In a proof-of-concept example, chemokine CXCL12 is used as the first binding domain of the bispecific binding agent provided herein. CXCL12 is known to specifically bind to two chemokine receptors, CXCR4 and CXCR7, and subsequently internalize via two distinct mechanisms (FIG. 1A). Binding to CXCR4 agonizes the receptor, leading to its internalization and shuttling to the lysosome for degradation. CXCR7, however, is constitutively internalized and recycled back to the cell surface, independent of ligand binding. Therefore, binding to CXCR7 causes the internalization of CXCL12 without subsequent degradation of CXCR7 or downstream signaling. Thus, the CXCL12/CXCR4/CXCR7 axis served as an ideal system to test our hypothesis. In some non-limiting exemplary embodiments, the present disclosure demonstrates the development of a new targeted degradation platform technology, termed cytokine receptor targeting chimeras (KineTACs), which comprise of fully recombinant bispecific binding agents that utilize CXCL12-mediated internalization of its cognate receptors to target various therapeutically relevant cell surface proteins for lysosomal degradation (FIG. 1B).

The first binding domain can specifically bind to at least one endogenous cell surface receptor. The first binding domain of the bispecific binding agents provided herein can specifically bind to one or more cell surface receptors. In some embodiments, the first binding domain specifically binds to one cell surface receptor. In some embodiments, the first binding domain specifically binds to no more than two cell surface receptors. In some embodiments, the first binding domain specifically binds to two cell surface receptors. In some embodiments, the endogenous cell surface receptor can be a monomeric receptor. In some embodiments, the endogenous cell surface receptor can form a complex with other molecules (e.g., an integrin).

The endogenous cell surface receptors can be targeting receptors or recycling receptors. A targeting receptor as used herein refers to an endogenous cell surface receptor that specifically binds to a ligand (e.g., a cytokine, growth factor or an isoform or a derivative capable of binding thereof), and such binding does not necessarily have functional consequences. In certain examples, the binding of the first binding domain to a targeting receptor on the cell surface may not have any functional consequences. In other examples, such binding may lead to internalization, but not necessarily degradation, of the endogenous cell surface receptor and/or the target protein discussed herein. In contrast, a recycling receptor as used herein refers to an endogenous cell surface receptor that specifically binds to a ligand, e.g., a cytokine, a chemokine, a growth factor or an isoform or a derivative capable of binding thereof, and leads to internalization and degradation of the endogenous cell surface receptor and the cell expressing the receptor. In some embodiments, the degradation can occur through delivery of the target protein discussed herein to a lysosome via either a targeting or a recycling receptor.

In some embodiments, the binding of the first binding domain to the at least one endogenous cell surface receptor results in the internalization of the endogenous cell surface receptor and the bispecific binding agent. In some embodiments, the binding of the first binding domain to the at least one endogenous cell surface receptor results in the degradation of the target protein bound to the bispecific binding agent described herein. In certain embodiments, the binding of the first binding domain to the at least one endogenous cell surface receptor results in the degradation of the target protein bound to the bispecific binding agent described herein, but not the bispecific binding agent.

In certain embodiments, the endogenous cell surface receptor is membrane associated. Membrane proteins represent about a third of the proteins in living organisms and many membrane proteins are known in the field. Based on their structure, membrane proteins can be largely categorized into three main types: (1) integral membrane protein (IMP), which is permanently anchored or part of the membrane, (2) peripheral membrane protein, which is temporarily attached to the lipid bilayer or to other integral proteins, and (3) lipid-anchored proteins. The most common type of IMP is the transmembrane protein (TM), which spans the entire biological membrane. The endogenous cell surface receptor of the present disclosure include single-pass and multi-pass membrane proteins. Single-pass membrane proteins cross the membrane only once, while multi-pass membrane proteins weave in and out, crossing several times.

Some non-limiting membrane proteins encompassed herein include cytokine receptors, insulin receptors, cell adhesion proteins or cell adhesion molecules (CAMs), receptor proteins, glycophorin, rhodopsin, Band 3, CD36, glucose permease, ion channels and gates, gap junction proteins, G protein coupled receptors (e.g., beta-adrenergic receptor), and seipin. In some exemplary embodiments, CAMs can include integrins, cadherins, neural cell adhesion molecules (NCAMs), or selectins, etc.

In some embodiments, the at least one endogenous cell surface receptor includes at least one cytokine receptor. A cytokine receptor can include single-pass and multi-pass membrane-associated receptors. For instance, the cytokine receptor can be a chemokine receptor. Chemokine receptors, which are seven transmembrane spanning proteins coupled to G-proteins, are similarly divided into subfamilies based on their cysteine residues pattern: CXC, CC, CX3C, where C stands for cysteine and X represents non-cysteine amino acids. It has been reported that there is a significant ligand promiscuity among certain chemokine receptors, as some chemokines can bind to and signal through several chemokine receptors, both canonical and atypical ones. In contrast, some chemokines (e.g., CXCL12) are more selective. Some non-limiting example of chemokine receptor include CXCR1, CXCR2, CXCR3, CXCR4, CXCR5, CXCR6, CXCR7 (or ACKR3), XCR1, XCR2, CCR1, CCR2, CCR3, CCR4, CCR5, CCR6, CCR7, CCR8, CCR9, CCR10, CCR11, CX3CR1, ACKR1, ACKR2, ACKR4, and ACKR5. In some embodiments, the chemokine receptor includes CCR1, CCR2, CCR3, CCR4, CCR5, CCR6, CCR7, CCR8, CCR9, CCR10, and CCR11. In some embodiments, the chemokine receptor includes CXCR7, CXCR4, CXCR3, CXCR1, CXCR2, CXCR5, CXCR6, CX3CR1, XCR1, XCR2. In some embodiments, the chemokine receptor includes ACKR1, ACKR2, CXCR7, ACKR4. In some embodiments, the first binding domain of the bispecific binding agent provided herein specifically binds to CXCR4. In some embodiments, the first binding domain of the bispecific binding agent provided herein specifically binds to CXCR7. In certain embodiments, the first binding domain of the bispecific binding agent provided herein specifically binds to CXCR4 and CXCR7.

In some embodiments, the cytokine receptor can be an interleukin receptor. Interleukin receptors are members of the immunoglobulin superfamily receptors and are transmembrane proteins defined by their structural similarity to immunoglobulins. They often contain an amino-terminal extracellular domain that holds the characteristic immunoglobulin fold. Interluekin receptors are typically involved with cell adhesion and the interaction between T cells and antigen presenting cells. Some non-limiting examples of interleukin receptors include CD25, IL2RB, IL2RG, IL3RA, IL4R, IL13RA1, IL13RA2, IL5RA, IL6R, IL7R, IL8R, IL9R, IL10RA, IL10RB, IL11RA, IL12RB1, IL12RB2, IL15RA, CD4, IL17RA, IL17RC, IL17RB, IL17RE, IL27RA, IL18R1, IL20RA, IL20RB, IL22RA1, IL21R, IL28RA, IL31RA, ST2, IL1RAP, CSF1R, IL1R1, IL1RL2, IL1R2.

In some embodiments, the cytokine receptor can be an interferon receptor. All receptors involved in interferon signal transduction are classified as class II helical cytokine receptors (hCRs) sharing homologous structural folds and basic structural elements with other proteins including tissue factor, and the receptors for IL-10, IL-20 and IL-22. 4 In the extracellular region, all members of this class of hCR have tandem domains consisting of ˜100 amino acids each housing a type III fibronectin (FBN-III) domain with topology analogous to the immunoglobulin constant domain. With the exception of IFNAR1, which has a four-domain architecture, all other IFN receptors consist of two FBN-III domains. Some non-limiting examples of interferon receptors include IFNAR1, IFNAR2, IFNGR1, IFNGR2.

In some embodiments, the cytokine receptor can be a prolactin receptor. The prolactin receptor (PRLR) is a membrane-bound, type I cytokine receptor. Some non-limiting examples of prolactin receptors include EPOR, GHR, PRLR, CSF3R, LEPR, CSF1R.

In some embodiments, the cytokine receptor can be a tumor necrosis factor (TNF) receptor. In their active form, the majority of TNF receptors form trimeric complexes in the plasma membrane. Most TNF receptors contain transmembrane domains (TMDs), although some can be cleaved into soluble forms (e.g. TNFR1), and some lack a TMD entirely (e.g. DcR3). TNF receptors are primarily involved in apoptosis and inflammation, but they can also take part in other signal transduction pathways, such as proliferation, survival, and differentiation. Some non-limiting examples of TNF receptors include TNFR1, TNFR2, DR4, DR5, DCR1, DCR2, DR3, LTBR, BAFFR, TACI, OPG, RANK, CD40, EDAR, DCR3, FAS, and CD27.

In other embodiments, the at least one endogenous cell surface receptor includes at least one growth factor receptor. All growth factor receptors are membrane-bound and include an extracellular domain, a transmembrane domain, and a cytoplasmic domain. Some non-limiting examples of growth factor receptors are FGFR2B, VEGFR2, PDGFRA, PDGFRB, NGFR, TRKC, TRKB, M6PR, and IGF1R.

The bispecific binding agents provided herein further include a second binding domain that can specifically bind to a target protein. The target protein can be a soluble target protein and a membrane-associated target protein. In some embodiments, the second binding domain of the bispecific binding agents provided herein can bind to an extracellular epitope of a membrane-associated target protein. The binding of the second binding domain to the membrane-associated target protein can result in the internalization of a target cell expressing the membrane-associated target protein.

In some embodiments, the target protein of the bispecific binding agents provided herein can be an immune checkpoint protein. Immune checkpoint proteins are known in the field, and generally refers to proteins that serve as checkpoints produced by some types of immune system cells, such as T cells, and some cancer cells. Some non-limiting examples of immune checkpoint proteins include PD-L1, PD-1, CTLA-4, B7-H3, B7-H4, BTLA, KIR, LAG3, NKG2D, TIM-3, VISTA, SIGLEC7, and SIGLEC15.

In some embodiments, the target protein of the bispecific binding agents provided herein can be a cancer antigen. In some embodiments, cancer antigens are proteins that are expressed on the surface of certain cancer cells. In other embodiments, cancer antigens are shed by the cancer cells and can be detected in blood and sometimes other body fluids. Thus, cancer antigens can include both cell membrane-associated target proteins and soluble target proteins. Some non-limiting examples of the cancer antigens include PD-L1, HER2, EGFR, A2AR, CDCP1, MMP14, and TROP2. In other embodiments, the second binding domain can be an antigen-binding domain from any antigen-binding molecules, such as any of the clinically approved antibodies, known or to be developed. Some exemplary therapeutic monoclonal antibodies approved or in review in the EU or US are provided in Table 1 below.

TABLE 1
Therapeutic monoclonal antibodies approved or in review in the EU or US.

| | | | 1st indication approved/ |
|---|---|---|---|
| INN | Brand name | Target; Format | reviewed |
| Toripalimab | Tuoyi | PD-1; Humanized IgG4 | Nasopharyneal carcinoma |
| Tisotumab | (Pending) | Tissue factor; Human IgG1 | Cervical cancer |
| vedotin | | ADC | |
| Retifanlimab | (Pending) | PD-1; Humanized IgG4 | Squamous cell carcinoma |
| | | | of the anal canal |
| Amivantamab | (Pending) | EGFR, cMET; Human | NSCLC w/EGFR exon 20 |
| | | bispecific IgG1 | insertion mutations |
| Ublituximab | (Pending) | CD20; Chimeric IgG1 | Chronic lymphocytic |
| | | | leukemia |
| Anifrolumab | (Pending) | IFNAR1; Human IgG1 | Systemic lupus |
| | | | erythematosus |
| Loncastuximab | (Pending) | CD19; Humanized IgG1 ADC | Diffuse large B-cell |
| tesirine | | | lymphoma |
| Balstilimab | (Pending) | PD-1; Human IgG4 | Cervical cancer |
| Bimekizumab | (Pending) | IL-17A, F; Humanized IgG1 | Psoriasis |
| Tralokinumab | (Pending) | IL-13; Human IgG4 | Atopic dermatitis |
| Evinacumab | Evkeeza | Angiopoietin-like 3; Human | Homozygous familial |
| | | IgG4 | hypercholesterolemia |
| Sutimlimab | (Pending) | C1s; Humanized IgG4 | Cold agglutinin disease |
| Aducanumab | (Pending) | Amyloid beta; Human IgG1 | Alzheimer's disease |
| Teplizumab | (Pending) | CD3; Humanized IgG1 | Type 1 diabetes |
| Dostarlimab | (Pending) | PD-1; Humanized IgG4 | Endometrial cancer |
| Tanezumab | (Pending) | Nerve growth factor; | Osteoarthritis pain |
| | | Humanized IgG2 | |
| Inolimomab | (Pending) | CD25; Mouse IgG1 | Host vs. graft disease |
| Oportuzumab | (Pending) | EpCAM; Humanized scFv | Bladder cancer |
| monatox | | immunotoxin | |
| Narsoplimab | (Pending) | MASP-2; Human IgG4 | Hematopoietic stem cell |
| | | | transplant-associated |
| | | | thrombotic |
| | | | microangiopathies |
| Ansuvimab | Ebanga | Ebola virus; Human IgG1 | Ebola infection |
| Margetuximab | MARGENZA | HER2; Chimeric IgG1 | HER2+ breast cancer |
| Naxitamab | DANYELZA | GD2; Humanized IgG1 | High-risk neuroblastoma |
| | | | and refractory |
| | | | osteomedullary disease |
| Atoltivimab, | Inmazeb | Ebola virus; mixture of 3 | Ebola virus infection |
| Maftivimab, and | | human IgG1 | |
| Odesivimab- | | | |
| ebgn | | | |
| Belantamab | BLENREP | BCMA; Humanized IgG1 ADC | Multiple myeloma |
| mafodotin | | | |
| Tafasitamab | Monjuvi | CD19; Humanized IgG1 | Diffuse large B-cell |
| | | | lymphoma |
| Satralizumab | Enspryng | IL-6R; Humanized IgG2 | Neuromyelitis optica and |
| | | | neuromyelitis optica |
| | | | spectrum disorders |
| Inebilizumab | Uplizna | CD19; Humanized IgG1 | Neuromyelitis optica and |
| | | | neuromyelitis optica |
| | | | spectrum disorders |
| Sacituzumab | Trodelvy | TROP-2; Humanized IgG1 | Triple-neg. breast cancer |
| govitecan | | ADC | |
| Teprotumumab | Tepezza | IGF-1R; Human IgG1 | Thyroid eye disease |
| Isatuximab | Sarclisa | CD38; Chimeric IgG1 | Multiple myeloma |
| Eptinezumab | Vyepti | CGRP; Humanized IgG1 | Migraine prevention |
| [fam]- | Enhertu | HER2; Humanized IgG1 ADC | HER2+ breast cancer |
| trastuzumab | | | |
| deruxtecan | | | |
| Enfortumab | Padcev | Nectin-4; Human IgG1 ADC | Urothelial cancer |
| vedotin | | | |
| Crizanlizumab | Adakveo | P-selectin; Humanized IgG2 | Sickle cell disease |
| Brolucizumab | BEOVU | VEGF-A; Humanized scFv | Macular degeneration |
| Polatuzumab | Polivy | CD79b; Humanized IgG1 ADC | Diffuse large B-cell |
| vedotin | | | lymphoma |
| Risankizumab | Skyrizi | IL-23p19; Humanized IgG1 | Plaque psoriasis |
| Romosozumab | Evenity | Sclerostin; Humanized IgG2 | Osteoporosis in |
| | | | postmenopausal women at |
| | | | risk of fracture |
| Caplacizumab | Cablivi | von Willebrand factor; | Acquired thrombotic |
| | | Humanized Nanobody | thrombo- cytopenic |
| | | | purpura |
| Ravulizumab | Ultomiris | C5; Humanized IgG2/4 | Paroxysmal nocturnal |
| | | | hemoglobinuria |
| Emapalumab | Gamifant | IFNgamma; Human IgG1 | Primary hemophagocytic |
| | | | lymphohistiocytosis |
| Cemiplimab | Libtayo | PD-1; Human IgG4 | Cutaneous squamous cell |
| | | | carcinoma |
| Fremanezumab | Ajovy | CGRP; Human IgG2 | Migraine prevention |
| Moxetumomab | Lumoxiti | CD22; Murine IgG1 dsFv | Hairy cell leukemia |
| pasudotox | | immunotoxin | |
| Galcanezumab | Emgality | CGRP; Human IgG4 | Migraine prevention |
| Lanadelumab | Takhzyro | Plasma kallikrein; Human IgG1 | Hereditary angioedema |
| | | | attacks |
| Mogamulizumab | Poteligeo | CCR4; Humanized IgG1 | Cutaneous T cell |
| | | | lymphoma |
| Erenumab | Aimovig | CGRP receptor; Human IgG2 | Migraine prevention |
| Tildrakizumab | Ilumya | IL-23p19; Humanized IgG1 | Plaque psoriasis |
| Ibalizumab | Trogarzo | CD4; Humanized IgG4 | HIV infection |
| Burosumab | Crysvita | FGF23; Human IgG1 | X-linked |
| | | | hypophosphatemia |
| Durvalumab | IMFINZI | PD-L1; Human IgG1 | Bladder cancer |
| Emicizumab | Hemlibra | Factor IXa, X; Humanized | Hemophilia A |
| | | IgG4, bispecific | |
| Benralizumab | Fasenra | IL-5Rα; Humanized IgG1 | Asthma |
| Ocrelizumab | OCREVUS | CD20; Humanized IgG1 | Multiple sclerosis |
| Guselkumab | TREMFYA | IL-23 P19; Human IgG1 | Plaque psoriasis |
| Inotuzumab | BESPONSA | CD22; Humanized IgG4, ADC | Hematological malignancy |
| ozogamicin | | | |
| Sarilumab | Kevzara | IL-6R; Human IgG1 | Rheumatoid arthritis |
| Dupilumab | Dupixent | IL-4Rα; Human IgG4 | Atopic dermatitis |
| Avelumab | Bavencio | PD-L1; Human IgG1 | Merkel cell carcinoma |
| Brodalumab | Siliq, | IL-17R; Human IgG2 | Plaque psoriasis |
| | LUMICEF | | |
| Atezolizumab | Tecentriq | PD-L1; Humanized IgG1 | Bladder cancer |
| Bezlotoxumab | Zinplava | Clostridium difficile | Prevention of Clostridium |
| | | enterotoxin B; Human | difficile infection |
| | | IgG1 | recurrence |
| Olaratumab | Lartruvo | PDGRFα; Human IgG1 | Soft tissue sarcoma |
| Reslizumab | Cinqaero, | IL-5; Humanized IgG4 | Asthma |
| | Cinqair | | |
| Obiltoxaximab | Anthim | Protective antigen of B. | Prevention of inhalational |
| | | anthracis exotoxin; Chimeric | anthrax |
| | | IgG1 | |
| Ixekizumab | Taltz | IL-17a; Humanized IgG4 | Psoriasis |
| Daratumumab | Darzalex | CD38; Human IgG1 | Multiple myeloma |
| Elotuzumab | Empliciti | SLAMF7; Humanized IgG1 | Multiple myeloma |
| Necitumumab | Portrazza | EGFR; Human IgG1 | Non-small cell lung cancer |
| Idarucizumab | Praxbind | Dabigatran; | Reversal of dabigatran- |
| | | Humanized Fab | induced anticoagulation |
| Alirocumab | Praluent | PCSK9; Human IgG1 | High cholesterol |
| Mepolizumab | Nucala | IL-5; Humanized IgG1 | Severe eosinophilic |
| | | | asthma |
| Evolocumab | Repatha | PCSK9; Human IgG2 | High cholesterol |
| Dinutuximab | Unituxin | GD2; Chimeric IgG1 | Neuroblastoma |
| Secukinumab | Cosentyx | IL-17a; Human IgG1 | Psoriasis |
| Nivolumab | Opdivo | PD1; Human IgG4 | Melanoma, non-small cell |
| | | | lung cancer |
| Blinatumomab | Blincyto | CD19, CD3; Murine bispecific | Acute lymphoblastic |
| | | tandem scFv | leukemia |
| Pembrolizumab | Keytruda | PD1; Humanized IgG4 | Melanoma |
| Ramucirumab | Cyramza | VEGFR2; Human IgG1 | Gastric cancer |
| Vedolizumab | Entyvio | α4β7 integrin; Humanized | Ulcerative colitis, Crohn |
| | | IgG1 | disease |
| Siltuximab | Sylvant | IL-6; Chimeric IgG1 | Castleman disease |
| Obinutuzumab | Gazyva | CD20; Humanized IgG1; | Chronic lymphocytic |
| | | Glycoengineered | leukemia |
| Ado- | Kadcyla | HER2; Humanized IgG1, ADC | Breast cancer |
| trastuzumab | | | |
| emtansine | | | |
| Raxibacumab | (Pending) | B. anthrasis PA; Human IgG1 | Anthrax infection |
| Pertuzumab | Perjeta | HER2; Humanized IgG1 | Breast Cancer |
| Brentuximab | Adcetris | CD30; Chimeric IgG1, ADC | Hodgkin lymphoma, |
| vedotin | | | systemic anaplastic large |
| | | | cell lymphoma |
| Belimumab | Benlysta | BLyS; Human IgG1 | Systemic lupus |
| | | | erythematosus |
| Ipilimumab | Yervoy | CTLA-4; Human IgG1 | Metastatic melanoma |
| Denosumab | Prolia | RANK-L; Human IgG2 | Bone Loss |
| Tocilizumab | RoActemra, | IL-6R; Humanized IgG1 | Rheumatoid arthritis |
| | Actemra | | |
| Ofatumumab | Arzerra | CD20; Human IgG1 | Chronic lymphocytic |
| | | | leukemia |
| Canakinumab | Ilaris | IL-1β; Human IgG1 | Muckle-Wells syndrome |
| Golimumab | Simponi | TNF; Human IgG1 | Rheumatoid and psoriatic |
| | | | arthritis, ankylosing |
| | | | spondylitis |
| Ustekinumab | Stelara | IL-12/23; Human IgG1 | Psoriasis |
| Certolizumab | Cimzia | TNF; Humanized Fab, | Crohn disease |
| pegol | | pegylated | |
| Catumaxomab | Removab | EPCAM/CD3; Rat/mouse | Malignant ascites |
| | | bispecific mAb | |
| Eculizumab | Soliris | C5; Humanized IgG2/4 | Paroxysmal nocturnal |
| | | | hemoglobinuria |
| Ranibizumab | Lucentis | VEGF; Humanized IgG1 Fab | Macular degeneration |
| Panitumumab | Vectibix | EGFR; Human IgG2 | Colorectal cancer |
| Natalizumab | Tysabri | a4 integrin; Humanized IgG4 | Multiple sclerosis |
| Bevacizumab | Avastin | VEGF; Humanized IgG1 | Colorectal cancer |
| Cetuximab | Erbitux | EGFR; Chimeric IgG1 | Colorectal cancer |
| Efalizumab | Raptiva | CD11a; Humanized IgG1 | Psoriasis |
| Omalizumab | Xolair | IgE; Humanized IgG1 | Asthma |
| Tositumomab- | Bexxar | CD20; Murine IgG2a | Non-Hodgkin lymphoma |
| I131 | | | |
| Ibritumomab | Zevalin | CD20; Murine IgG1 | Non-Hodgkin lymphoma |
| tiuxetan | | | |
| Adalimumab | Humira | TNF; Human IgG1 | Rheumatoid arthritis |
| Alemtuzumab | MabCampath, | CD52; Humanized IgG1 | Chronic myeloid |
| | Campath-1H; | | leukemia#; multiple |
| | Lemtrada | | sclerosis |
| Gemtuzumab | Mylotarg | CD33; Humanized IgG4, ADC | Acute myeloid leukemia |
| ozogamicin | | | |
| Trastuzumab | Herceptin | HER2; Humanized IgG1 | Breast cancer |
| Infliximab | Remicade | TNF; Chimeric IgG1 | Crohn disease |
| Palivizumab | Synagis | RSV; Humanized IgG1 | Prevention of respiratory |
| | | | syncytial virus infection |
| Basiliximab | Simulect | IL-2R; Chimeric IgG1 | Prevention of kidney |
| | | | transplant rejection |
| Daclizumab | Zenapax; | IL-2R; Humanized IgG1 | Prevention of kidney |
| | Zinbryta | | transplant rejection; |
| | | | multiple sclerosis |
| Rituximab | MabThera, | CD20; Chimeric IgG1 | Non-Hodgkin lymphoma |
| | Rituxan | | |
| Abciximab | Reopro | GPIIb/IIIa; Chimeric IgG1 Fab | Prevention of blood clots |
| | | | in angioplasty |
| Edrecolomab | Panorex | EpCAM; Murine IgG2a | Colorectal cancer |
| Nebacumab | Centoxin | Endotoxin; Human IgM | Gram-negative sepsis |
| Muromonab- | Orthoclone | CD3; Murine IgG2a | Reversal of kidney |
| CD3 | Okt3 | | transplant rejection |

In some embodiments, the target protein of the bispecific binding agents provided herein can be an immunomodulatory protein. Immunomodulatory proteins can refer to any proteins that have immunomodulatory activities. For instance, an immunomodulatory protein can have the signaling activity upon a certain stimulation that leads to either increased activity of immune cells (i.e., immune activation) or decreased activity of immune cells (i.e., immune suppression). Some immunomodulatory proteins may also have immune checkpoint activities. Thus, in some instances, immunomodulatory proteins could overlap with immune checkpoint proteins. Some non-limiting examples of the immuno-modulatory proteins include PD-L1, PD-1, CTLA-4, B7-H3, B7-H4, LAG3, NKG2D, TIM-3, VISTA, CD39, CD73 (NT5E), A2AR, SIGLEC7, and SIGLEC15.

In some embodiments, the target protein can be a B cell antigen. In some instances, the B cell antigen can be a B cell surface marker, e.g., a specific marker of B cell lineage. Some non-limiting examples of B cell antigens include CD19, CD20, D22, CD23, CD24, CD37, CD40, and HLA-DR. In some embodiments, the target protein can also be a T cell marker. T cell markers can be T cell surface bound or secreted (i.e., extracellular). Some non-limiting examples of T cell markers include CD27, CD28, CD127, PD-1, CD122, CD132, KLRG-1, HLA-DR, CD38, CD69, CD11a, CD58, CD99, CD62L, CD103, CCR4, CCR5, CCR6, CCR9, CCR10, CXCR3, CXCR4, CLA, Granzyme A, Granzyme B, Perforin, CD161, IL-18Ra, c-Kit, and CD130.

In some embodiments, the target protein can be an inflammation receptor. Some non-limiting exemplary inflammation receptors include TNFR, IL1R, IL2Ralpha, IL2Rbeta.

Other cancer antigens, immuno-modulatory proteins, inflammation receptors, B cell antigens, and T cell marker are known in the field and are also encompassed by the present disclosure. In certain embodiments, some non-limiting examples of the target proteins include PD-L1, HER2, EGFR, PD-1, CTLA-4, A2AR, B7-H3, B7-H4, BTLA, KIR, LAG3, NKG2D, TIM-3, VISTA, LAG3, NKG2D, TIM, SIGLEC7, SIGLEC15, CD19, CD20, CDCP1, MMP14, and TROP2.

In some embodiments, the bispecific binding agent comprises a first binding domain that binds to the same epitope on a cytokine receptor as a chemokine selected from CCL1, CCL2, CCL3, CCL3L1, CCL4, CC4L1, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, and CCL28 and a second binding domain that binds to a target protein selected from EGFR, PD-L1 and HER2. In some embodiments, the bispecific binding agent comprises a first binding domain that binds to the same epitope on a cytokine receptor as a chemokine selected from CXCL12, CXCL11, CXCL1, CXCL2, CXCL3, CXCL4, CXCL5, CXCL6, CXCL7, CXCL8, CXCL9, CXCL10, CXCL13, CXCL14, CXCL16, CXCL17, CX3CL1, XCL1, and XCL2 and a second binding domain that binds to protein selected from EGFR, PD-L1 and HER2. In some embodiments, the bispecific binding agent comprises a first binding domain that binds the same epitope on the cytokine receptor as CXCL12 and the second binding domain binds to a target protein selected from EGFR, PD-L1 and HER2. In some embodiments, the bispecific binding agent comprises a first binding domain that binds the same epitope on the cytokine receptor as CXCL12 and the second binding domain binds to a protein selected from EGFR. In some embodiments, the bispecific binding agent comprises a first binding domain binds the same epitope on the cytokine receptor as CXCL12 and the second binding domain binds to a protein selected from PD-L1. In certain embodiments, the bispecific binding agent comprises a first binding domain that binds CXCR7 and a second binding domain that binds to a protein selected from EGFR, PD-L1 and HER2. In certain embodiments, the bispecific binding agent described herein is a bispecific antibody.

In certain embodiments, the bispecific binding agent described herein is not an immunoconjugate or a portion thereof. For example, the bispecific binding agent is not an antibody-drug conjugate. In certain embodiments, the bispecific binding agent described herein does not comprise a cytotoxic agent or a small molecule immune modulatory agent. In certain embodiments, the bispecific binding agent does not comprise a small molecule therapeutic agent.

Without being bound by theory, in the cases where the target protein is a membrane-associated target protein, the target cell needs to express both the membrane-associated target protein and the endogenous cell surface receptor. For instance, a bispecific binding agent provided herein comprises (1) a first binding domain which has CXCL12 Fc or a variant thereof that specifically binds to CXCR4 and/or CXCR7, and (2) a second binding domain which includes a Fab targeting PD-L1. In this case, the target cell needs to express (1) CXCR4 and/or CXCR7 and (2) PD-L1.

As mentioned above, in some embodiments, the bispecific binding agents of the present disclosure can specifically bind to an extracellular epitope of a membrane-associated target protein, and such binding can result in the membrane-associated target protein bound to the bispecific binding agent. Thus, one skilled in the art would appreciate that any cell expressing a target protein could be a target cell for the purpose of the present disclosure. For example, the target cells encompassed by the present can be a neoplastic cell. A neoplasm is an abnormal growth of cells. Neoplastic cells are cells that are undergoing or have undergone an abnormal growth. In some instances, these abnormally growing cells can cause tumor growth and can be both benign and malignant.

Alternatively, the target cells encompassed by the present disclosure can be cancer cells. Some non-limiting examples of target cells include cancer cells, such as cells from breast cancer, B cell lymphoma, pancreatic cancer, Hodgkin's lymphoma, ovarian cancer, prostate cancer, mesothelioma, lung cancer, non-Hodgkin's B-cell (B-NHL), melanoma, chronic lymphocytic leukemia, acute lymphocytic leukemia, neuroblastoma, glioma, glioblastoma, bladder cancer, and colorectal cancer.

In other embodiments, the target cells can be immune cells. For instance, the immune cells can be monocytes, macrophages, lymphocytes (e.g., natural killer cells, T cells, and B cells), and monocytes.

The second binding domain of the bispecific binding agents provided herein can also bind to soluble target proteins. In certain embodiments, the soluble target proteins include soluble extracellular proteins. For example, the soluble target protein that can be targeted by the bispecific binding agents provided herein include an inflammatory cytokine, a growth factor (GF), a toxic enzyme, a target associated with metabolic diseases, a neuronal aggregate, or an autoantibody. These various soluble proteins are known in the art. In some embodiments, non-limiting examples of the inflammatory cytokine include lymphotoxin, interleukin-1 (IL-1), IL-2, IL-5, IL-6, IL-12, IL-13, IL-17, IL-18, IL-23, tumor necrosis factor alpha (TNF-α), interferon gamma (IFNγ), and granulocyte-macrophage colony stimulating factor (GM-CSF). In other embodiments, non-limiting examples of the growth factor comprises EGF, FGF, NGF, PDGF, VEGF, IGF, GMCSF, GCSF, TGF, RANK-L, erythropieitn, TPO, BMP, HGF, GDF, neurotrophins, MSF, SGF, GDF, and an isoform thereof. In some embodiments, non-limiting examples of the toxic enzyme comprises a protein arginine deiminase 1 (PAD1), PAD2, PAD3, PAD4, and PAD6, leucocidin, hemolysin, coagulase, treptokinase, hyaluronidase. In certain embodiments, the toxic enzyme comprises PAD2 or PAD4. In some embodiments, the target associated with a metabolic disease can be PCSK9, HRD1 T2DM, and MOGAT2. In other embodiments, non-limiting examples of the neuronal aggregate comprises Aβ, TTR, α-synuclein, TAO, and prion. In certain embodiments, the autoantibody comprises IgA, IgE, IgG, IgM, and IgD. Target proteins associated with the conditions described herein are known in the field and new targets are being discovered. All of the known and to be discovered targets are encompassed herein.

In some embodiments, once bound by the scond binding domain, the target protein is internalized at least 5%, 10%, 20%, 30%, 40%, 50%, 60%, 70%, 80%, 90% greater than a control. In one embodiment, the target protein is VEGF, when bound by the bispecific agent described herein, it is internalized 40% more than control.

The bispecific binding agents of the present disclosure can generally take the form of a protein, glycoprotein, lipoprotein, phosphoprotein, and the like. Some bispecific binding agent of the disclosure take the form of bispecific antibodies or antibody derivatives. In some embodiments, the first binding domain and the second binding domain of the bispecific binding agent provided herein can each be independently selected from the group consisting of natural ligands or a fragment, derivative, or small molecule mimetic thereof, antibodies, half antibodies, single-domain antibodies, nanobodies, Fabs, monospecific Fab2, Fc, scFv, minibodies, IgNAR, V-NAR, hcIgG, VHH domain, camelid antibodies, and peptibodies. In some embodiments, the first binding domain and the second binding domain of the bispecific binding agent provided herein together can form a bispecific antibody, a bispecific diabody, a bispecific Fab2, a bispecific camelid antibody, or a bispecific peptibody scFv-Fc, a bispecific IgG, and a knob and hole bispecific IgG, a Fc-Fab, and a knob and hole bispecific Fc-Fab, a cytokine-IgG fusion, a cytokine-Fab fusion, a cytokine-Fc-scFv fusion.

For example, one can employ known techniques such as phage display to generate and select for small proteins having a binding domain similar to an antibody complementarity-determining region (CDR). In some embodiments, the first or second binding domain includes a scFv. In other embodiments, the first or second binding domain includes a Fab. The first binding domain can also be derived from a natural or synthetic ligand that specifically binds to at least one endogenous cell surface receptor, for example, without limitation, a cytokine receptor, and the like. The second binding domain can be derived from any known or to be developed antigen binding agents, e.g., any therapeutic antibodies, that specifically binds to target protein, whether soluble or membrane-associated.

The binding domains can include naturally-occurring amino acid sequences or can be engineered, designed, or modified so as to provide desired and/or improved properties, e.g., binding affinity. Generally, the binding affinity of an antigen-binding moiety, e.g., an antibody, for a target antigen (e.g., PD-L1) can be calculated by the Scatchard method described by Frankel et al., Mol Immunol (1979) 16:101-06. In some embodiments, binding affinity is measured by an antigen/antibody dissociation rate. In some embodiments, binding affinity is measured by a competition radioimmunoassay. In some embodiments, binding affinity is measured by ELISA. In some embodiments, antibody affinity is measured by flow cytometry. In some embodiments, binding affinity is measured by bio-layer interferometry. An antibody that selectively binds an antigen (such as PD-L1) when it is capable of binding that antigen with high affinity, without significantly binding other antigens.

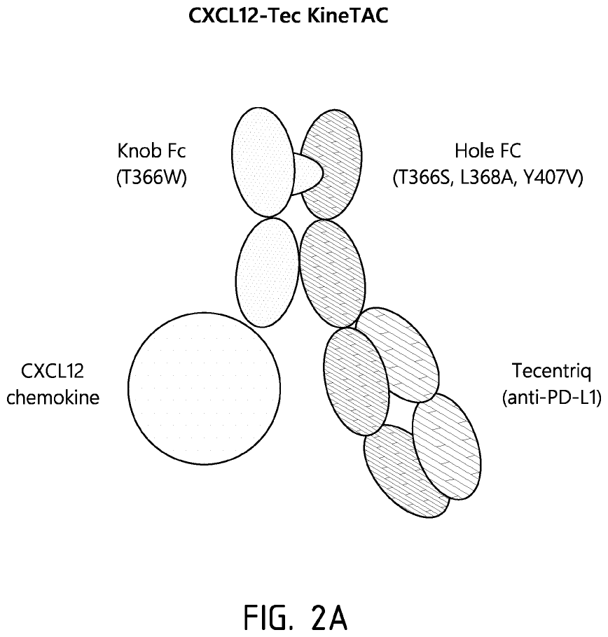

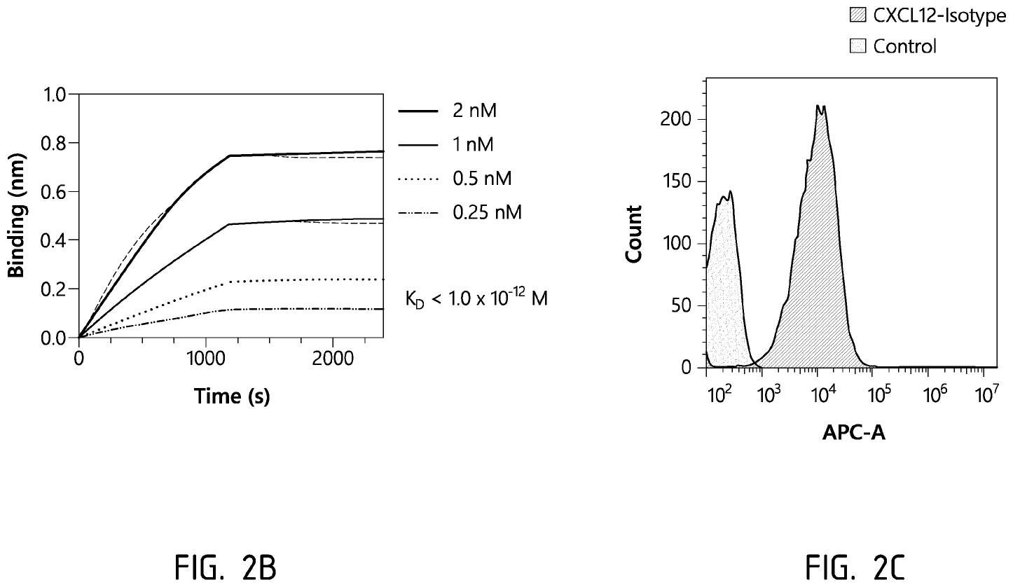

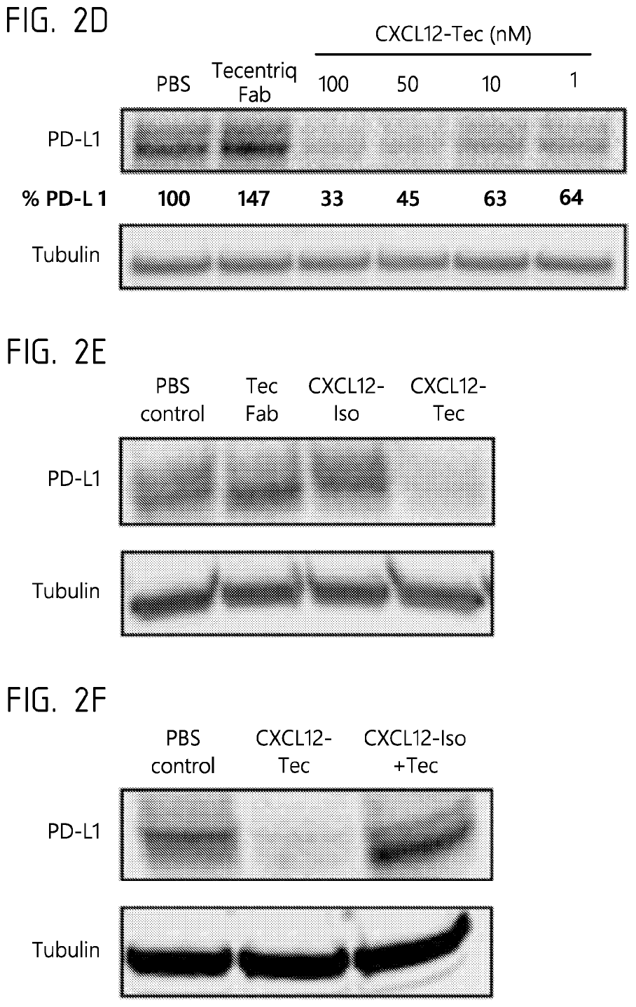

Bispecific antibodies can be prepared by known methods. Embodiments of the disclosure include “knob-into-hole” bispecific antibodies, wherein the otherwise symmetric dimerization region of a bispecific binding agent is altered so that it is asymmetric. For example, a knob-into-hole bispecific IgG that is specific for antigens A and B can be altered so that the Fc portion of the A-binding chain has one or more protrusions (“knobs”), and the Fc portion of the B-binding chain has one or more hollows (“holes”), where the knobs and holes are arranged to interact. This reduces the homodimerization (A-A and B-B antibodies), and promotes the heterodimerization desired for a bispecific binding agent. See, e.g., Y. Xu et al., mAbs (2015) 7(1):231-42. In some embodiments, the bispecific binding agent has a knob-into-hole design. In some embodiments, the “knob” comprises a T336W alteration of the CH3 domain, i.e., the threonine at position 336 is replaced by a tryptophan. In some embodiments, the “hole” comprises one or a combination of T366S, L368A, and Y407V. In some embodiments, the “hole” comprises T366S, L368A, and Y407V. For example, an illustration is provided in FIG. 2A. In some exemplary embodiments, the “knob” constant region comprises a sequence set forth in SEQ ID NO: 1, 8, 10, 12, 28, and 34 or a portion of any one thereof. In some embodiments, the heavy chain Fc “knob” constant region has a histidine tag. In some exemplary embodiments, the heavy chain Fc “hole” constant region comprises SEQ ID NO: 2, 3, 5, 6, 9, 11, 13-20, 22, 24, 26, 29, 35, and 38 or a portion of any one thereof. In certain embodiments, an exemplary CH2-CH3 domain sequence of a Knob construct comprises a N297G. In other embodiments, an exemplary CH2-CH3 domain sequence of a Hole construct comprises N297G.

In other embodiments, the “knob” and the “hole” constant regions comprise sequences that are about 70%, 75%, 80%, 85%, 90%, 95%, 99% identical to the sequences provided herein. For example, see Table 1 for exemplary constructs and sequences.

In some embodiments, the first binding domain of the bispecific binding agent provided herein comprises an Fc-fusion (e.g, a chemokine or variant fused to an Fc). In some embodiments, the second binding domain comprises an Fc-Fab. In some embodiments, the second binding domain comprises an scFv. In one exemplary embodiment, the bispecific binding agent includes a first binding domain that comprises an Fc-fusion, and the second binding domain that comprises an Fc-Fab. In another exemplary embodiment, the bispecific binding agent includes a a first binding domain that comprises an Fc-fusion, and the second binding domain comprises an scFv. By way of example, a cytokine can be N-terminally fused to an Fc domain, and the scFv can be fused to the C-terminus of the Fc. In an alternative embodiment, a cytokine can be C-terminally fused to an Fc domain, and the scFv can be fused to the N-terminus of the Fc. In some embodiments, the endogenous cell surface receptor to which the first binding domain binds to is referred to as a degrader, and the target protein of the second binding domain is referred to a victim.

In some embodiments, the first binding domain of the bispecific binding agent provided herein comprises e.g., a cytokine, and the second binding domain comprises an IgG. By way of example, a cytokine can be fused off the N-terminus of the heavy chain of the IgG, off the N-terminus of the light chain of the IgG, off the C-terminus of the light chain of the IgG, or off the C-terminus of the heavy chain of the IgG. In some embodiments, one to two cytokines can be fused per IgG.

In some embodiments, the first binding domain of the bispecific binding agent provided herein comprises e.g., a cytokine, which is fused to the second binding domain which comprises a Fab or scFv.

Without being bound by theory, the present disclosure provides some exemplary bispecific binding agents (a.k.a., KineTACs) that comprises chemokine CXCL12 or variants thereof in a Knob-Fc format as the first binding domain, and a Fab in a Hole-Fc format that specifically binds to various targets, including PD-L1, HER2, EGFR, and CDCP1, as the second binding domain. In certain embodiments, the CXCL12 variants comprise one or a combination of mutations selected from ΔKP at residues 1-2, ΔKPVS at residues 1-4, and R8E. Table 2 below provides some exemplary designs and sequences of the bispecific binding agents of the present disclosure.

TABLE 2
Exemplary design and sequences of the bispecific binding agents.

| | | | SEQ |
|---|---|---|---|
| Construct | | | ID |
| Name | | Construct Sequence | NO |
| CXCL12- | Knob: | KPVSLSYRCPCRFFESHVARANVKHLKILNTPNCALQIVARLKNNNRQVCIDPKLKW | 1 |
| Tecentriq | | IQEYLEKALNPWEPKSCDKTHTCPPCPAPELLGGPSVFLFPPKPKDTLMISRTPEVT | |
| | | CVVVDVSHEDPEVKFNWYVDGVEVHNAKTKPREEQYGSTYRVVSVLTVLHQDWLNGK | |
| | | EYKCKVSNKALPAPIEKTISKAKGQPREPQVYTLPPSRDELTKNQVSLWCLVKGFYP | |
| | | SDIAVEWESNGQPENNYKTTPPVLDSDGSFFLYSKLTVDKSRWQQGNVFSCSVMHEA | |
| | | LHNHYTQKSLSLSPGKGGSHHHHHH | |
| | Hole: | EVQLVESGGGLVQPGGSLRLSCAASGFTFSDSWIHWVRQAPGKGLEWVAWISPYGGS | 2 |
| | | TYYADSVKGRFTISADTSKNTAYLQMNSLRAEDTAVYYCARRHWPGGFDYWGQGTLV | |
| | | TVSSASTKGPSVFPLAPSSKSTSGGTAALGCLVKDYFPEPVTVSWNSGALTSGVHTF | |
| | | PAVLQSSGLYSLSSVVTVPSSSLGTQTYICNVNHKPSNTKVDKKVEPKSCEPKSCDK | |
| | | THTCPPCPAPELLGGPSVFLFPPKPKDTLMISRTPEVTCVVVDVSHEDPEVKENWYV | |
| | | DGVEVHNAKTKPREEQYGSTYRVVSVLTVLHQDWLNGKEYKCKVSNKALPAPIEKTI | |
| | | SKAKGQPREPQVYTLPPSRDELTKNQVSLSCAVKGFYPSDIAVEWESNGQPENNYKT | |
| | | TPPVLDSDGSFFLVSKLTVDKSRWQQGNVFSCSVMHEALHNHYTQKSLSLSPGKGGS | |
| | | GAWSHPQFEK | |
| | L: | DIQMTQSPSSLSASVGDRVTITCRASQDVSTAVAWYQQKPGKAPKLLIYSASFLYSG | 40 |
| | | VPSRFSGSGSGTDFTLTISSLQPEDFATYYCQQYLYHPATFGQGTKVEIKRTVAAPS | |
| | | VFIFPPSDEQLKSGTASVVCLLNNFYPREAKVQWKVDNALQSGNSQESVTEQDSKDS | |
| | | TYSLSSTLTLSKADYEKHKVYACEVTHQGLSSPVTKSENRGEC | |
| CXCL12- | Knob: | KPVSLSYRCPCRFFESHVARANVKHLKILNTPNCALQIVARLKNNNRQVCIDPKLKW | 1 |
| Trastuzumab | | IQEYLEKALNPWEPKSCDKTHTCPPCPAPELLGGPSVFLFPPKPKDTLMISRTPEVT | |
| | | CVVVDVSHEDPEVKFNWYVDGVEVHNAKTKPREEQYGSTYRVVSVLTVLHQDWLNGK | |
| | | EYKCKVSNKALPAPIEKTISKAKGQPREPQVYTLPPSRDELTKNQVSLWCLVKGFYP | |
| | | SDIAVEWESNGQPENNYKTTPPVLDSDGSFFLYSKLTVDKSRWQQGNVFSCSVMHEA | |
| | | LHNHYTQKSLSLSPGKGGSHHHHHH | |
| | Hole: | EVQLVESGGGLVQPGGSLRLSCAASGENIKDTYIHWVRQAPGKGLEWVARIYPTNGY | 3 |
| | | TRYADSVKGRFTISADTSKNTAYLQMNSLRAEDTAVYYCSRWGGDGFYAMDYWGQGT | |
| | | LVTVSSASTKGPSVFPLAPSSKSTSGGTAALGCLVKDYFPEPVTVSWNSGALTSGVH | |
| | | TFPAVLQSSGLYSLSSVVTVPSSSLGTQTYICNVNHKPSNTKVDKKVEPKSCDKTHT | |
| | | CPPCPAPELLGGPSVFLFPPKPKDTLMISRTPEVTCVVVDVSHEDPEVKENWYVDGV | |
| | | EVHNAKTKPREEQYGSTYRVVSVLTVLHQDWLNGKEYKCKVSNKALPAPIEKTISKA | |
| | | KGQPREPQVYTLPPSRDELTKNQVSLSCAVKGFYPSDIAVEWESNGQPENNYKTTPP | |
| | | VLDSDGSFFLVSKLTVDKSRWQQGNVFSCSVMHEALHNHYTQKSLSLSPGKGGSGAW | |
| | | SHPQFEK | |
| | L: | DIQMTQSPSSLSASVGDRVTITCRASQDVNTAVAWYQQKPGKAPKLLIYSASFLYSG | 4 |
| | | VPSRFSGSRSGTDFTLTISSLQPEDFATYYCQQHYTTPPTFGQGTKLEIKRTVAAPS | |
| | | VFIFPPSDEQLKSGTASVVCLLNNFYPREAKVQWKVDNALQSGNSQESVTEQDSKDS | |
| | | TYSLSSTLTLSKADYEKHKVYACEVTHQGLSSPVTKSFNRGEC | |
| CXCL12- | Knob: | KPVSLSYRCPCRFFESHVARANVKHLKILNTPNCALQIVARLKNNNRQVCIDPKLKW | 1 |
| Cetuximab | | IQEYLEKALNPWEPKSCDKTHTCPPCPAPELLGGPSVFLFPPKPKDTLMISRTPEVT | |
| | | CVVVDVSHEDPEVKFNWYVDGVEVHNAKTKPREEQYGSTYRVVSVLTVLHQDWLNGK | |
| | | EYKCKVSNKALPAPIEKTISKAKGQPREPQVYTLPPSRDELTKNQVSLWCLVKGFYP | |
| | | SDIAVEWESNGQPENNYKTTPPVLDSDGSFFLYSKLTVDKSRWQQGNVFSCSVMHEA | |
| | | LHNHYTQKSLSLSPGKGGSHHHHHH | |
| | Hole: | QVQLKQSGPGLVQPSQSLSITCTVSGFSLTNYGVHWVRQSPGKGLEWLGVIWSGGNT | 38 |
| | | DYNTPFTSRLSINKDNSKSQVFFKMNSLQSNDTAIYYCARALTYYDYEFAYWGQGTL | |
| | | VTVSAASTKGPSVFPLAPSSKSTSGGTAALGCLVKDYFPEPVTVSWNSGALTSGVHT | |
| | | FPAVLQSSGLYSLSSVVTVPSSSLGTQTYICNVNHKPSNTKVDKKVEPKSCDKTHTC | |
| | | PPCPAPELLGGPSVFLFPPKPKDTLMISRTPEVTCVVVDVSHEDPEVKENWYVDGVE | |
| | | VHNAKTKPREEQYGSTYRVVSVLTVLHQDWLNGKEYKCKVSNKALPAPIEKTISKAK | |
| | | GQPREPQVYTLPPSRDELTKNQVSLSCAVKGFYPSDIAVEWESNGQPENNYKTTPPV | |
| | | LDSDGSFFLVSKLTVDKSRWQQGNVFSCSVMHEALHNHYTQKSLSLSPGKGGSGAWS | |
| | | HPQFEK | |
| | L: | DILLTQSPVILSVSPGERVSFSCRASQSIGTNIHWYQQRTNGSPRLLIKYASESISG | 39 |
| | | IPSRFSGSGSGTDFTLSINSVESEDIADYYCQQNNNWPTTFGAGTKLELKRTVAAPS | |
| | | VFIFPPSDEQLKSGTASVVCLLNNFYPREAKVQWKVDNALQSGNSQESVTEQDSKDS | |
| | | TYSLSSTLTLSKADYEKHKVYACEVTHQGLSSPVTKSENRGEC | |
| CXCL12- | Knob: | KPVSLSYRCPCRFFESHVARANVKHLKILNTPNCALQIVARLKNNNRQVCIDPKLKW | 1 |
| 4A06 | | IQEYLEKALNPWEPKSCDKTHTCPPCPAPELLGGPSVFLFPPKPKDTLMISRTPEVT | |
| | | CVVVDVSHEDPEVKFNWYVDGVEVHNAKTKPREEQYGSTYRVVSVLTVLHQDWLNGK | |
| | | EYKCKVSNKALPAPIEKTISKAKGQPREPQVYTLPPSRDELTKNQVSLWCLVKGFYP | |
| | | SDIAVEWESNGQPENNYKTTPPVLDSDGSFFLYSKLTVDKSRWQQGNVFSCSVMHEA | |
| | | LHNHYTQKSLSLSPGKGGSHHHHHH | |
| | Hole: | EISEVQLVESGGGLVQPGGSLRLSCAASGENLSYYYIHWVRQAPGKGLEWVASIYSS | 5 |
| | | SSYTSYADSVKGRFTISADTSKNTAYLQMNSLRAEDTAVYYCARAYYGFDYWGQGTL | |
| | | VTVSSASTKGPSVFPLAPSSKSTSGGTAALGCLVKDYFPEPVTVSWNSGALTSGVHT | |
| | | FPAVLQSSGLYSLSSVVTVPSSSLGTQTYICNVNHKPSNTKVDKKVEPKSCDKTHTC | |
| | | PPCPAPELLGGPSVFLFPPKPKDTLMISRTPEVTCVVVDVSHEDPEVKENWYVDGVE | |
| | | VHNAKTKPREEQYNSTYRVVSVLTVLHQDWLNGKEYKCKVSNKALPAPIEKTISKAK | |
| | | GQPREPQVYTLPPIRELMTSNQVSLSCAVKGFYPSDIAVEWESNGQPENNYKTTPPV | |
| | | LDSDGSFFLVSKLTVDKSRWQQGNVFSCSVMHEALHN | |
| | | HYTQKSLSLSPGK | |
| | L: | DIQMTQSPSSLSASVGDRVTITCRASQSVSSAVAWYQQKPGKAPKLLIYSASSLYSG | 37 |
| | | VPSRFSGSRSGTDFTLTISSLQPEDFATYYCQQSYYYYPITFGQGTKVEIKRTVAAP | |
| | | SVFIFPPSDSQLKSGTASVVCLLNNFYPREAKVQWKVDNALQSGNSQESVTEQDSKD | |
| | | STYSLSSTLTLSKADYEKHKVYACEVTHQGLSSPVTKSFNRGECGGSDYKDDDDK | |
| CXCL12- | Knob: | KPVSLSYRCPCRFFESHVARANVKHLKILNTPNCALQIVARLKNNNRQVCIDPKLKW | 1 |
| Isotype | | IQEYLEKALNPWEPKSCDKTHTCPPCPAPELLGGPSVFLFPPKPKDTLMISRTPEVT | |
| | | CVVVDVSHEDPEVKFNWYVDGVEVHNAKTKPREEQYGSTYRVVSVLTVLHQDWLNGK | |
| | | EYKCKVSNKALPAPIEKTISKAKGQPREPQVYTLPPSRDELTKNQVSLWCLVKGFYP | |
| | | SDIAVEWESNGQPENNYKTTPPVLDSDGSFFLYSKLTVDKSRWQQGNVFSCSVMHEA | |
| | | LHNHYTQKSLSLSPGKGGSHHHHHH | |
| | Hole: | EVQLVESGGGLVQPGGSLRLSCAASGENISSSYMHWVRQAPGKGLEWVAYISSYYGS | 6 |
| | | TYYADSVKGRFTISRDNSKNTLYLQMNSLRAEDTAVYYCARYYSAYPRGFDYWGQGT | |
| | | LVTVSSASTKGPSVFPLAPSSKSTSGGTAALGCLVKDYFPEPVTVSWNSGALTSGVH | |
| | | TFPAVLQSSGLYSLSSVVTVPSSSLGTQTYICNVNHKPSNTKVDKKVEPKSCDKTHT | |
| | | CPPCPAPELLGGPSVFLFPPKPKDTLMISRTPEVTCVVVDVSHEDPEVKENWYVDGV | |
| | | EVHNAKTKPREEQYGSTYRVVSVLTVLHQDWLNGKEYKCKVSNKALPAPIEKTISKA | |
| | | KGQPREPQVYTLPPSRDELTKNQVSLSCAVKGFYPSDIAVEWESNGQPENNYKTTPP | |
| | | VLDSDGSFFLVSKLTVDKSRWQQGNVFSCSVMHEALHNHYTQKSLSLSPGKGGSGAW | |
| | | SHPQFEK | |
| | L: | DIQMTQSPSSLSASVGDRVTITCRASQSVGSALAWYQQKPGKAPKLLIYSASSLYSG | 7 |
| | | VPSRFSGSRSGTDFTLTISSLQPEDFATYYCQQSSYHHPITFGQGTKVEIKRTVAAP | |
| | | SVFIFPPSDSQLKSGTASVVCLLNNFYPREAKVQWKVDNALQSGNSQESVTEQDSKD | |
| | | STYSLSSTLTLSKADYEKHKVYACEVTHQGLSSPVTKSF | |
| CXCL12 | Knob: | VSLSYRCPCRFFESHVARANVKHLKILNTPNCALQIVARLKNNNRQVCIDPKLKWIQ | 8 |
| (AKP)- | | EYLEKALNPWEPKSCDKTHTCPPCPAPELLGGPSVFLFPPKPKDTLMISRTPEVTCV | |
| Tecentriq | | VVDVSHEDPEVKFNWYVDGVEVHNAKTKPREEQYGSTYRVVSVLTVLHQDWLNGKEY | |
| | | KCKVSNKALPAPIEKTISKAKGQPREPQVYTLPPSRDELTKNQVSLWCLVKGFYPSD | |
| | | IAVEWESNGQPENNYKTTPPVLDSDGSFFLYSKLTVDKSRWQQGNVFSCSVMHEALH | |
| | | NHYTQKSLSLSPGKGGSHHHHHH | |
| | Hole: | EVQLVESGGGLVQPGGSLRLSCAASGFTFSDSWIHWVRQAPGKGLEWVAWISPYGGS | 9 |
| | | TYYADSVKGRFTISADTSKNTAYLQMNSLRAEDTAVYYCARRHWPGGFDYWGQGTLV | |
| | | TVSSASTKGPSVFPLAPSSKSTSGGTAALGCLVKDYFPEPVTVSWNSGALTSGVHTF | |
| | | PAVLQSSGLYSLSSVVTVPSSSLGTQTYICNVNHKPSNTKVDKKVEPKSCEPKSCDK | |
| | | THTCPPCPAPELLGGPSVFLFPPKPKDTLMISRTPEVTCVVVDVSHEDPEVKENWYV | |
| | | DGVEVHNAKTKPREEQYGSTYRVVSVLTVLHQDWLNGKEYKCKVSNKALPAPIEKTI | |
| | | SKAKGQPREPQVYTLPPSRDELTKNQVSLSCAVKGFYPSDIAVEWESNGQPENNYKT | |
| | | TPPVLDSDGSFFLVSKLTVDKSRWQQGNVFSCSVMHEALHNHYTQKSLSLSPGKGGS | |
| | | GAWSHPQFEK | |
| | L: | DIQMTQSPSSLSASVGDRVTITCRASQDVSTAVAWYQQKPGKAPKLLIYSASFLYSG | 40 |
| | | VPSRFSGSGSGTDFTLTISSLQPEDFATYYCQQYLYHPATFGQGTKVEIKRTVAAPS | |
| | | VFIFPPSDEQLKSGTASVVCLLNNFYPREAKVQWKVDNALQSGNSQESVTEQDSKDS | |
| | | TYSLSSTLTLSKADYEKHKVYACEVTHQGLSSPVTKSENRGEC | |
| CXCL12 | Knob: | KPVSLSYECPCRFFESHVARANVKHLKILNTPNCALQIVARLKNNNRQVCIDPKLKW | 10 |
| (R8E)- | | IQEYLEKALNPWEPKSCDKTHTCPPCPAPELLGGPSVFLFPPKPKDTLMISRTPEVT | |
| Tecentriq | | CVVVDVSHEDPEVKFNWYVDGVEVHNAKTKPREEQYGSTYRVVSVLTVLHQDWLNGK | |
| | | EYKCKVSNKALPAPIEKTISKAKGQPREPQVYTLPPSRDELTKNQVSLWCLVKGFYP | |
| | | SDIAVEWESNGQPENNYKTTPPVLDSDGSFFLYSKLTVDKSRWQQGNVFSCSVMHEA | |
| | | LHNHYTQKSLSLSPGKGGSHHHHHH | |
| | Hole: | EVQLVESGGGLVQPGGSLRLSCAASGFTFSDSWIHWVRQAPGKGLEWVAWISPYGGS | 11 |
| | | TYYADSVKGRFTISADTSKNTAYLQMNSLRAEDTAVYYCARRHWPGGFDYWGQGTLV | |
| | | TVSSASTKGPSVFPLAPSSKSTSGGTAALGCLVKDYFPEPVTVSWNSGALTSGVHTF | |
| | | PAVLQSSGLYSLSSVVTVPSSSLGTQTYICNVNHKPSNTKVDKKVEPKSCEPKSCDK | |
| | | THTCPPCPAPELLGGPSVFLFPPKPKDTLMISRTPEVTCVVVDVSHEDPEVKENWYV | |
| | | DGVEVHNAKTKPREEQYGSTYRVVSVLTVLHQDWLNGKEYKCKVSNKALPAPIEKTI | |
| | | SKAKGQPREPQVYTLPPSRDELTKNQVSLSCAVKGFYPSDIAVEWESNGQPENNYKT | |
| | | TPPVLDSDGSFFLVSKLTVDKSRWQQGNVFSCSVMHEALHNHYTQKSLSLSPGKGGS | |
| | | GAWSHPQFEK | |
| | L: | DIQMTQSPSSLSASVGDRVTITCRASQDVSTAVAWYQQKPGKAPKLLIYSASFLYSG | 40 |
| | | VPSRFSGSGSGTDFTLTISSLQPEDFATYYCQQYLYHPATFGQGTKVEIKRTVAAPS | |
| | | VFIFPPSDEQLKSGTASVVCLLNNFYPREAKVQWKVDNALQSGNSQESVTEQDSKDS | |
| | | TYSLSSTLTLSKADYEKHKVYACEVTHQGLSSPVTKSENRGEC | |
| CXCL12 | Knob: | LSYRCPCRFFESHVARANVKHLKILNTPNCALQIVARLKNNNRQVCIDPKLKWIQEY | 12 |
| (AKPVS)- | | LEKALNPWEPKSCDKTHTCPPCPAPELLGGPSVFLFPPKPKDTLMISRTPEVTCVVV | |
| Tecentriq | | DVSHEDPEVKFNWYVDGVEVHNAKTKPREEQYGSTYRVVSVLTVLHQDWLNGKEYKC | |
| | | KVSNKALPAPIEKTISKAKGQPREPQVYTLPPSRDELTKNQVSLWCLVKGFYPSDIA | |
| | | VEWESNGQPENNYKTTPPVLDSDGSFFLYSKLTVDKSRWQQGNVFSCSVMHEALHNH | |
| | | YTQKSLSLSPGKGGSHHHHHH | |
| | Hole: | EVQLVESGGGLVQPGGSLRLSCAASGFTFSDSWIHWVRQAPGKGLEWVAWISPYGGS | 13 |
| | | TYYADSVKGRFTISADTSKNTAYLQMNSLRAEDTAVYYCARRHWPGGFDYWGQGTLV | |
| | | TVSSASTKGPSVFPLAPSSKSTSGGTAALGCLVKDYFPEPVTVSWNSGALTSGVHTF | |
| | | PAVLQSSGLYSLSSVVTVPSSSLGTQTYICNVNHKPSNTKVDKKVEPKSCEPKSCDK | |
| | | THTCPPCPAPELLGGPSVFLFPPKPKDTLMISRTPEVTCVVVDVSHEDPEVKENWYV | |
| | | DGVEVHNAKTKPREEQYGSTYRVVSVLTVLHQDWLNGKEYKCKVSNKALPAPIEKTI | |
| | | SKAKGQPREPQVYTLPPSRDELTKNQVSLSCAVKGFYPSDIAVEWESNGQPENNYKT | |
| | | TPPVLDSDGSFFLVSKLTVDKSRWQQGNVFSCSVMHEALHNHYTQKSLSLSPGKGGS | |
| | | GAWSHPQFEK | |
| | L: | DIQMTQSPSSLSASVGDRVTITCRASQDVSTAVAWYQQKPGKAPKLLIYSASFLYSG | 40 |
| | | VPSRFSGSGSGTDFTLTISSLQPEDFATYYCQQYLYHPATFGQGTKVEIKRTVAAPS | |
| | | VFIFPPSDEQLKSGTASVVCLLNNFYPREAKVQWKVDNALQSGNSQESVTEQDSKDS | |
| | | TYSLSSTLTLSKADYEKHKVYACEVTHQGLSSPVTKSENRGEC | |
| CXCL12- | Knob: | KPVSLSYRCPCRFFESHVARANVKHLKILNTPNCALQIVARLKNNNRQVCIDPKLKW | 1 |
| Tecentriq | | IQEYLEKALNPWEPKSCDKTHTCPPCPAPELLGGPSVFLFPPKPKDTLMISRTPEVT | |
| (HC S57A) | | CVVVDVSHEDPEVKFNWYVDGVEVHNAKTKPREEQYGSTYRVVSVLTVLHQDWLNGK | |
| | | EYKCKVSNKALPAPIEKTISKAKGQPREPQVYTLPPSRDELTKNQVSLWCLVKGFYP | |
| | | SDIAVEWESNGQPENNYKTTPPVLDSDGSFFLYSKLTVDKSRWQQGNVFSCSVMHEA | |
| | | LHNHYTQKSLSLSPGKGGSHHHHHH | |
| | Hole: | EVQLVESGGGLVQPGGSLRLSCAASGFTFSDSWIHWVRQAPGKGLEWVAWISPYGGA | 14 |
| | | TYYADSVKGRFTISADTSKNTAYLQMNSLRAEDTAVYYCARRHWPGGFDYWGQGTLV | |
| | | TVSSASTKGPSVFPLAPSSKSTSGGTAALGCLVKDYFPEPVTVSWNSGALTSGVHTF | |
| | | PAVLQSSGLYSLSSVVTVPSSSLGTQTYICNVNHKPSNTKVDKKVEPKSCEPKSCDK | |
| | | THTCPPCPAPELLGGPSVFLFPPKPKDTLMISRTPEVTCVVVDVSHEDPEVKENWYV | |
| | | DGVEVHNAKTKPREEQYGSTYRVVSVLTVLHQDWLNGKEYKCKVSNKALPAPIEKTI | |
| | | SKAKGQPREPQVYTLPPSRDELTKNQVSLSCAVKGFYPSDIAVEWESNGQPENNYKT | |
| | | TPPVLDSDGSFFLVSKLTVDKSRWQQGNVFSCSVMHEALHNHYTQKSLSLSPGKGGS | |
| | | GAWSHPQFEK | |
| | L: | DIQMTQSPSSLSASVGDRVTITCRASQDVSTAVAWYQQKPGKAPKLLIYSASFLYSG | 40 |
| | | VPSRFSGSGSGTDFTLTISSLQPEDFATYYCQQYLYHPATFGQGTKVEIKRTVAAPS | |
| | | VFIFPPSDEQLKSGTASVVCLLNNFYPREAKVQWKVDNALQSGNSQESVTEQDSKDS | |
| | | TYSLSSTLTLSKADYEKHKVYACEVTHQGLSSPVTKSENRGEC | |
| CXCL12- | Knob: | KPVSLSYRCPCRFFESHVARANVKHLKILNTPNCALQIVARLKNNNRQVCIDPKLKW | 1 |
| Tecentriq | | IQEYLEKALNPWEPKSCDKTHTCPPCPAPELLGGPSVFLFPPKPKDTLMISRTPEVT | |
| (HC D31A) | | CVVVDVSHEDPEVKFNWYVDGVEVHNAKTKPREEQYGSTYRVVSVLTVLHQDWLNGK | |
| | | EYKCKVSNKALPAPIEKTISKAKGQPREPQVYTLPPSRDELTKNQVSLWCLVKGFYP | |
| | | SDIAVEWESNGQPENNYKTTPPVLDSDGSFFLYSKLTVDKSRWQQGNVFSCSVMHEA | |
| | | LHNHYTQKSLSLSPGKGGSHHHHHH | |
| | Hole: | EVQLVESGGGLVQPGGSLRLSCAASGFTFSASWIHWVRQAPGKGLEWVAWISPYGGS | 15 |
| | | TYYADSVKGRFTISADTSKNTAYLQMNSLRAEDTAVYYCARRHWPGGFDYWGQGTLV | |
| | | TVSSASTKGPSVFPLAPSSKSTSGGTAALGCLVKDYFPEPVTVSWNSGALTSGVHTF | |
| | | PAVLQSSGLYSLSSVVTVPSSSLGTQTYICNVNHKPSNTKVDKKVEPKSCEPKSCDK | |
| | | THTCPPCPAPELLGGPSVFLFPPKPKDTLMISRTPEVTCVVVDVSHEDPEVKENWYV | |
| | | DGVEVHNAKTKPREEQYGSTYRVVSVLTVLHQDWLNGKEYKCKVSNKALPAPIEKTI | |
| | | SKAKGQPREPQVYTLPPSRDELTKNQVSLSCAVKGFYPSDIAVEWESNGQPENNYKT | |
| | | TPPVLDSDGSFFLVSKLTVDKSRWQQGNVFSCSVMHEALHNHYTQKSLSLSPGKGGS | |
| | | GAWSHPQFEK | |
| | L: | DIQMTQSPSSLSASVGDRVTITCRASQDVSTAVAWYQQKPGKAPKLLIYSASFLYSG | 40 |
| | | VPSRFSGSGSGTDFTLTISSLQPEDFATYYCQQYLYHPATFGQGTKVEIKRTVAAPS | |
| | | VFIFPPSDEQLKSGTASVVCLLNNFYPREAKVQWKVDNALQSGNSQESVTEQDSKDS | |
| | | TYSLSSTLTLSKADYEKHKVYACEVTHQGLSSPVTKSENRGEC | |
| CXCL12- | Knob: | KPVSLSYRCPCRFFESHVARANVKHLKILNTPNCALQIVARLKNNNRQVCIDPKLKW | 1 |
| Tecentriq | | IQEYLEKALNPWEPKSCDKTHTCPPCPAPELLGGPSVFLFPPKPKDTLMISRTPEVT | |
| (HC S30A) | | CVVVDVSHEDPEVKFNWYVDGVEVHNAKTKPREEQYGSTYRVVSVLTVLHQDWLNGK | |
| | | EYKCKVSNKALPAPIEKTISKAKGQPREPQVYTLPPSRDELTKNQVSLWCLVKGFYP | |
| | | SDIAVEWESNGQPENNYKTTPPVLDSDGSFFLYSKLTVDKSRWQQGNVFSCSVMHEA | |
| | | LHNHYTQKSLSLSPGKGGSHHHHHH | |
| | Hole: | EVQLVESGGGLVQPGGSLRLSCAASGFTFADSWIHWVRQAPGKGLEWVAWISPYGGS | 16 |
| | | TYYADSVKGRFTISADTSKNTAYLQMNSLRAEDTAVYYCARRHWPGGFDYWGQGTLV | |
| | | TVSSASTKGPSVFPLAPSSKSTSGGTAALGCLVKDYFPEPVTVSWNSGALTSGVHTE | |
| | | PAVLQSSGLYSLSSVVTVPSSSLGTQTYICNVNHKPSNTKVDKKVEPKSCEPKSCDK | |
| | | THTCPPCPAPELLGGPSVFLFPPKPKDTLMISRTPEVTCVVVDVSHEDPEVKENWYV | |
| | | DGVEVHNAKTKPREEQYGSTYRVVSVLTVLHQDWLNGKEYKCKVSNKALPAPIEKTI | |
| | | SKAKGQPREPQVYTLPPSRDELTKNQVSLSCAVKGFYPSDIAVEWESNGQPENNYKT | |
| | | TPPVLDSDGSFFLVSKLTVDKSRWQQGNVFSCSVMHEALHNHYTQKSLSLSPGKGGS | |
| | | GAWSHPQFEK | |
| | L: | DIQMTQSPSSLSASVGDRVTITCRASQDVSTAVAWYQQKPGKAPKLLIYSASFLYSG | 40 |
| | | VPSRFSGSGSGTDFTLTISSLQPEDFATYYCQQYLYHPATFGQGTKVEIKRTVAAPS | |
| | | VFIFPPSDEQLKSGTASVVCLLNNFYPREAKVQWKVDNALQSGNSQESVTEQDSKDS | |
| | | TYSLSSTLTLSKADYEKHKVYACEVTHQGLSSPVTKSENRGEC | |
| CXCL12- | Knob: | KPVSLSYRCPCRFFESHVARANVKHLKILNTPNCALQIVARLKNNNRQVCIDPKLKW | 1 |
| Tecentriq | | IQEYLEKALNPWEPKSCDKTHTCPPCPAPELLGGPSVFLFPPKPKDTLMISRTPEVT | |
| (HC W33A) | | CVVVDVSHEDPEVKFNWYVDGVEVHNAKTKPREEQYGSTYRVVSVLTVLHQDWLNGK | |
| | | EYKCKVSNKALPAPIEKTISKAKGQPREPQVYTLPPSRDELTKNQVSLWCLVKGFYP | |
| | | SDIAVEWESNGQPENNYKTTPPVLDSDGSFFLYSKLTVDKSRWQQGNVFSCSVMHEA | |
| | | LHNHYTQKSLSLSPGKGGSHHHHHH | |
| | Hole: | EVQLVESGGGLVQPGGSLRLSCAASGFTFSDSAIHWVRQAPGKGLEWVAWISPYGGS | 17 |
| | | TYYADSVKGRFTISADTSKNTAYLQMNSLRAEDTAVYYCARRHWPGGFDYWGQGTLV | |
| | | TVSSASTKGPSVFPLAPSSKSTSGGTAALGCLVKDYFPEPVTVSWNSGALTSGVHTF | |
| | | PAVLQSSGLYSLSSVVTVPSSSLGTQTYICNVNHKPSNTKVDKKVEPKSCEPKSCDK | |
| | | THTCPPCPAPELLGGPSVFLFPPKPKDTLMISRTPEVTCVVVDVSHEDPEVKENWYV | |
| | | DGVEVHNAKTKPREEQYGSTYRVVSVLTVLHQDWLNGKEYKCKVSNKALPAPIEKTI | |
| | | SKAKGQPREPQVYTLPPSRDELTKNQVSLSCAVKGFYPSDIAVEWESNGQPENNYKT | |
| | | TPPVLDSDGSFFLVSKLTVDKSRWQQGNVFSCSVMHEALHNHYTQKSLSLSPGKGGS | |
| | | GAWSHPQFEK | |
| | L: | DIQMTQSPSSLSASVGDRVTITCRASQDVSTAVAWYQQKPGKAPKLLIYSASFLYSG | 40 |
| | | VPSRFSGSGSGTDFTLTISSLQPEDFATYYCQQYLYHPATFGQGTKVEIKRTVAAPS | |
| | | VFIFPPSDEQLKSGTASVVCLLNNFYPREAKVQWKVDNALQSGNSQESVTEQDSKDS | |
| | | TYSLSSTLTLSKADYEKHKVYACEVTHQGLSSPVTKSFNRGEC | |
| CXCL12- | Knob: | KPVSLSYRCPCRFFESHVARANVKHLKILNTPNCALQIVARLKNNNRQVCIDPKLKW | 1 |
| Tecentriq | | IQEYLEKALNPWEPKSCDKTHTCPPCPAPELLGGPSVFLFPPKPKDTLMISRTPEVT | |
| (HC W50A) | | CVVVDVSHEDPEVKFNWYVDGVEVHNAKTKPREEQYGSTYRVVSVLTVLHQDWLNGK | |
| | | EYKCKVSNKALPAPIEKTISKAKGQPREPQVYTLPPSRDELTKNQVSLWCLVKGFYP | |
| | | SDIAVEWESNGQPENNYKTTPPVLDSDGSFFLYSKLTVDKSRWQQGNVFSCSVMHEA | |
| | | LHNHYTQKSLSLSPGKGGSHHHHHH | |
| | Hole: | EVQLVESGGGLVQPGGSLRLSCAASGFTFSDSWIHWVRQAPGKGLEWVAAISPYGGS | 18 |
| | | TYYADSVKGRFTISADTSKNTAYLQMNSLRAEDTAVYYCARRHWPGGFDYWGQGTLV | |
| | | TVSSASTKGPSVFPLAPSSKSTSGGTAALGCLVKDYFPEPVTVSWNSGALTSGVHTF | |
| | | PAVLQSSGLYSLSSVVTVPSSSLGTQTYICNVNHKPSNTKVDKKVEPKSCEPKSCDK | |
| | | THTCPPCPAPELLGGPSVFLFPPKPKDTLMISRTPEVTCVVVDVSHEDPEVKENWYV | |
| | | DGVEVHNAKTKPREEQYGSTYRVVSVLTVLHQDWLNGKEYKCKVSNKALPAPIEKTI | |
| | | SKAKGQPREPQVYTLPPSRDELTKNQVSLSCAVKGFYPSDIAVEWESNGQPENNYKT | |
| | | TPPVLDSDGSFFLVSKLTVDKSRWQQGNVFSCSVMHEALHNHYTQKSLSLSPGKGGS | |
| | | GAWSHPQFEK | |
| | L: | DIQMTQSPSSLSASVGDRVTITCRASQDVSTAVAWYQQKPGKAPKLLIYSASFLYSG | 40 |
| | | VPSRFSGSGSGTDFTLTISSLQPEDFATYYCQQYLYHPATFGQGTKVEIKRTVAAPS | |
| | | VFIFPPSDEQLKSGTASVVCLLNNFYPREAKVQWKVDNALQSGNSQESVTEQDSKDS | |
| | | TYSLSSTLTLSKADYEKHKVYACEVTHQGLSSPVTKSENRGEC | |
| CXCL12- | Knob: | KPVSLSYRCPCRFFESHVARANVKHLKILNTPNCALQIVARLKNNNRQVCIDPKLKW | 1 |
| Tecentriq | | IQEYLEKALNPWEPKSCDKTHTCPPCPAPELLGGPSVFLFPPKPKDTLMISRTPEVT | |
| (HC | | CVVVDVSHEDPEVKFNWYVDGVEVHNAKTKPREEQYGSTYRVVSVLTVLHQDWLNGK | |
| W101A) | | EYKCKVSNKALPAPIEKTISKAKGQPREPQVYTLPPSRDELTKNQVSLWCLVKGFYP | |
| | | SDIAVEWESNGQPENNYKTTPPVLDSDGSFFLYSKLTVDKSRWQQGNVFSCSVMHEA | |
| | | LHNHYTQKSLSLSPGKGGSHHHHHH | |
| | Hole: | EVQLVESGGGLVQPGGSLRLSCAASGFTFSDSWIHWVRQAPGKGLEWVAWISPYGGS | 19 |
| | | TYYADSVKGRFTISADTSKNTAYLQMNSLRAEDTAVYYCARRHAPGGFDYWGQGTLV | |
| | | TVSSASTKGPSVFPLAPSSKSTSGGTAALGCLVKDYFPEPVTVSWNSGALTSGVHTF | |
| | | PAVLQSSGLYSLSSVVTVPSSSLGTQTYICNVNHKPSNTKVDKKVEPKSCEPKSCDK | |
| | | THTCPPCPAPELLGGPSVFLFPPKPKDTLMISRTPEVTCVVVDVSHEDPEVKENWYV | |
| | | DGVEVHNAKTKPREEQYGSTYRVVSVLTVLHQDWLNGKEYKCKVSNKALPAPIEKTI | |
| | | SKAKGQPREPQVYTLPPSRDELTKNQVSLSCAVKGFYPSDIAVEWESNGQPENNYKT | |
| | | TPPVLDSDGSFFLVSKLTVDKSRWQQGNVFSCSVMHEALHNHYTQKSLSLSPGKGGS | |
| | | GAWSHPQFEK | |
| | L: | DIQMTQSPSSLSASVGDRVTITCRASQDVSTAVAWYQQKPGKAPKLLIYSASFLYSG | 40 |
| | | VPSRFSGSGSGTDFTLTISSLQPEDFATYYCQQYLYHPATFGQGTKVEIKRTVAAPS | |
| | | VFIFPPSDEQLKSGTASVVCLLNNFYPREAKVQWKVDNALQSGNSQESVTEQDSKDS | |
| | | TYSLSSTLTLSKADYEKHKVYACEVTHQGLSSPVTKSENRGEC | |
| CXCL12- | Knob: | KPVSLSYRCPCRFFESHVARANVKHLKILNTPNCALQIVARLKNNNRQVCIDPKLKW | 1 |
| Tecentriq | | IQEYLEKALNPWEPKSCDKTHTCPPCPAPELLGGPSVFLFPPKPKDTLMISRTPEVT | |
| (LC S30A) | | CVVVDVSHEDPEVKFNWYVDGVEVHNAKTKPREEQYGSTYRVVSVLTVLHQDWLNGK | |
| | | EYKCKVSNKALPAPIEKTISKAKGQPREPQVYTLPPSRDELTKNQVSLWCLVKGFYP | |
| | | SDIAVEWESNGQPENNYKTTPPVLDSDGSFFLYSKLTVDKSRWQQGNVFSCSVMHEA | |
| | | LHNHYTQKSLSLSPGKGGSHHHHHH | |
| | Hole: | EVQLVESGGGLVQPGGSLRLSCAASGFTFSDSWIHWVRQAPGKGLEWVAWISPYGGS | 20 |
| | | TYYADSVKGRFTISADTSKNTAYLQMNSLRAEDTAVYYCARRHWPGGFDYWGQGTLV | |
| | | TVSSASTKGPSVFPLAPSSKSTSGGTAALGCLVKDYFPEPVTVSWNSGALTSGVHTF | |
| | | PAVLQSSGLYSLSSVVTVPSSSLGTQTYICNVNHKPSNTKVDKKVEPKSCEPKSCDK | |
| | | THTCPPCPAPELLGGPSVFLFPPKPKDTLMISRTPEVTCVVVDVSHEDPEVKENWYV | |
| | | DGVEVHNAKTKPREEQYGSTYRVVSVLTVLHQDWLNGKEYKCKVSNKALPAPIEKTI | |
| | | SKAKGQPREPQVYTLPPSRDELTKNQVSLSCAVKGFYPSDIAVEWESNGQPENNYKT | |
| | | TPPVLDSDGSFFLVSKLTVDKSRWQQGNVFSCSVMHEALHNHYTQKSLSLSPGKGGS | |
| | | GAWSHPQFEK | |
| | L: | DIQMTQSPSSLSASVGDRVTITCRASQDVATAVAWYQQKPGKAPKLLIYSASFLYSG | 21 |
| | | VPSRFSGSGSGTDFTLTISSLQPEDFATYYCQQYLYHPATFGQGTKVEIKRTVAAPS | |
| | | VFIFPPSDEQLKSGTASVVCLLNNFYPREAKVQWKVDNALQSGNSQESVTEQDSKDS | |
| | | TYSLSSTLTLSKADYEKHKVYACEVTHQGLSSPVTKSENRGEC | |
| CXCL12- | Knob: | KPVSLSYRCPCRFFESHVARANVKHLKILNTPNCALQIVARLKNNNRQVCIDPKLKW | 1 |
| Tecentriq | | IQEYLEKALNPWEPKSCDKTHTCPPCPAPELLGGPSVFLFPPKPKDTLMISRTPEVT | |
| (LC Y93A) | | CVVVDVSHEDPEVKFNWYVDGVEVHNAKTKPREEQYGSTYRVVSVLTVLHQDWLNGK | |
| | | EYKCKVSNKALPAPIEKTISKAKGQPREPQVYTLPPSRDELTKNQVSLWCLVKGFYP | |
| | | SDIAVEWESNGQPENNYKTTPPVLDSDGSFFLYSKLTVDKSRWQQGNVFSCSVMHEA | |
| | | LHNHYTQKSLSLSPGKGGSHHHHHH | |
| | Hole: | EVQLVESGGGLVQPGGSLRLSCAASGFTFSDSWIHWVRQAPGKGLEWVAWISPYGGS | 22 |
| | | TYYADSVKGRFTISADTSKNTAYLQMNSLRAEDTAVYYCARRHWPGGFDYWGQGTLV | |
| | | TVSSASTKGPSVFPLAPSSKSTSGGTAALGCLVKDYFPEPVTVSWNSGALTSGVHTF | |
| | | PAVLQSSGLYSLSSVVTVPSSSLGTQTYICNVNHKPSNTKVDKKVEPKSCEPKSCDK | |
| | | THTCPPCPAPELLGGPSVFLFPPKPKDTLMISRTPEVTCVVVDVSHEDPEVKENWYV | |
| | | DGVEVHNAKTKPREEQYGSTYRVVSVLTVLHQDWLNGKEYKCKVSNKALPAPIEKTI | |
| | | SKAKGQPREPQVYTLPPSRDELTKNQVSLSCAVKGFYPSDIAVEWESNGQPENNYKT | |
| | | TPPVLDSDGSFFLVSKLTVDKSRWQQGNVFSCSVMHEALHNHYTQKSLSLSPGKGGS | |
| | | GAWSHPQFEK | |
| | L: | DIQMTQSPSSLSASVGDRVTITCRASQDVSTAVAWYQQKPGKAPKLLIYSASFLYSG | 23 |
| | | VPSRFSGSGSGTDFTLTISSLQPEDFATYYCQQYLAHPATFGQGTKVEIKRTVAAPS | |
| | | VFIFPPSDEQLKSGTASVVCLLNNFYPREAKVQWKVDNALQSGNSQESVTEQDSKDS | |
| | | TYSLSSTLTLSKADYEKHKVYACEVTHQGLSSPVTKSENRGEC | |
| CXCL12- | Knob: | KPVSLSYRCPCRFFESHVARANVKHLKILNTPNCALQIVARLKNNNRQVCIDPKLKW | 1 |
| Tecentriq | | IQEYLEKALNPWEPKSCDKTHTCPPCPAPELLGGPSVFLFPPKPKDTLMISRTPEVT | |
| (LC L92A) | | CVVVDVSHEDPEVKFNWYVDGVEVHNAKTKPREEQYGSTYRVVSVLTVLHQDWLNGK | |
| | | EYKCKVSNKALPAPIEKTISKAKGQPREPQVYTLPPSRDELTKNQVSLWCLVKGFYP | |
| | | SDIAVEWESNGQPENNYKTTPPVLDSDGSFFLYSKLTVDKSRWQQGNVFSCSVMHEA | |
| | | LHNHYTQKSLSLSPGKGGSHHHHHH | |
| | Hole: | EVQLVESGGGLVQPGGSLRLSCAASGFTFSDSWIHWVRQAPGKGLEWVAWISPYGGS | 24 |
| | | TYYADSVKGRFTISADTSKNTAYLQMNSLRAEDTAVYYCARRHWPGGFDYWGQGTLV | |
| | | TVSSASTKGPSVFPLAPSSKSTSGGTAALGCLVKDYFPEPVTVSWNSGALTSGVHTF | |
| | | PAVLQSSGLYSLSSVVTVPSSSLGTQTYICNVNHKPSNTKVDKKVEPKSCEPKSCDK | |
| | | THTCPPCPAPELLGGPSVFLFPPKPKDTLMISRTPEVTCVVVDVSHEDPEVKENWYV | |
| | | DGVEVHNAKTKPREEQYGSTYRVVSVLTVLHQDWLNGKEYKCKVSNKALPAPIEKTI | |
| | | SKAKGQPREPQVYTLPPSRDELTKNQVSLSCAVKGFYPSDIAVEWESNGQPENNYKT | |
| | | TPPVLDSDGSFFLVSKLTVDKSRWQQGNVFSCSVMHEALHNHYTQKSLSLSPGKGGS | |
| | | GAWSHPQFEK | |
| | L: | DIQMTQSPSSLSASVGDRVTITCRASQDVSTAVAWYQQKPGKAPKLLIYSASFLYSG | 25 |
| | | VPSRFSGSGSGTDFTLTISSLQPEDFATYYCQQYAYHPATFGQGTKVEIKRTVAAPS | |
| | | VFIFPPSDEQLKSGTASVVCLLNNFYPREAKVQWKVDNALQSGNSQESVTEQDSKDS | |
| | | TYSLSSTLTLSKADYEKHKVYACEVTHQGLSSPVTKSENRGEC | |
| CXCL12- | Knob: | KPVSLSYRCPCRFFESHVARANVKHLKILNTPNCALQIVARLKNNNRQVCIDPKLKW | 1 |
| BMS936559 | | IQEYLEKALNPWEPKSCDKTHTCPPCPAPELLGGPSVFLFPPKPKDTLMISRTPEVT | |
| | | CVVVDVSHEDPEVKFNWYVDGVEVHNAKTKPREEQYGSTYRVVSVLTVLHQDWLNGK | |
| | | EYKCKVSNKALPAPIEKTISKAKGQPREPQVYTLPPSRDELTKNQVSLWCLVKGFYP | |
| | | SDIAVEWESNGQPENNYKTTPPVLDSDGSFFLYSKLTVDKSRWQQGNVFSCSVMHEA | |
| | | LHNHYTQKSLSLSPGKGGSHHHHHH | |
| | Hole: | QVQLVQSGAEVKKPGSSVKVSCKTSGDTESTYAISWVRQAPGQGLEWMGGIIPIFGK | 26 |
| | | AHYAQKFQGRVTITADESTSTAYMELSSLRSEDTAVYFCARKFHFVSGSPFGMDVWG | |
| | | QGTTVTVSSASTKGPSVFPLAPSSKSTSGGTAALGCLVKDYFPEPVTVSWNSGALTS | |
| | | GVHTFPAVLQSSGLYSLSSVVTVPSSSLGTQTYICNVNHKPSNTKVDKKVEPKSCDK | |
| | | THTCPPCPAPELLGGPSVFLFPPKPKDTLMISRTPEVTCVVVDVSHEDPEVKENWYV | |
| | | DGVEVHNAKTKPREEQYGSTYRVVSVLTVLHQDWLNGKEYKCKVSNKALPAPIEKTI | |
| | | SKAKGQPREPQVYTLPPSRDELTKNQVSLSCAVKGFYPSDIAVEWESNGQPENNYKT | |
| | | TPPVLDSDGSFFLVSKLTVDKSRWQQGNVFSCSVMHEALHNHYTQKSLSLSPGKGGS | |
| | | GAWSHPQFEK | |
| | L: | EIVLTQSPATLSLSPGERATLSCRASQSVSSYLAWYQQKPGQAPRLLIYDASNRATG | 27 |
| | | IPARFSGSGSGTDFTLTISSLEPEDFAVYYCQQRSNWPTFGQGTKVEIKRTVAAPSV | |
| | | FIFPPSDEQLKSGTASVVCLLNNFYPREAKVQWKVDNALQSGNSQESVTEQDSKDST | |
| | | YSLSSTLTLSKADYEKHKVYACEVTHQGLSSPVTKSENRGEC | |
| CXCL12- | Knob: | KPVSLSYRCPCRFFESHVARANVKHLKILNTPNCALQIVARLKNNNRQVCIDPKLKW | 28 |
| Tecentriq | | IQEYLEKALNPWEPKSCDKTHTCPPCPAPELLGGPSVFLFPPKPKDTLMISRTPEVT | |
| glycosylated | | CVVVDVSHEDPEVKFNWYVDGVEVHNAKTKPREEQYNSTYRVVSVLTVLHQDWLNGK | |
| | | EYKCKVSNKALPAPIEKTISKAKGQPREPQVYTLPPSRDELTKNQVSLWCLVKGFYP | |
| | | SDIAVEWESNGQPENNYKTTPPVLDSDGSFFLYSKLTVDKSRWQQGNVFSCSVMHEA | |
| | | LHNHYTQKSLSLSPGKGGSHHHHHH | |
| | Hole: | EVQLVESGGGLVQPGGSLRLSCAASGFNISSSYMHWVRQAPGKGLEWVAYISSYYGS | 29 |
| | | TYYADSVKGRFTISRDNSKNTLYLQMNSLRAEDTAVYYCARYYSAYPRGFDYWGQGT | |
| | | LVTVSSASTKGPSVFPLAPSSKSTSGGTAALGCLVKDYFPEPVTVSWNSGALTSGVH | |
| | | TFPAVLQSSGLYSLSSVVTVPSSSLGTQTYICNVNHKPSNTKVDKKVEPKSCDKTHT | |
| | | CPPCPAPELLGGPSVFLFPPKPKDTLMISRTPEVTCVVVDVSHEDPEVKENWYVDGV | |
| | | EVHNAKTKPREEQYNSTYRVVSVLTVLHQDWLNGKEYKCKVSNKALPAPIEKTISKA | |
| | | KGQPREPQVYTLPPSRDELTKNQVSLSCAVKGFYPSDIAVEWESNGQPENNYKTTPP | |
| | | VLDSDGSFFLVSKLTVDKSRWQQGNVFSCSVMHEALHNHYTQKSLSLSPGKGGSGAW | |
| | | SHPQFEK | |
| | L: | DIQMTQSPSSLSASVGDRVTITCRASQDVSTAVAWYQQKPGKAPKLLIYSASFLYSG | 40 |
| | | VPSRFSGSGSGTDFTLTISSLQPEDFATYYCQQYLYHPATFGQGTKVEIKRTVAAPS | |
| | | VFIFPPSDEQLKSGTASVVCLLNNFYPREAKVQWKVDNALQSGNSQESVTEQDSKDS | |
| | | TYSLSSTLTLSKADYEKHKVYACEVTHQGLSSPVTKSENRGEC | |
| CXCL12- | H: | KPVSLSYRCPCRFFESHVARANVKHLKILNTPNCALQIVARLKNNNRQVCIDPKLKW | 30 |
| avi- | | IQEYLEKALNKRFKMPWDKTHTGGSGSAGGLNDIFEAQKIEWHESSGSEVQLVESGG | |
| Tecentriq | | GLVQPGGSLRLSCAASGFTFSDSWIHWVRQAPGKGLEWVAWISPYGGSTYYADSVKG | |
| diabody | | RFTISADTSKNTAYLQMNSLRAEDTAVYYCARRHWPGGFDYWGQGTLVTVSSASTKG | |
| | | PSVFPLAPSSKSTSGGTAALGCLVKDYFPEPVTVSWNSGALTSGVHTFPAVLQSSGL | |
| | | YSLSSVVTVPSSSLGTQTYICNVNHKPSNTKVDKKVEPKSC | |
| | L: | DIQMTQSPSSLSASVGDRVTITCRASQDVSTAVAWYQQKPGKAPKLLIYSASFLYSG | 40 |
| | | VPSRFSGSGSGTDFTLTISSLQPEDFATYYCQQYLYHPATFGQGTKVEIKRTVAAPS | |
| | | VFIFPPSDEQLKSGTASVVCLLNNFYPREAKVQWKVDNALQSGNSQESVTEQDSKDS | |
| | | TYSLSSTLTLSKADYEKHKVYACEVTHQGLSSPVTKSENRGEC | |
| Trastuzumab | H: | EISEVQLVESGGGLVQPGGSLRLSCAASGFNIKDTYIHWVRQAPGKGLEWVARIYPT | 31 |
| Fab | | NGYTRYADSVKGRFTISADTSKNTAYLQMNSLRAEDTAVYYCSRWGGDGFYAMDYWG | |
| | | QGTLVTVSSASTKGPSVFPLAPSSKSTSGGTAALGCLVKDYFPEPVTVSWNSGALTS | |
| | | GVHTFPAVLQSSGLYSLSSVVTVPSSSLGTQTYICNVNHKPSNTKVDKKVDKTHTGG | |
| | | SGSAGGLNDIFEAQKIEWHE | |
| | L: | DIQMTQSPSSLSASVGDRVTITCRASQDVNTAVAWYQQKPGKAPKLLIYSASFLYSG | 4 |
| | | VPSRFSGSRSGTDFTLTISSLQPEDFATYYCQQHYTTPPTFGQGTKLEIKRTVAAPS | |
| | | VFIFPPSDEQLKSGTASVVCLLNNFYPREAKVQWKVDNALQSGNSQESVTEQDSKDS | |
| | | TYSLSSTLTLSKADYEKHKVYACEVTHQGLSSPVTKSENRGEC | |
| Trastuzumab | H: | EISEVQLVESGGGLVQPGGSLRLSCAASGENIKDTYIHWVRQAPGKGLEWVARIYPT | 32 |
| IgG | | NGYTRYADSVKGRFTISADTSKNTAYLQMNSLRAEDTAVYYCSRWGGDGFYAMDYWG | |
| | | QGTLVTVSSASTKGPSVFPLAPSSKSTSGGTAALGCLVKDYFPEPVTVSWNSGALTS | |
| | | GVHTFPAVLQSSGLYSLSSVVTVPSSSLGTQTYICNVNHKPSNTKVDKKVEPKSCDK | |
| | | THTCPPCPAPELLGGPSVFLFPPKPKDTLMISRTPEVTCVVVDVSHEDPEVKENWYV | |
| | | DGVEVHNAKTKPREEQYNSTYRVVSVLTVLHQDWLNGKEYKCKVSNKALPAPIEKTI | |
| | | SKAKGQPREPQVYTLPPSRDELTKNQVSLTCLVKGFYPSDIAVEWESNGQPENNYKT | |
| | | TPPVLDSDGSFFLYSKLTVDKSRWQQGNVFSCSVMHE | |
| | | ALHNHYTQKSLSLSPGK | |
| | L: | DIQMTQSPSSLSASVGDRVTITCRASQDVNTAVAWYQQKPGKAPKLLIYSASFLYSG | 4 |
| | | VPSRFSGSRSGTDFTLTISSLQPEDFATYYCQQHYTTPPTFGQGTKLEIKRTVAAPS | |
| | | VFIFPPSDEQLKSGTASVVCLLNNFYPREAKVQWKVDNALQSGNSQESVTEQDSKDS | |
| | | TYSLSSTLTLSKADYEKHKVYACEVTHQGLSSPVTKSENRGEC | |
| Tecentriq | H: | EVQLVESGGGLVQPGGSLRLSCAASGFTFSDSWIHWVRQAPGKGLEWVAWISPYGGS | 33 |
| Fab | | TYYADSVKGRFTISADTSKNTAYLQMNSLRAEDTAVYYCARRHWPGGFDYWGQGTLV | |
| | | TVSSASTKGPSVFPLAPSSKSTSGGTAALGCLVKDYFPEPVTVSWNSGALTSGVHTF | |
| | | PAVLQSSGLYSLSSVVTVPSSSLGTQTYICNVNHKPSNTKVDKKVDKTHTGGSGSAG | |
| | | GLNDIFEAQKIEWHE | |
| | L: | DIQMTQSPSSLSASVGDRVTITCRASQDVSTAVAWYQQKPGKAPKLLIYSASFLYSG | 40 |
| | | VPSRFSGSGSGTDFTLTISSLQPEDFATYYCQQYLYHPATFGQGTKVEIKRTVAAPS | |
| | | VFIFPPSDEQLKSGTASVVCLLNNFYPREAKVQWKVDNALQSGNSQESVTEQDSKDS | |
| | | TYSLSSTLTLSKADYEKHKVYACEVTHQGLSSPVTKSENRGEC | |
| Tecentriq- | Knob: | EVQLVESGGGLVQPGGSLRLSCAASGFRIYSYYSYIGWVRRAPGKGEELVARIYPSS | 34 |
| Isotype | | GYTYYADSVKGRFTISADTSKNTAYLQMNSLRAEDTAVYYCARWDFASPYYPGSSGL | |
| | | DYWGQGTLVTVSSEPKSCDKTHTCPPCPAPELLGGPSVFLFPPKPKDTLMISRTPEV | |
| | | TCVVVDVSHEDPEVKFNWYVDGVEVHNAKTKPREEQYGSTYRVVSVLTVLHQDWING | |
| | | KEYKCKVSNKALPAPIEKTISKAKGQPREPQVYTLPPSRDELTKNQVSLWCLVKGFY | |
| | | PSDIAVEWESNGQPENNYKTTPPVLDSDGSFFLYSKLTVDKSRWQQGNVFSCSVMHE | |
| | | ALHNHYTQKSLSLSPGKGGSHHHHHH | |
| | Hole: | EVQLVESGGGLVQPGGSLRLSCAASGFTFSDSWIHWVRQAPGKGLEWVAWISPYGGS | 35 |
| | | TYYADSVKGRFTISADTSKNTAYLQMNSLRAEDTAVYYCARRHWPGGFDYWGQGTLV | |
| | | TVSSASTKGPSVFPLAPSSKSTSGGTAALGCLVKDYFPEPVTVSWNSGALTSGVHTF | |
| | | PAVLQSSGLYSLSSVVTVPSSSLGTQTYICNVNHKPSNTKVDKKVEPKSCEPKSCDK | |
| | | THTCPPCPAPELLGGPSVFLFPPKPKDTLMISRTPEVTCVVVDVSHEDPEVKENWYV | |
| | | DGVEVHNAKTKPREEQYGSTYRVVSVLTVLHQDWLNGKEYKCKVSNKALPAPIEKTI | |
| | | SKAKGQPREPQVYTLPPSRDELTKNQVSLSCAVKGFYPSDIAVEWESNGQPENNYKT | |
| | | TPPVLDSDGSFFLVSKLTVDKSRWQQGNVFSCSVMHEALHNHYTQKSLSLSPGKGGS | |
| | | GAWSHPQFEK | |
| | L: | DIQMTQSPSSLSASVGDRVTITCRASQDVSTAVAWYQQKPGKAPKLLIYSASFLYSG | 40 |
| | | VPSRFSGSGSGTDFTLTISSLQPEDFATYYCQQYLYHPATFGQGTKVEIKRTVAAPS | |
| | | VFIFPPSDEQLKSGTASVVCLLNNFYPREAKVQWKVDNALQSGNSQESVTEQDSKDS | |
| | | TYSLSSTLTLSKADYEKHKVYACEVTHQGLSSPVTKSENRGEC | |
| 4A06 Fab | H: | EISEVQLVESGGGLVQPGGSLRLSCAASGENLSYYYIHWVRQAPGKGLEWVASIYSS | 36 |
| | | SSYTSYADSVKGRFTISADTSKNTAYLQMNSLRAEDTAVYYCARAYYGFDYWGQGTL | |
| | | VTVSSASTKGPSVFPLAPSSKSTSGGTAALGCLVKDYFPEPVTVSWNSGALTSGVHT | |
| | | FPAVLQSSGLYSLSSVVTVPSSSLGTQTYICNVNHKPSNTKVDKKVDKTHTGGSGSA | |
| | | GGLNDIFEAQKIEWHE | |
| | L: | DIQMTQSPSSLSASVGDRVTITCRASQSVSSAVAWYQQKPGKAPKLLIYSASSLYSG | 37 |
| | | VPSRFSGSRSGTDFTLTISSLQPEDFATYYCQQSYYYYPITFGQGTKVEIKRTVAAP | |
| | | SVFIFPPSDSQLKSGTASVVCLLNNFYPREAKVQWKVDNALQSGNSQESVTEQDSKD | |
| | | STYSLSSTLTLSKADYEKHKVYACEVTHQGLSSPVTKSFNRGECGGSDYKDDDDK | |

| Construct | | |
|---|---|---|
| Name | Construct Sequence | |
| CXCL12 | Knob: | 41 |
| (LGGG) | LGGGSLSYRCPCRFFESHVARANVKHLKILNTPNCALQIVARLKNNNRQVCIDPKLKWI | |
| Tecentriq | QEYLEKALNPWEPKSCDKTHTCPPCPAPELLGGPSVFLFPPKPKDTLMISRTPEVTCVV | |
| | VDVSHEDPEVKFNWYVDGVEVHNAKTKPREEQYGSTYRVVSVLTVLHQDWLNGKEYKCK | |
| | VSNKALPAPIEKTISKAKGQPREPQVYTLPPSRDELTKNQVSLWCLVKGFYPSDIAVEW | |
| | ESNGQPENNYKTTPPVLDSDGSFFLYSKLTVDKSRWQQGNVFSCSVMHEALHNHYTQKS | |
| | LSLSPGKGGSHHHHHH | |
| | Hole: | 42 |
| | EVQLVESGGGLVQPGGSLRLSCAASGFTFSDSWIHWVRQAPGKGLEWVAWISPYGGSTY | |
| | YADSVKGRFTISADTSKNTAYLQMNSLRAEDTAVYYCARRHWPGGFDYWGQGTLVTVSS | |
| | ASTKGPSVFPLAPSSKSTSGGTAALGCLVKDYFPEPVTVSWNSGALTSGVHTFPAVLQS | |
| | SGLYSLSSVVTVPSSSLGTQTYICNVNHKPSNTKVDKKVEPKSCEPKSCDKTHTCPPCP | |
| | APELLGGPSVFLFPPKPKDTLMISRTPEVTCVVVDVSHEDPEVKENWYVDGVEVHNAKT | |
| | KPREEQYGSTYRVVSVLTVLHQDWLNGKEYKCKVSNKALPAPIEKTISKAKGQPREPQV | |
| | YTLPPSRDELTKNQVSLSCAVKGFYPSDIAVEWESNGQPENNYKTTPPVLDSDGSFFLV | |
| | SKLTVDKSRWQQGNVFSCSVMHEALHNHYTQKSLSLSPGKGGSGAWSHPQFEK | |
| | L: | 43 |
| | DIQMTQSPSSLSASVGDRVTITCRASQDVSTAVAWYQQKPGKAPKLLIYSASFLYSGVP | |
| | SRFSGSGSGTDFTLTISSLQPEDFATYYCQQYLYHPATFGQGTKVEIKRTVAAPSVFIF | |
| | PPSDEQLKSGTASVVCLLNNFYPREAKVQWKVDNALQSGNSQESVTEQDSKDSTYSLSS | |
| | TLTLSKADYEKHKVYACEVTHQGLSSPVTKSENRGEC | |
| CXCL12 | Knob: | 44 |
| (K1R)- | RPVSLSYRCPCRFFESHVARANVKHLKILNTPNCALQIVARLKNNNRQVCIDPKLKWIQ | |
| Tecentriq | EYLEKALNPWEPKSCDKTHTCPPCPAPELLGGPSVFLFPPKPKDTLMISRTPEVTCVVV | |
| | DVSHEDPEVKFNWYVDGVEVHNAKTKPREEQYGSTYRVVSVLTVLHQDWLNGKEYKCKV | |
| | SNKALPAPIEKTISKAKGQPREPQVYTLPPSRDELTKNQVSLWCLVKGFYPSDIAVEWE | |
| | SNGQPENNYKTTPPVLDSDGSFFLYSKLTVDKSRWQQGNVFSCSVMHEALHNHYTQKSL | |
| | SLSPGKGGSHHHHHH | |
| | Hole: | 45 |
| | EVQLVESGGGLVQPGGSLRLSCAASGFTFSDSWIHWVRQAPGKGLEWVAWISPYGGSTY | |
| | YADSVKGRFTISADTSKNTAYLQMNSLRAEDTAVYYCARRHWPGGFDYWGQGTLVTVSS | |
| | ASTKGPSVFPLAPSSKSTSGGTAALGCLVKDYFPEPVTVSWNSGALTSGVHTFPAVLQS | |
| | SGLYSLSSVVTVPSSSLGTQTYICNVNHKPSNTKVDKKVEPKSCEPKSCDKTHTCPPCP | |
| | APELLGGPSVFLFPPKPKDTLMISRTPEVTCVVVDVSHEDPEVKFNWYVDGVEVHNAKT | |
| | KPREEQYGSTYRVVSVLTVLHQDWLNGKEYKCKVSNKALPAPIEKTISKAKGQPREPQV | |
| | YTLPPSRDELTKNQVSLSCAVKGFYPSDIAVEWESNGQPENNYKTTPPVLDSDGSFFLV | |
| | SKLTVDKSRWQQGNVFSCSVMHEALHNHYTQKSLSLSPGKGGSGAWSHPQFEK | |
| | L: | 70 |
| | DIQMTQSPSSLSASVGDRVTITCRASQDVSTAVAWYQQKPGKAPKLLIYSASFLYSGVP | |
| | SRFSGSGSGTDFTLTISSLQPEDFATYYCQQYLYHPATFGQGTKVEIKRTVAAPSVFIF | |
| | PPSDEQLKSGTASVVCLLNNFYPREAKVQWKVDNALQSGNSQESVTEQDSKDSTYSLSS | |
| | TLTLSKADYEKHKVYACEVTHQGLSSPVTKSENRGEC | |
| CXCL12 | Knob: | 47 |
| (P2G)- | KGVSLSYRCPCRFFESHVARANVKHLKILNTPNCALQIVARLKNNNRQVCIDPKLKWIQ | |
| Tecentriq | EYLEKALNPWEPKSCDKTHTCPPCPAPELLGGPSVFLFPPKPKDTLMISRTPEVTCVVV | |
| | DVSHEDPEVKFNWYVDGVEVHNAKTKPREEQYGSTYRVVSVLTVLHQDWLNGKEYKCKV | |
| | SNKALPAPIEKTISKAKGQPREPQVYTLPPSRDELTKNQVSLWCLVKGFYPSDIAVEWE | |
| | SNGQPENNYKTTPPVLDSDGSFFLYSKLTVDKSRWQQGNVFSCSVMHEALHNHYTQKSL | |
| | SLSPGKGGSHHHHHH | |
| | Hole: | 48 |
| | EVQLVESGGGLVQPGGSLRLSCAASGFTFSDSWIHWVRQAPGKGLEWVAWISPYGGSTY | |
| | YADSVKGRFTISADTSKNTAYLQMNSLRAEDTAVYYCARRHWPGGFDYWGQGTLVTVSS | |
| | ASTKGPSVFPLAPSSKSTSGGTAALGCLVKDYFPEPVTVSWNSGALTSGVHTFPAVLQS | |
| | SGLYSLSSVVTVPSSSLGTQTYICNVNHKPSNTKVDKKVEPKSCEPKSCDKTHTCPPCP | |
| | APELLGGPSVFLFPPKPKDTLMISRTPEVTCVVVDVSHEDPEVKENWYVDGVEVHNAKT | |
| | KPREEQYGSTYRVVSVLTVLHQDWLNGKEYKCKVSNKALPAPIEKTISKAKGQPREPQV | |
| | YTLPPSRDELTKNQVSLSCAVKGFYPSDIAVEWESNGQPENNYKTTPPVLDSDGSFFLV | |
| | SKLTVDKSRWQQGNVFSCSVMHEALHNHYTQKSLSLSPGKGGSGAWSHPQFEK | |
| | L: | 49 |
| | DIQMTQSPSSLSASVGDRVTITCRASQDVSTAVAWYQQKPGKAPKLLIYSASFLYSGVP | |
| | SRFSGSGSGTDFTLTISSLQPEDFATYYCQQYLYHPATFGQGTKVEIKRTVAAPSVFIF | |
| | PPSDEQLKSGTASVVCLLNNFYPREAKVQWKVDNALQSGNSQESVTEQDSKDSTYSLSS | |
| | TLTLSKADYEKHKVYACEVTHQGLSSPVTKSENRGEC | |
| CXCL12 | Knob: | 50 |
| (VPGA)- | VPGASLSYRCPCRFFESHVARANVKHLKILNTPNCALQIVARLKNNNRQVCIDPKLKWI | |
| Tecentriq | QEYLEKALNPWEPKSCDKTHTCPPCPAPELLGGPSVFLFPPKPKDTLMISRTPEVTCVV | |
| | VDVSHEDPEVKFNWYVDGVEVHNAKTKPREEQYGSTYRVVSVLTVLHQDWLNGKEYKCK | |
| | VSNKALPAPIEKTISKAKGQPREPQVYTLPPSRDELTKNQVSLWCLVKGFYPSDIAVEW | |
| | ESNGQPENNYKTTPPVLDSDGSFFLYSKLTVDKSRWQQGNVFSCSVMHEALHNHYTQKS | |
| | LSLSPGKGGSHHHHHH | |
| | Hole: | 51 |
| | EVQLVESGGGLVQPGGSLRLSCAASGFTFSDSWIHWVRQAPGKGLEWVAWISPYGGSTY | |
| | YADSVKGRFTISADTSKNTAYLQMNSLRAEDTAVYYCARRHWPGGFDYWGQGTLVTVSS | |
| | ASTKGPSVFPLAPSSKSTSGGTAALGCLVKDYFPEPVTVSWNSGALTSGVHTFPAVLQS | |
| | SGLYSLSSVVTVPSSSLGTQTYICNVNHKPSNTKVDKKVEPKSCEPKSCDKTHTCPPCP | |
| | APELLGGPSVFLFPPKPKDTLMISRTPEVTCVVVDVSHEDPEVKFNWYVDGVEVHNAKT | |
| | KPREEQYGSTYRVVSVLTVLHQDWLNGKEYKCKVSNKALPAPIEKTISKAKGQPREPQV | |
| | YTLPPSRDELTKNQVSLSCAVKGFYPSDIAVEWESNGQPENNYKTTPPVLDSDGSFFLV | |
| | SKLTVDKSRWQQGNVFSCSVMHEALHNHYTQKSLSLSPGKGGSGAWSHPQFEK | |
| | L: | 52 |
| | DIQMTQSPSSLSASVGDRVTITCRASQDVSTAVAWYQQKPGKAPKLLIYSASFLYSGVP | |
| | SRFSGSGSGTDFTLTISSLQPEDFATYYCQQYLYHPATFGQGTKVEIKRTVAAPSVFIF | |
| | PPSDEQLKSGTASVVCLLNNFYPREAKVQWKVDNALQSGNSQESVTEQDSKDSTYSLSS | |
| | TLTLSKADYEKHKVYACEVTHQGLSSPVTKSENRGEC | |
| CXCL12 | Knob: | 53 |
| (QWVA)- | QWVASLSYRCPCRFFESHVARANVKHLKILNTPNCALQIVARLKNNNRQVCIDPKLKWI | |
| Tecentriq | QEYLEKALNPWEPKSCDKTHTCPPCPAPELLGGPSVFLFPPKPKDTLMISRTPEVTCVV | |
| | VDVSHEDPEVKFNWYVDGVEVHNAKTKPREEQYGSTYRVVSVLTVLHQDWLNGKEYKCK | |
| | VSNKALPAPIEKTISKAKGQPREPQVYTLPPSRDELTKNQVSLWCLVKGFYPSDIAVEW | |
| | ESNGQPENNYKTTPPVLDSDGSFFLYSKLTVDKSRWQQGNVFSCSVMHEALHNHYTQKS | |
| | LSLSPGKGGSHHHHHH | |
| | Hole: | 54 |
| | EVQLVESGGGLVQPGGSLRLSCAASGFTFSDSWIHWVRQAPGKGLEWVAWISPYGGSTY | |
| | YADSVKGRFTISADTSKNTAYLQMNSLRAEDTAVYYCARRHWPGGFDYWGQGTLVTVSS | |
| | ASTKGPSVFPLAPSSKSTSGGTAALGCLVKDYFPEPVTVSWNSGALTSGVHTFPAVLQS | |
| | SGLYSLSSVVTVPSSSLGTQTYICNVNHKPSNTKVDKKVEPKSCEPKSCDKTHTCPPCP | |
| | APELLGGPSVFLFPPKPKDTLMISRTPEVTCVVVDVSHEDPEVKENWYVDGVEVHNAKT | |
| | KPREEQYGSTYRVVSVLTVLHQDWLNGKEYKCKVSNKALPAPIEKTISKAKGQPREPQV | |
| | YTLPPSRDELTKNQVSLSCAVKGFYPSDIAVEWESNGQPENNYKTTPPVLDSDGSFFLV | |
| | SKLTVDKSRWQQGNVFSCSVMHEALHNHYTQKSLSLSPGKGGSGAWSHPQFEK | |
| | L: | 55 |
| | DIQMTQSPSSLSASVGDRVTITCRASQDVSTAVAWYQQKPGKAPKLLIYSASFLYSGVP | |
| | SRFSGSGSGTDFTLTISSLQPEDFATYYCQQYLYHPATFGQGTKVEIKRTVAAPSVFIF | |
| | PPSDEQLKSGTASVVCLLNNFYPREAKVQWKVDNALQSGNSQESVTEQDSKDSTYSLSS | |
| | TLTLSKADYEKHKVYACEVTHQGLSSPVTKSENRGEC | |
| CXCL12 | Knob: | 56 |
| (SQSQ)- | SQSQSLSYRCPCRFFESHVARANVKHLKILNTPNCALQIVARLKNNNRQVCIDPKLKWI | |
| Tecentriq | QEYLEKALNPWEPKSCDKTHTCPPCPAPELLGGPSVFLFPPKPKDTLMISRTPEVTCVV | |
| | VDVSHEDPEVKFNWYVDGVEVHNAKTKPREEQYGSTYRVVSVLTVLHQDWLNGKEYKCK | |
| | VSNKALPAPIEKTISKAKGQPREPQVYTLPPSRDELTKNQVSLWCLVKGFYPSDIAVEW | |
| | ESNGQPENNYKTTPPVLDSDGSFFLYSKLTVDKSRWQQGNVFSCSVMHEALHNHYTQKS | |
| | LSLSPGKGGSHHHHHH | |
| | Hole: | 57 |
| | EVQLVESGGGLVQPGGSLRLSCAASGFTFSDSWIHWVRQAPGKGLEWVAWISPYGGSTY | |
| | YADSVKGRFTISADTSKNTAYLQMNSLRAEDTAVYYCARRHWPGGFDYWGQGTLVTVSS | |
| | ASTKGPSVFPLAPSSKSTSGGTAALGCLVKDYFPEPVTVSWNSGALTSGVHTFPAVLQS | |
| | SGLYSLSSVVTVPSSSLGTQTYICNVNHKPSNTKVDKKVEPKSCEPKSCDKTHTCPPCP | |
| | APELLGGPSVFLFPPKPKDTLMISRTPEVTCVVVDVSHEDPEVKFNWYVDGVEVHNAKT | |
| | KPREEQYGSTYRVVSVLTVLHQDWLNGKEYKCKVSNKALPAPIEKTISKAKGQPREPQV | |
| | YTLPPSRDELTKNQVSLSCAVKGFYPSDIAVEWESNGQPENNYKTTPPVLDSDGSFFLV | |
| | SKLTVDKSRWQQGNVFSCSVMHEALHNHYTQKSLSLSPGKGGSGAWSHPQFEK | |
| | L: | 58 |
| | DIQMTQSPSSLSASVGDRVTITCRASQDVSTAVAWYQQKPGKAPKLLIYSASFLYSGVP | |
| | SRFSGSGSGTDFTLTISSLQPEDFATYYCQQYLYHPATFGQGTKVEIKRTVAAPSVFIF | |
| | PPSDEQLKSGTASVVCLLNNFYPREAKVQWKVDNALQSGNSQESVTEQDSKDSTYSLSS | |
| | TLTLSKADYEKHKVYACEVTHQGLSSPVTKSENRGEC | |
| CXCL12- | Knob: | 59 |
| Nivolumab | KPVSLSYRCPCRFFESHVARANVKHLKILNTPNCALQIVARLKNNNRQVCIDPKLKWIQ | |
| | EYLEKALNPWEPKSCDKTHTCPPCPAPELLGGPSVFLFPPKPKDTLMISRTPEVTCVVV | |
| | DVSHEDPEVKFNWYVDGVEVHNAKTKPREEQYGSTYRVVSVLTVLHQDWLNGKEYKCKV | |
| | SNKALPAPIEKTISKAKGQPREPQVYTLPPSRDELTKNQVSLWCLVKGFYPSDIAVEWE | |
| | SNGQPENNYKTTPPVLDSDGSFFLYSKLTVDKSRWQQGNVFSCSVMHEALHNHYTQKSL | |
| | SLSPGKGGSHHHHHH | |
| | Hole: | 60 |
| | QVQLVESGGGVVQPGRSLRLDCKASGITFSNSGMHWVRQAPGKGLEWVAVIWYDGSKRY | |
| | YADSVKGRFTISRDNSKNTLFLQMNSLRAEDTAVYYCATNDDYWGQGTLVTVSSASTKG | |
| | PSVFPLAPSSKSTSGGTAALGCLVKDYFPEPVTVSWNSGALTSGVHTFPAVLQSSGLYS | |
| | LSSVVTVPSSSLGTQTYICNVNHKPSNTKVDKKVEPKSCEPKSCDKTHTCPPCPAPELL | |
| | GGPSVFLFPPKPKDTLMISRTPEVTCVVVDVSHEDPEVKENWYVDGVEVHNAKTKPREE | |
| | QYGSTYRVVSVLTVLHQDWLNGKEYKCKVSNKALPAPIEKTISKAKGQPREPQVYTLPP | |
| | SRDELTKNQVSLSCAVKGFYPSDIAVEWESNGQPENNYKTTPPVLDSDGSFFLVSKLTV | |
| | DKSRWQQGNVFSCSVMHEALHNHYTQKSLSLSPGKGGSGAWSHPQFEK | |
| | L: | 61 |
| | EIVLTQSPATLSLSPGERATLSCRASQSVSSYLAWYQQKPGQAPRLLIYDASNRATGIP | |
| | ARFSGSGSGTDFTLTISSLEPEDFAVYYCQQSSNWPRTFGQGTKVEIKRTVAAPSVFIF | |
| | PPSDEQLKSGTASVVCLLNNFYPREAKVQWKVDNALQSGNSQESVTEQDSKDSTYSLSS | |
| | TLTLSKADYEKHKVYACEVTHQGLSSPVTKSENRGEC | |
| IL2- | Knob: | 62 |
| Nivolumab | APTSSSTKKTQLQLEHLLLDLQMILNGINNYKNPKLTRMLTFKFYMPKKATELKHLQCL | |
| | EEELKPLEEVLNLAQSKNFHLRPRDLISNINVIVLELKGSETTFMCEYADETATIVEFL | |
| | NRWITFCQSIISTLTEPKSCDKTHTCPPCPAPELLGGPSVFLFPPKPKDTLMISRTPEV | |
| | TCVVVDVSHEDPEVKFNWYVDGVEVHNAKTKPREEQYGSTYRVVSVLTVLHQDWLNGKE | |
| | YKCKVSNKALPAPIEKTISKAKGQPREPQVYTLPPSRDELTKNQVSLWCLVKGFYPSDI | |
| | AVEWESNGQPENNYKTTPPVLDSDGSFFLYSKLTVDKSRWQQGNVFSCSVMHEALHNHY | |
| | TQKSLSLSPGKGGSHHHHHH | |
| | Hole: | 63 |
| | QVQLVESGGGVVQPGRSLRLDCKASGITFSNSGMHWVRQAPGKGLEWVAVIWYDGSKRY | |
| | YADSVKGRFTISRDNSKNTLFLQMNSLRAEDTAVYYCATNDDYWGQGTLVTVSSASTKG | |
| | PSVFPLAPSSKSTSGGTAALGCLVKDYFPEPVTVSWNSGALTSGVHTFPAVLQSSGLYS | |
| | LSSVVTVPSSSLGTQTYICNVNHKPSNTKVDKKVEPKSCEPKSCDKTHTCPPCPAPELL | |
| | GGPSVFLFPPKPKDTLMISRTPEVTCVVVDVSHEDPEVKFNWYVDGVEVHNAKTKPREE | |
| | QYGSTYRVVSVLTVLHQDWLNGKEYKCKVSNKALPAPIEKTISKAKGQPREPQVYTLPP | |
| | SRDELTKNQVSLSCAVKGFYPSDIAVEWESNGQPENNYKTTPPVLDSDGSFFLVSKLTV | |
| | DKSRWQQGNVFSCSVMHEALHNHYTQKSLSLSPGKGGSGAWSHPQFEK | |
| | L: | 64 |
| | EIVLTQSPATLSLSPGERATLSCRASQSVSSYLAWYQQKPGQAPRLLIYDASNRATGIP | |
| | ARFSGSGSGTDFTLTISSLEPEDFAVYYCQQSSNWPRTFGQGTKVEIKRTVAAPSVFIF | |
| | PPSDEQLKSGTASVVCLLNNFYPREAKVQWKVDNALQSGNSQESVTEQDSKDSTYSLSS | |
| | TLTLSKADYEKHKVYACEVTHQGLSSPVTKSENRGEC | |
| CXCL12- | Knob: | 65 |
| Necitumumab | KPVSLSYRCPCRFFESHVARANVKHLKILNTPNCALQIVARLKNNNRQVCIDPKLKWIQ | |
| | EYLEKALNPWEPKSCDKTHTCPPCPAPELLGGPSVFLFPPKPKDTLMISRTPEVTCVVV | |
| | DVSHEDPEVKFNWYVDGVEVHNAKTKPREEQYGSTYRVVSVLTVLHQDWLNGKEYKCKV | |
| | SNKALPAPIEKTISKAKGQPREPQVYTLPPSRDELTKNQVSLWCLVKGFYPSDIAVEWE | |
| | SNGQPENNYKTTPPVLDSDGSFFLYSKLTVDKSRWQQGNVFSCSVMHEALHNHYTQKSL | |
| | SLSPGKGGSHHHHHH | |
| | Hole: | 66 |
| | QVQLQESGPGLVKPSQTLSLTCTVSGGSISSGDYYWSWIRQPPGKGLEWIGYIYYSGST | |
| | DYNPSLKSRVTMSVDTSKNQFSLKVNSVTAADTAVYYCARVSIFGVGTFDYWGQGTLVT | |
| | VSSASTKGPSVLPLAPSSKSTSGGTAALGCLVKDYFPEPVTVSWNSGALTSGVHTFPAV | |
| | LQSSGLYSLSSVVTVPSSSLGTQTYICNVNHKPSNTKVDKKV | |
| | EPKSCDKTHTCPPCPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKEYKCKVSNKALPAPIE | |
| | KTISKAKGQPREPQVYTLPPSRDELTKNQVSLSCAVKGFYPSDIAVEWESNGQPENNYK | |
| | TTPPVLDSDGSFFLVSKLTVDKSRWQQGNVFSCSVMHEALHNHYTQKSLSLSPGK | |
| | L: | 67 |
| | EIVMTQSPATLSLSPGERATLSCRASQSVSSYLAWYQQKPGQAPRLLIYDASNRATGIP | |
| | ARFSGSGSGTDFTLTISSLEPEDFAVYYCHQYGSTPLTFGGGTKAEIKRTVAAPSVFIF | |
| | PPSDEQLKSGTASVVCLLNNFYPREAKVQWKVDNALQSGNSQESVTEQDSKDSTYSLSS | |
| | TLTLSKADYEKHKVYACEVTHQGLSSPVTKSENRGEC | |
| CXCL12- | Knob: | 1000 |
| Panitumumab | KPVSLSYRCPCRFFESHVARANVKHLKILNTPNCALQIVARLKNNNRQVCIDPKLKWIQ | |
| | EYLEKALNPWEPKSCDKTHTCPPCPAPELLGGPSVFLFPPKPKDTLMISRTPEVTCVVV | |
| | DVSHEDPEVKFNWYVDGVEVHNAKTKPREEQYGSTYRVVSVLTVLHQDWLNGKEYKCKV | |
| | SNKALPAPIEKTISKAKGQPREPQVYTLPPSRDELTKNQVSLWCLVKGFYPSDIAVEWE | |
| | SNGQPENNYKTTPPVLDSDGSFFLYSKLTVDKSRWQQGNVFSCSVMHEALHNHYTQKSL | |
| | SLSPGKGGSHHHHHH | |
| | Hole: | 69 |
| | QVQLQESGPGLVKPSETLSLTCTVSGGSVSSGDYYWTWIRQSPGKGLEWIGHIYYSGNT | |
| | NYNPSLKSRLTISIDTSKTQFSLKLSSVTAADTAIYYCVRDRVTGAFDIWGQGTMVTVS | |
| | SASTKGPSVFPLAPCSRSTSESTAALGCLVKDYFPEPVTVSWNSGALTSGVHTFPAVLQ | |
| | SSGLYSLSSVVTVPSSSLGTQTYICNVNHKPSNTKVDKKVEPKSCDKTHTCPPCPAPEL | |
| | LGGPSVFLFPPKPKDTLMISRTPEVTCVVVDVSHEDPEVKENWYVDGVEVHNAKTKPRE | |
| | EQYNSTYRVVSVLTVLHQDWLNGKEYKCKVSNKALPAPIEKTISKAKGQPREPQVYTLP | |
| | PSRDELTKNQVSLSCAVKGFYPSDIAVEWESNGQPENNYKTTPPVLDSDGSFFLVSKLT | |
| | VDKSRWQQGNVFSCSVMHEALHNHYTQKSLSLSPGK | |
| | L: | |
| | DIQMTQSPSSLSASVGDRVTITCQASQDISNYLNWYQQKPGKAPKLLIYDASNLETGVP | |
| | SRFSGSGSGTDFTFTISSLQPEDIATYFCQHFDHLPLAFGGGTKVEIKRTVAAPSVFIF | |
| | PPSDEQLKSGTASVVCLLNNFYPREAKVQWKVDNALQSGNSQESVTEQDSKDSTYSLSS | |
| | TLTLSKADYEKHKVYACEVTHQGLSSPVTKSENRGEC | |
| CXCL12- | Knob: | 70 |
| Matuzumab | KPVSLSYRCPCRFFESHVARANVKHLKILNTPNCALQIVARLKNNNRQVCIDPKLKWIQ | |
| | EYLEKALNPWEPKSCDKTHTCPPCPAPELLGGPSVFLFPPKPKDTLMISRTPEVTCVVV | |
| | DVSHEDPEVKFNWYVDGVEVHNAKTKPREEQYGSTYRVVSVLTVLHQDWLNGKEYKCKV | |
| | SNKALPAPIEKTISKAKGQPREPQVYTLPPSRDELTKNQVSLWCLVKGFYPSDIAVEWE | |
| | SNGQPENNYKTTPPVLDSDGSFFLYSKLTVDKSRWQQGNVFSCSVMHEALHNHYTQKSL | |
| | SLSPGKGGSHHHHHH | |
| | Hole: | 71 |
| | QVQLVQSGAEVKKPGASVKVSCKASGYTFTSHWMHWVRQAPGQGLEWIGEFNPSNGRTN | |
| | YNEKFKSKATMTVDTSTNTAYMELSSLRSEDTAVYYCASRDYDYDGRYFDYWGQGTLVT | |
| | VSSASTKGPSVFPLAPSSKSTSGGTAALGCLVKDYFPEPVTVSWNSGALTSGVHTFPAV | |
| | LQSSGLYSLSSVVTVPSSSLGTQTYICNVNHKPSNTKVDKKVEPKSCDKTHTCPPCPAP | |
| | ELLGGPSVFLFPPKPKDTLMISRTPEVTCVVVDVSHEDPEVKFNWYVDGVEVHNAKTKP | |
| | REEQYNSTYRVVSVLTVLHQDWLNGKEYKCKVSNKALPAPIEKTISKAKGQPREPQVYT | |
| | LPPSRDELTKNQVSLSCAVKGFYPSDIAVEWESNGQPENNYKTTPPVLDSDGSFFLVSK | |
| | LTVDKSRWQQGNVFSCSVMHEALHNHYTQKSLSLSPGK | |
| | L: | 72 |
| | DIQMTQSPSSLSASVGDRVTITCSASSSVTYMYWYQQKPGKAPKLLIYDTSNLASGVPS | |
| | RFSGSGSGTDYTFTISSLQPEDIATYYCQQWSSHIFTFGQGTKVEIKRTVAAPSVFIFP | |
| | PSDEQLKSGTASVVCLLNNFYPREAKVQWKVDNALQSGNSQESVTEQDSKDSTYSLSST | |
| | LTLSKADYEKHKVYACEVTHQGLSSPVTKSENRGEC | |
| CXCL12- | Knob: | 73 |
| Nimotuzumab | KPVSLSYRCPCRFFESHVARANVKHLKILNTPNCALQIVARLKNNNRQVCIDPKLKWIQ | |
| | EYLEKALNPWEPKSCDKTHTCPPCPAPELLGGPSVFLFPPKPKDTLMISRTPEVTCVVV | |
| | DVSHEDPEVKFNWYVDGVEVHNAKTKPREEQYGSTYRVVSVLTVLHQDWLNGKEYKCKV | |
| | SNKALPAPIEKTISKAKGQPREPQVYTLPPSRDELTKNQVSLWCLVKGFYPSDIAVEWE | |
| | SNGQPENNYKTTPPVLDSDGSFFLYSKLTVDKSRWQQGNVFSCSVMHEALHNHYTQKSL | |
| | SLSPGKGGSHHHHHH | |
| | Hole: | 74 |
| | QVQLQQSGAEVKKPGSSVKVSCKASGYTFTNYYIYWVRQAPGQGLEWIGGINPTSGGSN | |
| | FNEKFKTRVTITVDESTNTAYMELSSLRSEDTAFYFCARQGLWFDSDGRGFDFWGQGST | |
| | VTVSSASTKGPSVFPLAPSSKSTSGGTAALGCLVKDYFPEPVTVSWNSGALTSGVHTFP | |
| | AVLQSSGLYSLSSVVTVPSSSLGTQTYICNVNHKPSNTKVDKKVEPKSCDKTHTCPPCP | |
| | APELLGGPSVFLFPPKPKDTLMISRTPEVTCVVVDVSHEDPEVKFNWYVDGVEVHNAKT | |
| | KPREEQYGSTYRVVSVLTVLHQDWLNGKEYKCKVSNKALPAPIEKTISKAKGQPREPQV | |
| | YTLPPSRDELTKNQVSLSCAVKGFYPSDIAVEWESNGQPENNYKTTPPVLDSDGSFFLV | |
| | SKLTVDKSRWQQGNVFSCSVMHEALHNHYTQKSLSLSPGKGGSGAWSHPQFEK | |
| | L: | 75 |
| | DIQMTQSPSSLSASVGDRVTITCRSSQNIVHSNGNTYLDWYQQTPGKAPKLLIYKVSNR | |
| | FSGVPSRFSGSGSGTDFTFTISSLQPEDIATYYCFQYSHVPWTFGQGTKLQITREVAAP | |
| | SVFIFPPSDEQLKSGTASVVCLLNNFYPREAKVQWKVDNALQSGNSQESVTEQDSKDST | |
| | YSLSSTLTLSKADYEKHKVYACEVTHQGLSSPVTKSFNRGECGGSDYKDDDDK | |
| CXCL12- | Knob: | 76 |
| Depatuxizumab | KPVSLSYRCPCRFFESHVARANVKHLKILNTPNCALQIVARLKNNNRQVCIDPKLKWIQ | |
| | EYLEKALNPWEPKSCDKTHTCPPCPAPELLGGPSVFLFPPKPKDTLMISRTPEVTCVVV | |
| | DVSHEDPEVKFNWYVDGVEVHNAKTKPREEQYGSTYRVVSVLTVLHQDWLNGKEYKCKV | |
| | SNKALPAPIEKTISKAKGQPREPQVYTLPPSRDELTKNQVSLWCLVKGFYPSDIAVEWE | |
| | SNGQPENNYKTTPPVLDSDGSFFLYSKLTVDKSRWQQGNVFSCSVMHEALHNHYTQKSL | |
| | SLSPGKGGSHHHHHH | |
| | Hole: | 77 |
| | QVQLQESGPGLVKPSQTLSLTCTVSGYSISSDFAWNWIRQPPGKGLEWMGYISYSGNTR | |
| | YQPSLKSRITISRDTSKNQFFLKLNSVTAADTATYYCVTAGRGFPYWGQGTLVTVSSAS | |
| | TKGPSVFPLAPSSKSTSGGTAALGCLVKDYFPEPVTVSWNSGALTSGVHTFPAVLQSSG | |
| | LYSLSSVVTVPSSSLGTQTYICNVNHKPSNTKVDKKVEPKSCDKTHTCPPCPAPELLGG | |
| | PSVFLFPPKPKDTLMISRTPEVTCVVVDVSHEDPEVKFNWYVDGVEVHNAKTKPREEQY | |
| | GSTYRVVSVLTVLHQDWLNGKEYKCKVSNKALPAPIEKTISKAKGQPREPQVYTLPPSR | |
| | DELTKNQVSLSCAVKGFYPSDIAVEWESNGQPENNYKTTPPVLDSDGSFFLVSKLTVDK | |
| | SRWQQGNVFSCSVMHEALHNHYTQKSLSLSPGKGGSGAWSHPQFEK | |
| | L: | 78 |
| | DIQMTQSPSSMSVSVGDRVTITCHSSQDINSNIGWLQQKPGKSFKGLIYHGTNLDDGVP | |
| | SRFSGSGSGTDYTLTISSLQPEDFATYYCVQYAQFPWTFGGGTKLEIKRTVAAPSVFIF | |
| | PPSDEQLKSGTASVVCLLNNFYPREAKVQWKVDNALQSGNSQESVTEQDSKDSTYSLSS | |
| | TLTLSKADYEKHKVYACEVTHQGLSSPVTKSFNRGECGGSDYKDDDDK | |
| CXCL11- | Knob: | 79 |
| Tecentriq | FPMFKRGRCLCIGPGVKAVKVADIEKASIMYPSNNCDKIEVIITLKENKGQRCLNPKSK | |
| | QARLIIKKVERKNFEPKSCDKTHTCPPCPAPELLGGPSVFLFPPKPKDTLMISRTPEVT | |
| | CVVVDVSHEDPEVKFNWYVDGVEVHNAKTKPREEQYGSTYRVVSVLTVLHQDWLNGKEY | |
| | KCKVSNKALPAPIEKTISKAKGQPREPQVYTLPPSRDELTKNQVSLWCLVKGFYPSDIA | |
| | VEWESNGQPENNYKTTPPVLDSDGSFFLYSKLTVDKSRWQQGNVFSCSVMHEALHNHYT | |
| | QKSLSLSPGKGGSHHHHHH | |
| | Hole: | 80 |
| | EVQLVESGGGLVQPGGSLRLSCAASGFTFSDSWIHWVRQAPGKGLEWVAWISPYGGSTY | |
| | YADSVKGRFTISADTSKNTAYLQMNSLRAEDTAVYYCARRHWPGGFDYWGQGTLVTVSS | |
| | ASTKGPSVFPLAPSSKSTSGGTAALGCLVKDYFPEPVTVSWNSGALTSGVHTFPAVLQS | |
| | SGLYSLSSVVTVPSSSLGTQTYICNVNHKPSNTKVDKKVEPKSCEPKSCDKTHTCPPCP | |
| | APELLGGPSVFLFPPKPKDTLMISRTPEVTCVVVDVSHEDPEVKFNWYVDGVEVHNAKT | |
| | KPREEQYGSTYRVVSVLTVLHQDWLNGKEYKCKVSNKALPAPIEKTISKAKGQPREPQV | |
| | YTLPPSRDELTKNQVSLSCAVKGFYPSDIAVEWESNGQPENNYKTTPPVLDSDGSFFLV | |
| | SKLTVDKSRWQQGNVFSCSVMHEALHNHYTQKSLSLSPGKGGSGAWSHPQFEK | |
| | L: | 81 |
| | DIQMTQSPSSLSASVGDRVTITCRASQDVSTAVAWYQQKPGKAPKLLIYSASFLYSGVP | |
| | SRFSGSGSGTDFTLTISSLQPEDFATYYCQQYLYHPATFGQGTKVEIKRTVAAPSVFIF | |
| | PPSDEQLKSGTASVVCLLNNFYPREAKVQWKVDNALQSGNSQESVTEQDSKDSTYSLSS | |
| | TLTLSKADYEKHKVYACEVTHQGLSSPVTKSENRGEC | |
| CXCL11- | Knob: | 82 |
| Cetuximab | FPMFKRGRCLCIGPGVKAVKVADIEKASIMYPSNNCDKIEVIITLKENKGQRCLNPKSK | |
| | QARLIIKKVERKNFEPKSCDKTHTCPPCPAPELLGGPSVFLFPPKPKDTLMISRTPEVT | |
| | CVVVDVSHEDPEVKFNWYVDGVEVHNAKTKPREEQYGSTYRVVSVLTVLHQDWLNGKEY | |
| | KCKVSNKALPAPIEKTISKAKGQPREPQVYTLPPSRDELTKNQVSLWCLVKGFYPSDIA | |
| | VEWESNGQPENNYKTTPPVLDSDGSFFLYSKLTVDKSRWQQGNVFSCSVMHEALHNHYT | |
| | QKSLSLSPGKGGSHHHHHH | |
| | Hole: | |
| | QVQLKQSGPGLVQPSQSLSITCTVSGFSLTNYGVHWVRQSPGKGLEWLGVIWSGGNTDY | |
| | NTPFTSRLSINKDNSKSQVFFKMNSLQSNDTAIYYCARALTYYDYEFAYWGQGTLVTVS | |
| | AASTKGPSVFPLAPSSKSTSGGTAALGCLVKDYFPEPVTVSWNSGALTSGVHTFPAVLQ | |
| | SSGLYSLSSVVTVPSSSLGTQTYICNVNHKPSNTKVDKKVEPK | |
| | SCDKTHTCPPCPAPELLGGPSVFLFPPKPKDTLMISRTPEVTCVVVDVSHEDPEVKENW | |
| | YVDGVEVHNAKTKPREEQYGSTYRVVSVLTVLHQDWLNGKEYKCKVSNKALPAPIEKTI | |
| | SKAKGQPREPQVYTLPPSRDELTKNQVSLSCAVKGFYPSDIAVEWESNGQPENNYKTTP | |
| | PVLDSDGSFFLVSKLTVDKSRWQQGNVFSCSVMHEALHNHYTQKSLSLSPGKGGSGAWS | |
| | HPQFEK | |
| | L: | |
| | DILLTQSPVILSVSPGERVSFSCRASQSIGTNIHWYQQRTNGSPRLLIKYASESISGIP | |
| | SRFSGSGSGTDFTLSINSVESEDIADYYCQQNNNWPTTFGAGTKLELKRTVAAPSVFIF | |
| | PPSDEQLKSGTASVVCLLNNFYPREAKVQWKVDNALQSGNSQESVTEQDSKDSTYSLSS | |
| | TLTLSKADYEKHKVYACEVTHQGLSSPVTKSENRGEC | |
| VMIPII- | Knob: | |
| Tecentriq | GDTLGASWHRPDKCCLGYQKRPLPQVLLSSWYPTSQLCSKPGVIFLTKRGRQVCADKSK | |
| | DWVKKLMQQLPVTAREPKSCDKTHTCPPCPAPELLGGPSVFLFPPKPKDTLMISRTPEV | |
| | TCVVVDVSHEDPEVKFNWYVDGVEVHNAKTKPREEQYGSTYRVVSVLTVLHQDWLNGKE | |
| | YKCKVSNKALPAPIEKTISKAKGQPREPQVYTLPPSRDELTKNQVSLWCLVKGFYPSDI | |
| | AVEWESNGQPENNYKTTPPVLDSDGSFFLYSKLTVDKSRWQQGNVFSCSVMHEALHNHY | |
| | TQKSLSLSPGKGGSHHHHHH | |
| | Hole: | |
| | EVQLVESGGGLVQPGGSLRLSCAASGFTFSDSWIHWVRQAPGKGLEWVAWISPYGGSTY | |
| | YADSVKGRFTISADTSKNTAYLQMNSLRAEDTAVYYCARRHWPGGFDYWGQGTLVTVSS | |
| | ASTKGPSVFPLAPSSKSTSGGTAALGCLVKDYFPEPVTVSWNSGALTSGVHTFPAVLQS | |
| | SGLYSLSSVVTVPSSSLGTQTYICNVNHKPSNTKVDKKVEPKSCEPKSCDKTHTCPPCP | |
| | APELLGGPSVFLFPPKPKDTLMISRTPEVTCVVVDVSHEDPEVKFNWYVDGVEVHNAKT | |
| | KPREEQYGSTYRVVSVLTVLHQDWLNGKEYKCKVSNKALPAPIEKTISKAKGQPREPQV | |
| | YTLPPSRDELTKNQVSLSCAVKGFYPSDIAVEWESNGQPENNYKTTPPVLDSDGSFFLV | |
| | SKLTVDKSRWQQGNVFSCSVMHEALHNHYTQKSLSLSPGKGGSGAWSHPQFEK | |
| | L: | |
| | DIQMTQSPSSLSASVGDRVTITCRASQDVSTAVAWYQQKPGKAPKLLIYSASFLYSGVP | |
| | SRFSGSGSGTDFTLTISSLQPEDFATYYCQQYLYHPATFGQGTKVEIKRTVAAPSVFIF | |
| | PPSDEQLKSGTASVVCLLNNFYPREAKVQWKVDNALQSGNSQESVTEQDSKDSTYSLSS | |
| | TLTLSKADYEKHKVYACEVTHQGLSSPVTKSENRGEC | |
| VMIPII- | Knob: | |
| Cetuximab | GDTLGASWHRPDKCCLGYQKRPLPQVLLSSWYPTSQLCSKPGVIFLTKRGRQVCADKSK | |
| | DWVKKLMQQLPVTAREPKSCDKTHTCPPCPAPELLGGPSVFLFPPKPKDTLMISRTPEV | |
| | TCVVVDVSHEDPEVKFNWYVDGVEVHNAKTKPREEQYGSTYRVVSVLTVLHQDWLNGKE | |
| | YKCKVSNKALPAPIEKTISKAKGQPREPQVYTLPPSRDELTKNQVSLWCLVKGFYPSDI | |
| | AVEWESNGQPENNYKTTPPVLDSDGSFFLYSKLTVDKSRWQQGNVFSCSVMHEALHNHY | |
| | TQKSLSLSPGKGGSHHHHHH | |
| | Hole: | |
| | QVQLKQSGPGLVQPSQSLSITCTVSGFSLTNYGVHWVRQSPGKGLEWLGVIWSGGNTDY | |
| | NTPFTSRLSINKDNSKSQVFFKMNSLQSNDTAIYYCARALTYYDYEFAYWGQGTLVTVS | |
| | AASTKGPSVFPLAPSSKSTSGGTAALGCLVKDYFPEPVTVSWNSGALTSGVHTFPAVLQ | |
| | SSGLYSLSSVVTVPSSSLGTQTYICNVNHKPSNTKVDKKVEPK | |
| | SCDKTHTCPPCPAPELLGGPSVFLFPPKPKDTLMISRTPEVTCVVVDVSHEDPEVKENW | |
| | YVDGVEVHNAKTKPREEQYGSTYRVVSVLTVLHQDWLNGKEYKCKVSNKALPAPIEKTI | |
| | SKAKGQPREPQVYTLPPSRDELTKNQVSLSCAVKGFYPSDIAVEWESNGQPENNYKTTP | |
| | PVLDSDGSFFLVSKLTVDKSRWQQGNVFSCSVMHEALHNHYTQKSLSLSPGKGGSGAWS | |
| | HPQFEK | |
| | L: | |
| | DILLTQSPVILSVSPGERVSFSCRASQSIGTNIHWYQQRTNGSPRLLIKYASESISGIP | |
| | SRFSGSGSGTDFTLSINSVESEDIADYYCQQNNNWPTTFGAGTKLELKRTVAAPSVFIF | |
| | PPSDEQLKSGTASVVCLLNNFYPREAKVQWKVDNALQSGNSQESVTEQDSKDSTYSLSS | |
| | TLTLSKADYEKHKVYACEVTHQGLSSPVTKSENRGEC | |
| CXCL12- | Knob: | |
| Pertuzumab | KPVSLSYRCPCRFFESHVARANVKHLKILNTPNCALQIVARLKNNNRQVCIDPKLKWIQ | |
| | EYLEKALNPWEPKSCDKTHTCPPCPAPELLGGPSVFLFPPKPKDTLMISRTPEVTCVVV | |
| | DVSHEDPEVKFNWYVDGVEVHNAKTKPREEQYGSTYRVVSVLTVLHQDWLNGKEYKCKV | |
| | SNKALPAPIEKTISKAKGQPREPQVYTLPPSRDELTKNQVSLWCLVKGFYPSDIAVEWE | |
| | SNGQPENNYKTTPPVLDSDGSFFLYSKLTVDKSRWQQGNVFSCSVMHEALHNHYTQKSL | |
| | SLSPGKGGSHHHHHH | |
| | Hole: | |
| | EVQLVESGGGLVQPGGSLRLSCAASGFTFTDYTMDWVRQAPGKGLEWVADVNPNSGGSI | |
| | YNQRFKGRFTLSVDRSKNTLYLQMNSLRAEDTAVYYCARNLGPSFYFDYWGQGTLVTVS | |
| | SASTKGPSVFPLAPSSKSTSGGTAALGCLVKDYFPEPVTVSWNSGALTSGVHTFPAVLQ | |
| | SSGLYSLSSVVTVPSSSLGTQTYICNVNHKPSNTKVDKKVEPKSCEPKSCDKTHTCPPC | |
| | PAPELLGGPSVFLFPPKPKDTLMISRTPEVTCVVVDVSHEDPEVKENWYVDGVEVHNAK | |
| | TKPREEQYGSTYRVVSVLTVLHQDWLNGKEYKCKVSNKALPAPIEKTISKAKGQPREPQ | |
| | VYTLPPSRDELTKNQVSLSCAVKGFYPSDIAVEWESNGQPENNYKTTPPVLDSDGSFEL | |
| | VSKLTVDKSRWQQGNVFSCSVMHEALHNHYTQKSLSLSPGKGGSGAWSHPQFEK | |
| | L: | |
| | DIQMTQSPSSLSASVGDRVTITCKASQDVSIGVAWYQQKPGKAPKLLIYSASYRYTGVP | |
| | SRFSGSGSGTDFTLTISSLQPEDFATYYCQQYYIYPYTFGQGTKVEIKRTVAAPSVFIF | |
| | PPSDEQLKSGTASVVCLLNNFYPREAKVQWKVDNALQSGNSQESVTEQDSKDSTYSLSS | |
| | TLTLSKADYEKHKVYACEVTHQGLSSPVTKSENRGEC | |
| CXCL12- | Knob: | |
| Bevacizumab | KPVSLSYRCPCRFFESHVARANVKHLKILNTPNCALQIVARLKNNNRQVCIDPKLKWIQ | |
| | EYLEKALNPWEPKSCDKTHTCPPCPAPELLGGPSVFLFPPKPKDTLMISRTPEVTCVVV | |
| | DVSHEDPEVKFNWYVDGVEVHNAKTKPREEQYGSTYRVVSVLTVLHQDWLNGKEYKCKV | |
| | SNKALPAPIEKTISKAKGQPREPQVYTLPPSRDELTKNQVSLWCLVKGFYPSDIAVEWE | |
| | SNGQPENNYKTTPPVLDSDGSFFLYSKLTVDKSRWQQGNVFSCSVMHEALHNHYTQKSL | |
| | SLSPGKGGSHHHHHH | |
| | Hole: | |
| | EVQLVESGGGLVQPGGSLRLSCAASGYTFTNYGMNWVRQAPGKGLEWVGWINTYTGEPT | |
| | YAADFKRRFTFSLDTSKSTAYLQMNSLRAEDTAVYYCAKYPHYYGSSHWYFDVWGQGTL | |
| | VTVSSASTKGPSVFPLAPSSKSTSGGTAALGCLVKDYFPEPVTVSWNSGALTSGVHTFP | |
| | AVLQSSGLYSLSSVVTVPSSSLGTQTYICNVNHKPSNTKVDKKVEPKSCDKTHTCPPCP | |
| | APELLGGPSVFLFPPKPKDTLMIS | |
| | RTPEVTCVVVDVSHEDPEVKFNWYVDGVEVHNAKTKPREEQYGSTYRVVSVLTVLHQDW | |
| | LNGKEYKCKVSNKALPAPIEKTISKAKGQPREPQVYTLPPSRDELTKNQVSLSCAVKGF | |
| | YPSDIAVEWESNGQPENNYKTTPPVLDSDGSFFLVSKLTVDKSRWQQGNVFSCSVMHEA | |
| | LHNHYTQKSLSLSPGKGGSGAWSHPQFEK | |
| | L: | |
| | ASDIQMTQSPSSLSASVGDRVTITCSASQDISNYLNWYQQKPGKAPKVLIYFTSSLHSG | |
| | VPSRFSGSGSGTDFTLTISSLQPEDFATYYCQQYSTVPWTFGQGTKVEIKRTVAAPSVE | |
| | IFPPSDEQLKSGTASVVCLLNNFYPREAKVQWKVDNALQSGNSQESVTEQDSKDSTYSL | |
| | SSTLTLSKADYEKHKVYACEVTHQGLSSPVTKSENRGEC | |
| CXCL12- | Knob: | |
| Adalimumab | KPVSLSYRCPCRFFESHVARANVKHLKILNTPNCALQIVARLKNNNRQVCIDPKLKWIQ | |
| | EYLEKALNPWEPKSCDKTHTCPPCPAPELLGGPSVFLFPPKPKDTLMISRTPEVTCVVV | |
| | DVSHEDPEVKFNWYVDGVEVHNAKTKPREEQYGSTYRVVSVLTVLHQDWLNGKEYKCKV | |
| | SNKALPAPIEKTISKAKGQPREPQVYTLPPSRDELTKNQVSLWCLVKGFYPSDIAVEWE | |
| | SNGQPENNYKTTPPVLDSDGSFFLYSKLTVDKSRWQQGNVFSCSVMHEALHNHYTQKSL | |
| | SLSPGKGGSHHHHHH | |
| | Hole: | |
| | EVQLVESGGGLVQPGRSLRLSCAASGFTFDDYAMHWVRQAPGKGLEWVSAITWNSGHID | |
| | YADSVEGRFTISRDNAKNSLYLQMNSLRAEDTAVYYCAKVSYLSTASSLDYWGQGTLVT | |
| | VSSASTKGPSVFPLAPSSKSTSGGTAALGCLVKDYFPEPVTVSWNSGALTSGVHTFPAV | |
| | LQSSGLYSLSSVVTVPSSSLGTQTYICNVNHKPSNTKVDKKVEPKSCDKTHTCPPCPAP | |
| | ELLGGPSVFLFPPKPKDTLMISRTPEVTCVVVDVSHEDPEVKENWYVDGVEVHNAKTKP | |
| | REEQYGSTYRVVSVLTVLHQDWLNGKEYKCKVSNKALPAPIEKTISKAKGQPREPQVYT | |
| | LPPSRDELTKNQVSLSCAVKGFYPSDIAVEWESNGQPENNYKTTPPVLDSDGSFFLVSK | |
| | LTVDKSRWQQGNVFSCSVMHEALHNHYTQKSLSLSPGKGGSGAWSHPQFEK | |
| | L: | |
| | DIQMTQSPSSLSASVGDRVTITCRASQGIRNYLAWYQQKPGKAPKLLIYAASTLQSGVP | |
| | SRFSGSGSGTDFTLTISSLQPEDVATYYCQRYNRAPYTFGQGTKVEIKRTVAAPSVFIF | |
| | PPSDEQLKSGTASVVCLLNNFYPREAKVQWKVDNALQSGNSQESVTEQDSKDSTYSLSS | |
| | TLTLSKADYEKHKVYACEVTHQGLSSPVTKSENRGEC | |
| CXCL12- | H: | |
| Tecentriq | KPVSLSYRCPCRFFESHVARANVKHLKILNTPNCALQIVARLKNNNRQVCIDPKLKWIQ | |
| IgG HC | EYLEKALNKRFKMPWDKTHTGGSGSAGGLNDIFEAQKIEWHESSNSEVQLVESGGGLVQ | |
| Fusion | PGGSLRLSCAASGFTFSDSWIHWVRQAPGKGLEWVAWISPYGGSTYYADSVKGRFTISA | |
| | DTSKNTAYLQMNSLRAEDTAVYYCARRHWPGGFDYWGQGTLVTVSSASTKGPSVFPLAP | |
| | SSKSTSGGTAALGCLVKDYFPEPVTVSWNSGALTSGVHTFPAVLQSSGLYSLSSVVTVP | |
| | SSSLGTQTYICNVNHKPSNTKVDKKVEPKSCEPKSCDKTHTCPPCPAPELLGGPSVFLF | |
| | PPKPKDTLMISRTPEVTCVVVDVSHEDPEVKFNWYVDGVEVHNAKTKPREEQYGSTYRV | |
| | VSVLTVLHQDWLNGKEYKCKVSNKALPAPIEKTISKAKGQPREPQVYTLPPSRDELTKN | |
| | QVSLSCAVKGFYPSDIAVEWESNGQPENNYKTTPPVLDSDGSFFLVSKLTVDKSRWQQG | |
| | NVFSCSVMHEALHNHYTQKSLSLSPGKGGSGAWSHPQFEK | |
| | L: | |
| | DIQMTQSPSSLSASVGDRVTITCRASQDVSTAVAWYQQKPGKAPKLLIYSASFLYSGVP | |
| | SRFSGSGSGTDFTLTISSLQPEDFATYYCQQYLYHPATFGQGTKVEIKRTVAAPSVFIF | |
| | PPSDEQLKSGTASVVCLLNNFYPREAKVQWKVDNALQSGNSQESVTEQDSKDSTYSLSS | |
| | TLTLSKADYEKHKVYACEVTHQGLSSPVTKSENRGEC | |
| CXCL12- | H: | |
| Tecentriq | EVQLVESGGGLVQPGGSLRLSCAASGFTFSDSWIHWVRQAPGKGLEWVAWISPYGGSTY | |
| IgG LC | YADSVKGRFTISADTSKNTAYLQMNSLRAEDTAVYYCARRHWPGGFDYWGQGTLVTVSS | |
| Fusion | ASTKGPSVFPLAPSSKSTSGGTAALGCLVKDYFPEPVTVSWNSGALTSGVHTFPAVLQS | |
| | SGLYSLSSVVTVPSSSLGTQTYICNVNHKPSNTKVDKKVEPKSCEPKSCDKTHTCPPCP | |
| | APELLGGPSVFLFPPKPKDTLMISRTPEVTCVVVDVSHEDPEVKENWYVDGVEVHNAKT | |
| | KPREEQYGSTYRVVSVLTVLHQDWLNGKEYKCKVSNKALPAPIEKTISKAKGQPREPQV | |
| | YTLPPSRDELTKNQVSLSCAVKGFYPSDIAVEWESNGQPENNYKTTPPVLDSDGSFFLV | |
| | SKLTVDKSRWQQGNVFSCSVMHEALHNHYTQKSLSLSPGKGGSGAWSHPQFEK | |
| | L: | |
| | KPVSLSYRCPCRFFESHVARANVKHLKILNTPNCALQIVARLKNNNRQVCIDPKLKWIQ | |
| | EYLEKALNKRFKMPWDKTHTGGSGSAGGLNDIFEAQKIEWHESSNSDIQMTQSPSSLSA | |
| | SVGDRVTITCRASQDVSTAVAWYQQKPGKAPKLLIYSASFLYSGVPSRFSGSGSGTDFT | |
| | LTISSLQPEDFATYYCQQYLYHPATFGQGTKVEIKRTVAAPSVFIFPPSDEQLKSGTAS | |
| | VVCLLNNFYPREAKVQWKVDNALQSGNSQESVTEQDSKDSTYSLSSTLTLSKADYEKHK | |
| | VYACEVTHQGLSSPVTKSENRGEC | |
| Pertuzumab | Knob: | |
| Isotype | EVQLVESGGGLVQPGGSLRLSCAASGFRIYSYYSYIGWVRRAPGKGEELVARIYPSSGY | |
| | TYYADSVKGRFTISADTSKNTAYLQMNSLRAEDTAVYYCARWDFASPYYPGSSGLDYWG | |
| | QGTLVTVSSEPKSCDKTHTCPPCPAPELLGGPSVFLFPPKPKDTLMISRTPEVTCVVVD | |
| | VSHEDPEVKFNWYVDGVEVHNAKTKPREEQYGSTYRVVSVLTVLHQDWLNGKEYKCKVS | |
| | NKALPAPIEKTISKAKGQPREPQVYTLPPSRDELTKNQVSLWCLVKGFYPSDIAVEWES | |
| | NGQPENNYKTTPPVLDSDGSFFLYSKLTVDKSRWQQGNVFSCSVMHEALHNHYTQKSLS | |
| | LSPGKGGSHHHHHH | |
| | Hole: | |
| | EVQLVESGGGLVQPGGSLRLSCAASGFTFTDYTMDWVRQAPGKGLEWVADVNPNSGGSI | |
| | YNQRFKGRFTLSVDRSKNTLYLQMNSLRAEDTAVYYCARNLGPSFYFDYWGQGTLVTVS | |
| | SASTKGPSVFPLAPSSKSTSGGTAALGCLVKDYFPEPVTVSWNSGALTSGVHTFPAVLQ | |
| | SSGLYSLSSVVTVPSSSLGTQTYICNVNHKPSNTKVDKKVEPKSCEPKSCDKTHTCPPC | |
| | PAPELLGGPSVFLFPPKPKDTLMISRTPEVTCVVVDVSHEDPEVKENWYVDGVEVHNAK | |
| | TKPREEQYGSTYRVVSVLTVLHQDWLNGKEYKCKVSNKALPAPIEKTISKAKGQPREPQ | |
| | VYTLPPSRDELTKNQVSLSCAVKGFYPSDIAVEWESNGQPENNYKTTPPVLDSDGSFFL | |
| | VSKLTVDKSRWQQGNVFSCSVMHEALHNHYTQKSLSLSPGKGGSGAWSHPQFEK | |
| | L: | |
| | DIQMTQSPSSLSASVGDRVTITCKASQDVSIGVAWYQQKPGKAPKLLIYSASYRYTGVP | |
| | SRFSGSGSGTDFTLTISSLQPEDFATYYCQQYYIYPYTFGQGTKVEIKRTVAAPSVFIF | |
| | PPSDEQLKSGTASVVCLLNNFYPREAKVQWKVDNALQSGNSQESVTEQDSKDSTYSLSS | |
| | TLTLSKADYEKHKVYACEVTHQGLSSPVTKSENRGEC | |
| Nivolumab | Knob: | |
| Isotype | EVQLVESGGGLVQPGGSLRLSCAASGFRIYSYYSYIGWVRRAPGKGEELVARIYPSSGY | |
| | TYYADSVKGRFTISADTSKNTAYLQMNSLRAEDTAVYYCARWDFASPYYPGSSGLDYWG | |
| | QGTLVTVSSEPKSCDKTHTCPPCPAPELLGGPSVFLFPPKPKDTLMISRTPEVTCVVVD | |
| | VSHEDPEVKFNWYVDGVEVHNAKTKPREEQYGSTYRVVSVLTVLHQDWLNGKEYKCKVS | |
| | NKALPAPIEKTISKAKGQPREPQVYTLPPSRDELTKNQVSLWCLVKGFYPSDIAVEWES | |
| | NGQPENNYKTTPPVLDSDGSFFLYSKLTVDKSRWQQGNVFSCSVMHEALHNHYTQKSLS | |
| | LSPGKGGSHHHHHH | |
| | Hole: | |
| | QVQLVESGGGVVQPGRSLRLDCKASGITFSNSGMHWVRQAPGKGLEWVAVIWYDGSKRY | |
| | YADSVKGRFTISRDNSKNTLFLQMNSLRAEDTAVYYCATNDDYWGQGTLVTVSSASTKG | |
| | PSVFPLAPSSKSTSGGTAALGCLVKDYFPEPVTVSWNSGALTSGVHTFPAVLQSSGLYS | |
| | LSSVVTVPSSSLGTQTYICNVNHKPSNTKVDKKVEPKSCEPKSCDKTHTCPPCPAPELL | |
| | GGPSVFLFPPKPKDTLMISRTPEVTCVVVDVSHEDPEVKFNWYVDGVEVHNAKTKPREE | |
| | QYGSTYRVVSVLTVLHQDWLNGKEYKCKVSNKALPAPIEKTISKAKGQPREPQVYTLPP | |
| | SRDELTKNQVSLSCAVKGFYPSDIAVEWESNGQPENNYKTTPPVLDSDGSFFLVSKLTV | |
| | DKSRWQQGNVFSCSVMHEALHNHYTQKSLSLSPGKGGSGAWSHPQFEK | |
| | L: | |
| | EIVLTQSPATLSLSPGERATLSCRASQSVSSYLAWYQQKPGQAPRLLIYDASNRATGIP | |
| | ARFSGSGSGTDFTLTISSLEPEDFAVYYCQQSSNWPRTFGQGTKVEIKRTVAAPSVFIF | |
| | PPSDEQLKSGTASVVCLLNNFYPREAKVQWKVDNALQSGNSQESVTEQDSKDSTYSLSS | |
| | TLTLSKADYEKHKVYACEVTHQGLSSPVTKSENRGEC | |
| Nimotuzumab | Knob: | |
| Isotype | EVQLVESGGGLVQPGGSLRLSCAASGFRIYSYYSYIGWVRRAPGKGEELVARIYPSSGY | |
| | TYYADSVKGRFTISADTSKNTAYLQMNSLRAEDTAVYYCARWDFASPYYPGSSGLDYWG | |
| | QGTLVTVSSEPKSCDKTHTCPPCPAPELLGGPSVFLFPPKPKDTLMISRTPEVTCVVVD | |
| | VSHEDPEVKFNWYVDGVEVHNAKTKPREEQYGSTYRVVSVLTVLHQDWLNGKEYKCKVS | |
| | NKALPAPIEKTISKAKGQPREPQVYTLPPSRDELTKNQVSLWCLVKGFYPSDIAVEWES | |
| | NGQPENNYKTTPPVLDSDGSFFLYSKLTVDKSRWQQGNVFSCSVMHEALHNHYTQKSLS | |
| | LSPGKGGSHHHHHH | |
| | Hole: | |
| | QVQLQQSGAEVKKPGSSVKVSCKASGYTFTNYYIYWVRQAPGQGLEWIGGINPTSGGSN | |
| | FNEKFKTRVTITVDESTNTAYMELSSLRSEDTAFYFCARQGLWFDSDGRGFDFWGQGST | |
| | VTVSSASTKGPSVFPLAPSSKSTSGGTAALGCLVKDYFPEPVTVSWNSGALTSGVHTFP | |
| | AVLQSSGLYSLSSVVTVPSSSLGTQTYICNVNHKPSNTKVDKKVEPKSCDKTHTCPPCP | |
| | APELLGGPSVFLFPPKPKDTLMISRTPEVTCVVVDVSHEDPEVKENWYVDGVEVHNAKT | |
| | KPREEQYGSTYRVVSVLTVLHQDWLNGKEYKCKVSNKALPAPIEKTISKAKGQPREPQV | |
| | YTLPPSRDELTKNQVSLSCAVKGFYPSDIAVEWESNGQPENNYKTTPPVLDSDGSFFLV | |
| | SKLTVDKSRWQQGNVFSCSVMHEALHNHYTQKSLSLSPGKGGSGAWSHPQFEK | |
| | L: | |
| | DIQMTQSPSSLSASVGDRVTITCRSSQNIVHSNGNTYLDWYQQTPGKAPKLLIYKVSNR | |
| | FSGVPSRFSGSGSGTDFTFTISSLQPEDIATYYCFQYSHVPWTFGQGTKLQITREVAAP | |
| | SVFIFPPSDEQLKSGTASVVCLLNNFYPREAKVQWKVDNALQSGNSQESVTEQDSKDST | |
| | YSLSSTLTLSKADYEKHKVYACEVTHQGLSSPVTKSFNRGECGGSDYKDDDDK | |
| Necitumumab | Knob: | |
| Isotype | EVQLVESGGGLVQPGGSLRLSCAASGFRIYSYYSYIGWVRRAPGKGEELVARIYPSSGY | |
| | TYYADSVKGRFTISADTSKNTAYLQMNSLRAEDTAVYYCARWDFASPYYPGSSGLDYWG | |
| | QGTLVTVSSEPKSCDKTHTCPPCPAPELLGGPSVFLFPPKPKDTLMISRTPEVTCVVVD | |
| | VSHEDPEVKFNWYVDGVEVHNAKTKPREEQYGSTYRVVSVLTVLHQDWLNGKEYKCKVS | |
| | NKALPAPIEKTISKAKGQPREPQVYTLPPSRDELTKNQVSLWCLVKGFYPSDIAVEWES | |
| | NGQPENNYKTTPPVLDSDGSFFLYSKLTVDKSRWQQGNVFSCSVMHEALHNHYTQKSLS | |
| | LSPGKGGSHHHHHH | |
| | Hole: | |
| | QVQLQESGPGLVKPSQTLSLTCTVSGGSISSGDYYWSWIRQPPGKGLEWIGYIYYSGST | |
| | DYNPSLKSRVTMSVDTSKNQFSLKVNSVTAADTAVYYCARVSIFGVGTFDYWGQGTLVT | |
| | VSSASTKGPSVLPLAPSSKSTSGGTAALGCLVKDYFPEPVTVSWNSGALTSGVHTFPAV | |
| | LQSSGLYSLSSVVTVPSSSLGTQTYICNVNHKPSNTKVDKKV | |
| | EPKSCDKTHTCPPCPAPELLGGPSVFLFPPKPKDTLMISRTPEVTCVVVDVSHEDPEVK | |
| | FNWYVDGVEVHNAKTKPREEQYNSTYRVVSVLTVLHQDWLNGKEYKCKVSNKALPAPIE | |
| | KTISKAKGQPREPQVYTLPPSRDELTKNQVSLSCAVKGFYPSDIAVEWESNGQPENNYK | |
| | TTPPVLDSDGSFFLVSKLTVDKSRWQQGNVFSCSVMHEALHNHYTQKSLSLSPGK | |
| | L: | |
| | EIVMTQSPATLSLSPGERATLSCRASQSVSSYLAWYQQKPGQAPRLLIYDASNRATGIP | |
| | ARFSGSGSGTDFTLTISSLEPEDFAVYYCHQYGSTPLTFGGGTKAEIKRTVAAPSVFIF | |
| | PPSDEQLKSGTASVVCLLNNFYPREAKVQWKVDNALQSGNSQESVTEQDSKDSTYSLSS | |
| | TLTLSKADYEKHKVYACEVTHQGLSSPVTKSENRGEC | |
| Matuzumab | Knob: | |
| Isotype | EVQLVESGGGLVQPGGSLRLSCAASGFRIYSYYSYIGWVRRAPGKGEELVARIYPSSGY | |
| | TYYADSVKGRFTISADTSKNTAYLQMNSLRAEDTAVYYCARWDFASPYYPGSSGLDYWG | |
| | QGTLVTVSSEPKSCDKTHTCPPCPAPELLGGPSVFLFPPKPKDTLMISRTPEVTCVVVD | |
| | VSHEDPEVKFNWYVDGVEVHNAKTKPREEQYGSTYRVVSVLTVLHQDWLNGKEYKCKVS | |
| | NKALPAPIEKTISKAKGQPREPQVYTLPPSRDELTKNQVSLWCLVKGFYPSDIAVEWES | |
| | NGQPENNYKTTPPVLDSDGSFFLYSKLTVDKSRWQQGNVFSCSVMHEALHNHYTQKSLS | |
| | LSPGKGGSHHHHHH | |
| | Hole: | |
| | QVQLVQSGAEVKKPGASVKVSCKASGYTFTSHWMHWVRQAPGQGLEWIGEFNPSNGRTN | |
| | YNEKFKSKATMTVDTSTNTAYMELSSLRSEDTAVYYCASRDYDYDGRYFDYWGQGTLVT | |
| | VSSASTKGPSVFPLAPSSKSTSGGTAALGCLVKDYFPEPVTVSWNSGALTSGVHTFPAV | |
| | LQSSGLYSLSSVVTVPSSSLGTQTYICNVNHKPSNTKVDKKVEPKSCDKTHTCPPCPAP | |
| | ELLGGPSVFLFPPKPKDTLMISRTPEVTCVVVDVSHEDPEVKENWYVDGVEVHNAKTKP | |
| | REEQYNSTYRVVSVLTVLHQDWLNGKEYKCKVSNKALPAPIEKTISKAKGQPREPQVYT | |
| | LPPSRDELTKNQVSLSCAVKGFYPSDIAVEWESNGQPENNYKTTPPVLDSDGSFFLVSK | |
| | LTVDKSRWQQGNVFSCSVMHEALHNHYTQKSLSLSPGK | |
| | L: | |
| | DIQMTQSPSSLSASVGDRVTITCSASSSVTYMYWYQQKPGKAPKLLIYDTSNLASGVPS | |
| | RFSGSGSGTDYTFTISSLQPEDIATYYCQQWSSHIFTFGQGTKVEIKRTVAAPSVFIFP | |
| | PSDEQLKSGTASVVCLLNNFYPREAKVQWKVDNALQSGNSQESVTEQDSKDSTYSLSST | |
| | LTLSKADYEKHKVYACEVTHQGLSSPVTKSENRGEC | |
| Depatuxizumab | Knob: | |
| Isotype | EVQLVESGGGLVQPGGSLRLSCAASGFRIYSYYSYIGWVRRAPGKGEELVARIYPSSGY | |
| | TYYADSVKGRFTISADTSKNTAYLQMNSLRAEDTAVYYCARWDFASPYYPGSSGLDYWG | |
| | QGTLVTVSSEPKSCDKTHTCPPCPAPELLGGPSVFLFPPKPKDTLMISRTPEVTCVVVD | |
| | VSHEDPEVKFNWYVDGVEVHNAKTKPREEQYGSTYRVVSVLTVLHQDWLNGKEYKCKVS | |
| | NKALPAPIEKTISKAKGQPREPQVYTLPPSRDELTKNQVSLWCLVKGFYPSDIAVEWES | |
| | NGQPENNYKTTPPVLDSDGSFFLYSKLTVDKSRWQQGNVFSCSVMHEALHNHYTQKSLS | |
| | LSPGKGGSHHHHHH | |
| | Hole: | |
| | QVQLQESGPGLVKPSQTLSLTCTVSGYSISSDFAWNWIRQPPGKGLEWMGYISYSGNTR | |
| | YQPSLKSRITISRDTSKNQFFLKLNSVTAADTATYYCVTAGRGFPYWGQGTLVTVSSAS | |
| | TKGPSVFPLAPSSKSTSGGTAALGCLVKDYFPEPVTVSWNSGALTSGVHTFPAVLQSSG | |
| | LYSLSSVVTVPSSSLGTQTYICNVNHKPSNTKVDKKVEPKSCDKTHTCPPCPAPELLGG | |
| | PSVFLFPPKPKDTLMISRTPEVTCVVVDVSHEDPEVKENWYVDGVEVHNAKTKPREEQY | |
| | GSTYRVVSVLTVLHQDWLNGKEYKCKVSNKALPAPIEKTISKAKGQPREPQVYTLPPSR | |
| | DELTKNQVSLSCAVKGFYPSDIAVEWESNGQPENNYKTTPPVLDSDGSFFLVSKLTVDK | |
| | SRWQQGNVFSCSVMHEALHNHYTQKSLSLSPGKGGSGAWSHPQFEK | |
| | L: | |
| | DIQMTQSPSSMSVSVGDRVTITCHSSQDINSNIGWLQQKPGKSFKGLIYHGTNLDDGVP | |
| | SRFSGSGSGTDYTLTISSLQPEDFATYYCVQYAQFPWTFGGGTKLEIKRTVAAPSVFIF | |
| | PPSDEQLKSGTASVVCLLNNFYPREAKVQWKVDNALQSGNSQESVTEQDSKDSTYSLSS | |
| | TLTLSKADYEKHKVYACEVTHQGLSSPVTKSFNRGECGGSDYKDDDDK | |
| Panitumumab | Knob: | |
| Isotype | EVQLVESGGGLVQPGGSLRLSCAASGFRIYSYYSYIGWVRRAPGKGEELVARIYPSSGY | |
| | TYYADSVKGRFTISADTSKNTAYLQMNSLRAEDTAVYYCARWDFASPYYPGSSGLDYWG | |
| | QGTLVTVSSEPKSCDKTHTCPPCPAPELLGGPSVFLFPPKPKDTLMISRTPEVTCVVVD | |
| | VSHEDPEVKFNWYVDGVEVHNAKTKPREEQYGSTYRVVSVLTVLHQDWLNGKEYKCKVS | |
| | NKALPAPIEKTISKAKGQPREPQVYTLPPSRDELTKNQVSLWCLVKGFYPSDIAVEWES | |
| | NGQPENNYKTTPPVLDSDGSFFLYSKLTVDKSRWQQGNVFSCSVMHEALHNHYTQKSLS | |
| | LSPGKGGSHHHHHH | |
| | Hole: | |
| | QVQLQESGPGLVKPSETLSLTCTVSGGSVSSGDYYWTWIRQSPGKGLEWIGHIYYSGNT | |
| | NYNPSLKSRLTISIDTSKTQFSLKLSSVTAADTAIYYCVRDRVTGAFDIWGQGTMVTVS | |
| | SASTKGPSVFPLAPCSRSTSESTAALGCLVKDYFPEPVTVSWNSGALTSGVHTFPAVLQ | |
| | SSGLYSLSSVVTVPSSSLGTQTYICNVNHKPSNTKVDKKVEPKSCDKTHTCPPCPAPEL | |
| | LGGPSVFLFPPKPKDTLMISRTPEVTCVVVDVSHEDPEVKENWYVDGVEVHNAKTKPRE | |
| | EQYNSTYRVVSVLTVLHQDWLNGKEYKCKVSNKALPAPIEKTISKAKGQPREPQVYTLP | |
| | PSRDELTKNQVSLSCAVKGFYPSDIAVEWESNGQPENNYKTTPPVLDSDGSFFLVSKLT | |
| | VDKSRWQQGNVFSCSVMHEALHNHYTQKSLSLSPGK | |
| | L: | |
| | DIQMTQSPSSLSASVGDRVTITCQASQDISNYLNWYQQKPGKAPKLLIYDASNLETGVP | |
| | SRFSGSGSGTDFTFTISSLQPEDIATYFCQHFDHLPLAFGGGTKVEIKRTVAAPSVFIF | |
| | PPSDEQLKSGTASVVCLLNNFYPREAKVQWKVDNALQSGNSQESVTEQDSKDSTYSLSS | |
| | TLTLSKADYEKHKVYACEVTHQGLSSPVTKSENRGEC | |
| Bevacizumab | Knob: | |
| Isotype | EVQLVESGGGLVQPGGSLRLSCAASGFRIYSYYSYIGWVRRAPGKGEELVARIYPSSGY | |
| | TYYADSVKGRFTISADTSKNTAYLQMNSLRAEDTAVYYCARWDFASPYYPGSSGLDYWG | |
| | QGTLVTVSSEPKSCDKTHTCPPCPAPELLGGPSVFLFPPKPKDTLMISRTPEVTCVVVD | |
| | VSHEDPEVKFNWYVDGVEVHNAKTKPREEQYGSTYRVVSVLTVLHQDWLNGKEYKCKVS | |
| | NKALPAPIEKTISKAKGQPREPQVYTLPPSRDELTKNQVSLWCLVKGFYPSDIAVEWES | |
| | NGQPENNYKTTPPVLDSDGSFFLYSKLTVDKSRWQQGNVFSCSVMHEALHNHYTQKSLS | |
| | LSPGKGGSHHHHHH | |
| | Hole: | |
| | EVQLVESGGGLVQPGGSLRLSCAASGYTFTNYGMNWVRQAPGKGLEWVGWINTYTGEPT | |
| | YAADFKRRFTFSLDTSKSTAYLQMNSLRAEDTAVYYCAKYPHYYGSSHWYFDVWGQGTL | |
| | VTVSSASTKGPSVFPLAPSSKSTSGGTAALGCLVKDYFPEPVTVSWNSGALTSGVHTFP | |
| | AVLQSSGLYSLSSVVTVPSSSLGTQTYICNVNHKPSNTKVDKKVEPKSCDKTHTCPPCP | |
| | APELLGGPSVFLFPPKPKDTLMIS | |
| | RTPEVTCVVVDVSHEDPEVKENWYVDGVEVHNAKTKPREEQYGSTYRVVSVLTVLHQDW | |
| | LNGKEYKCKVSNKALPAPIEKTISKAKGQPREPQVYTLPPSRDELTKNQVSLSCAVKGF | |
| | YPSDIAVEWESNGQPENNYKTTPPVLDSDGSFFLVSKLTVDKSRWQQGNVFSCSVMHEA | |
| | LHNHYTQKSLSLSPGKGGSGAWSHPQFEK | |
| | L: | |
| | ASDIQMTQSPSSLSASVGDRVTITCSASQDISNYLNWYQQKPGKAPKVLIYFTSSLHSG | |
| | VPSRFSGSGSGTDFTLTISSLQPEDFATYYCQQYSTVPWTFGQGTKVEIKRTVAAPSVE | |
| | IFPPSDEQLKSGTASVVCLLNNFYPREAKVQWKVDNALQSGNSQESVTEQDSKDSTYSL | |
| | SSTLTLSKADYEKHKVYACEVTHQGLSSPVTKSENRGEC | |
| Adalimumab | Knob: | |
| Isotype | EVQLVESGGGLVQPGGSLRLSCAASGFRIYSYYSYIGWVRRAPGKGEELVARIYPSSGY | |
| | TYYADSVKGRFTISADTSKNTAYLQMNSLRAEDTAVYYCARWDFASPYYPGSSGLDYWG | |
| | QGTLVTVSSEPKSCDKTHTCPPCPAPELLGGPSVFLFPPKPKDTLMISRTPEVTCVVVD | |
| | VSHEDPEVKFNWYVDGVEVHNAKTKPREEQYGSTYRVVSVLTVLHQDWLNGKEYKCKVS | |
| | NKALPAPIEKTISKAKGQPREPQVYTLPPSRDELTKNQVSLWCLVKGFYPSDIAVEWES | |
| | NGQPENNYKTTPPVLDSDGSFFLYSKLTVDKSRWQQGNVFSCSVMHEALHNHYTQKSLS | |
| | LSPGKGGSHHHHHH | |
| | Hole: | |
| | EVQLVESGGGLVQPGRSLRLSCAASGFTFDDYAMHWVRQAPGKGLEWVSAITWNSGHID | |
| | YADSVEGRFTISRDNAKNSLYLQMNSLRAEDTAVYYCAKVSYLSTASSLDYWGQGTLVT | |
| | VSSASTKGPSVFPLAPSSKSTSGGTAALGCLVKDYFPEPVTVSWNSGALTSGVHTFPAV | |
| | LQSSGLYSLSSVVTVPSSSLGTQTYICNVNHKPSNTKVDKKVEPKSCDKTHTCPPCPAP | |
| | ELLGGPSVFLFPPKPKDTLMISRTPEVTCVVVDVSHEDPEVKFNWYVDGVEVHNAKTKP | |
| | REEQYGSTYRVVSVLTVLHQDWLNGKEYKCKVSNKALPAPIEKTISKAKGQPREPQVYT | |
| | LPPSRDELTKNQVSLSCAVKGFYPSDIAVEWESNGQPENNYKTTPPVLDSDGSFFLVSK | |
| | LTVDKSRWQQGNVFSCSVMHEALHNHYTQKSLSLSPGKGGSGAWSHPQFEK | |
| | L: | |
| | DIQMTQSPSSLSASVGDRVTITCRASQGIRNYLAWYQQKPGKAPKLLIYAASTLQSGVP | |
| | SRFSGSGSGTDFTLTISSLQPEDVATYYCQRYNRAPYTFGQGTKVEIKRTVAAPSVFIF | |
| | PPSDEQLKSGTASVVCLLNNFYPREAKVQWKVDNALQSGNSQESVTEQDSKDSTYSLSS | |
| | TLTLSKADYEKHKVYACEVTHQGLSSPVTKSENRGEC | |

### Immunoconjugates

The present disclosure further comprises immunoconjugates comprising any of the bispecific binding agents disclosed herein. The term “immunoconjugate” or “conjugate” as used herein refers to a compound or a derivative thereof that is linked to a binding agent, such as the bispecific binding agents provided herein. The immunoconjugate of the present disclosure generally comprises a binding agent, such as the bispecific binding agents provided herein and a small molecule. In some embodiments, the immunoconjugate further comprises a linker.

A “linker” is any chemical moiety that is capable of linking a compound, for example, the small molecule disclosed herein, to a binding agent, such as the bispecific binding agents provided herein in a stable and covalent manner. Linkers can be susceptible to or be substantially resistant to acid-induced cleavage, light-induced cleavage, peptidase-induced cleavage, esterase-induced cleavage, and disulfide bond cleavage, at conditions under which the compound or the antibody remains active. Suitable linkers are well known in the art and include, for example, disulfide groups, thioether groups, acid labile groups, photolabile groups, peptidase labile groups and esterase labile groups. Linkers also include charged linkers, and hydrophilic forms thereof as described herein and known in the art. In certain embodiments, the linker is selected from the group consisting of a cleavable linker, a non-cleavable linker, a hydrophilic linker, and a dicarboxylic acid based linker. In an exemplary embodiment, the linker is a non-cleavable linker. In another exemplary embodiment, the linker is a spacer, such as PEG4. In other embodiments, the small molecule does not dissociate from the binding agent.

The small molecule encompassed by the present disclosure can be any small molecule one skilled in the art deems suitable for the use, for example, targeted degradation of a protein of interest. The small molecules can be conjugated to the binding agent, such as the bispecific binding agents provided herein by methods known in the art. Some exemplary conjugation methods include, without limitations, methionine using oxaziridine based reagents, cysteine labeling with a maleimide based reagent or disulfide exchange reagent, lysine reactive activated esters, utilizing incorporation of an unnatural amino acid containing a reactive handle for conjugation, and N-Terminal or C-terminal conjugation. Some methods use engineered amino acids, such as aldehydes, for reactive conjugation. Other methods include Tag based bioconjugation methods. It is understood that the present disclosure is not limited by the few examples listed here, and other commonly known conjugation methods can also be used in making the immunoconjugates disclosed herein.

### Nucleic Acid Molecules

In one aspect, some embodiments disclosed herein relate to nucleic acid molecules comprising nucleotide sequences encoding the bispecific binding agents of the disclosure, including expression cassettes, and expression vectors containing these nucleic acid molecules operably linked to heterologous nucleic acid sequences such as, for example, regulatory sequences which direct in vivo expression of the bispecific binding agents in a host cell. In some embodiments, the bispecific binding agent descaled herein is expressed from a single genetic construct.

Nucleic acid molecules of the present disclosure can be nucleic acid molecules of any length, including nucleic acid molecules that are generally between about 5 Kb and about 50 Kb, for example between about 5 Kb and about 40 Kb, between about 5 Kb and about 30 Kb, between about 5 Kb and about 20 Kb, or between about 10 Kb and about 50 Kb, for example between about 15 Kb to 30 Kb, between about 20 Kb and about 50 Kb, between about 20 Kb and about 40 Kb, about 5 Kb and about 25 Kb, or about 30 Kb and about 50 Kb.

In some embodiments, the nucleotide sequence is incorporated into an expression cassette or an expression vector. It will be understood that an expression cassette generally includes a construct of genetic material that contains coding sequences and enough regulatory information to direct proper transcription and/or translation of the coding sequences in a recipient cell, in vivo and/or ex vivo. Generally, the expression cassette may be inserted into a vector for targeting to a desired host cell or tissue and/or into an individual. Thus, in some embodiments, an expression cassette of the disclosure comprises a nucleotide sequence encoding a bispecific binding agent operably linked to expression control elements sufficient to guide expression of the cassette in vivo. In some embodiments, the expression control element comprises a promoter and/or an enhancer and optionally, any or a combination of other nucleic acid sequences capable of effecting transcription and/or translation of the coding sequence.

In some embodiments, the nucleotide sequence is incorporated into an expression vector. Vectors generally comprise a recombinant polynucleotide construct designed for transfer between host cells, which may be used for the purpose of transformation, i.e., the introduction of heterologous DNA into a host cell. As such, in some embodiments, the vector can be a replicon, such as a plasmid, phage, or cosmid, into which another DNA segment may be inserted so as to bring about the replication of the inserted segment. Expression vectors further include a promoter operably linked to the recombinant polynucleotide, such that the recombinant polynucleotide is expressed in appropriate cells, under appropriate conditions. In some embodiments, the expression vector is an integrating vector, which can integrate into host nucleic acids.

In some embodiments, the expression vector is a viral vector, which further includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s). The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. Retroviral vectors contain structural and functional genetic elements, or portions thereof, that are primarily derived from a retrovirus. Lentiviral vectors are viral vectors or plasmids containing structural and functional genetic elements, or portions thereof, including LTRs that are primarily derived from a lentivirus.

The nucleic acid sequences can be optimized for expression in the host cell of interest. For example, the G-C content of the sequence can be adjusted to levels average for a given cellular host, as calculated by reference to known genes expressed in the host cell. Methods for codon optimization are known in the art. Codon usages within the coding sequence of the proteins disclosed herein can be optimized to enhance expression in the host cell, such that about 1%, about 5%, about 10%, about 25%, about 50%, about 75%, or up to 100% of the codons within the coding sequence have been optimized for expression in a particular host cell.

Some embodiments disclosed herein relate to vectors or expression cassettes including a recombinant nucleic acid molecule encoding the proteins disclosed herein. The expression cassette generally contains coding sequences and sufficient regulatory information to direct proper transcription and/or translation of the coding sequences in a recipient cell, in vivo and/or ex vivo. The expression cassette may be inserted into a vector for targeting to a desired host cell and/or into an individual. An expression cassette can be inserted into a plasmid, cosmid, virus, autonomously replicating polynucleotide molecule, or bacteriophage, as a linear or circular, single-stranded or double-stranded, DNA or RNA polynucleotide, derived from any source, capable of genomic integration or autonomous replication, including a nucleic acid molecule where one or more nucleic acid sequences has been linked in a functionally operative manner, i.e., operably linked.

Also provided herein are vectors, plasmids, or viruses containing one or more of the nucleic acid molecules encoding any bispecific binding agent or engineered protein disclosed herein. The nucleic acid molecules can be contained within a vector that is capable of directing their expression in, for example, a cell that has been transformed/transduced with the vector. Suitable vectors for use in eukaryotic and prokaryotic cells are known in the art and are commercially available, or readily prepared by a skilled artisan. See for example, Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

DNA vectors can be introduced into eukaryotic cells via conventional transformation or transfection techniques. Suitable methods for transforming or transfecting host cells can be found in Sambrook et al. (2012, supra) and other standard molecular biology laboratory manuals, such as, calcium phosphate transfection, DEAE-dextran mediated transfection, transfection, microinjection, cationic lipid-mediated transfection, electroporation, transduction, scrape loading, ballistic introduction, nucleoporation, hydrodynamic shock, and infection.

Viral vectors that can be used in the disclosure include, for example, retrovirus vectors, adenovirus vectors, and adeno-associated virus vectors, lentivirus vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.).

The precise components of the expression system are not critical. For example, a bispecific binding agent as disclosed herein can be produced in a eukaryotic host, such as a mammalian cells (e.g., COS cells, NIH 3T3 cells, or HeLa cells). These cells are available from many sources, including the American Type Culture Collection (Manassas, Va.). In selecting an expression system, it matters only that the components are compatible with one another. Artisans or ordinary skill are able to make such a determination. Furthermore, if guidance is required in selecting an expression system, skilled artisans may consult P. Jones, “Vectors: Cloning Applications”, John Wiley and Sons, New York, N.Y., 2009).

The nucleic acid molecules provided can contain naturally occurring sequences, or sequences that differ from those that occur naturally but encode the same gene product because the genetic code is degenerate. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (e.g., comprising either a sense or an antisense strand).

The nucleic acid molecules are not limited to sequences that encode polypeptides (e.g., antibodies); some or all of the non-coding sequences that lie upstream or downstream from a coding sequence (e.g., the coding sequence of a bispecific binding agent) can also be included. Those of ordinary skill in the art of molecular biology are familiar with routine procedures for isolating nucleic acid molecules. They can, for example, be generated by treatment of genomic DNA with restriction endonucleases, or by the polymerase chain reaction (PCR). In the event the nucleic acid molecule is a ribonucleic acid (RNA), transcripts can be produced, for example, by in vitro transcription.

### Recombinant Cells and Cell Cultures

The nucleic acid of the present disclosure can be introduced into a host cell, such as a human B lymphocyte, to produce a recombinant cell containing the nucleic acid molecule. Accordingly, some embodiments of the disclosure relate to methods for making recombinant cells, including the steps of: (a) providing a cell capable of protein expression and (b) contacting the provided cell with any of the recombinant nucleic acids described herein.

Introduction of the nucleic acid molecules of the disclosure into cells can be achieved by viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Accordingly, in some embodiments, the nucleic acid molecules are delivered to cells by viral or non-viral delivery vehicles known in the art. For example, the nucleic acid molecule can be stably integrated in the host genome, or can be episomally replicating, or present in the recombinant host cell as a mini-circle expression vector for a stable or transient expression. Accordingly, in some embodiments disclosed herein, the nucleic acid molecule is maintained and replicated in the recombinant host cell as an episomal unit. In some embodiments, the nucleic acid molecule is stably integrated into the genome of the recombinant cell. Stable integration can be completed using classical random genomic recombination techniques or with more precise genome editing techniques such as using guide RNA directed CRISPR/Cas9, or DNA-guided endonuclease genome editing NgAgo (Natronobacterium gregoryi Argonaute), or TALENs genome editing (transcription activator-like effector nucleases). In some embodiments, the nucleic acid molecule present in the recombinant host cell as a mini-circle expression vector for a stable or transient expression.

The nucleic acid molecules can be encapsulated in a viral capsid or a lipid nanoparticle. For example, introduction of nucleic acids into cells may be achieved by viral transduction. In a non-limiting example, adeno-associated virus (AAV) is a non-enveloped virus that can be engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral systems are also suitable for nucleic acid delivery and gene therapy via viral transduction. Lentiviral vectors offer several attractive properties as gene-delivery vehicles, including: (i) sustained gene delivery through stable vector integration into host genome; (ii) the ability to infect both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) potentially safer integration site profile; and (vii) a relatively easy system for vector manipulation and production.

In some embodiments, host cells are genetically engineered (e.g., transduced, transformed, or transfected) with, for example, a vector comprising a nucleic acid sequence encoding a bispecific binding agent as described herein, either a virus-derived expression vector or a vector for homologous recombination further comprising nucleic acid sequences homologous to a portion of the genome of the host cell. Host cells can be either untransformed cells or cells that have already been transfected with one or more nucleic acid molecules.

In some embodiments, the recombinant cell is a prokaryotic cell or a eukaryotic cell. In some embodiments, the cell is transformed in vivo. In some embodiments, the cell is transformed ex vivo. In some embodiments, the cell is transformed in vitro. In some embodiments, the recombinant cell is a eukaryotic cell. In some embodiments, the recombinant cell is an animal cell. In some embodiments, the animal cell is a mammalian cell. In some embodiments, the animal cell is a human cell. In some embodiments, the cell is a non-human primate cell. In some embodiments, the mammalian cell is an immune cell, a neuron, an epithelial cell, and endothelial cell, or a stem cell. In some embodiments, the recombinant cell is an immune system cell, e.g., a lymphocyte (e.g., a T cell or NK cell), or a dendritic cell. In some embodiments, the immune cell is a B cell, a monocyte, a natural killer (NK) cell, a basophil, an eosinophil, a neutrophil, a dendritic cell, a macrophage, a regulatory T cell, a helper T cell, a cytotoxic T cell, or other T cell. In some embodiments, the immune system cell is a T lymphocyte.

In some embodiments, the cell is a stem cell. In some embodiments, the cell is a hematopoietic stem cell. In some embodiments of the cell, the cell is a lymphocyte. In some embodiments, the cell is a precursor T cell or a T regulatory (Treg) cell. In some embodiments, the cell is a CD34+, CD8+, or a CD4+ cell. In some embodiments, the cell is a CD8+T cytotoxic lymphocyte cell selected from the group consisting of naive CD8+ T cells, central memory CD8+ T cells, effector memory CD8+ T cells, and bulk CD8+ T cells. In some embodiments of the cell, the cell is a CD4+T helper lymphocyte cell selected from the group consisting of naive CD4+ T cells, central memory CD4+ T cells, effector memory CD4+ T cells, and bulk CD4+ T cells. In some embodiments, the cell can be obtained by leukapheresis performed on a sample obtained from a human subject.

In another aspect, provided herein are various cell cultures including at least one recombinant cell as disclosed herein, and a culture medium. Generally, the culture medium can be any one of suitable culture media for the cell cultures described herein. Techniques for transforming a wide variety of the above-mentioned host cells and species are known in the art and described in the technical and scientific literature. Accordingly, cell cultures including at least one recombinant cell as disclosed herein are also within the scope of this application. Methods and systems suitable for generating and maintaining cell cultures are known in the art.

### Synthesis

Bispecific binding agents can be synthesized using the techniques of recombinant DNA and protein expression. For example, for the synthesis of DNA encoding a bispecific IgG of the disclosure, suitable DNA sequences encoding the constant domains of the heavy and light chains are widely available. Sequences encoding the selected variable domains are inserted by standard methods, and the resulting nucleic acids encoding full-length heavy and light chains are transduced into suitable host cells and expressed. Alternatively, the nucleic acids can be expressed in a cell-free expression system, which can provide more control over oxidation and reduction conditions, pH, folding, glycosylation, and the like.

In some embodiments, the bispecific binding agents can have two different complementary determining regions (CDRs), each specific for either the target protein or endogenous cell surface receptor. Thus, two different heavy chains and two different light chains are required. In other embodiments, the bispecific binding agents can have one or more CDRs specific for the target protein and a binding domain (e.g., the second binding domain which can be a chemokine) specific for the endogenous cell surface receptor. See, e.g., FIGS. 1B and 2A. These may be expressed in the same host cell, and the resulting product will contain a mixture of homodimers and bispecific heterodimers. Homodimers can be separated from the bispecific antibodies by affinity purification (for example, first using beads coated with one antigen, then beads coated with the other antigen), reduced to monomers, and reassociated. Alternatively, one can employ a “knobs into holes” design, in which a dimerization region of a heavy chain constant region is altered so that the surface either protrudes (“knob”) from the surface (as compared to the wild type structure) or forms a cavity (“hole”) in such a way that the two modified surfaces are still capable of dimerizing. The knob heavy chain and its associated light chain are then expressed in one host cell, and the hole heavy chain and associated light chain are expressed in a different host cell, and the expressed proteins are combined. The asymmetry in the dimerization regions promotes the formation of heterodimers. To obtain dimerization, the two “monomers” (each consisting of a heavy chain and a light chain) are combined under reducing conditions at a moderately basic pH (e.g., about pH 8 to about pH 9) to promote disulfide bond formation between the appropriate heavy chain domains. See, e.g., U.S. Pat. No. 8,216,805 and EP 1870459A1, incorporated herein by reference.

Other methods can be used to promote heavy-chain heterodimerization of the first and second polypeptide chains of bispecific antibodies. For example, in some embodiments, the heavy-chain heterodimerization of the first and second polypeptide chains of the engineered antibodies as disclosed herein can be achieved by a controlled Fab arm exchange method as described by F. L. Aran et al., Proc Natl Acad Sci USA (2013) 110(13):5145-50.

The dimerization process can result in exchange of the light chains between different heavy chain monomers. One method for avoiding this outcome is to replace the binding region of the antibody with a “single chain Fab”, e.g., wherein the light chain CDR is fused to the heavy chain CDR by a linking polypeptide. The Fab region of an IgG (or other antibody) may also be replaced with a scFv, nanobody, and the like.

The binding activity of the engineered antibodies of the disclosure can be assayed by any suitable method known in the art. For example, the binding activity of the engineered antibodies of the disclosure can be determined by, e.g., Scatchard analysis (Munsen et al., Analyt Biochem (1980) 107:220-39). Specific binding may be assessed using techniques known in the art including but not limited to competition ELISA, BIACORE® assays and/or KINEXA® assays. An antibody that preferentially or specifically binds (used interchangeably herein) to a target antigen or target epitope is a term well understood in the art, and methods to determine such specific or preferential binding are also known in the art. An antibody is said to exhibit specific or preferential binding if it reacts or associates more frequently, more rapidly, with greater duration and/or with greater affinity with a particular antigen or epitope than it does with alternative antigens or epitopes. An antibody specifically or preferentially binds to a target if it binds with greater affinity, avidity, more readily, and/or with greater duration than it binds to other substances. Also, an antibody specifically or preferentially binds to a target if it binds with greater affinity, avidity, more readily, and/or with greater duration to that target in a sample than it binds to other substances present in the sample. For example, an antibody that specifically or preferentially binds to a HER2 epitope is an antibody that binds this epitope with greater affinity, avidity, more readily, and/or with greater duration than it binds to other HER2 epitopes or non-HER2 epitopes. It is also understood by reading this definition, for example, that an antibody which specifically or preferentially binds to a first target antigen may or may not specifically or preferentially bind to a second target antigen. As such, specific binding and preferential binding do not necessarily require (although it can include) exclusive binding.

### Pharmaceutical Compositions

In some embodiments, the bispecific binding agents, nucleic acids, and recombinant cells of the disclosure can be incorporated into compositions, including pharmaceutical compositions. Such compositions typically include the bispecific binding agents, nucleic acids, and/or recombinant cells, and a pharmaceutically acceptable excipient, e.g., a carrier.

Bispecific binding agents of the disclosure can be administered using formulations used for administering antibodies and antibody-based therapeutics, or formulations based thereon. Nucleic acids of the disclosure are administered using formulations used for administering oligonucleotides, antisense RNA agents, and/or gene therapies such as CRISPR/Cas9 based therapeutics.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.), or phosphate buffered saline (PBS). In all cases, the composition should be sterile and should be fluid to the extent that it can be administered by syringe. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

In some embodiments, the bispecific binding agents of the disclosure are administered by transfection or infection with nucleic acids encoding them, using methods known in the art, including but not limited to the methods described in McCaffrey et al., Nature (2002) 418:6893, Xia et al., Nature Biotechnol (2002) 20:1006-10, and Putnam, Am J Health Syst Pharm (1996) 53:151-60, erratum at Am J Health Syst Pharm (1996) 53:325.

Bispecific binding agents of the disclosure can be administered using a formulation comprising a fusogenic carrier. These are carriers capable of fusing with the plasma membrane of a mammalian cell. Fusogenic carriers include, without limitation, membrane-encapsulated viral particles and carriers based thereon, exosomes and microvesicles (see, e.g., Y. Yang et al., J Extracellular Vessicles (2018) 7:144131), fusogenic liposomes (see, e.g., Bailey et al., U.S. Pat. No. 5,552,155; Martin et al., U.S. Pat. No. 5,891,468; Holland et al., U.S. Pat. No. 5,885,613; and Leamon, U.S. Pat. No. 6,379,698).

### Methods of the Disclosure

The present disclosure provides, among others, a method of treating a disorder in a subject. The method includes administering to a subject in need thereof, a therapeutically effective amount of the bispecific binding agent, the nucleic acid, the vector, the engineered cell, the immunoconjugate, or the pharmaceutical composition provided herein. The disorder that can be treated by the various compositions described herein can be a neoplastic disorder, an inflammatory disease, metabolic disorder, an endocrine disorder, and a neurological disorder.

In some embodiments, the condition to be treated includes a neoplastic disorder. Some non-limiting neoplastic disorders that can be treated by the various compositions described herein include, without being limited to, breast cancer, B cell lymphoma, pancreatic cancer, Hodgkin's lymphoma, ovarian cancer, prostate cancer, mesothelioma, lung cancer, non-Hodgkin's B-cell (B-NHL), melanoma, chronic lymphocytic leukemia, acute lymphocytic leukemia, neuroblastoma, glioma, glioblastoma, bladder cancer, and colorectal cancer.

In some embodiments, the condition to be treated includes an inflammatory disease. Some non-limiting inflammatory diseases that can be treated by the various compositions described herein include, without being limited to, inflammatory intestinal disease, rheumatoid arthritis, lupus, Crohn's disease, and ulcerative colitis.

In some embodiments, the condition to be treated includes a metabolic disorder. A metabolic disorder generally refers to a disorder that negatively alters the body's processing and distribution of macronutrients such as proteins, lipids, and carbohydrates. For example, metabolic disorders can happen when abnormal chemical reactions in the body alter the normal metabolic process. Metabolic disorders can also include inherited single gene anomalies, most of which are autosomal recessive. Further, metabolic disorders can be complications of severe diseases or conditions, including liver or respiratory failure, cancer, chronic obstructive pulmonary disease (COPD, includes emphysema and chronic bronchitis), and HIV/AIDS. Some non-limiting metabolic disorders that can be treated by the various compositions described herein include, without being limited to, diabetes, Gaucher disease, Hunter syndrome, Krabbe disease, maple syrup urine disease, metachromatic leukodystrophy, mitochondrial encephalopathy, lactic acidosis, stroke-like episodes (MELAS), Niemann-Pick, phenylketonuria (PKU), porphyria, Tay-Sachs disease, and Wilson's disease.

In some embodiments, the condition to be treated includes an endocrine disorder. Some non-limiting neurological disorders that can be treated by the various compositions described herein include, without diabetes mellitus, acromegaly (overproduction of growth hormone), Addison's disease (decreased production of hormones by the adrenal glands), Cushing's syndrome (high cortisol levels for extended periods of time), Graves' disease (type of hyperthyroidism resulting in excessive thyroid hormone production), Hashimoto's thyroiditis (autoimmune disease resulting in hypothyroidism and low production of thyroid hormone), hyperthyroidism (overactive thyroid), hypothyroidism (underactive thyroid), and prolactinoma (overproduction of prolactin by the pituitary gland).

In some embodiments, the condition to be treated includes a neurological disorder. Some non-limiting neurological disorders that can be treated by the various compositions described herein include, without being limited to, neurodegenerative disorders (e.g., Parkinson's, or Alzheimer's) or autoimmune disorders (e.g., multiple sclerosis) of the central nervous system; memory loss; long term and short term memory disorders; learning disorders; autism, depression, benign forgetfulness, childhood learning disorders, close head injury, and attention deficit disorder; autoimmune disorders of the brain, neuronal reaction to viral infection; brain damage; depression; psychiatric disorders such as bi-polarism, schizophrenia and the like; narcolepsy/sleep disorders (including circadian rhythm disorders, insomnia and narcolepsy); severance of nerves or nerve damage; severance of the cerebrospinal nerve cord (CNS) and any damage to brain or nerve cells; neurological deficits associated with AIDS; tics (e.g. Giles de la Tourette's syndrome); Huntington's chorea, schizophrenia, traumatic brain injury, tinnitus, neuralgia, especially trigeminal neuralgia, neuropathic pain, inappropriate neuronal activity resulting in neurodysthesias in diseases such as diabetes, MS and motor neurone disease, ataxias, muscular rigidity (spasticity) and temporomandibular joint dysfunction; Reward Deficiency Syndrome (RDS) behaviors in a subject. In some exemplary embodiments, the neurological disorders encompassed herein includes Parkinson's disease, Alzheimer's disease, and multiple sclerosis.

### Administration of Bispecific Binding Agents

Administration of any one or more of the therapeutic compositions described herein, e.g., bispecific binding agents, nucleic acids, recombinant cells, and pharmaceutical compositions, can be used to treat individuals having a condition described herein. In some embodiments, the bispecific binding agents, nucleic acids, recombinant cells, and pharmaceutical compositions are incorporated into therapeutic compositions for use in methods down-regulating or inactivating T cells, such as CAR-T cells.

Accordingly, in one aspect, provided herein are methods for inhibiting an activity of a target cell in an individual, the methods comprising the step of administering to the individual a first therapy including one or more of the bispecific binding agents, nucleic acids, recombinant cells, and pharmaceutical compositions provided herein, wherein the first therapy inhibits an activity of the target cell by degrading a target surface protein. For example, an activity of the target cell may be inhibited if its proliferation is reduced, if its pathologic or pathogenic behavior is reduced, if it is destroyed or killed, or the like. Inhibition includes a reduction of the measured quantity of at least about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the methods include administering to the individual an effective number of the recombinant cell as disclosed herein, wherein the recombinant cell inhibits the target cell in the individual by expression of bispecific binding agents. Generally, the target cell of the disclosed methods can be any cell such as, for example an acute myeloma leukemia cell, an anaplastic lymphoma cell, an astrocytoma cell, a B-cell cancer cell, a breast cancer cell, a colon cancer cell, an ependymoma cell, an esophageal cancer cell, a glioblastoma cell, a bladder cancer cell, a glioma cell, a leiomyosarcoma cell, a liposarcoma cell, a liver cancer cell, a lung cancer cell, a mantle cell lymphoma cell, a melanoma cell, a neuroblastoma cell, a non-small cell lung cancer cell, an oligodendroglioma cell, an ovarian cancer cell, a pancreatic cancer cell, a peripheral T-cell lymphoma cell, a renal cancer cell, a sarcoma cell, a stomach cancer cell, a carcinoma cell, a mesothelioma cell, or a sarcoma cell. In some embodiments, the target cell is a pathogenic cell.

Bispecific binding agents of the disclosure are typically administered in solution or suspension formulation by injection or infusion. In an embodiment, a bispecific binding agent is administered by injection directly into a tumor mass. In another embodiment, a bispecific binding agent is administered by systemic infusion.

The effective dose of the bispecific binding agents can be determined by a skilled person in the field, e.g. a physician. The effective dose of any given bispecific binding agent may depend on the binding affinity for each of the ligands, and the degree of expression of each of the ligands. The range of effective concentrations, however, can be determined by one of ordinary skill in the art, using the disclosure and the experimental protocols provided herein. Similarly, using the effective concentration one can determine the effective dose or range of dosages required for administration.

Depending on the disease or disorder to be treated, the severity and extent of the disease, the subject's health, and the co-administration of other therapies, repeated doses may be administered. Alternatively, a continuous administration may be required. It is expected, however, that the bispecific binding agent will remain in proximity to the cell so that each molecule of bispecific binding agent can ubiquitinate and degrade multiple molecules of target surface protein. Thus, the bispecific binding agents of the disclosure may require lower doses, or less frequent administration, than therapies based on antibody competitive binding.

### Administration of Recombinant Cells to an Individual

In some embodiments, the methods involve administering the recombinant cells to an individual who is in need of such method. This administering step can be accomplished using any method of implantation known in the art. For example, the recombinant cells can be injected directly into the individual's bloodstream by intravenous infusion or otherwise administered to the individual.

The terms “administering”, “introducing”, and “transplanting” are used interchangeably herein to refer to methods of delivering recombinant cells expressing the bispecific binding agents provided herein to an individual. In some embodiments, the methods comprise administering recombinant cells to an individual by a method or route of administration that results in at least partial localization of the introduced cells at a desired site such that a desired effect(s) is/are produced. The recombinant cells or their differentiated progeny can be administered by any appropriate route that results in delivery to a desired location in the individual where at least a portion of the administered cells or components of the cells remain viable. The period of viability of the cells after administration to an individual can be as short as a few hours, e.g., twenty-four hours, to a few days, to as long as several years, or even long-term engraftment for the life time of the individual.

When provided prophylactically, in some embodiments, the recombinant cells described herein are administered to an individual in advance of any symptom of a disease or condition to be treated. Accordingly, in some embodiments the prophylactic administration of a recombinant stem cell population serves to prevent the occurrence of symptoms of the disease or condition.

When provided therapeutically in some embodiments, recombinant stem cells are provided at (or after) the onset of a symptom or indication of a disease or condition, e.g., upon the onset of disease or condition.

For use in the various embodiments described herein, an effective amount of recombinant cells as disclosed herein, can be at least 10² cells, at least 5×10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, or multiples thereof. The recombinant cells can be derived from one or more donors or can be obtained from an autologous source (i.e., the human subject being treated). In some embodiments, the recombinant cells are expanded in culture prior to administration to an individual in need thereof.

In some embodiments, the delivery of a composition comprising recombinant cells (i.e., a composition comprising a plurality of recombinant cells a bispecific binding agent provided herein) into an individual by a method or route results in at least partial localization of the cell composition at a desired site. A cell composition can be administered by any appropriate route that results in effective treatment in the individual, e.g., administration results in delivery to a desired location in the individual where at least a portion of the composition delivered, e.g., at least 1×10⁴ cells, is delivered to the desired site for a period of time. Modes of administration include injection, infusion, instillation, and the like. Injection modes include, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebrospinal, and intrasternal injection and infusion. In some embodiments, the route is intravenous. For the delivery of cells, administration by injection or infusion can be made.

In some embodiments, the recombinant cells are administered systemically, in other words a population of recombinant cells are administered other than directly into a target site, tissue, or organ, such that it enters, instead, the individual's circulatory system and, thus, is subject to metabolism and other like processes.

The efficacy of a treatment with a composition for the treatment of a disease or condition can be determined by the skilled clinician. However, one skilled in the art will appreciate that a treatment is considered effective treatment if any one or all of the signs or symptoms or markers of disease are improved or ameliorated. Efficacy can also be measured by failure of an individual to worsen as assessed by hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting disease progression, e.g., arresting, or slowing the progression of symptoms; or (2) relieving the disease, e.g., causing regression of symptoms; and (3) preventing or reducing the likelihood of the development of symptoms.

As discussed above, a therapeutically effective amount includes an amount of a therapeutic composition that is sufficient to promote a particular effect when administered to an individual, such as one who has, is suspected of having, or is at risk for a disease. In some embodiments, an effective amount includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

In some embodiments, the individual is a mammal. In some embodiments, the mammal is human. In some embodiments, the individual has or is suspected of having a disease associated with cell signaling mediated by a cell surface protein (e.g., a membrane-associated target protein) or a soluble target protein. In some embodiments, the disorder is a neoplastic disorder, an inflammatory disease, and a neurological disorder.

### Systems and Kits

Also provided herein are systems and kits including the bispecific binding agents, recombinant nucleic acids, recombinant cells, or pharmaceutical compositions provided and described herein as well as written instructions for making and using the same. For example, provided herein, in some embodiments, are systems and/or kits that include one or more of: a bispecific binding agent as described herein, a recombinant nucleic acid as described herein, a recombinant cell as described herein, or a pharmaceutical composition as described herein. In some embodiments, the systems and/or kits of the disclosure further include one or more syringes (including pre-filled syringes) and/or catheters used to administer one any of the provided bispecific binding agents, recombinant nucleic acids, recombinant cells, or pharmaceutical compositions to an individual. In some embodiments, a kit can have one or more additional therapeutic agents that can be administered simultaneously or sequentially with the other kit components for a desired purpose, e.g., for modulating an activity of a cell, inhibiting a target cancer cell, or treating a disease in an individual in need thereof.

Any of the above-described systems and kits can further include one or more additional reagents, where such additional reagents can be selected from: dilution buffers; reconstitution solutions, wash buffers, control reagents, control expression vectors, negative control polypeptides, positive control polypeptides, reagents for in vitro production of the bispecific binding agents.

In some embodiments, a system or kit can further include instructions for using the components of the kit to practice the methods. The instructions for practicing the methods are generally recorded on a suitable recording medium. For example, the instructions can be printed on a substrate, such as paper or plastic, and the like. The instructions can be present in the kits as a package insert, in the labeling of the container of the kit or components thereof (i.e., associated with the packaging or sub-packaging), and the like. The instructions can be present as an electronic storage data file present on a suitable computer readable storage medium, e.g. CD-ROM, diskette, flash drive, and the like. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

### EXAMPLES

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

### Example 1: KineTACs Mediate the Degradation of PD-L1

This Example demonstrates an exemplary bispecific binder that targets endogenous cell-surface receptors of CXCL12 and PD-L1.

To demonstrate proof-of-concept that KineTACs can degrade cell surface proteins, PD-L1 was chosen as a first target. Overexpression of PD-L1 on cancer cells leads to the inhibition of checkpoint protein PD-1 and suppression of cytotoxic T cell activity. PD-L1 has been successfully degraded by both AbTACs and LYTACs. Further, a recent paper demonstrated that homodimerization-induced internalization of PD-L1 could offer efficacy of checkpoint blockade comparable to anti-PD-L1 blocking antibodies. Thus, PD-L1 was an ideal first target to test our KineTAC platform. First, knob-in-hole bispecifics were generated in which CXCL12 chemokine was N-terminally fused to the knob Fc domain and the antibody sequence for Tecentriq (atezolizumab), an FDA approved inhibitor of PD-L1, was fused to the hole Fc (FIG. 2A). CXCL12-bispecifics are not limited by the light chain mispairing problem, which is common to bispecific IgGs with Fabs on both arms, enabling full assembly of KineTACs during expression. A histidine tag was introduced on the knob arm to allow purification of the formed bispecific away from hole-hole homodimers that may form. Next, it was tested whether our PD-L1 targeting KineTAC (herein termed CXCL12-Tec or CXCL12-Atz) can bind both PD-L1 and CXCR4/CXCR7. Biolayer interferometry (BLI) was used to determine whether CXCL12-Tec binds PD-L1. Briefly, PD-L1 ectodomain fused to an Fc domain was immobilized on a BLI tip, after which varying concentrations of CXCL12-Tec was added. It was observed that CXCL12-Tec binds with pico-molar affinity to PD-L1 Fc fusion (FIG. 2B). Next, flow cytometry was used to assess binding of the CXCL12 arm on cells known to express CXCR4 and CXCR7. To eliminate off-target binding of the hole arm, an isotype control of the CXCL12 bispecific was expressed in which the Fab arm comprised of a viral protein binder. It was observed that CXCL12 isotype binds triple negative breast cancer cell line MDA-MB-231, suggesting that CXCL12 is functional in the bispecific construct (FIG. 2C).

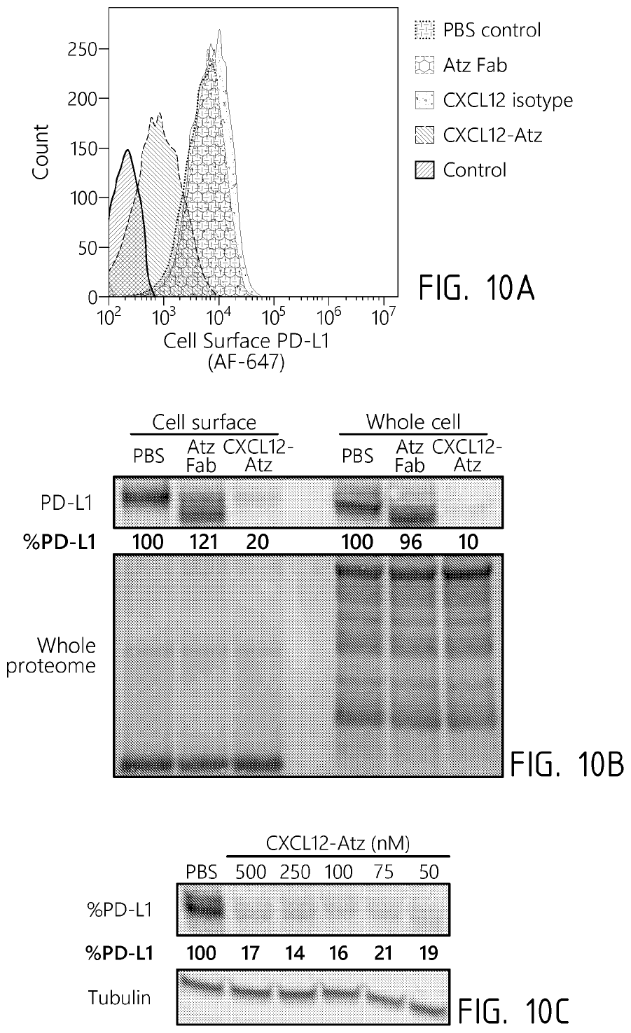

To determine whether CXCL12-Tec, also termed CXCL12-Atz herein, could degrade PD-L1, MDA-MB-231 cells was treated, which endogenously co-express PD-L1, CXCR4, and CXCR7, with varying concentrations of CXCL12-Tec. After 24 hr treatment, levels of PD-L1 protein were quantified using western blotting. Excitingly, it was observed that both glycosylated forms of PD-L1 were substantially degraded after 24 hr treatment (FIG. 2D), with a maximal percent degradation (Dₘₐₓ) of 67% occurring at 100 nM CXCL12-Tec. Interestingly, no “hook effect” was observed within the concentration regime tested and at higher concentrations, demonstrating a potential advantage of KineTACs as compared to other bifunctional degrader technologies (FIG. 10C). Control antibodies, including Tecentriq Fab or CXCL12-Isotype, do not induce degradation of PD-L1 either alone or in combination (FIGS. 2E-F). Finally, flow cytometry and western blotting were used to verify that the PD-L1 degradation observed is due to depletion of cell surface PD-L1 (Extended Data FIG. 1c-d) FIGS. 10A-10B. Thus, PD-L1 degradation is dependent on the bispecific KineTAC scaffold and occurs in a dose-dependent manner.

### Example 2: KineTAC Platform is Generalizable to Targeting Various Therapeutically Relevant Cell Surface Proteins

This Example sought to determine whether the KineTAC platform could be applied towards the degradation of other therapeutically relevant cell surface proteins.

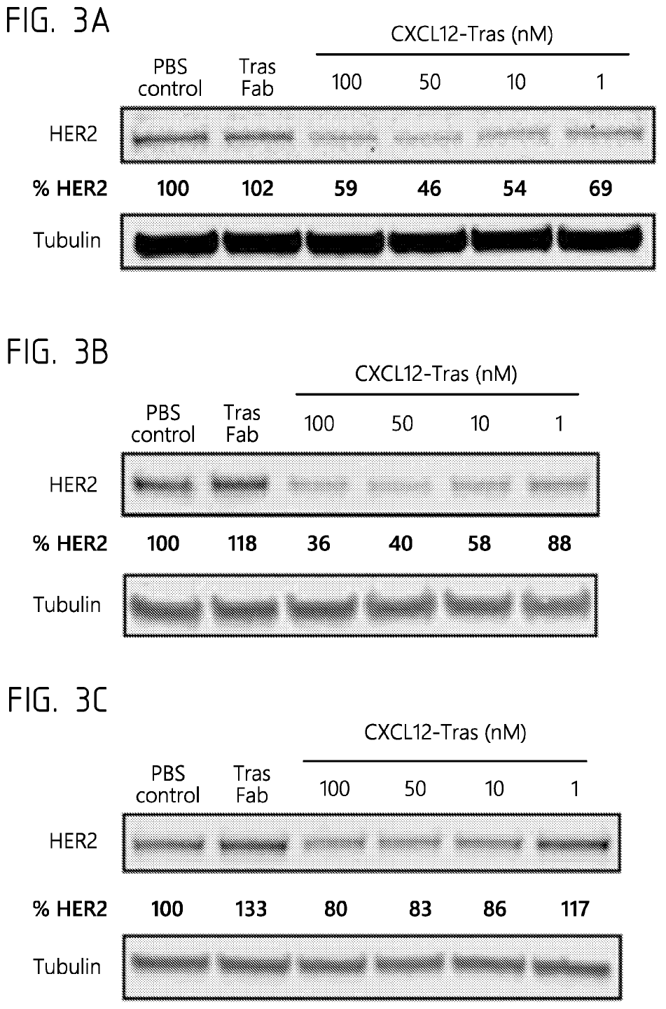

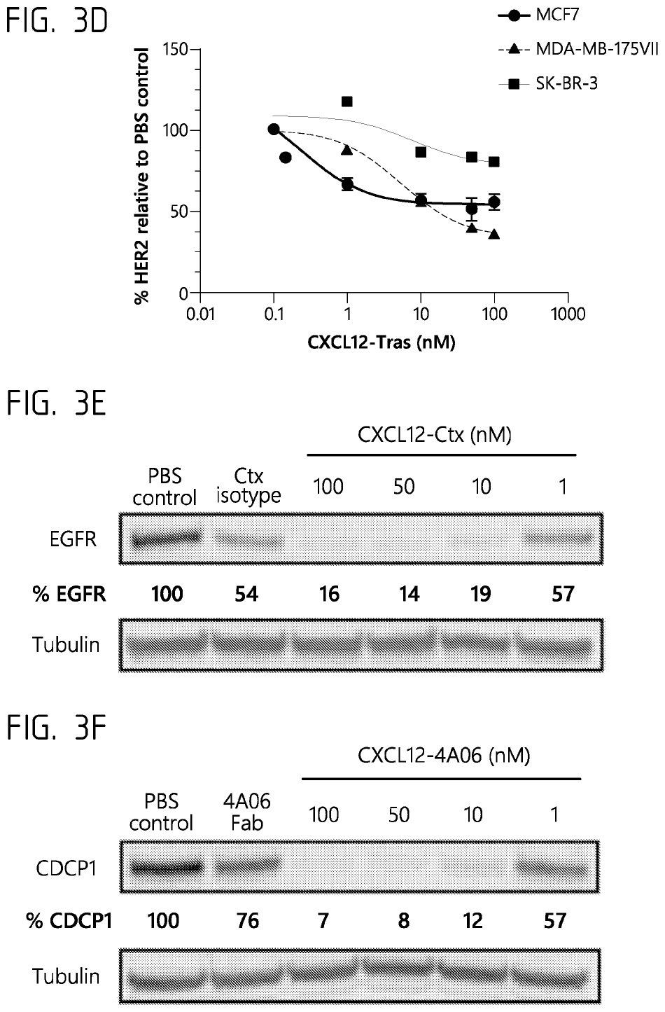

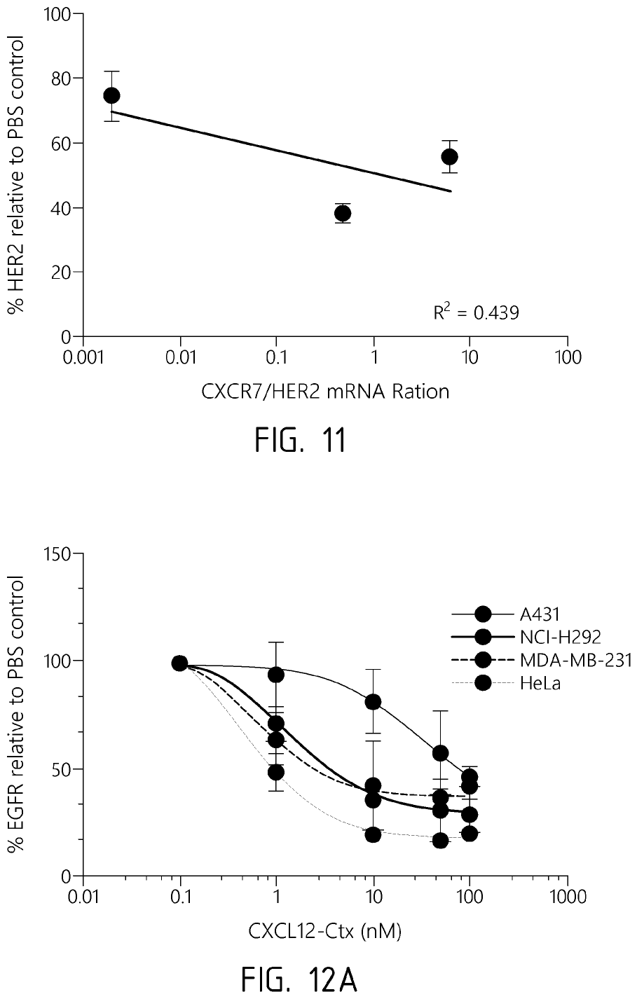

First, human epidermal growth factor receptor 2 (HER2) was targeted, which is frequently upregulated in cancer. In particular, HER2 overexpression is linked to breast cancer invasiveness and tumor progression. As such, numerous small molecule and biologic inhibitors of HER2 have been developed to inhibit breast cancer cell growth. To develop a KineTAC targeting HER2, the antibody sequence for Herceptin (trastuzumab) was incorporated, an FDA approved HER2 inhibitor, into the KineTAC scaffold (herein termed CXCL12-Tras). The ability of CXCL12-Tras to degrade HER2 in breast cancer cell line MCF-7 was tested and found that after 24 hrs HER2 was significantly degraded compared to Trastuzumab Fab treatment alone, with a Dₘₐₓ of 54% (FIG. 3A). Next step was to determine how the ratio of HER2 and CXCR4/CXCR7 expression levels may impact the maximal levels of degradation. Breast cancer cell lines MDA-MB-175VII and SK-BR-3, which express increasing levels of HER2, were treated with CXCL12-Tras for 24 hrs. Indeed, increasing the ratio of HER2:CXCR4/CXCR7 decreased the levels of HER2 degradation, with a Dₘₐₓ of 64% and 20% observed in MDA-MB-175VII and SK-BR-3, respectively (FIGS. 3B-D). Furthermore, we find that the maximal percent degradation mediated by KineTACs is slightly correlated to the expression of the target protein relative to CXCR7 (R²=0.439) (Extended Data FIG. 2c) FIG. 11. Thus, the maximal levels of degradation mediated by KineTACs is dependent on the expression level of the target protein compared to CXCR4/CXCR7.

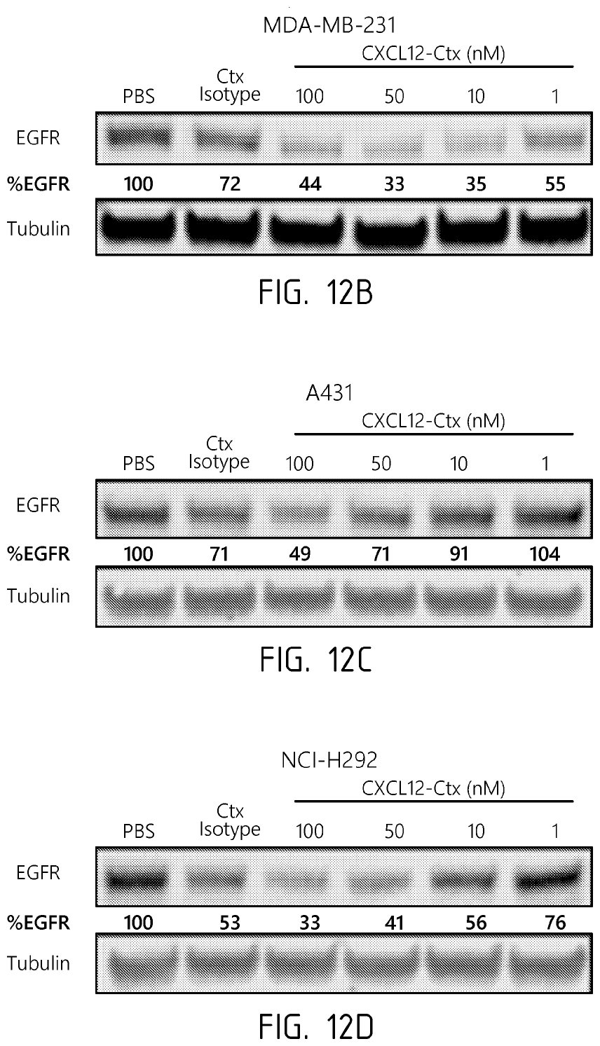

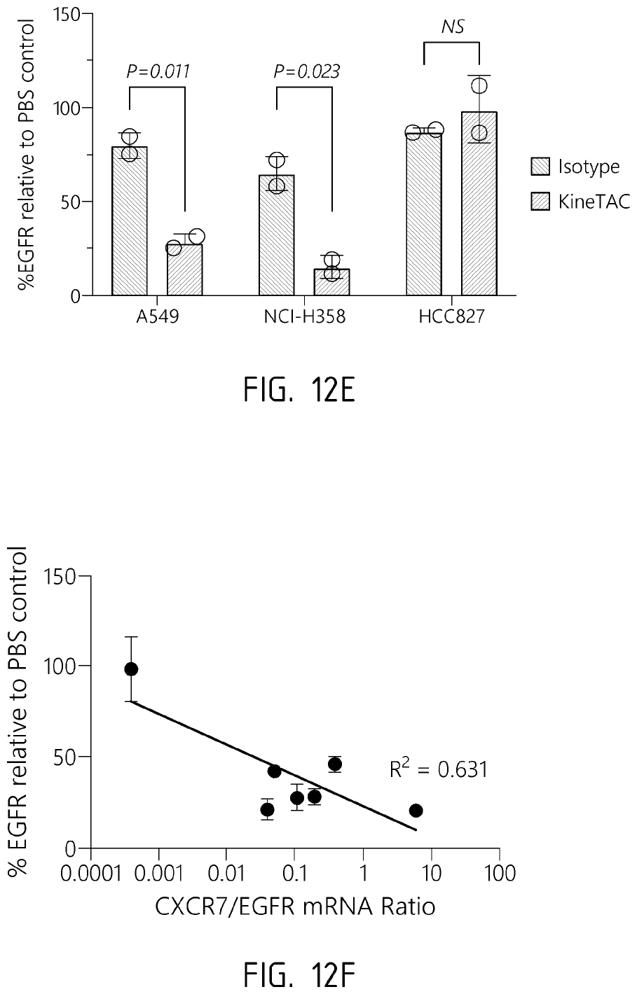

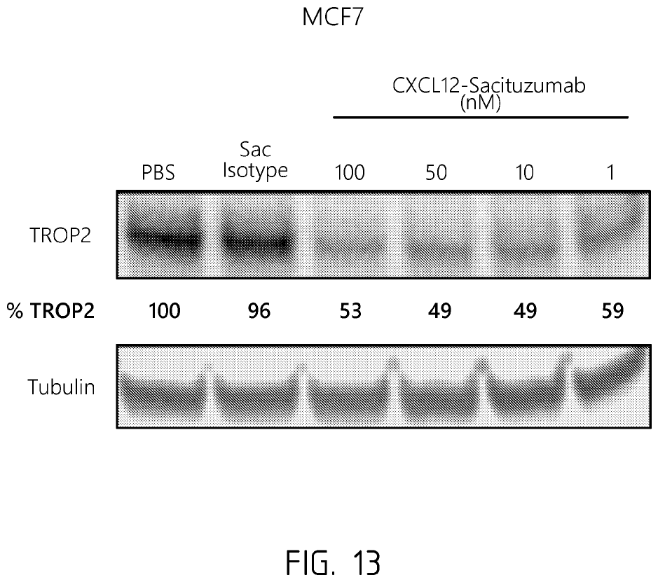

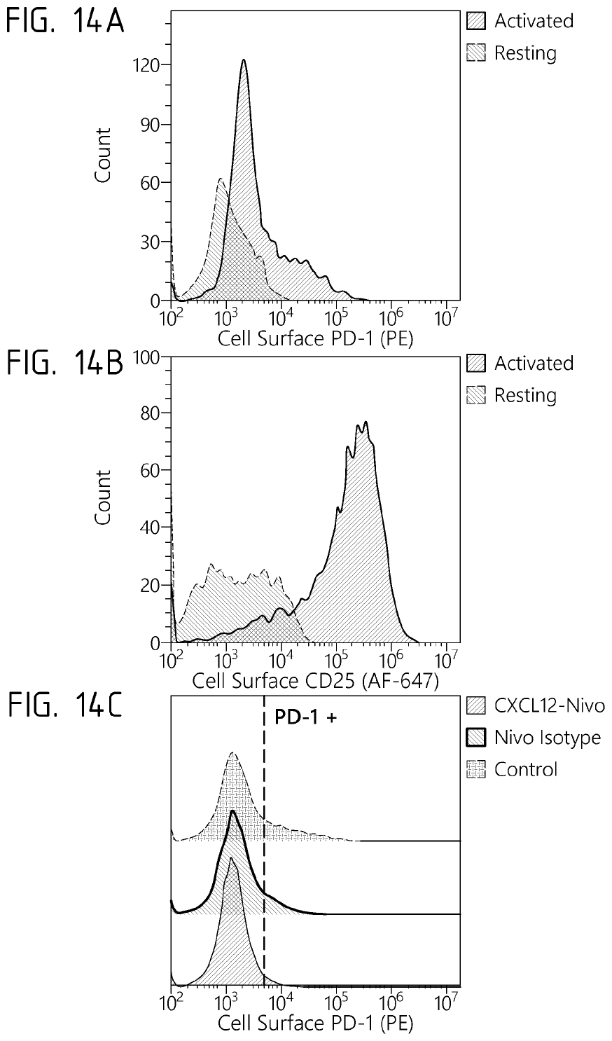

Next, it was sought to expand KineTACs to targeting epidermal growth factor receptor (EGFR) for degradation. EGFR is implicated as a driver of cancer progression, and EGFR inhibitors are approved for use in non-small cell lung, colorectal, and gastric cancers. KineTACs targeting EGFR was developed by incorporating Erbitux (cetuximab), an FDA approved EGFR inhibitor, into the KineTAC scaffold (herein termed CXCL12-Ctx). It was then tested for degradation in HeLa cells after 24 hr treatment and found that EGFR levels were dramatically reduced, with a Dₘₐₓ of 86% observed (FIG. 3E). This result was recapitulated in various breast and lung cancer cell lines, including MDA-MB-231, A431, NCI-H292, A549, NCI-H358, and HCC827, and the levels of maximal percent degradation were slightly correlated to the expression of EGFR relative to CXCR7 (R²=0.631) (FIG. 1j, Extended Data FIG. 2d-h). FIGS. 12A-12F. Finally, it was tested whether KineTACs could be used to degrade CUB domain-containing protein 1 (CDCP1), which had been previously identified to be upregulated on the surface of KRAS^{G12V} transformed cells. Previously described 4A06 antibody, which selectively binds CDCP1, was introduced into the KineTAC construct (herein termed CXCL12-4A06). Next, HeLa cells known to express CDCP1 were treated with CXCL12-4A06 and observed near complete degradation of CDCP1 after 24 hrs, with a Dₘₐₓ of 93% (FIG. 3F). KineTACs also enabled the degradation of tumor-associated calcium signal transducer 2 (TROP2), the overexpression of which has been linked to tumor progression in a variety of tumors.^{29,30} In MCF7 cells, we observed a Dₘₐₓ of 51% after treatment with TROP2 targeting KineTAC (Extended Data FIG. 2j) FIG. 13. We then tested whether KineTACs can degrade the checkpoint protein PD-1 in CD8+ T cells isolated from primary human peripheral blood mononuclear cells. T cells were first activated, causing overexpression of PD-1 on the cell surface along with other activation markers (Extended Data FIG. 3a-b) FIGS. 14A-14B. Activated T cells were then treated for 24 hr with a PD-1 targeting KineTAC, which incorporates the antibody sequence for nivolumab (Opdivo), an FDA approved PD-1 inhibitor (herein termed CXCL12-Nivo). Following treatment with CXCL12-Nivo, cell surface PD-1 levels were dramatically reduced, with a Dₘₐₓ of 82%, compared to nivolumab isotype control, which is known to induce slight internalization of PD-1 (FIG. 1k, Extended Data FIG. 3c) FIG. 14C.³¹. Overall, these results demonstrate the generality of the KineTAC platform for degrading various cell surface proteins for degradation. Furthermore, it was found that different protein targets display varying levels of degradation following KineTAC treatment.

Table 3 below shows ratios of CXCR4 and HER2 expression (TPM) in MCF-7, MDA-MB-175VII, and SK-BR-3 cells. Values were obtained from Victoria.ucsf.edu online database.

TABLE 3
Ratios of CXCR4 and HER2 expression
(TPM) in exemplary cell lines.

| Cell line | CXCR4 TPM | HER2 TPM | RATIO |
|---|---|---|---|
| MCF7 | 85 | 45 | 1.89 |
| MDA-MB-175VII | 290 | 290 | 1 |
| SK-BR-3 | 15 | 1000 | 0.02 |

Table 4 below shows a summary of CXCR7:target ratios and maximal degradation (Dₘₐₓ) for each cell surface KineTAC target.

TABLE 4
Ssummary of CXCR7:target ratios and maximal degradation
(Dₘₐₓ) for each cell surface KineTAC target

| | | CXCR7:Target | |
|---|---|---|---|
| Target Protein | Cell Line | mRNA Ratio¹ | Dₘₐₓ (%) |
| PD-L1 | MDA-MB-231 | 0.5 | 73 |
| HER2 | MCF7 | 8.6 | 51 |
| HER2 | MDA-MB-175VII | 1.3 | 62 |
| HER2 | SK-BR-3 | 0.03 | 26 |
| EGFR | HeLa | 6.0 | 84 |
| EGFR | A431 | 0.4 | 54 |
| EGFR | MDA-MB-231 | 0.2 | 66 |
| EGFR | NCI-H292 | 0.1 | 74 |
| EGFR | A549 | 0.2 | 72 |
| EGFR | NCI-H358 | 0.04 | 79 |
| EGFR | HCC827 | 0.0004 | N/A |
| PD-1 | Primary CD8+ T cells | N/A | 82 |
| CDCP1 | HeLa | 5.6 | 94 |
| TROP2 | MCF7 | 1.8 | 51 |

### Example 3: Mechanism of KineTAC-Mediated Degradation

Following demonstration of proof-of-concept, this Example sought to evaluate the mechanism of KineTAC-mediated degradation.

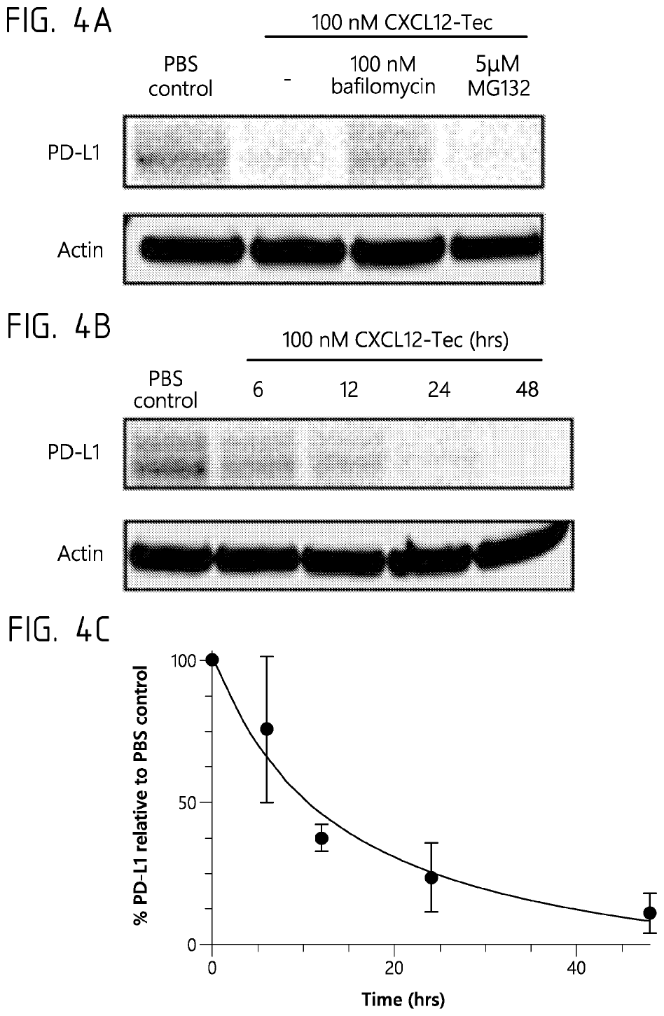

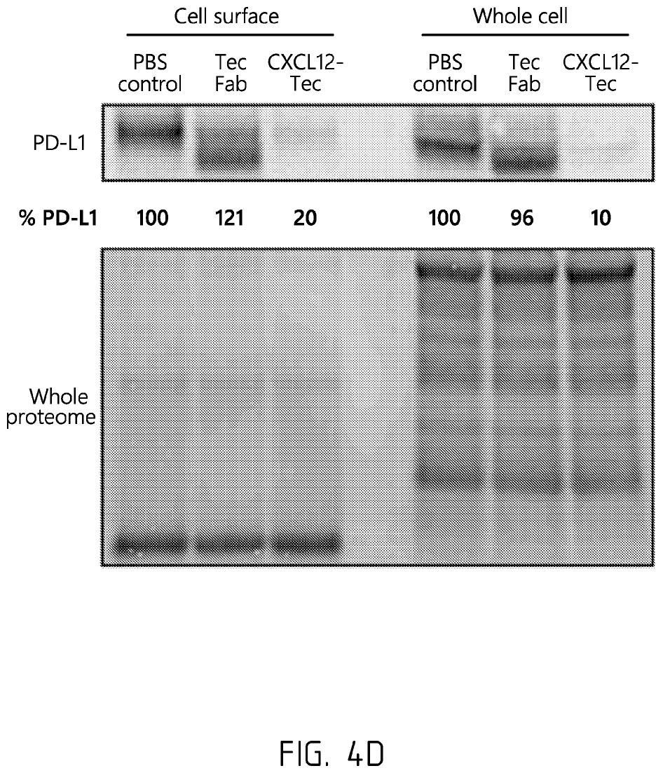

To determine whether KineTACs catalyze degradation via the lysosome or proteasome, MDA-MB-231 cells were pre-treated with either media alone, bafilomycin (an inhibitor of lysosome acidification), or MG-132 (a proteasome inhibitor). After 1 hr pre-treatment, cells were treated with 100 nM CXCL12-Tec for 24 hrs. It was observed that bafilomycin pre-treatment inhibited degradation of PD-L1, while MG-132 had no effect (FIG. 4A), thus demonstrating that KineTACs mediate the degradation via delivery of target proteins to the lysosome. Immunofluorescent microscopy revealed complete removal of EGFR from the cell surface following 24 hr CXCL12-Ctx treatment as compared to the cetuximab isotype, further highlighting that KineTACs induce robust internalization of target proteins (FIG. 3b) FIG. 31. Next, it was tested whether KineTAC-mediated degradation was time-dependent. Indeed, degradation begins as early as 6 hrs post-treatment, with the levels of PD-L1 continuing to decrease over time to near complete degradation at 48 hrs (FIGS. 4B-C). One possibility for the lack of complete PD-L1 degradation at 24 hrs could be that an intracellular pool of PD-L1 is not accessible for recognition by the KineTAC. To verify this possibility, a membrane impermeable biotin derivative was used to label cell surface proteins post-KineTAC treatment. The cell surface levels of PD-L1 were then compared to the whole cell levels using western blotting, revealing that the level of PD-L1 degradation is similar between these two samples (FIG. 4D). Thus, it is unlikely that an intracellular pool of PD-L1 exists that is escaping degradation.

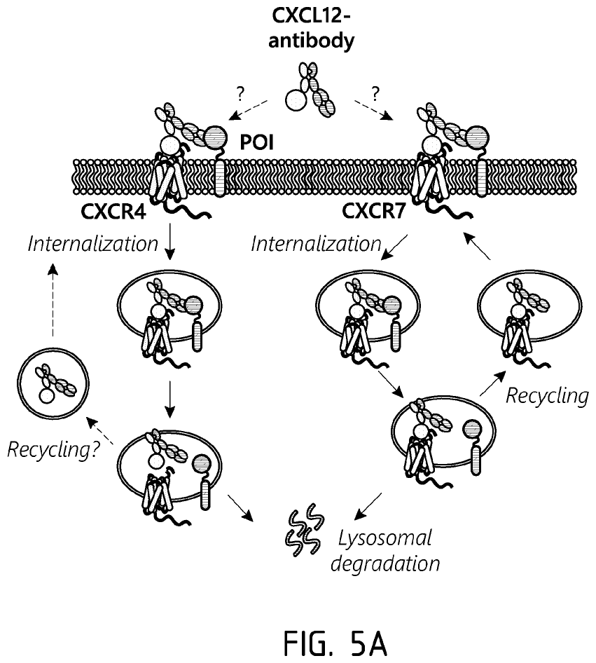

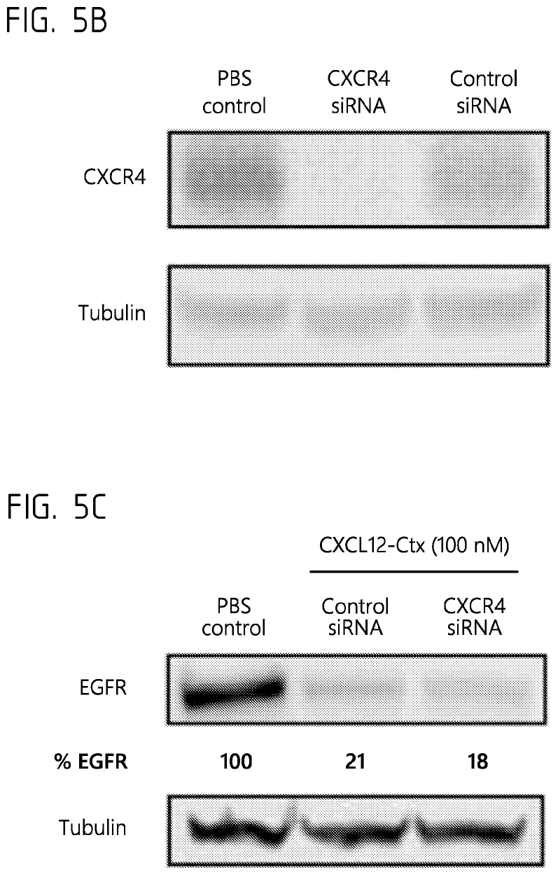

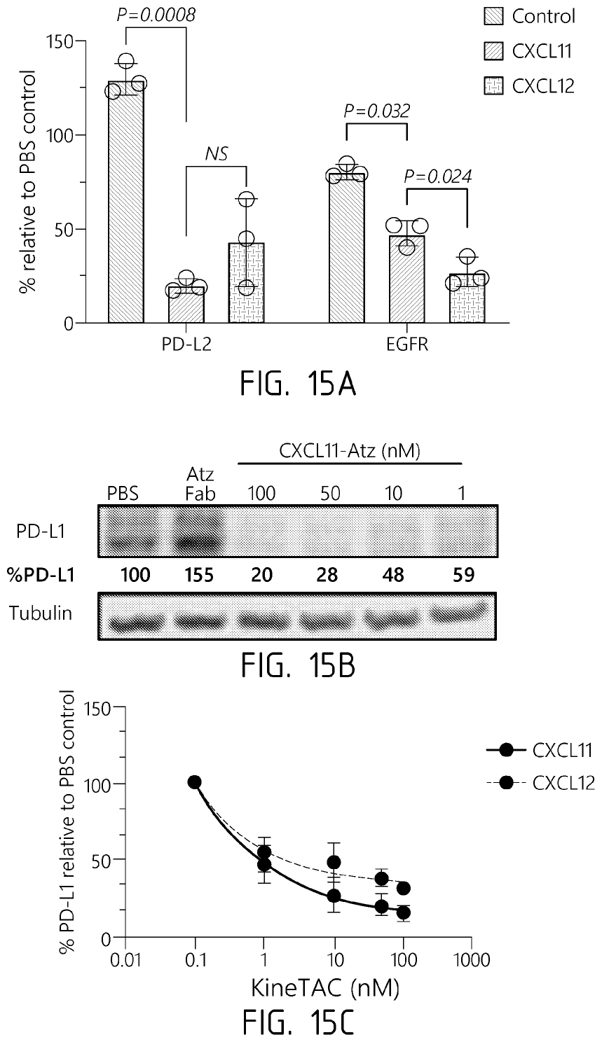

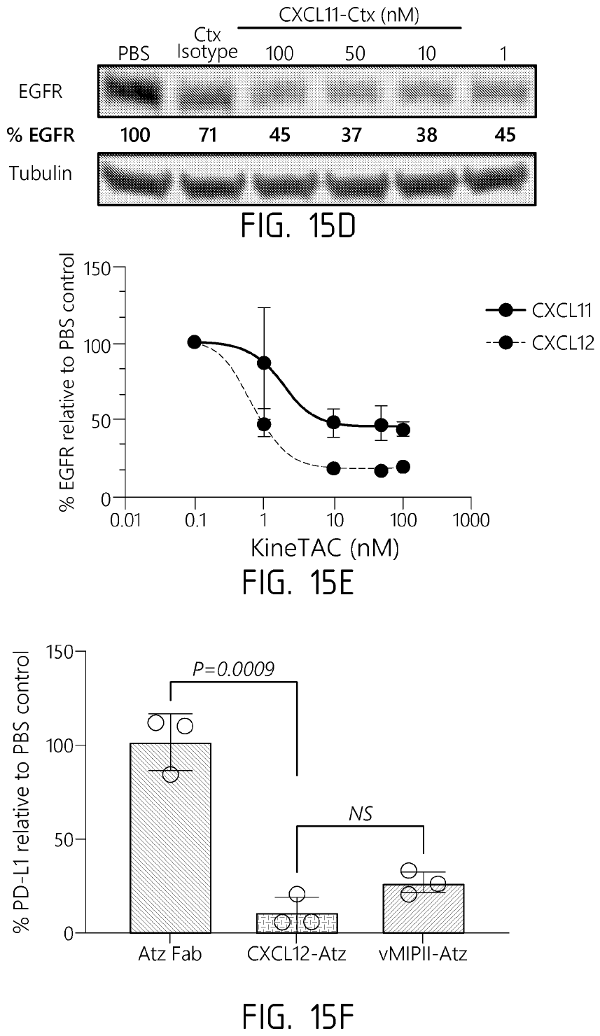

Next, it was sought to elucidate the mechanism of CXCL12 internalization that enables KineTAC-mediated degradation. As mentioned, CXCL12 binds both CXCR4 and CXCR7, and the outcome of cytokine-receptor binding is different depending on which receptor is engaged. Three possibilities for the mechanism exist: 1) CXCR4 only, 2) CXCR7 only, or 3) both CXCR4 and CXCR7 are being engaged (FIG. 5A). To begin to address these possibilities, RNA interference was used to knockdown levels of CXCR4 in HeLa cells expressing EGFR (FIG. 5B). After 48 hr transfection with CXCR4-targeting or control siRNA pools, cells were treated with 100 nM CXCL12-Ctx for 24 hrs. Western blotting analysis revealed that EGFR degradation levels are unchanged when CXCR4 is knocked down (FIG. 5C). This data does not rule out CXCR4 involvement in KineTAC degradation but does indicate that degradation is not only mediated by CXCR4. Furthermore, KineTACs bearing CXCL11, a chemokine that specifically binds CXCR7 and CXCR3 but not CXCR4⁴², are capable of efficiently degrading both PD-L1 and EGFR (FIG. 3g, Extended Data FIG. 5a-d) FIGS. 15A-15F. vMIPII, a viral chemokine that targets CXCR7 along with other chemokine receptors, was also active as a KineTAC in efficiently degrading PD-L1 (Extended Data FIG. 5e) FIG. 15F. Our results suggest that CXCR7 is the receptor responsible for KineTAC-mediated degradation and demonstrates the exciting opportunity for using alternative cytokines, such as CXCL11, in the KineTAC scaffold to degrade target proteins.

### Example 4: KineTACs do not Induce Major Cellular Perturbations

This Example explores whether proteome-wide changes occurred following KineTAC treatment.

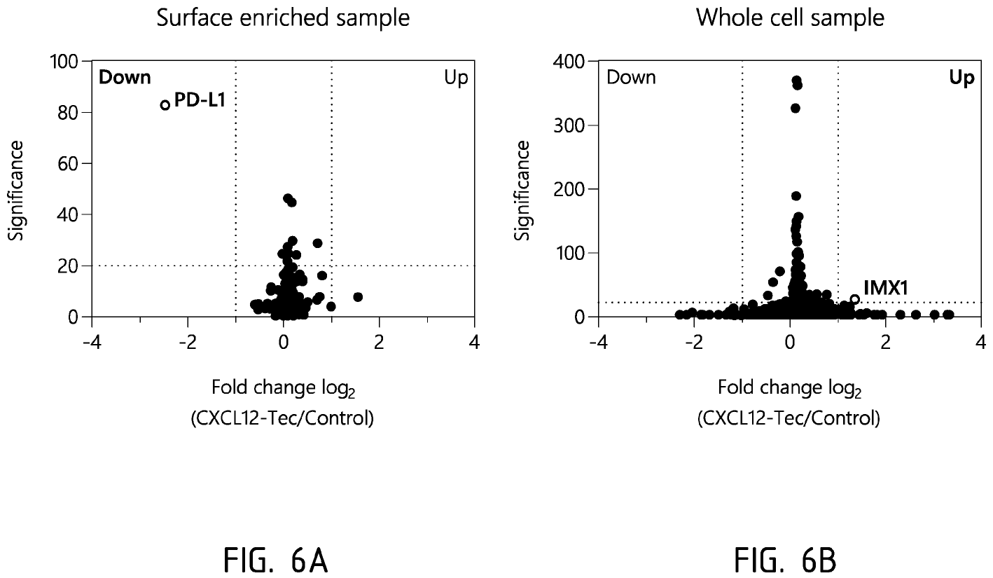

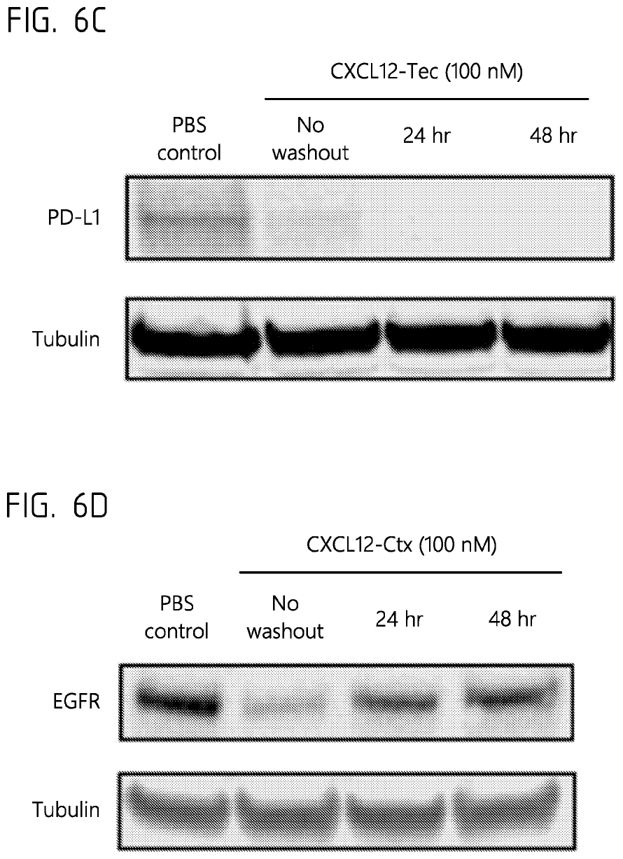

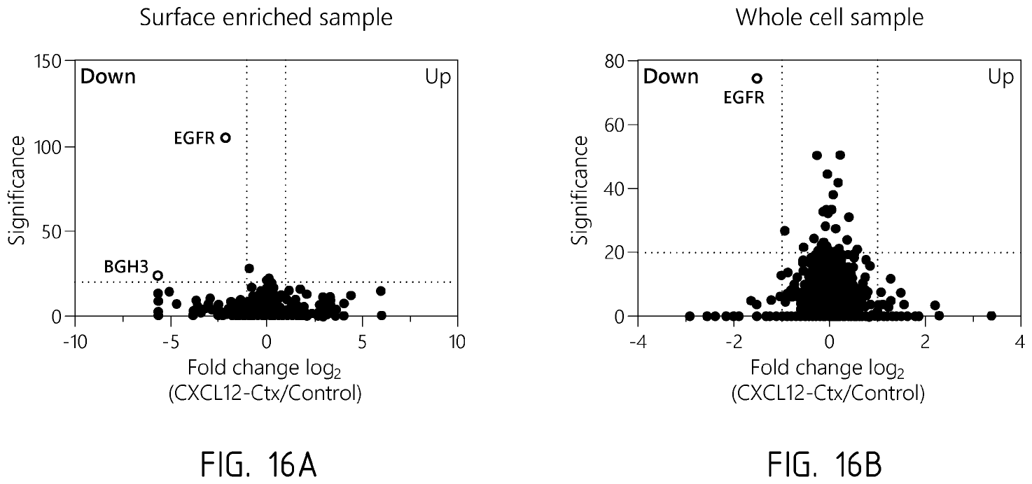

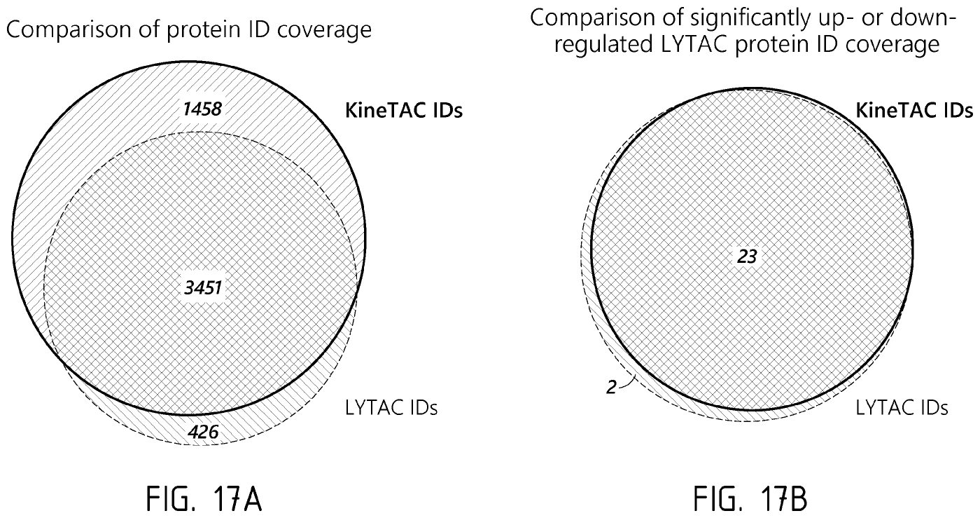

First, quantitative mass spectrometry was used to determine whether proteome-wide changes occurred following KineTAC treatment. Both the surface-enriched and whole cell lysates were analyzed following 48 hr CXCL12-Tec treatment compared to PBS treated control. The surface-enriched sample revealed no significant changes to the proteome, with PD-L1 being the only protein downregulated in CXCL12-Tec treatment compared to control (FIG. 6A). Whole cell proteomics also revealed that no major changes are occurring (FIG. 6B). PD-L1 was not detected in the whole cell sample, likely due to low abundance of cell surface proteins relative to cytosolic proteins. Similar results were observed for EGFR degradation, with no major proteome-wide changes occurring and EGFR being virtually the only protein significantly downregulated in CXCL12-Ctx treatment compared to control in both the surface-enriched and whole cell proteomics (FIG. 3j-k,) FIG. 16A-16B. Moreover, CXCR4 and CXCR7 peptide IDs were not altered in the surface-enriched sample, and CXCR4 IDs were also unchanged in the whole cell sample, indicating that treatment with KineTAC does not significantly impact CXCR4 or CXCR7 levels. CXCR7 peptide IDs were not identified in the whole cell sample due to low abundance relative to cytosolic proteins. Furthermore, protein levels of GRB2 and SHC1, which are known interacting partners of EGFR43,44, were also not significantly changed. We do observe ABCD1 and BGH3 significantly downregulated in the PD-L1 and EGFR datasets, respectively. However, these appear to be unrelated to either target protein or CXCR7, suggesting that non-specific downregulation occurred. These data suggest that KineTACs are highly selective for the target protein without altering levels of the degrading receptor or known interacting partners to the protein of interest. Interestingly, a previously published proteomics dataset of LYTAC-mediated EGFR degradation identified about two dozen proteins significantly up- or down-regulated following LYTAC treatment.4 In our dataset, we observed 89% of the proteins identified in the total LYTAC dataset, including 23 of the 25 proteins seen to significantly change after LYTAC treatment (Extended Data FIG. 6a-b) FIGS. 17A-17B. This suggests that KineTACs may be more selective in degrading EGFR.

The next question is whether KineTAC-mediated degradation was reversible, thereby not imparting permanent changes to the proteome. A washout experiment was performed in which cells were treated with KineTAC for 24 hrs, at which point the media was removed and fresh media added for the duration of the experiment. Interestingly, PD-L1 levels continued to decrease up to 48 hrs post-washout in MDA-MB-231 cells treated with 100 nM CXCL12-Tec (FIG. 6C). This result contrasts with washout experiments done both with AbTACs and small molecule internalizers of PD-L1, where PD-L1 levels recover between 24-48 hr post-washout. However, this effect appears to be target-specific. In the same experiment, EGFR levels begin to recover 24 hrs post-washout, and are fully recovered at 48 hrs post-washout in HeLa cells treated with 100 nM CXCL12-Ctx (FIG. 6D). These data could suggest that recovery of the target protein is dependent on the resynthesis rate of that protein. Another possibility could be the internalization and recycling kinetics of CXCR4 and CXCR7 in the different cell types.

### Example 5: Requirements for Efficient KineTAC-Mediated Degradation

This Example explored the requirements for efficient KineTAC-mediated degradation.

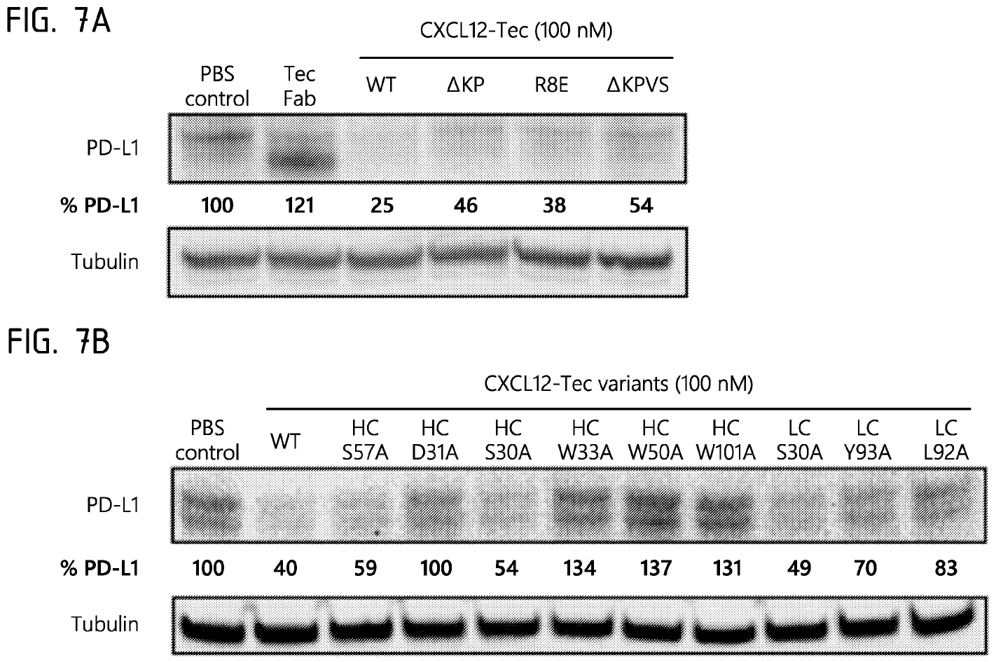

Multiple aspects of the KineTAC construct scaffold could be altered to optimize the platform for maximal levels of degradation. These include the cytokine activity, antibody binding affinity to target, construct, pH sensitivity, and Fc domain glycosylation. To determine whether CXCL12 activity impacts degradation, previously described antagonistic variants were created. Two variants, CXCL12^{ΔKP} and CXCL12^{R8E}, are reported antagonists of CXCR4. Though the activity of the CXCL12^{R8E} towards CXCR7 is unknown, the CXCL12^{ΔKP} variant retains reduced agonism of CXCR7. The third variant, CXCL12^{ΔKPVS}, is a reported antagonist of both CXCR4 and CXCR7. These different variants were then incorporated into the KineTAC scaffold with Tecentriq and levels of PD-L1 post-treatment determined by western blotting. It was observed that all three CXCR4 antagonistic variants retain the ability to degrade PD-L1, albeit at a slightly decreased level compared to the CXCL12^{WT} KineTAC. Further, the antagonist of both CXCR4 and CXCR7, CXCL12^{ΔKPVS}, can also degrade PD-L1 with a Dₘₐₓ of 46% (FIG. 7A). Thus, converting CXCL12 into an antagonist does not rescue PD-L1 levels, suggesting that our KineTAC continues to be internalized without agonism of either receptor. This continued internalization is likely due to binding CXCR7, as the receptor is known to constitutively internalize and recycle in a ligand-independent manner.

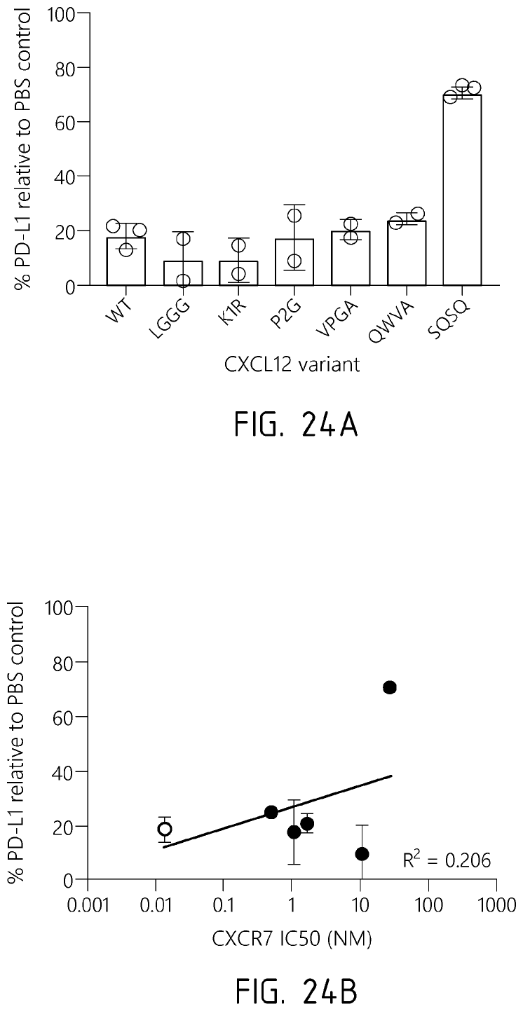

Next, it was probed whether altering the antibody to target binding affinity affected the levels of KineTAC-mediated degradation. To this end, alanine mutations were introduced into key interacting residues of Tecentriq's complementary determining regions (CDRs) to engineer Tecentriq mutants with a range of binding affinities (K_{D}) to PD-L1. BLI was used to measure the K_{D}s of these alanine mutants in Fab format (Table 4). These Fabs were then converted into our bispecific KineTAC scaffold with CXCL12^{WT} and tested for their ability to degrade PD-L1 in MDA-MB-231 cells after 24 hr treatment at 100 nM (FIG. 7B). Correlating the PD-L1 levels post-treatment to the different kinetic parameters of these binders, it was observed that degradation is correlated to the K_{D} (R²=0.6376) and the dissociation rate (K_{off}, R²=0.8035) but not to the association rate (Kₒₙ, R²=0.0362) (FIG. 7C). Of the mutants tested, wild-type Tecentriq had the highest binding affinity and induced the greatest level of PD-L1 degradation. Next, to test dependence on affinity to CXCR7, previously described N-terminal antagonistic variants of CXCL12 with a range of binding affinities to CXCR7 (0.014-28 nM) were introduced into the KineTAC scaffold with atezolizumab and tested for their ability to degrade PD-L1 (Extended Data FIG. 4b). FIG. 24A³⁵ Here, we find that degradation is not highly correlated to the IC50 against CXCR7 (R²=0.206) (Extended Data FIG. 4c) FIG. 24B. Therefore, the levels of degradation are dependent on the binding affinity of the antibody arm to the target protein.

TABLE 5
In vitro binding affinities of Tecentriq alanine mutants.

| Mutant | K_{D} (nM) | Kₒₙ (M⁻¹s⁻¹) | K_{off} (s⁻¹) |
|---|---|---|---|
| WT | 0.334 | 1.87 × 10⁵ | 6.24 × 10⁻⁵ |
| S57A HC | 0.518 | 2.53 × 10⁵ | 1.31 × 10⁻⁴ |
| D31A HC | 4.93 | 1.10 × 10⁵ | 5.44 × 10⁻⁴ |
| S30A HC | 1.40 | 4.36 × 10⁴ | 6.09 × 10⁻⁵ |
| W33A HC | N.D. | N.D. | N.D. |
| W50A HC | 63.1 | 1.38 × 10⁵ | 8.69 × 10⁻³ |
| W101A HC | 458 | 1.10 × 10⁴ | 5.02 × 10⁻³ |
| S30A LC | 1.23 | 8.48 × 10⁴ | 1.05 × 10⁻⁴ |
| Y93A LC | 5.12 | 2.16 × 10⁵ | 1.10 × 10⁻³ |
| L92A LC | 2.73 | 2.88 × 10⁵ | 7.86 × 10⁻⁴ |

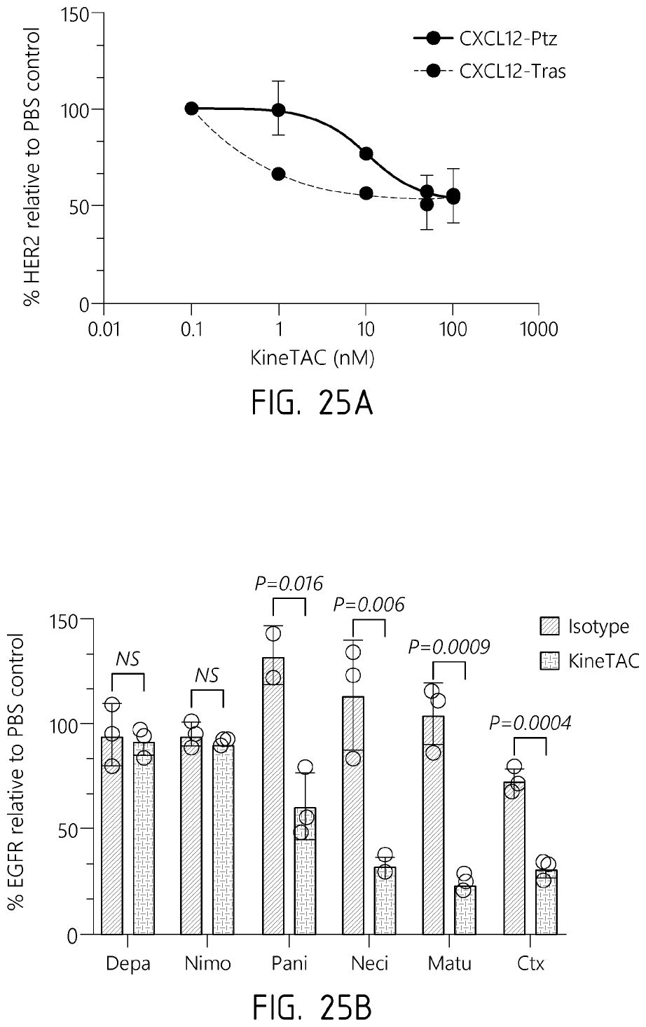

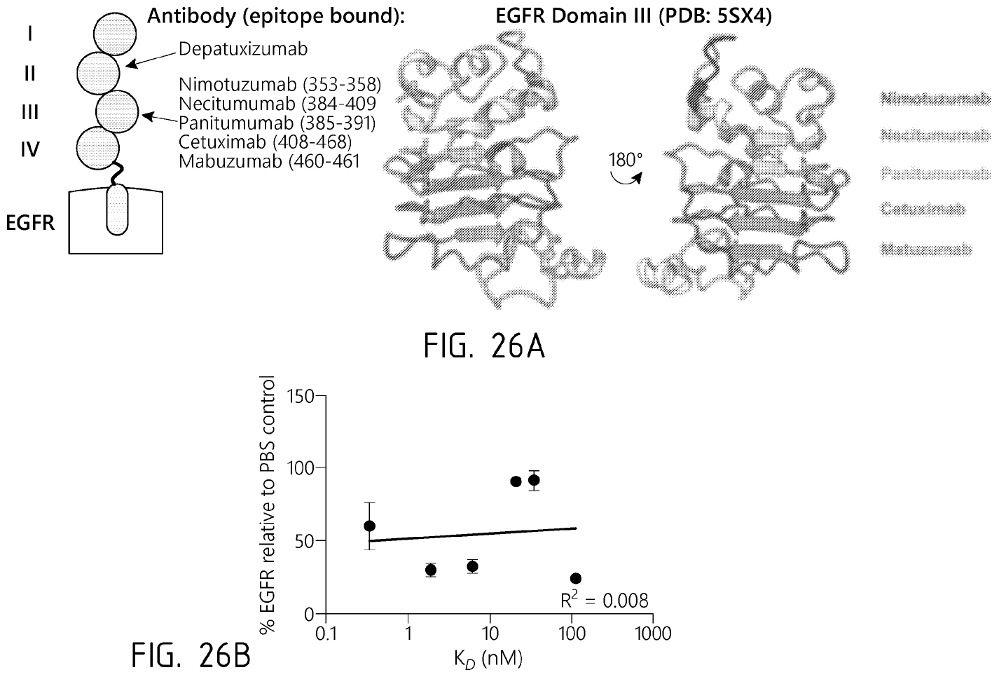

To determine whether a pH-dependent antibody binder against the target protein would affect degradation, BMS936559, an anti-PD-L1 antibody currently being tested in the clinic and reported to release PD-L1 in acidic (pH<6.0) conditions, was introduced into the KineTAC scaffold. Treatment with CXCL12-BSM936559 compared to CXCL12-Tec showed that pH-dependent release of PD-L1 slightly decreases the maximal level of degradation observed (FIG. 7D). As Tecentriq and BMS936559 are reported to have similar binding affinities to PD-L1, this result is not due to differences in K_{D}. Therefore, premature release of the target protein could impair the ability of the KineTAC to mediate its internalization and lysosomal degradation. Additionally, to investigate whether the binding epitope on the protein of interest could impact degradation, we tested additional HER2 and EGFR targeting antibodies into the KineTAC scaffold that have been described to bind different epitopes. For HER2, pertuzumab (Perjeta), which is known to bind a distinct epitope from trastuzumab on HER2³⁸, was introduced into the KineTAC scaffold (herein termed CXCL12-Ptz). Following 24 hr treatment of MCF7 cells, we find that CXCL12-Tras is superior to CXCL12-Ptz at lower concentrations while reaching the same Dₘₐₓ, indicating that epitope can alter the dose response to KineTACs (FIG. 2h) FIG. 25A. This effect is not due to differences in binding affinity, as trastuzumab and pertuzumab have reported in vitro K_{D}s of 1.43 and 1.92 nM, respectively.³⁹ For EGFR, we introduced five different anti-EGFR binders (depatuxizumab, nimotuzumab, panitumumab (Vectibix), necitumumab (Portrazza), and matuzumab)⁴⁰ into the KineTAC scaffold that bind different epitopes on EGFR domains II and III (Extended Data FIG. 4d) FIG. 26A. Following 24 hr treatment of HeLa cells, we observed that some epitope binders, such as necitumumab and matuzumab, retained similar levels of EGFR degradation compared to CXCL12-Ctx, while other epitope binders, such as depatuxizumab, nimotuzumab, and panitumumab, abrogated or impaired the ability to degrade EGFR (FIG. 2i) FIG. 25B. Lack of degradation for CXCL12-Depa and CXCL12-Nimo could be due to these antibodies binding further from the cell surface, which may impair the ability of the KineTAC to simultaneously bind both EGFR and CXCR7. Further, the degradation observed for each binder is not correlated to their respective binding affinities (R²=0.008, Extended Data FIG. 4e) FIG. 26B. This data highlights the dependence of KineTAC-mediated degradation on target binding epitope. Next, it was sought to determine whether glycosylation of the KineTAC Fc domain at N297 would impact degradation. IgGs are commonly mutated to N297G when creating an aglycosylated form to eliminate effector function. However, glycosylation at N297 can impart greater stability and favorable pharmacokinetic properties. The glycosylation site at N297 was re-introduced to the CXCL12-Tec scaffold and the degradation efficiency between the glycosylated and aglycosylated forms compared. It was observed that glycosylation at N297 does not significantly impact PD-L1 degradation levels in vitro (FIG. 7E). Thus, the stability and pharmacokinetic properties of KineTACs can be improved for in vivo use without major disruption to degradation efficiency.

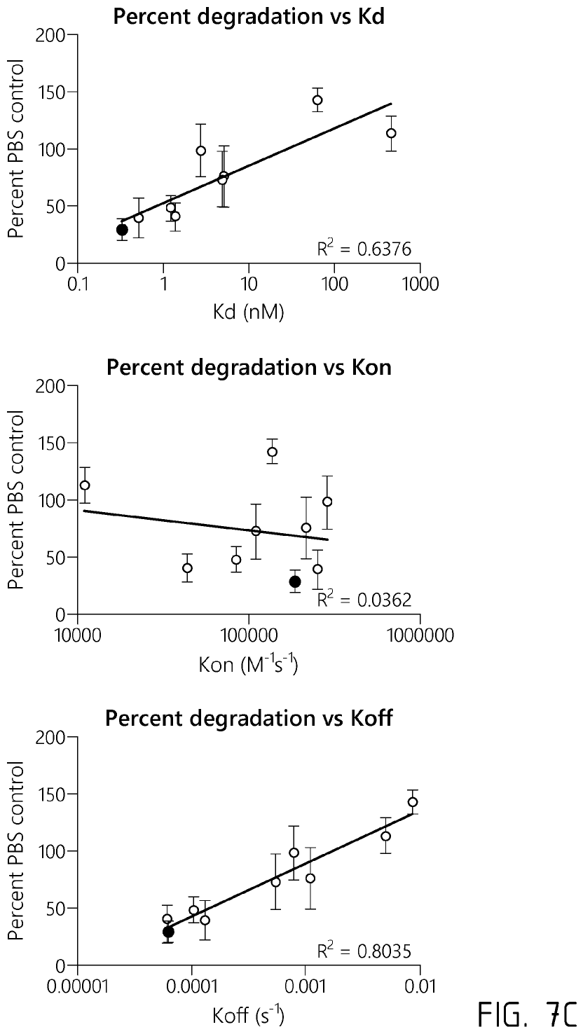

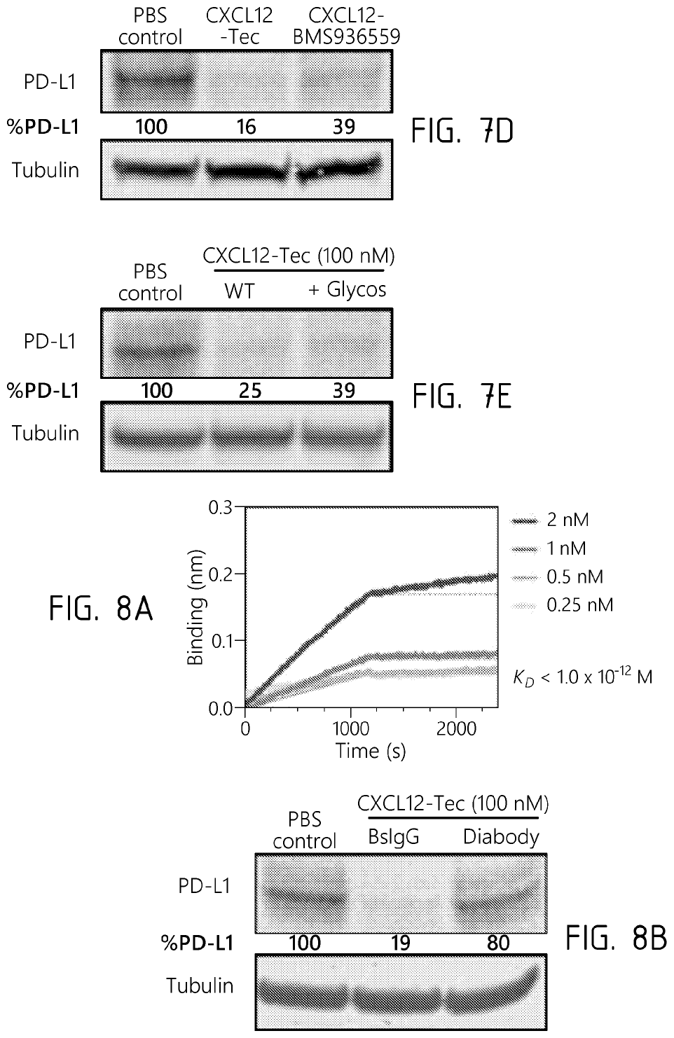

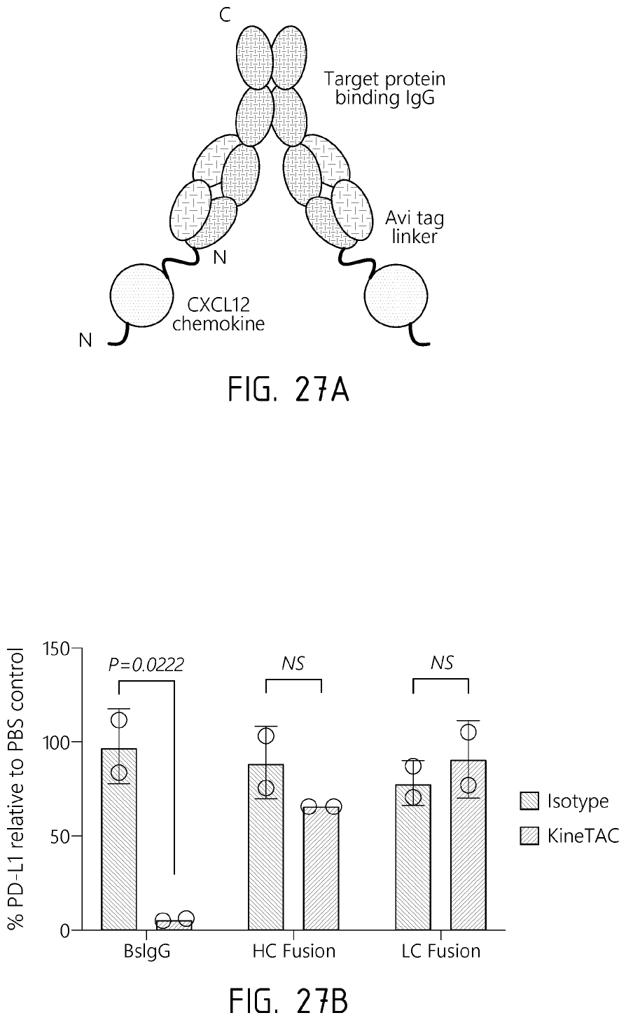

Finally, it was determined whether the bispecific antibody construct used could influence levels of degradation. To this end, a diabody construct in which CXCL12 was N-terminally fused to the heavy chain of Tecentriq Fab via a flexible Avidin tag linker was co-expressed with Tecentriq Fab light chain. The CXCL12-Tec diabody construct retained binding to PD-L1 Fc fusion as measured by BLI (FIG. 8A). After 24 hr treatment in MDA-MB-231, the levels of PD-L1 were measured by western blotting. While the bispecific IgG construct caused significant degradation of PD-L1, the diabody construct was unable to induce significant degradation, with a Dₘₐₓ of only 20% observed (FIG. 8B). We next tested whether an IgG fusion construct where CXCL12 is N-terminally fused to N-terminus of either the heavy chain or light chain of the Fab arm could mediate degradation (Extended Data FIG. 4j) FIG. 27A. Here, we again found that the bispecific IgG construct was preferred as the IgG fusions were unable to induce significant degradation of PD-L1 relative to isotype controls (Extended Data FIG. 4k) FIG. 27B. The differences in degradation between these constructs could be due to a number of factors, including construct rigidity and linker length. Overall, this data demonstrates that the bispecific knob-in hole IgG is a privileged KineTAC scaffold.

### Example 6: Functional Consequences to Degradation with KineTACs

This Example explores the functional consequences to degradation with KineTACs.

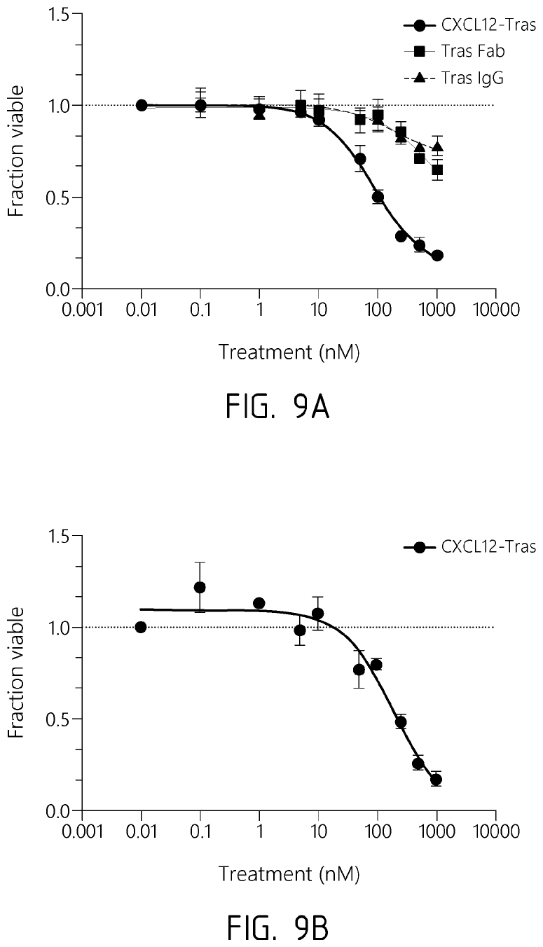

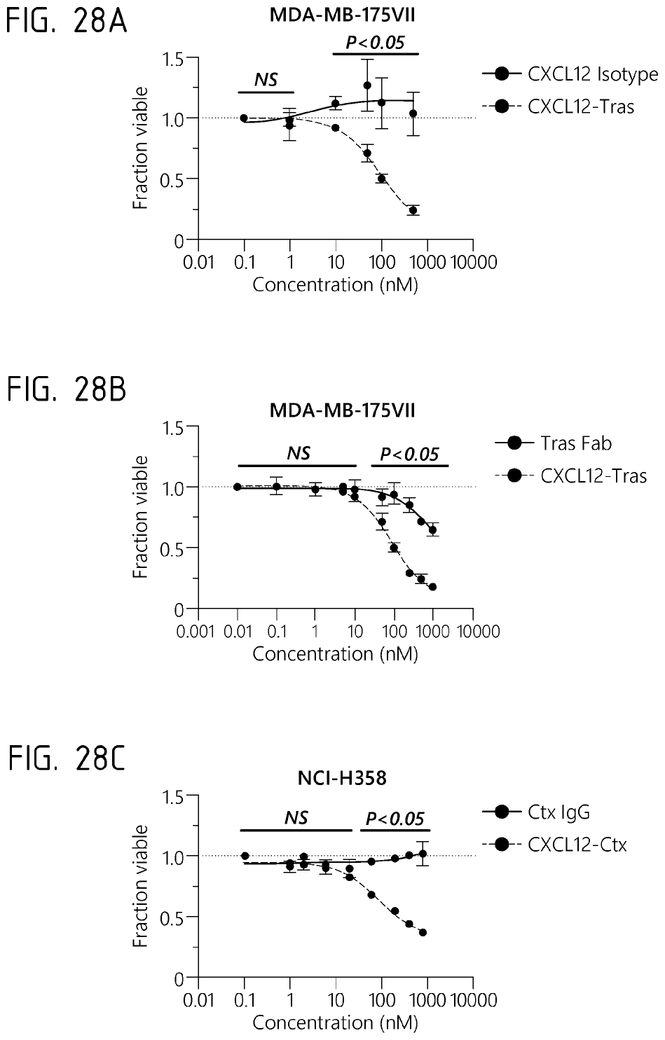

To elucidate whether KineTAC-mediated degradation could impart functional cellular consequences, cell viability of HER2 expressing cells was measured after treatment with CXCL12-Tras. MDA-MB-175VII and SK-BR-3 are breast cancer cell lines that are both reported to be sensitive to Trastuzumab treatment. These cell lines served as ideal models to test the functional consequence of HER2 degradation compared to inhibition with Trastuzumab Fab or IgG. To this end, cells were treated with either CXCL12-Tras, Fab, or IgG for 5 days, after which the cell viability was determined using a modified MTT assay. In MDA-MB-175VII cells, reduction in cell viability was observed at higher concentrations of CXCL12-Tras (IC₅₀=86.8 nM) and was significantly greater than Trastuzumab Fab or IgG alone (FIG. 9A). Although the Dₘₐₓ of HER2 degradation in SK-BR-3 was much lower than in MDA-MB-175VII (20% vs. 64%, respectively), treatment of SK-BR-3 cells with CXCL12-Tras also induced significant reduction in cell viability at high concentrations (IC₅₀=198.9 nM) (FIG. 9B). Reduction in cell viability was observed at higher concentrations of CXCL12-Tras and was significantly greater than trastuzumab IgG, trastuzumab Fab, or CXCL12 isotype alone (FIG. 31, Extended Data. FIG. 6c-d) FIGS. 28A-28B. We also found significant differences in cell viability in non-small cell lung cancer NCI-H358 cells treated with either CXCL12-Ctx or cetuximab IgG (Extended Data FIG. 6e) FIG. 28C. These data demonstrate that KineTAC-mediated degradation can cause functional consequences in reducing cancer cell viability in vitro. Furthermore, significant degradation of the HER2 is not needed to induce major reductions in cell viability.

### Example 7: KineTACs have Favorable PK Properties Despite Cross-Reactivity with Mouse

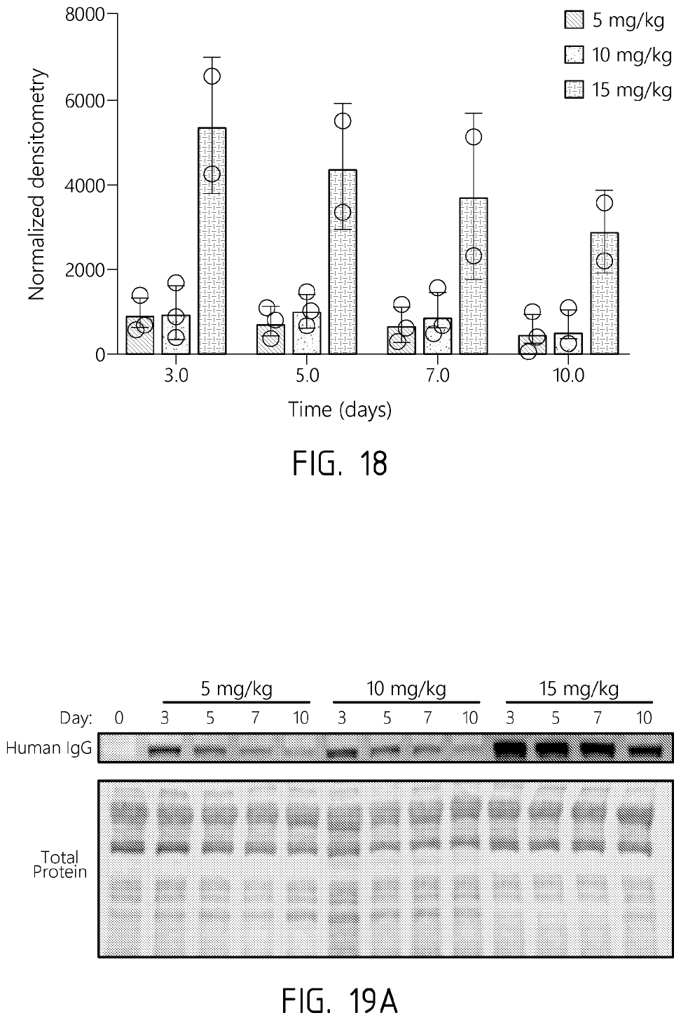

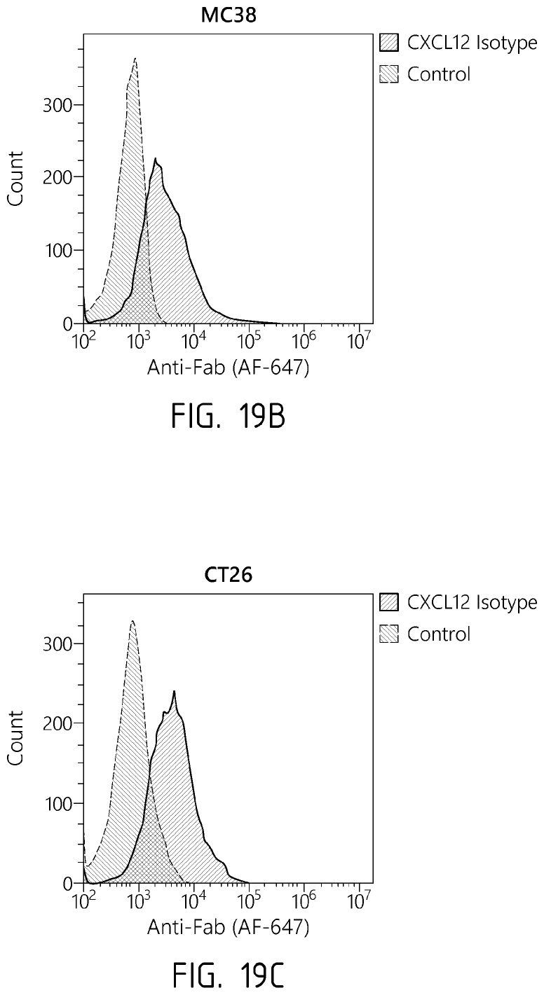

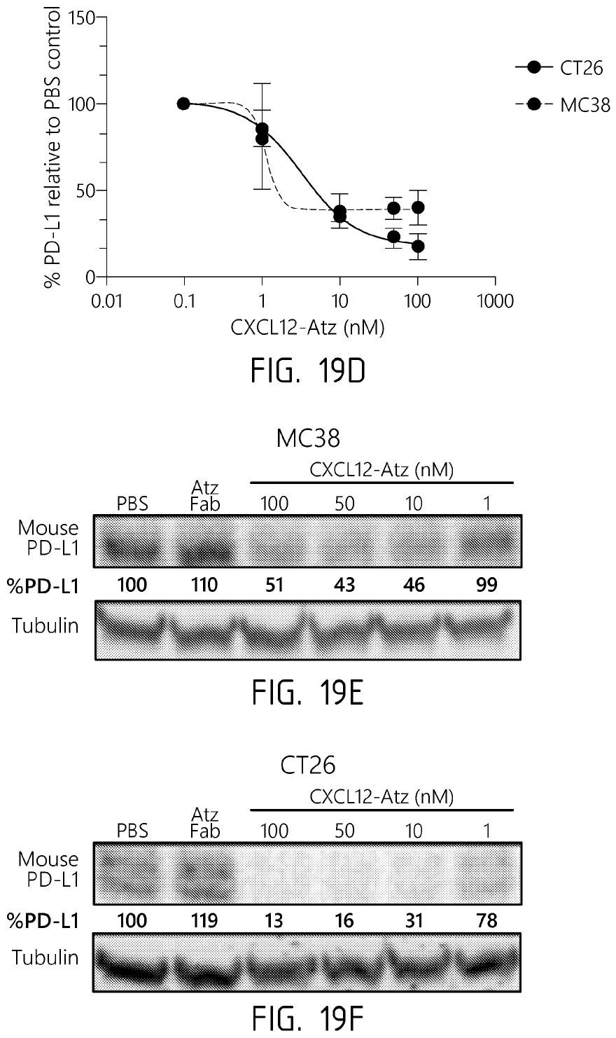

We next asked whether KineTACs would have similar antibody clearance to IgGs in vivo. To this end, male nude mice were injected intravenously with 5, 10, or 15 mg/kg CXCL12-Tras, which is a typical dose range for antibody xenograft studies. Western blotting analysis of plasma antibody levels revealed that the KineTAC remained in plasma up to 10 days post-injection with a half-life of 8.7 days, which is comparable to the reported half-life of IgGs in mice (FIG. 3m FIG. 18, Extended Data FIG. 7a) FIG. 19A.46 Given the high homology between human and mouse CXCL12, we tested whether human CXCL12 isotype was cross-reactive. In fact, human CXCL12 isotype bound to mouse cell lines MC38 and CT26 that endogenously express mouse CXCR7 (Extended Data FIG. 7b-c) FIGS. 19B-19C. Together, these results demonstrate that KineTACs have favorable stability and are not being rapidly cleared despite cross-reactivity with mouse CXCR7 receptors. Since atezolizumab is also known to be cross-reactive, CXCL12-Atz ability to degrade mouse PD-L1 was tested in both MC38 and CT26. Indeed, CXCL12-Atz mediates near complete degradation of mouse PD-L1 in both cell lines (Extended Data FIG. 7d-f) FIGS. 19D-19F. Thus, we observe a long in vivo half-life of KineTACs despite cross-reactivity and binding to mouse CXCR7 receptors.

### Example 8: KineTACs can Target Extracellular Soluble Proteins

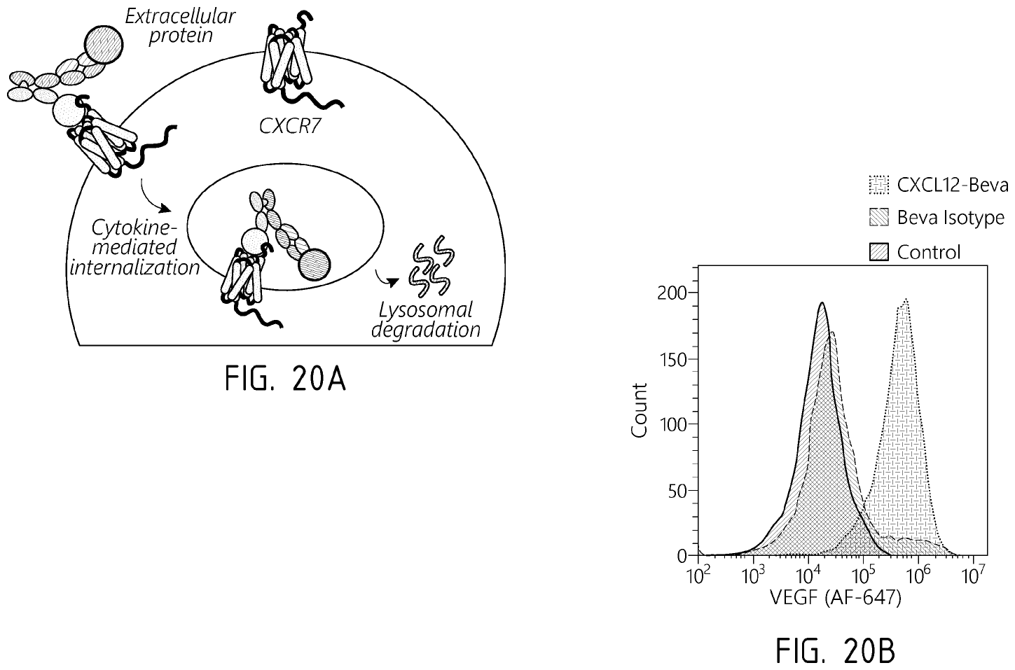

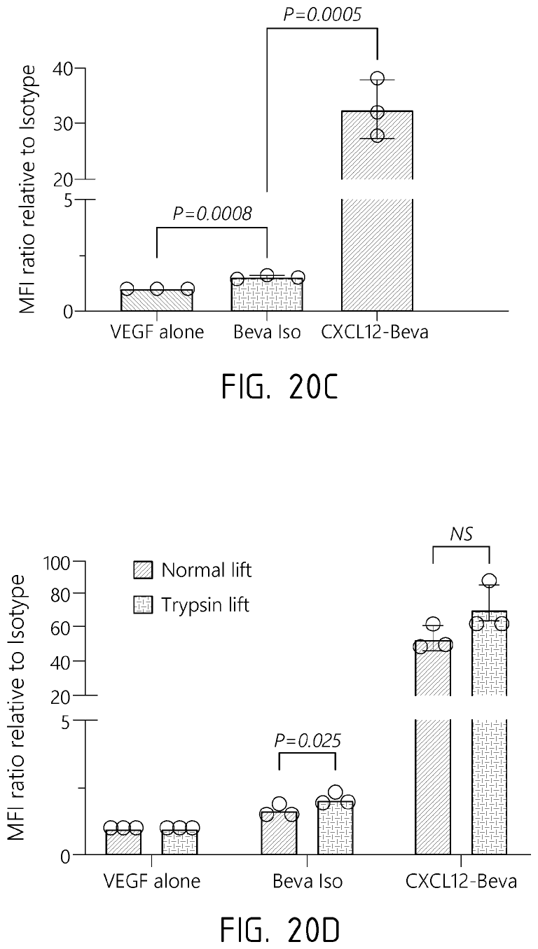

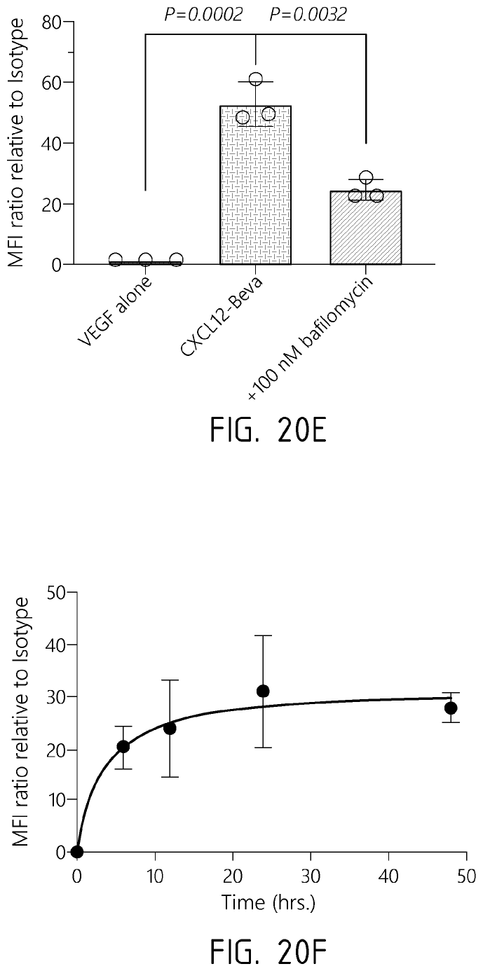

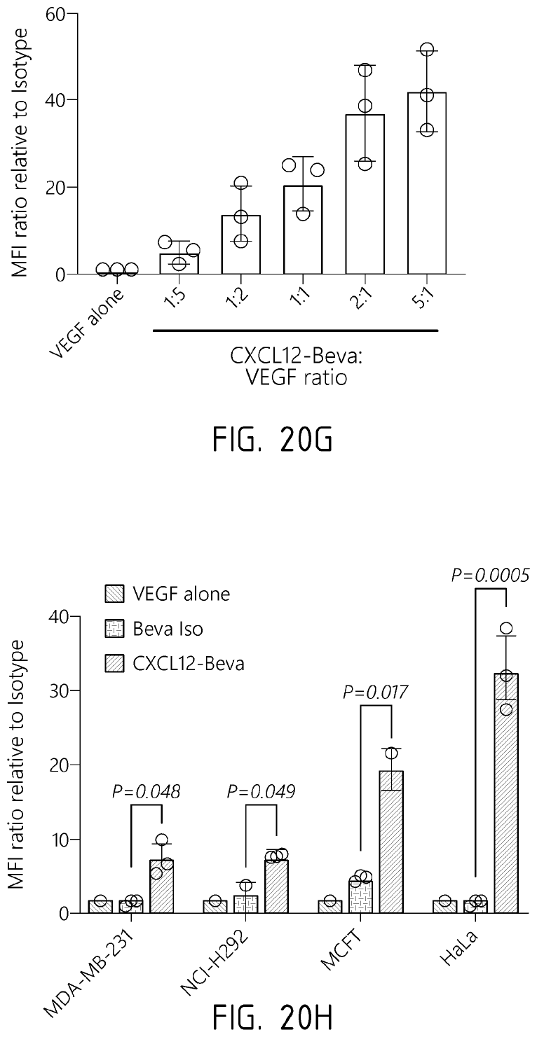

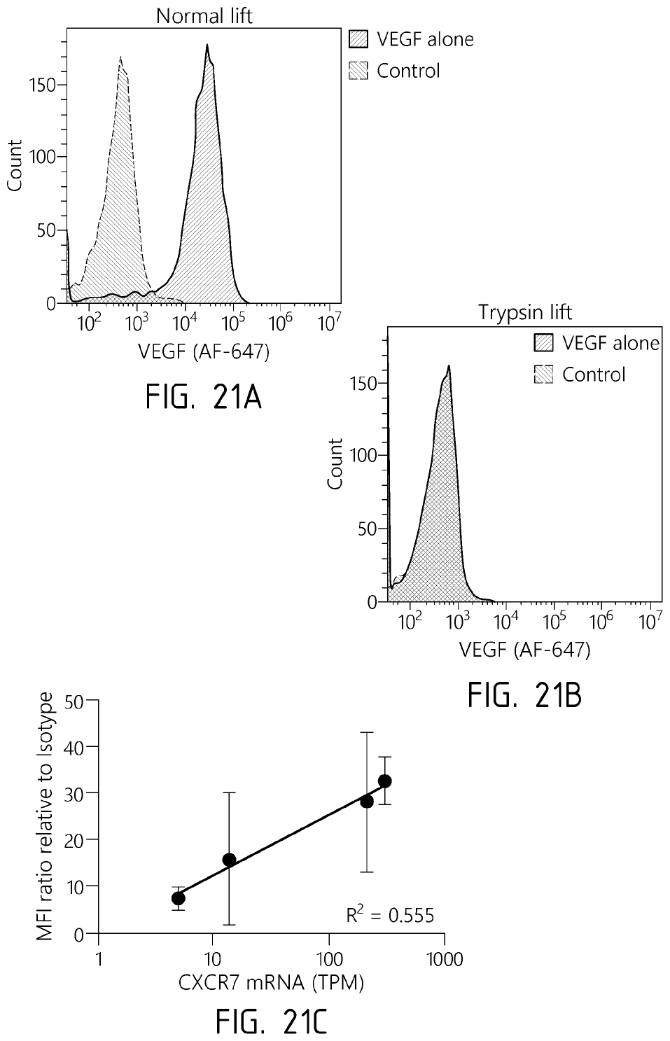

Having demonstrated the ability of KineTACs to mediate cell surface protein degradation, we next asked whether KineTACs could also be applied towards the degradation of soluble extracellular proteins. Antibodies binding to soluble ligands, such as vascular endothelial growth factor (VEGF) and tumor necrosis factor alpha (TNFa), are of tremendous therapeutic importance.47 Thus, we investigated whether KineTACs could promote cellular uptake of either VEGF or TNFa (FIG. 4a) FIG. 20A. VEGF was targeted by incorporating bevacizumab (Avastin), an FDA approved VEGF inhibitor, into the KineTAC scaffold (herein termed CXCL12-Beva). HeLa cells were incubated with VEGF-647 or VEGF-647 plus CXCL12-Beva for 24 hr. Following treatment, flow cytometry analysis showed a robust, 32-fold increase in cellular fluorescence when VEGF-647 was co-incubated with CXCL12-Beva, but not bevacizumab isotype which lacks the CXCL12 arm (FIG. 4b-c) FIGS. 20B-20C. To ensure that the increased cellular fluorescence was due to intracellular uptake of VEGF-647 and not surface binding, we determined the effect of a trypsin lift post-treatment which removes any cell surface VEGF-647 binding (Extended Data FIG. 8a-b) FIGS. 21A-21B. We do observe a difference between Beva isotype treated cells, which may reflect slight non-specific internalization that occurs with the trypsin lift. However, this difference was not observed for CXCL12-Beva. We found that there was no significant difference in cellular fluorescence levels between trypsin and normal lifted cells treated for 24 hr with CXCL12-Beva (FIG. 4d) FIG. 20D. Pre-treatment with bafilomycin, a lysosome inhibitor, also impairs the ability of CXCL12-Beva to uptake extracellular VEGF-647 as observed by a decrease in cellular fluorescence (FIG. 4e) FIG. 20E. VEGF-647 uptake was not completely impaired, likely due to bafilomycin blocking the conversion of endosome to lysosome, which would still allow some VEGF-647 to be endocytosed but not degraded. Together, these data suggest that KineTACs successfully mediate the intracellular uptake of extracellular VEGF and delivery to the lysosome. Similar to membrane protein degradation, KineTAC-mediated uptake of VEGF occurs in a time-dependent manner, with robust internalization occurring before 6 hrs and reaching steady state by 24 hrs (FIG. 4f) FIG. 20F. Furthermore, the levels of VEGF uptake are dependent on the KineTAC:ligand ratio and saturate at ratios greater than 1:1 (FIG. 4g) FIG. 20G. We next tested the ability of CXCL12-Beva to promote uptake on other cell lines, including breast and lung cancer lines, and find that these cells also significantly uptake VEGF (FIG. 4h) FIG. 20H. Moreover, the extent of uptake is correlated with the transcript levels of CXCR7 in these cells (R2=0.555, Extended Data FIG. 8c) FIG. 21C. These data suggest that KineTACs directed against soluble ligands can promote uptake of extracellular soluble ligands and that cell lines expressing higher levels of CXCR7 lead to greater uptake.

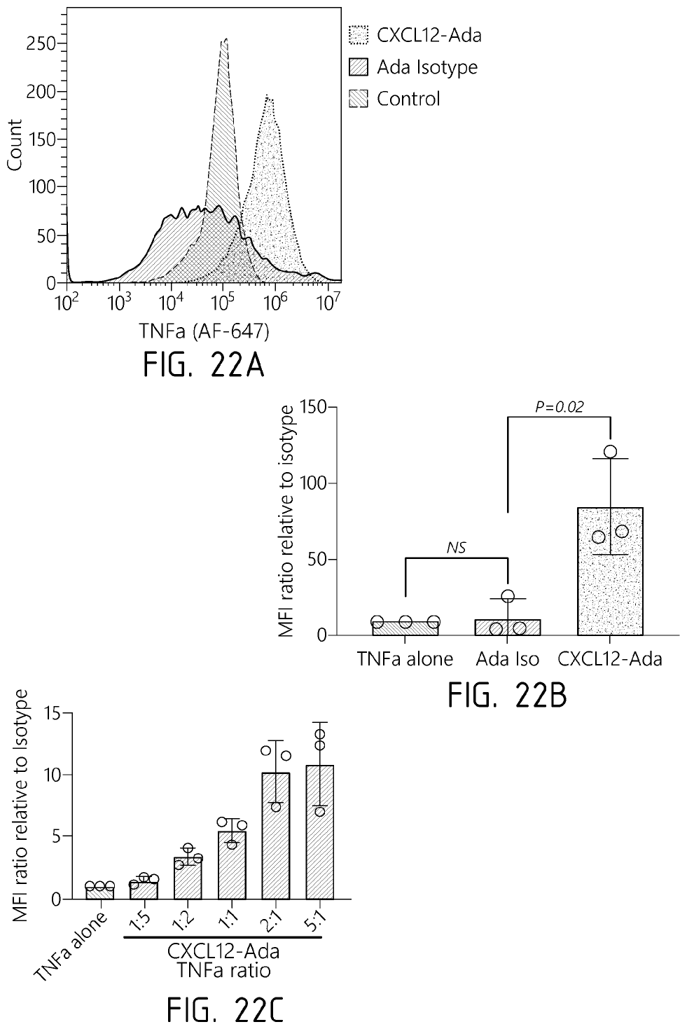

We next targeted TNFa by incorporating adalimumab (Humira), an FDA approved TNFa inhibitor, into the KineTAC scaffold (herein termed CXCL12-Ada). Following 24 hr treatment of HeLa cells, significant, 8.5-fold increase in cellular fluorescence was observed when TNFa-647 was co-incubated with CXCL12-Ada compared to adalimumab isotype (Extended Data FIG. 9a-b) FIGS. 22A-22B. Consistent with the VEGF uptake experiments, TNFa uptake was dependent on the KineTAC:ligand ratio (Extended Data FIG. 9c) FIG. 22C. Thus, KineTACs are generalizable in mediating the intracellular uptake of soluble ligands, significantly expanding the target scope of KineTAC-mediated targeted degradation.

### Example 9: Other Receptors can be Used for KineTAC-Mediated Degradation

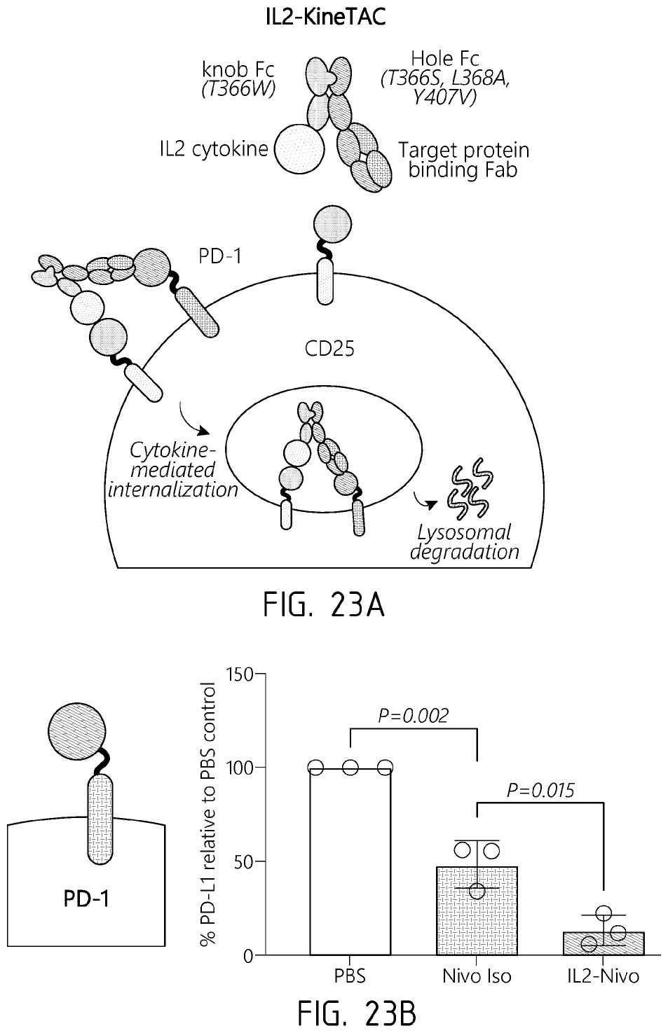

Finally, we asked whether alternative cytokine receptors could be co-opted by KineTACs to mediate the clearance of target proteins. We hypothesized that KineTACs bearing IL2 cytokine could engage CD25 as a degrader receptor, which is known to internalize and recycle back to the cell surface upon heterotrimer formation with IL2RB and IL2RG (Extended Data FIG. 10a) FIG. 23A. We generated knob-in-hole KineTACs in which human IL2 cytokine was N-terminally fused to the knob IgG1 Fc domain and the second arm contained the Fab antibody sequence for nivolumab. Activated T cells were then treated for 24 hr with IL2-bearing PD-1 KineTAC (herein termed IL2-Nivo). Following treatment with IL2-Nivo, cell surface PD-1 levels were significantly reduced, with a Dmax of 86.7%, compared to nivolumab isotype control (Extended Data FIG. 10b) FIG. 23B. This data demonstrates the generalizability of KineTACs to successfully co-opt alternative cytokine receptors, such as the interleukin receptor class CD25 is part of, for targeted protein degradation. Together, these data demonstrate that our KineTACs are exquisitely selective, degrading only the target protein.

### Example 10: Materials and Methods

This Example describes the materials and methods used in the Examples described above.

Cell lines: Cell lines were grown and maintained in T75 (Thermo Fisher Scientific) flasks at 37° C. and 5% CO₂. MDA-MB-231, MDA-MB-175VII, and MDA-MB-361 cells were grown in DMEM supplemented with 10% fetal bovine serum (FBS) and 1% penicillin/streptomycin. HeLa cells were grown in EMEM supplemented with 10% FBS and 1% penicillin/streptomycin. MCF-7 cells were grown in RPMI-1640 supplemented with 10% FBS and 1% penicillin/streptomycin. SK-BR-3 cells were grown in McCoy's 5A supplemented with 10% FBS and 1% penicillin/streptomycin.

Protein expression: Fabs were expressed in E. coli C43(DE3) Pro+ grown in an optimized TB autoinduction media at 37° C. for 6 hrs, then cooled to 30° C. for 18 hrs, and purified by Protein A affinity chromatography. IgGs and bispecifics were expressed and purified from Expi293 BirA cells using transient transfection (Expifectamine, Thermo Fisher Scientific). Enhancers were added 20 hrs after transfection. Cells were incubated for 5 days at 37° C. and 8% CO₂. Media was then harvested by centrifugation at 4,000×g for 20 min. IgGs were purified by Protein A affinity chromatography and buffer exchanged into PBS by spin concentration and flash frozen for storage at −80° C. Bispecifics were purified by Ni-NTA affinity chromatography and buffer exchanged into PBS containing 20% glycerol, concentrated, and flash frozen for storage at −80° C. Purity and integrity of all proteins were assessed by SDS-PAGE.

Bio-layer interferometry: Bio-layer interferometry (BLI) data were measured using an Octet RED384 (ForteBio) instrument. Biotinylated antigens were immobilized on a streptavidin biosensor and loaded until 0.4 nm signal was achieved. After blocking with 10 μM biotin, purified antibodies in solution was used as the analyte. PBSTB was used for all buffers. Data were analyzed using the ForteBio Octet analysis software and kinetic parameters were determined using a 1:1 monovalent binding model.

Degradation experiments: Cells were plated in 6- or 12-well plates and grown to ˜70% confluency before treatment. Media was aspirated and cells were treated with bispecifics or control antibodies in complete growth medium. After incubation at 37° C. for the designated time point, cells were washed with phosphate-buffered saline (PBS), lifted with versene, and harvested by centrifugation at 300×g for 5 min at 4° C. Samples were then tested by western blotting or flow cytometry to quantify protein levels.

Western blotting: Cell pellets were lysed with 1× RIPA buffer containing cOmplete mini protease inhibitor cocktail (Sigma-Aldrich) at 4° C. for 40 min. Lysates were centrifuged at 16,000×g for 10 min at 4° C. and protein concentrations were normalized using BCA assay (Pierce). 4× NuPAGE LDS sample buffer (Invitrogen) and 2-mercaptoethanol (BME) was added to the lysates and boiled for 10 min. Equal amounts of lysates were loaded onto a 4-12% Bis-Tris gel and ran at 200V for 37 min. The gel was incubated in 20% ethanol for 10 min and transferred onto a polyvinylidene difluoride (PVDF) membrane. The membrane was blocked in PBS with 0.1% Tween-20+5% bovine serum albumin (BSA) for 30 min at room temperature with gentle shaking. Membranes were incubated overnight with primary antibodies at respective dilutions at 4° C. with gentle shaking in PBS+0.2% Tween-20+5% BSA. Membranes were washed four times with tris-buffered saline (TBS)+0.1% Tween-20 and then co-incubated with HRP-anti-rabbit IgG (Cell Signaling Technologies, 7074A, 1:2000) and 680RD goat anti-mouse IgG (LI-COR, 926-68070, 1:10000) in PBS+0.2% Tween-20+5% BSA for 1 hr at room temperature. Membranes were washed four times with TBS+0.1% Tween-20, then washed with PBS. Membranes were imaged using an OdysseyCLxImager (L1-COR). SuperSignal West Pico PLUS Chemiluminescent Substrate (Thermo Fisher Scientific) was then added and imaged using a ChemiDoc Imager (BioRad). Band intensities were quantified using Image Studio Software (L1-COR).

Flow cytometry: Cell pellets were washed with cold PBS and centrifuged at 300×g for 5 min. Cells were blocked with cold PBS+3% BSA and centrifuged (300×g for 5 min). Cells were incubated with primary antibodies diluted in PBS+3% BSA for 30 min at 4° C. Cells were washed three times with cold PBS+3% BSA and secondary antibodies (if applicable) diluted in PBS+3% BSA added and incubated for 30 min at 4° C. Cells were washed three times with cold PBS+3% BSA and resuspended in cold PBS. Flow cytometry was performed on a CytoFLEX cytometer (Beckman Coulter) and gating was performed on single cells and live cells before acquisition of 10,000 cells. Analysis was performed using the FlowJo software package.

siRNA knockdown: HeLa cells were plated in a 6-well plate and grown to confluency. Cells were transfected with 20 pmol of siRNA (ON-TARGETplus siRNA SMARTPool, Dharmacon) and DharmaFECT 4 reagent (Dharmacon) according to manufacturer's instruction. Cells were incubated for 48 hrs at 37° C. under 5% CO₂ and siRNA knockdown was validated by western blotting.

Cell culture/SILAC labeling and treatment: MDA-MB-231 cells were grown in DMEM for SILAC (Thermo Fisher) with 10% dialyzed FBS (Gemini). Media was also supplemented with either light L-[12C6,14N2] lysine/L-[12C6,14N4] arginine (Sigma) or heavy L-[13C6,15N2] lysine/L-[13C6,15N4] arginine (Cambridge Isotope Laboratories, Tewksbury, MA). Cells were maintained in SILAC media for five passages to ensure complete isotopic labeling. Cells were then treated with either PBS control or 100 nM bispecific for 48 hours before cells were collected and heavy/light-labeled cells mixed at a 1:1 ratio in both forward and reverse mode. A small portion of these cells were set aside for whole cell proteomic analysis, and the remainder were used to prepare surface proteome enrichment.

Mass spectrometry sample preparation: Cell surface glycoproteins were captured largely as previously described, but using a modified protocol to facilitate small sample input. Briefly, cells were first washed in PBS, pH 6.5 before the glycoproteins were oxidized with 1.6 mM NaIO₄ (Sigma) in PBS, pH 6.5 for 20 minutes at 4° C. Cells were then biotinylated via the oxidized vicinal diols with 1 mM biocytin hydrazide (Biotium) in the presence of 10 mM aniline (Sigma) in PBS, pH 6.5 for 90 minutes at 4° C. Cell pellets were lysed with a 2× dilution of commercial RIPA buffer (Millipore) supplemented with 1× Protease Inhibitor Cocktail (Sigma) and 2 mM EDTA (Sigma) for 10 minutes at 4° C. Cells were further disrupted with probe sonication and the cell lysates were then incubated with NeutrAvidin coated agarose beads (Thermo) in Poly-Prep chromatography columns (Bio-Rad) for two hours at 4° C. to isolate biotinylated glycoproteins. After this incubation, cells were washed sequentially with 1× RIPA (Millipore) plus 1 mM EDTA, high salt PBS (PBS pH 7.4, 2 M NaCl [Sigma]), and denaturing urea buffer (50 mM ammonium bicarbonate, 2 M Urea). All wash buffers were heated to 42° C. prior to use. Proteins on the beads were next digested and desalted using the Preomics iST mass spectrometry sample preparation kit (Preomics) per the manufacturer's recommendations with few modifications. First, samples were resuspended in the “LYSE” solution and transferred to fresh tubes. After incubating in “LYSE” for 10 minutes at 55° C., the “DIGEST” solution was added and beads incubated for 90 minutes at 37° C. with mixing at 500 rpm. Following on-bead digest, the peptide eluate was isolated using SnapCap spin columns (Pierce) and the “STOP” solution added. The sample was then transferred to the Preomics cartridge and desalted using the manufacturer's protocol. Samples were dried, resuspended in 0.1% formic acid, 2% acetonitrile (Fisher), and quantified using the Pierce Peptide Quantification Kit prior to LC-MS/MS analysis. Whole cell lysate samples were prepared using the Preomics kit protocol for whole lysate samples. Resulting peptides were dried and quantified in the same manner as the surface enriched samples.

Mass spectrometry: LC-MS/MS was performed using a Bruker NanoElute chromatography system coupled to a Bruker timsTOF Pro mass spectrometer. Peptides were separated using a pre-packed IonOpticks Aurora (25 cm×75 μm) C18 reversed phase column (1.6 μm pore size, Thermo) fitted with a CaptiveSpray emitter for the timsTOF Pro CaptiveSpray source. For all samples, 200 ng of resuspended peptides were injected and separated using a linear gradient of 2-23% solvent B (solvent A: 0.1% formic acid+2% acetonitrile, solvent B: acetonitrile with 0.1% formic acid) over 90 minutes at 400 μL/minute with a final ramp to 34% B over 10 minutes. Separations were performed at a column temperature of 50° C. Data-dependent acquisition was performed using a timsTOF PASEF MS/MS method (TIMS mobility scan range 0.70-1.50 V·s/cm²; mass scan range 100-1700 m/z; ramp time 100 milliseconds; 10 PASEF scans per 1.17 seconds; active exclusion 24 seconds; charge range 0-5; minimum MS1 intensity 500). The normalized collision energy was set at 20.

Data analysis/Statistics: SILAC proteomics data were analyzed using PEAKSOnline (v1.4). For all samples, searches were performed with a precursor mass error tolerance of 20 ppm and a fragment mass error tolerance of 0.03 Da. The digest was considered to be semi-specific and up to 3 missed cleavages were allowed. For whole cell proteome data, the reviewed SwissProt database for the human proteome (downloaded Dec. 12, 2020) was used. For surface enriched samples, a database composed of SwissProt proteins annotated “membrane” but not “nuclear” or “mitochondrial” was used to ensure accurate unique peptide identification for surface proteins, as previously described. Carbamidomethylation of cystine was used as a fixed modification, whereas the isotopic labels for arginine and lysine, acetylation of the N-terminus, oxidation of methionine, and deamidation of asparagine and glutamine were set as variable modifications. Only PSMs and protein groups with an FDR of less than 1% were considered for downstream analysis. SILAC analysis was performed using the forward and reverse samples, and at least 2 labels for the ID and features were required. Proteins showing a >2-fold change from PBS control with a significance of P<0.01 were considered to be significantly changed.

Cell viability experiments: Cell viability assays were performed using an MTT modified assay. In brief, on day 0 15,000 MDA-MB-175VII, 7,000 NCI-H358, 2,000 HCC4006 and SK-BR-3 cells were plated in each well of a 96-well plate. On day 1, bispecifics or control antibodies were added in a dilution series. Cells were incubated at 37° C. under 5% CO₂ for 5 days. On day 6, 40 μL of 2.5 mg/mL thiazolyl blue tetrazolium bromide (GoldBio) was added to each well and incubated at 37° C. under 5% CO₂ for 4 hrs. 100 μL of 10% SDS in 0.01M HCl was then added to lyse cells and release MTT product. After 4 hrs at room temperature, absorbance at 600 nm was quantified using an Infinite M200 PRO plate reader (Tecan). Data was plotted using GraphPad Prism software (version 9.0) and curves were generated using non-linear regression with sigmoidal 4PL parameters.

Primary human CD8+ T cell isolation: Primary human T cells were isolated from leukoreductin chamber residuals following Trima Apheresis (Blood Centers of the Pacific) using established protocols.⁴⁸ Briefly, peripheral blood mononuclear cells (PBMCs) were isolated using Ficoll separation in SepMate tubes (STEMCELL Technologies) according to manufacturer's instructions. CD8+ T cells were isolated from PBMCs using the EasySep™ Human CD8+ T cell Isolation Kit following the manufacturer's protocol. Isolated cell populations were then analyzed for purity by flow cytometry on a Beckman Coulter CytoFlex flow cytometer using a panel of antibodies (anti-CD3, anti-CD4, anti-CD8a, all from BioLegend).

CD8+ T cell activation: Following CD8+ T cell isolation, cells were stimulated with recombinant IL-2 (GoldBio), IL-15 (GoldBio), and ImmunoCult Human CD3/CD28 T cell Activation (STEMCELL Technologies) for 4 days at 37° C. Activated CD8+ T cells were then analyzed for the upregulation of activation markers CD25 and PD-1 by flow cytometry using anti-CD25 and anti-PD-1 antibodies (BioLegend). Once activation was confirmed, cells were dosed as described above and levels of target protein analyzed by flow cytometry.

Flow cytometry for soluble ligand uptake: Cell pellets were washed three times with cold PBS and centrifuged at 300×g for 5 min. Cells were then resuspended in cold PBS. Flow cytometry was performed on a CytoFLEX cytometer (Beckman Coulter) and gating was performed on single cells and live cells before acquisition of 10,000 cells. Analysis was performed using the FlowJo software package.

Trypsin lift for soluble ligand uptake: Cell pellets were washed three times with cold PBS and centrifuged at 300×g for 5 min. Cells were then resuspended in cold PBS. Flow cytometry was performed on a CytoFLEX cytometer (Beckman Coulter) and gating was performed on single cells and live cells before acquisition of 10,000 cells. Analysis was performed using the FlowJo software package.

Confocal microscopy: Cells were plated onto Mat-Tek 35 mm glass bottom petri dishes pre-treated with poly-D-lysine and grown to ˜70% confluency before treatment. Media was aspirated and cells were treated with bispecifics or control antibodies in complete growth medium. For soluble ligand uptake experiments, biotinylated soluble ligand was pre-incubated with streptavidin-647 at 37° C. for 30 min, then mixed with bispecific or control antibodies and added to cells. After 24 hr incubation at 37° C., media was aspirated, and cells were washed with PBS. Cells were then stained using standard protocols for LysoTracker Deep Red (Invitrogen), DAPI (Cell Signaling Technologies), and primary antibody. Samples were imaged using a Nikon Ti Microscope with Yokogawa CSU-22 spinning disk confocal and a 100× objective lens. 405, 488, and 647 nm lasers were used to image DAPI, primary antibody, and LysoTracker, respectively. Images were deconvoluted and processed using NIS-Element and FIJI software packages.

Antibody in vivo stability study: Male nude nu/nu mice (8-10 weeks old, bred at the UCSF MZ Breeding Facility) were treated with 5, 10, or 15 mg/kg CXCL12-Tras via intravenous injection (3 mice per group). Blood was collected from the lateral saphenous vein using EDTA capillary tubes at day 0 prior to intravenous injection and at days 3, 5, 7, and 10 post injection. Plasma was separated after centrifugation at 700×g at 4° C. for 15 min. To determine the levels of CXCL12-Tras, 1 μL of plasma was diluted into 30 μL of NuPAGE LDS sample buffer (Invitrogen) and loaded onto a 4-12% Bis-Tris gel and ran at 200V for 37 mi. The gel was incubated in 20% ethanol for 10 min and transferred onto a polyvinylidene difluoride (PVDF) membrane. The membrane was washed with water followed by incubation for 5 mi with REVERT 700 Total Protein Stain (L1-COR). The blot was then washed twice with REVERT 700 Wash Solution (L1-COR) and imaged using an OdysseyCLxmager (L1-COR). The membrane was then blocked in PBS with 0.1% Tween-20+5% bovine serum albumin (BSA) for 30 m at room temperature with gentle shaking. Membranes were incubated overnight with 800 CW goat ant-human IgG (L1-COR, 1:10000) at 4° C. with gentle shaking in PBS+0.2% Tween-20+5% BSA. Membranes were washed four times with tris-buffered saline (TBS)+0.1% Tween-20 and then washed with PBS. Membranes were imaged using an OdysseyCLxImager (L1-COR). Band intensities were quantified using Image Studio Software (L1-COR).

Antibodies and conditions used in the Examples related to the present disclosure are provided below in Table 6.

TABLE 6
Antibodies and usage conditions

| Antibody | Source (Cat. #) | Usage, Dilution |
|---|---|---|
| Rabbit anti-PD-L1 | Cell Signaling Technologies | WB, 1:1000 |
| | (13684S) | |
| Rabbit anti-EGFR | Cell Signaling Technologies | WB, 1:1000 |
| | (4267S) | |
| Rabbit anti-HER2 | Cell Signaling Technologies | WB, 1:1000 |
| | (4290S) | |
| Rabbit anti-CDCP1 | Cell Signaling Technologies | WB, 1:1000 |
| | (13794S) | |
| Mouse anti-Tubulin | Cell Signaling Technologies | WB, 1:1600 |
| | (3873S) | |
| Mouse anti-β-Actin | Cell Signaling Technologies | WB, 1:1000 |
| | (3700S) | |
| Rabbit anti-CXCR7 | Abcam (ab138509) | WB, 1:1000 |
| Rabbit anti-CXCR4 | Abcam (ab124824) | WB, 1:1000 |
| 680RD Goat anti-Mouse IgG | LI-COR (926-68070) | WB, 1:10000 |
| 800CW Goat anti-Rabbit IgG | LI-COR (926-32211) | WB, 1:10000 |
| HRP anti-Rabbit IgG | Cell Signaling Technologies | WB, 1:2000 |
| | (7074A) | |
| Rabbit anti-PD-L1 Alexa 488 | Cell Signaling Technologies | IF, 1:50 |
| Conjugate | (25048S) | |
| Rabbit anti-EGFR Alexa 488 | Cell Signaling Technologies | IF, 1:100 |
| Conjugate | (5616S) | |
| Rabbit anti-EGFR Alexa 647 | Cell Signaling Technologies | IF, 1:50 |
| Conjugate | (5588S) | |
| Rabbit anti-PD-L1 Alexa 647 | Cell Signaling Technologies | FC, 1:50; IF, 1:400 |
| Conjugate | (41726S) | |
| Rabbit IgG Isotype Control | Cell Signaling Technologies | FC, 1:50 |
| Alexa 647 Conjugate | (3452S) | |
| Cetuximab | Selleck Chemicals (A2000) | Functional |

### Example 11: Other Chemokines, Cytokines, and Growth Factors Useful as KineTACS

This Example describes the expansion of KineTACS beyond CXCL12, CXCL11, vMIPII, and IL2.

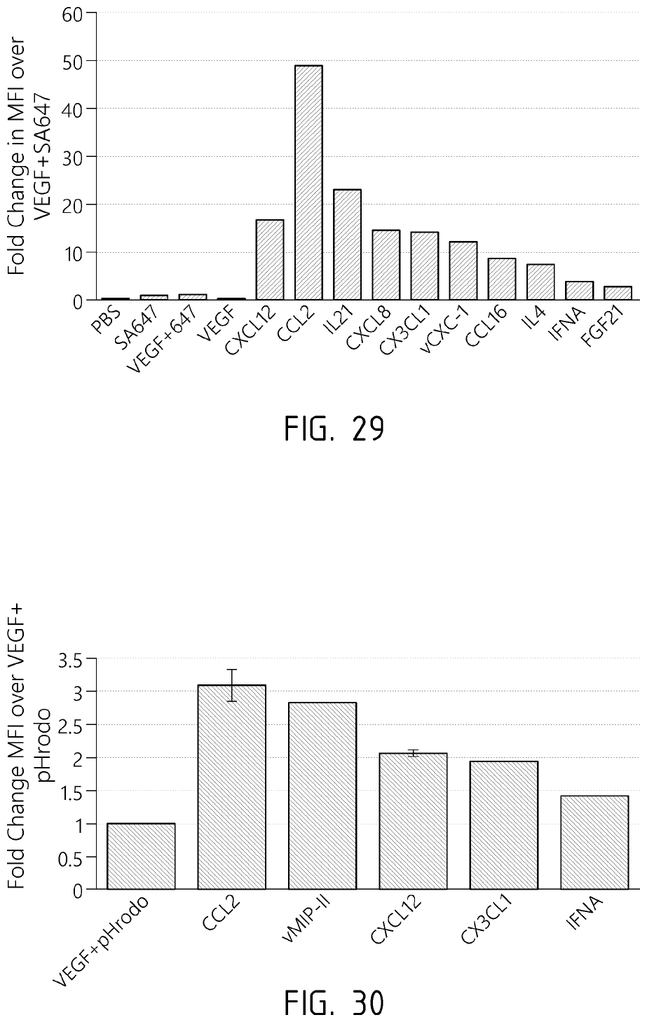

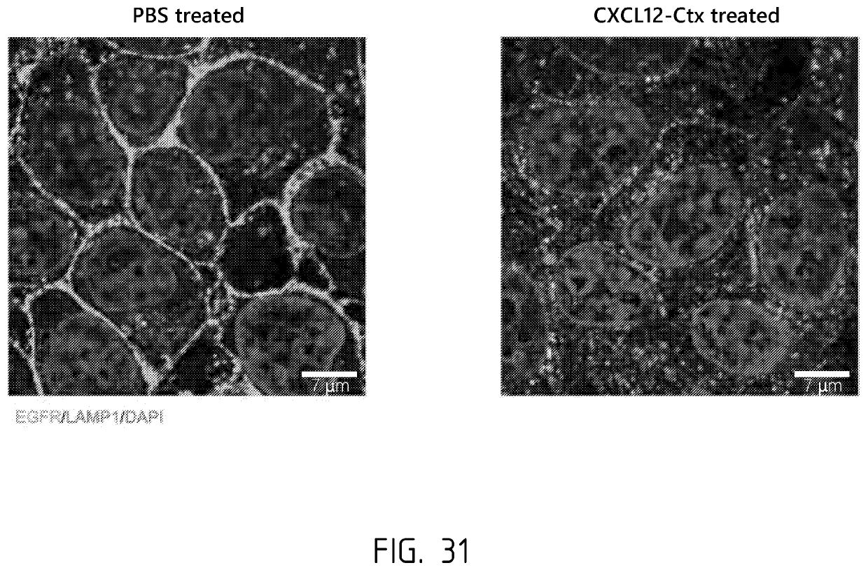

First, 100,000 cells were incubated for 24 hrs at 37° C. in 500 μL conditioned media containing control compounds or KineTACs and VEGF647. 100 nM biotinylated VEGF was added to 200 nM SA647 and allowed to bind for 15 mins at 37° C. KineTACs (CXCL12, CCL2, IL21, CXCL8, CX3CL1, vCXC-1, CCL16, IL4, IFNA, FGF21) were then added for a final concentration of 25 nM KineTAC and 50 nM VEGF647. The mixture was then immediately dosed onto cells and allowed to incubate for 24 hrs at 37° C. before harvesting by centrifugation at 1000×g, washing 3× with PBS, and performing flow cytometry. Plots were gated for live and singlet cells and the median fluorescence intensity (MFI) in the APC channel was used to quantify the ability of the KineTAC to mediate VEGF647 uptake. Fold change was measured over solely VEGF647 alone (FIG. 29). Next, 100,000 cells were incubated for 24 hrs at 37° C. in 500 μL conditioned media containing control compounds or KineTACs and VEGF647. 100 nM biotinylated VEGF was added to 200 nM SA-pHrodoRed conjugate and allowed to bind for 15 mins at 37° C. KineTACs (CCL2, vMIP-II, CXCL12, CX3CL1, and IFNA) were then added to VEGF-pHrodoRed for a final concentration of 25 nM KineTAC and 50 nM VEGF-pHrodoRed. pHrodo Red is a pH sensitive dye that fluorescenes under acidic conditions. Thus, this dye was used to determine whether VEGF was being localized with acidic compartments, such as the lysosome and late endosome, after KineTAC treatment. The mixture was then immediately dosed onto cells and allowed to incubate for 24 hrs at 37° C. before harvesting by centrifugation at 1000×g, washing 3× with PBS, and performing flow cytometry. Plots were gated for live and singlet cells and MFI in the PE channel was used to quantify the ability of the KineTAC to mediate VEGF-pHrodoRed uptake. Fold change was measured over VEGF-pHrodoRed alone (FIG. 30). It was also found that CCL20 exhibited a 40% increase in VEGF uptake as compared to control.

The data does show that in most cases, VEGF is localized to an acidic intracellular compartment, such as the lysosome or late endosome, as a result of KineTAC treatment. This occurs within 24 hrs and is greater than VEGF alone (FIGS. 29-30). The constructs produced and purified tend to direct a fluorescently labeled VEGF onto or inside of cells within 24 hrs.

Some constructs shown below in Table 7 have been generated and successfully expressed. The remainder will be completed. All of these constructs will be tested using protocols described in Example 12.

TABLE 7

| Con- | | | SEQ |
|---|---|---|---|
| struct | | | ID |
| Name | | Construct Sequence | NO |
| vCXC-1 | Knob: | MRLIFGALIIFLAYVYHYEVNGTELRCRCL | |
| | | HRKWPPNKIILGNYWLHRDPRGPGCDKNEH | |
| | | LLYPDGRKPPGPGVCLSPDHLFSKWLDKHN | |
| | | DNRWYNVNITKSPGPRRINITLIGVRGEPK | |
| | | SCDKTHTCPPCPAPELLGGPSVFLFPPKPK | |
| | | DTLMISRTPEVTCVVVDVSHEDPEVKFNWY | |
| | | VDGVEVHNAKTKPREEQYGSTYRVVSVLTV | |
| | | LHQDWLNGKEYKCKVSNKALPAPIEKTISK | |
| | | AKGQPREPQVYTLPPSRDELTKNQVSLWCL | |
| | | VKGFYPSDIAVEWESNGQPENNYKTTPPVL | |
| | | DSDGSFFLYSKLTVDKSRWQQGNVFSCSVM | |
| | | HEALHNHYTQKSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| vMIP-II | Knob: | GDTLGASWHRPDKCCLGYQKRPLPQVLLSS | |
| | | WYPTSQLCSKPGVIFLTKRGRQVCADKSKD | |
| | | WVKKLMQQLPVTAREPKSCDKTHTCPPCPA | |
| | | PELLGGPSVFLFPPKPKDTLMISRTPEVTC | |
| | | VVVDVSHEDPEVKFNWYVDGVEVHNAKTKP | |
| | | REEQYGSTYRVVSVLTVLHQDWLNGKEYKC | |
| | | KVSNKALPAPIEKTISKAKGQPREPQVYTL | |
| | | PPSRDELTKNQVSLWCLVKGFYPSDIAVEW | |
| | | ESNGQPENNYKTTPPVLDSDGSFFLYSKLT | |
| | | VDKSRWQQGNVFSCSVMHEALHNHYTQKSL | |
| | | SLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| U83 | Knob: | MAIGFICSSPDAELFSEKSRMSSSVLLGCL | |
| | | LCCMDWSAAVPGKTEPFRKLFDAIMIKKLK | |
| | | SCSAAYPSDLDEGSMCDMADASPTSLELGL | |
| | | SKLDKESEPKSCDKTHTCPPCPAPELLGGP | |
| | | SVFLFPPKPKDTLMISRTPEVTCVVVDVSH | |
| | | EDPEVKFNWYVDGVEVHNAKTKPREEQYGS | |
| | | TYRVVSVLTVLHQDWLNGKEYKCKVSNKAL | |
| | | PAPIEKTISKAKGQPREPQVYTLPPSRDEL | |
| | | TKNQVSLWCLVKGFYPSDIAVEWESNGQPE | |
| | | NNYKTTPPVLDSDGSFFLYSKLTVDKSRWQ | |
| | | QGNVFSCSVMHEALHNHYTQKSLSLSPGKG | |
| | | GSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RESGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| IL-3 | Knob: | APMTQTTPLKTSWVNCSNMIDEIITHLKQP | |
| | | PLPLLDENNLNGEDQDILMENNLRRPNLEA | |
| | | FNRAVKSLQNASAIESILKNLLPCLPLATA | |
| | | APTRHPIHIKDGDWNEFRRKLTFYLKTLEN | |
| | | AQAQQTTLSLAIFEPKSCDKTHTCPPCPAP | |
| | | ELLGGPSVFLFPPKPKDTLMISRTPEVTCV | |
| | | VVDVSHEDPEVKFNWYVDGVEVHNAKTKPR | |
| | | EEQYGSTYRVVSVLTVLHQDWLNGKEYKCK | |
| | | VSNKALPAPIEKTISKAKGQPREPQVYTLP | |
| | | PSRDELTKNQVSLWCLVKGFYPSDIAVEWE | |
| | | SNGQPENNYKTTPPVLDSDGSFFLYSKLTV | |
| | | DKSRWQQGNVFSCSVMHEALHNHYTQKSLS | |
| | | LSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| IL-4 | Knob: | HKCDITLQEIIKTLNSLTEQKTLCTELTVT | |
| | | DIFAASKNTTEKETFCRAATVLRQFYSHHE | |
| | | KDTRCLGATAQQFHRHKQLIRFLKRLDRNL | |
| | | WGLAGLNSCPVKEANQSTLENFLERLKTIM | |
| | | REKYSKCSSEPKSCDKTHTCPPCPAPELLG | |
| | | GPSVFLFPPKPKDTLMISRTPEVTCVVVDV | |
| | | SHEDPEVKFNWYVDGVEVHNAKTKPREEQY | |
| | | GSTYRVVSVLTVLHQDWLNGKEYKCKVSNK | |
| | | ALPAPIEKTISKAKGQPREPQVYTLPPSRD | |
| | | ELTKNQVSLWCLVKGFYPSDIAVEWESNGQ | |
| | | PENNYKTTPPVLDSDGSFFLYSKLTVDKSR | |
| | | WQQGNVFSCSVMHEALHNHYTQKSLSLSPG | |
| | | KGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | Q | |
| | | YGSTYRVVSVLTVLHQDWLNGKEYKCKVSN | |
| | | KALPAPIEKTISKAKGQPREPQVYTLPPSR | |
| | | DELTKNQVSLSCAVKGFYPSDIAVEWESNG | |
| | | QPENNYKTTPPVLDSDGSFFLVSKLTVDKS | |
| | | RWQQGNVFSCSVMHEALHNHYTQKSLSLSP | |
| | | GKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| IL-5 | Knob: | IPTEIPTSALVKETLALLSTHRTLLIANET | |
| | | LRIPVPVHKNHQLCTEEIFQGIGTLESQTV | |
| | | QGGTVERLFKNLSLIKKYIDGQKKKCGEER | |
| | | RRVNQFLDYLQEFLGVMNTEWIIESEPKSC | |
| | | DKTHTCPPCPAPELLGGPSVFLFPPKPKDT | |
| | | LMISRTPEVTCVVVDVSHEDPEVKENWYVD | |
| | | GVEVHNAKTKPREEQYGSTYRVVSVLTVLH | |
| | | QDWLNGKEYKCKVSNKALPAPIEKTISKAK | |
| | | GQPREPQVYTLPPSRDELTKNQVSLWCLVK | |
| | | GFYPSDIAVEWESNGQPENNYKTTPPVLDS | |
| | | DGSFFLYSKLTVDKSRWQQGNVFSCSVMHE | |
| | | ALHNHYTQKSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| IL-6 | Knob: | VPPGEDSKDVAAPHRQPLTSSERIDKQIRY | |
| | | ILDGISALRKETCNKSNMCESSKEALAENN | |
| | | LNLPKMAEKDGCFQSGFNEETCLVKIITGL | |
| | | LEFEVYLEYLQNRFESSEEQARAVQMSTKV | |
| | | LIQFLQKKAKNLDAITTPDPTTNASLLTKL | |
| | | QAQNQWLQDMTTHLILRSFKEFLQSSLRAL | |
| | | RQMEPKSCDKTHTCPPCPAPELLGGPSVFL | |
| | | FPPKPKDTLMISRTPEVTCVVVDVSHEDPE | |
| | | VKFNWYVDGVEVHNAKTKPREEQYGSTYRV | |
| | | VSVLTVLHQDWLNGKEYKCKVSNKALPAPI | |
| | | EKTISKAKGQPREPQVYTLPPSRDELTKNQ | |
| | | VSLWCLVKGFYPSDIAVEWESNGQPENNYK | |
| | | TTPPVLDSDGSFFLYSKLTVDKSRWQQGNV | |
| | | FSCSVMHEALHNHYTQKSLSLSPGKGGSHH | |
| | | HHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNS | |
| | | LRAEDTAVYYCAKYPHYYGSSHWYFDVWGQ | |
| | | GTLVTVSSASTKGPSVFPLAPSSKSTSGGT | |
| | | AALGCLVKDYFPEPVTVSWNSGALTSGVHT | |
| | | FPAVLQSSGLYSLSSVVTVPSSSLGTQTYI | |
| | | CNVNHKPSNTKVDKKVEPKSCDKTHTCPPC | |
| | | PAPELLGGPSVFLFPPKPKDTLMISRTPEV | |
| | | TCVVVDVSHEDPEVKFNWYVDGVEVHNAKT | |
| | | KPREEQYGSTYRVVSVLTVLHQDWLNGKEY | |
| | | KCKVSNKALPAPIEKTISKAKGQPREPQVY | |
| | | TLPPSRDELTKNQVSLSCAVKGFYPSDIAV | |
| | | EWESNGQPENNYKTTPPVLDSDGSFFLVSK | |
| | | LTVDKSRWQQGNVFSCSVMHEALHNHYTQK | |
| | | SLSLSPGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| IL-7 | Knob: | DCDIEGKDGKQYESVLMVSIDQLLDSMKEI | |
| | | GSNCLNNEFNFFKRHICDANKEGMFLFRAA | |
| | | RKLRQFLKMNSTGDFDLHLLKVSEGTTILL | |
| | | NCTGQVKGRKPAALGEAQPTKSLEENKSLK | |
| | | EQKKLNDLCFLKRLLQEIKTCWNKILMGTK | |
| | | EHEPKSCDKTHTCPPCPAPELLGGPSVFLF | |
| | | PPKPKDTLMISRTPEVTCVVVDVSHEDPEV | |
| | | KFNWYVDGVEVHNAKTKPREEQYGSTYRVV | |
| | | SVLTVLHQDWLNGKEYKCKVSNKALPAPIE | |
| | | KTISKAKGQPREPQVYTLPPSRDELTKNQV | |
| | | SLWCLVKGFYPSDIAVEWESNGQPENNYKT | |
| | | TPPVLDSDGSFFLYSKLTVDKSRWQQGNVF | |
| | | SCSVMHEALHNHYTQKSLSLSPGKGGSHHH | |
| | | HHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| IL-8 | Knob: | AKELRCQCIKTYSKPFHPKFIKELRVIESG | |
| | | PHCANTEIIVKLSDGRELCLDPKENWVQRV | |
| | | VEKFLKRAENSEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLWCLVKGFYPSDIAVEWESN | |
| | | GQPENNYKTTPPVLDSDGSFF | |
| | | LYSKLTVDKSRWQQGNVFSCSVMHEALHNH | |
| | | YTQKSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| IL-9 | Knob: | QGCPTLAGILDINFLINKMQEDPASKCHCS | |
| | | ANVTSCLCLGIPSDNCTRPCFSERLSQMTN | |
| | | TTMQTRYPLIFSRVKKSVEVLKNNKCPYFS | |
| | | CEQPCNQTTAGNALTFLKSLLEIFQKEKMR | |
| | | GMRGKIEPKSCDKTHTCPPCPAPELLGGPS | |
| | | VFLFPPKPKDTLMISRTPEVTCVVVDVSHE | |
| | | DPEVKFNWYVDGVEVHNAKTKPREEQYGST | |
| | | YRVVSVLTVLHQDWLNGKEYKCKVSNKALP | |
| | | APIEKTISKAKGQPREPQVYTLPPSRDELT | |
| | | KNQVSLWCLVKGFYPSDIAVEWESNGQPEN | |
| | | NYKTTPPVLDSDGSFFLYSKLTVDKSRWQQ | |
| | | GNVFSCSVMHEALHNHYTQKSLSLSPGKGG | |
| | | SHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| IL-10 | Knob: | SPGQGTQSENSCTHFPGNLPNMLRDLRDAF | |
| | | SRVKTFFQMKDQLDNLLLKESLLEDFKGYL | |
| | | GCQALSEMIQFYLEEVMPQAENQDPDIKAH | |
| | | VNSLGENLKTLRLRLRRCHRFLPCENKSKA | |
| | | VEQVKNAFNK | |
| | | LQEKGIYKAMSEFDIFINYIEAYMTMKIRN | |
| | | EPKSCDKTHTCPPCPAPELLGGPSVFLFPP | |
| | | KPKDTLMISRTPEVTCVVVDVSHEDPEVKF | |
| | | NWYVDGVEVHNAKTKPREEQYGSTYRVVSV | |
| | | LTVLHQDWLNGKEYKCKVSNKALPAPIEKT | |
| | | ISKAKGQPREPQVYTLPPSRDELTKNQVSL | |
| | | WCLVKGFYPSDIAVEWESNGQPENNYKTTP | |
| | | PVLDSDGSFFLYSKLTVDKSRWQQGNVFSC | |
| | | SVMHEALHNHYTQKSLSLSPGKGGSHHHHH | |
| | | H | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| IL-11 | Knob: | PGPPPGPPRVSPDPRAELDSTVLLTRSLLA | |
| | | DTRQLAAQLRDKFPADGDHNLDSLPTLAMS | |
| | | AGALGALQLPGVLTRLRADLLSYLRHVQWL | |
| | | RRAGGSSLKTLEPELGTLQARLDRLLRRLQ | |
| | | LLMSRLALPQPPPDPPAPPLAPPSSAWGGI | |
| | | RAAHAILGGLHLTLDWAVRGLLLLKTRLEP | |
| | | KSCDKTHTCPPCPAPELLGGPSVFLFPPKP | |
| | | KDTLMISRTPEVTCVVVDVSHEDPEVKFNW | |
| | | YVDGVEVHNAKTKPREEQYGSTYRVVSVLT | |
| | | VLHQDWLNGKEYKCKVSNKALPAPIEKTIS | |
| | | KAKGQPREPQVYTLPPSRDELTKNQVSLWC | |
| | | LVKGFYPSDIAVEWESNGQPENNYKTTPPV | |
| | | LDSDGSFFLYSKLTVDKSRWQQGNVFSCSV | |
| | | MHEALHNHYTQKSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| IL-13 | Knob: | SPGPVPPSTALRELIEELVNITQNQKAPLC | |
| | | NGSMVWSINLTAGMYCAALESLINVSGCSA | |
| | | IEKTQRMLSGFCPHKVSAGQFSSLHVRDTK | |
| | | IEVAQFVKDLLLHLKKLFREGRFNEPKSCD | |
| | | KTHTCPPCPAPELLGGPSVFLFPPKPKDTL | |
| | | MISRTPEVTCVVVDVSHEDPEVKFNWYVDG | |
| | | VEVHNAKTKPREEQYGSTYRVVSVLTVLHQ | |
| | | DWLNGKEYKCKVSNKALPAPIEKTISKAKG | |
| | | QPREPQVYTLPPSRDELTKNQVSLWCLVKG | |
| | | FYPSDIAVEWESNGQPENNYKTTPPVLDSD | |
| | | GSFFLYSKLTVDKSRWQQGNVFSCSVMHEA | |
| | | LHNHYTQKSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| IL-15 | Knob: | NWVNVISDLKKIEDLIQSMHIDATLYTESD | |
| | | VHPSCKVTAMKCFLLELQVISLESGDASIH | |
| | | DTVENLIILANNSLSSNGNVTESGCKECEE | |
| | | LEEKNIKEFLQSFVHIVQMFINTSEPKSCD | |
| | | KTHTCPPCPAPELLGGPSVFLFPPKPKDTL | |
| | | MISRTPEVTCVVVDVSHEDPEVKFNWYVDG | |
| | | VEVHNAKTKPREEQYGSTYRVVSVLTVLHQ | |
| | | DWLNGKEYKCKVSNKALPAPIEKTISKAKG | |
| | | QPREPQVYTLPPSRDELTKNQVSLWCLVKG | |
| | | FYPSDIAVEWESNGQPENNYKTTPPVLDSD | |
| | | GSFFLYSKLTVDKSRWQQGNVFSCSVMHEA | |
| | | LHNHYTQKSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEW | |
| | | ESNGQPENNYKTTPPVLDSDGSFFLVSKLT | |
| | | VDKSRWQQGNVFSCSVMHEALHNHYTQKSL | |
| | | SLSPGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| IL-17A | Knob: | IVKAGITIPRNPGCPNSEDKNFPRTVMVNL | |
| | | NIHNRNTNTNPKRSSDYYNRSTSPWNLHRN | |
| | | EDPERYPSVIWEAKCRHLGCINADGNVDYH | |
| | | MNSVPIQQEILVLRREPPHCPNSFRLEKIL | |
| | | VSVGCTCVTPIVHHVAdEPKSCDKTHTCPP | |
| | | CPAPELLGGPSVFLFPPKPKDTLMISRTPE | |
| | | VTCVVVDVSHEDPEVKFNWYVDGVEVHNAK | |
| | | TKPREEQYGSTYRVVSVLTVLHQDWLNGKE | |
| | | YKCKVSNKALPAPIEKTISKAKGQPREPQV | |
| | | YTLPPSRDELTKNQVSLWCLVKGFYPSDIA | |
| | | VEWESNGQPENNYKTTPPVLDSDGSFFLYS | |
| | | KLTVDKSRWQQGNVFSCSVMHEALHNHYTQ | |
| | | KSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| IL-17B | Knob: | QPRSPKSKRKGQGRPGPLAPGPHQVPLDLV | |
| | | SRMKPYARMEEYERNIEEMVAQLRNSSELA | |
| | | QRKCEVNLQLWMSNKRSLSPWGYSINHDPS | |
| | | RIPVDLPEARCLCLGCVNPFTMQEDRSMVS | |
| | | VPVFSQVPVRRRLCPPPPRTGPCRQRAVME | |
| | | TIAVGCTCIFEPKSCDKTHTCPPCPAPELL | |
| | | GGPSVFLFPPKPKDTLMISRTPEVTCVVVD | |
| | | VSHEDPEVKFNWYVDGVEVHNAKTKPREEQ | |
| | | YGSTYRVVSVLTVLHQDWLNGKEYKCKVSN | |
| | | KALPAPIEKTISKAKGQPREPQVYTLPPSR | |
| | | DELTKNQVSLWCLVKGFYPSDIAVEWESNG | |
| | | QPENNYKTTPPVLDSDGSFFLYSKLTVDKS | |
| | | RWQQGNVFSCSVMHEALHNHYTQKSLSLSP | |
| | | GKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTS | |
| | | GVHTFPAVLQSSGLYSLSSVVTVPSSSLGT | |
| | | QTYICNVNHKPSNTKVDKKVEPKSCDKTHT | |
| | | CPPCPAPELLGGPSVFLFPPKPKDTLMISR | |
| | | TPEVTCVVVDVSHEDPEVKFNWYVDGVEVH | |
| | | NAKTKPREEQYGSTYRVVSVLTVLHQDWLN | |
| | | GKEYKCKVSNKALPAPIEKTISKAKGQPRE | |
| | | PQVYTLPPSRDELTKNQVSLSCAVKGFYPS | |
| | | DIAVEWESNGQPENNYKTTPPVLDSDGSFF | |
| | | LVSKLTVDKSRWQQGNVFSCSVMHEALHNH | |
| | | YTQKSLSLSPGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| IL-17C | Knob: | HHDPSLRGHPHSHGTPHCYSAEELPLGQAP | |
| | | PHLLARGAKWGQALPVALVSSLEAASHRGR | |
| | | HERPSATTQCPVLRPEEVLEADTHQRSISP | |
| | | WRYRVDTDEDRYPQKLAFAECLCRGCIDAR | |
| | | TGRETAALNSVRLLQSLLVLRRRPCSRDGS | |
| | | GLPTPGAFAFHTEFIHVPVGCTCVLPRSVE | |
| | | PKSCDKTHTCPPCPAPELLGGPSVFLFPPK | |
| | | PKDTLMISRTPEVTCVVVDVSHEDPEVKFN | |
| | | WYVDGVEVHNAKTKPREEQYGSTYRVVSVL | |
| | | TVLHQDWLNGKEYKCKVSNKALPAPIEKTI | |
| | | SKAKGQPREPQVYTLPPSRDELTKNQVSLW | |
| | | CLVKGFYPSDIAVEWESNGQPENNYKTTPP | |
| | | VLDSDGSFFLYSKLTVDKSRWQQGNVFSCS | |
| | | VMHEALHNHYTQKSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| IL-17D | Knob: | AGAPRAGRRPARPRGCADRPEELLEQLYGR | |
| | | LAAGVLSAFHHTLQLGPREQARNASCPAGG | |
| | | RPADRRFRPPTNLRSVSPWAYRISYDPARY | |
| | | PRYLPEAYCLCRGCLTGLFGEEDVRFRSAP | |
| | | VYMPTVVLRRTPACAGGRSVYTEAYVTIPV | |
| | | GCTCVPEPEKDADSINSSIDKQGAKLLLGP | |
| | | NDAPAGPEPKSCDKTHTCPPCPAPELLGGP | |
| | | SVFLFPPKPKDTLMISRTPEVTCVVVDVSH | |
| | | EDPEVKFNWYVDGVEVHNAKTKPREEQYGS | |
| | | TYRVVSVLTVLHQDWLNGKEYKCKVSNKAL | |
| | | PAPIEKTISKAKGQPREPQVYTLPPSRDEL | |
| | | TKNQVSLWCLVKGFYPSD | |
| | | IAVEWESNGQPENNYKTTPPVLDSDGSFFL | |
| | | YSKLTVDKSRWQQGNVFSCSVMHEALHNHY | |
| | | TQKSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| IL-17F | Knob: | RKIPKVGHTFFQKPESCPPVPGGSMKLDIG | |
| | | IINENQRVSMSRNIESRSTSPWNYTVTWDP | |
| | | NRYPSEVVQAQCRNLGCINAQGKEDISMNS | |
| | | VPIQQETLVVRRKHQGCSVSFQLEKVLVTV | |
| | | GCTCVTPVIHHVQEPKSCDKTHTCPPCPAP | |
| | | ELLGGPSVFLFPPKPKDTLMISRTPEVTCV | |
| | | VVDVSHEDPEVKFNWYVDGVEVHNAKTKPR | |
| | | EEQYGSTYRVVSVLTVLHQDWLNGKEYKCK | |
| | | VSNKALPAPIEKTISKAKGQPREPQVYTLP | |
| | | PSRDELTKNQVSLWCLVKGFYPSDIAVEWE | |
| | | SNGQPENNYKTTPPVLDSDGSFFLYSKLTV | |
| | | DKSRWQQGNVFSCSVMHEALHNHYTQKSLS | |
| | | LSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| IL-18 | Knob: | YFGKLESKLSVIRNLNDQVLFIDQGNRPLF | |
| | | EDMTDSDCRDNAPRTIFIISMYKDSQPRGM | |
| | | AVTISVKCEKISTLSCENKIISFKEMNPPD | |
| | | NIKDTKSDIIFFQRSVPGHDNKMQFESSSY | |
| | | EGYFLACEKERDLFK | |
| | | LILKKEDELGDRSIMFTVQNEDEPKSCDKT | |
| | | HTCPPCPAPELLGGPSVFLFPPKPKDTLMI | |
| | | SRTPEVTCVVVDVSHEDPEVKFNWYVDGVE | |
| | | VHNAKTKPREEQYGSTYRVVSVLTVLHQDW | |
| | | LNGKEYKCKVSNKALPAPIEKTISKAKGQP | |
| | | REPQVYTLPPSRDELTKNQVSLWCLVKGFY | |
| | | PSDIAVEWESNGQPENNYKTTPPVLDSDGS | |
| | | FFLYSKLTVDKSRWQQGNVFSCSVMHEALH | |
| | | NHYTQKSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| IL-19 | Knob: | LRRCLISTDMHHIEESFQEIKRAIQAKDTF | |
| | | PNVTILSTLETLQIIKPLDVCCVTKNLLAF | |
| | | YVDRVFKDHQEPNPKILRKISSIANSFLYM | |
| | | QKTLRQCQEQRQCHCRQEATNATRVIHDNY | |
| | | DQLEVHAAAIKSLGELDVFLAWINKNHEVM | |
| | | FSAEPKSCDKTHTCPPCPAPELLGGPSVFL | |
| | | FPPKPKDTLMISRTPEVTCVVVDVSHEDPE | |
| | | VKFNWYVDGVEVHNAKTKPREEQYGSTYRV | |
| | | VSVLTVLHQDWLNGKEYKCKVSNKALPAPI | |
| | | EKTISKAKGQPREPQVYTLPPSRDELTKNQ | |
| | | VSLWCLVKGFYPSDIAVEWESNGQPENNYK | |
| | | TTPPVLDSDGSFFLYSKLTVDKSRWQQGNV | |
| | | FSCSVMHEALHNHYTQKSLSLSPGKGGSHH | |
| | | HHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| IL-20 | Knob: | LKTLNLGSCVIATNLQEIRNGFSEIRGSVQ | |
| | | AKDGNIDIRILRRTESLQDTKPANRCCLLR | |
| | | HLLRLYLDRVFKNYQTPDHYTLRKISSLAN | |
| | | SFLTIKKDLRLCHAHMTCHCGEEAMKKYSQ | |
| | | ILSHFEKLEPQAAVVKALGELDILLQWMEE | |
| | | TEEPKSCDKTHTCPPCPAPELLGGPSVFLF | |
| | | PPKPKDTLMISRTPEVTCVVVDVSHEDPEV | |
| | | KFNWYVDGVEVHNAKTKPREEQYGSTYRVV | |
| | | SVLTVLHQDWLNGKEYKCKVSNKALPAPIE | |
| | | KTISKAKGQPREPQVYTLPPSRDELTKNQV | |
| | | SLWCLVKGFYPSDIAVEWESNGQPENNYKT | |
| | | TPPVLDSDGSFFLYSKLTVDKSRWQQGNVF | |
| | | SCSVMHEALHNHYTQKSLSLSPGKGGSHHH | |
| | | HHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| IL-21 | Knob: | QDRHMIRMRQLIDIVDQLKNYVNDLVPEFL | |
| | | PAPEDVETNCEWSAFSCFQKAQLKSANTGN | |
| | | NERIINVSIKKLKRKPPSTNAGRRQKHRLT | |
| | | CPSCDSYEKKPPKEFLERFKSLLQKMIHQH | |
| | | LSSRTHGSEDSEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLWCLVKGFYPSDIAVEWESN | |
| | | GQPENNYKTTPPVLDSDGSFFLYSKLTVDK | |
| | | SRWQQGNVFSCSVMHEALHNHYTQKSLSLS | |
| | | PGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISK | |
| | | AKGQPREPQVYTLPPSRDELTKNQVSLSCA | |
| | | VKGFYPSDIAVEWESNGQPENNYKTTPPVL | |
| | | DSDGSFFLVSKLTVDKSRWQQGNVFSCSVM | |
| | | HEALHNHYTQKSLSLSPGKGGSGAWSHPQF | |
| | | EK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| IL-22 | Knob: | APISSHCRLDKSNFQQPYITNRTFMLAKEA | |
| | | SLADNNTDVRLIGEKLFHGVSMSERCYLMK | |
| | | QVLNFTLEEVLFPQSDRFQPYMQEVVPFLA | |
| | | RLSNRLSTCHIEGDDLHIQRNVQKLKDTVK | |
| | | KLGESGEIKAIGELDLLFMSLRNACIEPKS | |
| | | CDKTHTCPPCPAPELLGGPSVFLFPPKPKD | |
| | | TLMISRTPEVTCVVVDVSHEDPEVKFNWYV | |
| | | DGVEVHNAKTKPREEQYGSTYRVVSVLTVL | |
| | | HQDWLNGKEYKCKVSNKALPAPIEKTISKA | |
| | | KGQPREPQVYTLPPSRDELTKNQVSLWCLV | |
| | | KGFYPSDIAVEWESNGQPENNYKTTPPVLD | |
| | | SDGSFFLYSKLTVDKSRWQQGNVFSCSVMH | |
| | | EALHNHYTQKSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| IL-24 | Knob: | QEFHFGPCQVKGVVPQKLWEAFWAVKDTMQ | |
| | | AQDNITSARLLQQEVLQNVSDAESCYLVHT | |
| | | LLEFYLKTVFKNYHNRTVEVRTLKSFSTLA | |
| | | NNFVLIVSQLQPSQENEMFSIRDSAHRRFL | |
| | | LFRRAFKQLDVEAALTKALGEVDILLTWMQ | |
| | | KFYKLEPKSCDKTHTCPPCPAPELLGGPSV | |
| | | FLFPPKPKDTLMISRTPEVTCVVVDVSHED | |
| | | PEVKFNWYVDGVEVHNAKTKPREEQYGSTY | |
| | | RVVSVLTVLHQDWLNGKEYKCKVSNKALPA | |
| | | PIEKTISKAKGQPREPQVYTLPPSRDELTK | |
| | | NQVSLWCLVKGFYPSDIAVEWESNGQPENN | |
| | | YKTTPPVLDSDGSFFLYSKLTVDKSRWQQG | |
| | | NVFSCSVMHEALHNHYTQKSLSLSPGKGGS | |
| | | HHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTK | |
| | | GPSVFPLAPSSKSTSGGTAALGCLVKDYFP | |
| | | EPVTVSWNSGALTSGVHTFPAVLQSSGLYS | |
| | | LSSVVTVPSSSLGTQTYICNVNHKPSNTKV | |
| | | DKKVEPKSCDKTHTCPPCPAPELLGGPSVF | |
| | | LFPPKPKDTLMISRTPEVTCVVVDVSHEDP | |
| | | EVKFNWYVDGVEVHNAKTKPREEQYGSTYR | |
| | | VVSVLTVLHQDWLNGKEYKCKVSNKALPAP | |
| | | IEKTISKAKGQPREPQVYTLPPSRDELTKN | |
| | | QVSLSCAVKGFYPSDIAVEWESNGQPENNY | |
| | | KTTPPVLDSDGSFFLVSKLTVDKSRWQQGN | |
| | | VFSCSVMHEALHNHYTQKSLSLSPGKGGSG | |
| | | AWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| IL-25 | Knob: | YSHWPSCCPSKGQDTSEELLRWSTVPVPPL | |
| | | EPARPNRHPESCRASEDGPLNSRAISPWRY | |
| | | ELDRDLNRLPQDLYHARCLCPHCVSLQTGS | |
| | | HMDPRGNSELLYHNQTVFYRRPCHGEKGTH | |
| | | KGYCLERRLYRVSLACVCVRPRVMGEPKSC | |
| | | DKTHTCPPCPAPELLGGPSVFLFPPKPKDT | |
| | | LMISRTPEVTCVVVDVSHEDPEVKFNWYVD | |
| | | GVEVHNAKTKPREEQYGSTYRVVSVLTVLH | |
| | | QDWLNGKEYKCKVSNKALPAPIEKTISKAK | |
| | | GQPREPQVYTLPPSRDELTKNQVSLWCLVK | |
| | | GFYPSDIAVEWESNGQPENNYKTTPPVLDS | |
| | | DGSFFLYSKLTVDKSRWQQGNVFSCSVMHE | |
| | | ALHNHYTQKSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| IL-26 | Knob: | KHKQSSFTKSCYPRGTLSQAVDALYIKAAW | |
| | | LKATIPEDRIKNIRLLKKKTKKQFMKNCQF | |
| | | QEQLLSFFMEDVFGQLQLQGCKKIRFVEDF | |
| | | HSLRQKLSHCISCASSAREMKSITRMKRIF | |
| | | YRIGNKGIYKAISELDILLSWIKKLLESSQ | |
| | | EPKSCDKTHTCPPCPAPELLGGPSVFLFPP | |
| | | KPKDTLMISRTPEVTCVVVDVSHEDPEVKF | |
| | | NWYVDGVEVHNAKTKPREEQYGSTYRVVSV | |
| | | LTVLHQDWLNGKEYKCKVSNKALPAPIEKT | |
| | | ISKAKGQPREPQVYTLPPSRDELTKNQVSL | |
| | | WCLVKGFYPSDIAVEWESNGQPENNYKTTP | |
| | | PVLDSDGSFFLYSKLTVDKSRWQQGNVFSC | |
| | | SVMHEALHNHYTQKSLSLSPGKGGSHHHHH | |
| | | H | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| IL-28A | Knob: | VPVARLHGALPDARGCHIAQFKSLSPQELQ | |
| | | AFKRAKDALEESLLLKDCRCHSRLFPRTWD | |
| | | LRQLQVRERPMALEAELALTLKVLEATADT | |
| | | DPALVDVLDQPLHTLHHILSQFRACIQPQP | |
| | | TAGPRTRGRLHHWLYRLQEAPKKESPGCLE | |
| | | ASVTFNLFRLLTRDLNCVASGDLCVEPKSC | |
| | | DKTHTCPPCPAPELLGGPSVFLFPPKPKDT | |
| | | LMISRTPEVTCVVVDVSHEDPEVKFNWYVD | |
| | | GVEVHNAKTKPREEQYGSTYRVVSVLTVLH | |
| | | QDWLNGKEYKCKVSNKALPAPIEKTISKAK | |
| | | GQPREPQVYTLPPSRDELTKNQVSLWCLVK | |
| | | GFYPSDIAVEWESNGQPENNYKTTPPVLDS | |
| | | DGSFFLYSKLTVDKSRWQQGNVFSCSVMHE | |
| | | ALHNHYTQKSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| IL-31 | Knob: | SHTLPVRLLRPSDDVQKIVEELQSLSKMLL | |
| | | KDVEEEKGVLVSQNYTLPCLSPDAQPPNNI | |
| | | HSPAIRAYLKTIRQLDNKSVIDEIIEHLDK | |
| | | LIFQDAPETNISVPTDTHECKRFILTISQQ | |
| | | FSECMDLALKSLTSGAQQATTEPKSCDKTH | |
| | | TCPPCPAPELLGGPSVFLFPPKPKDTLMIS | |
| | | RTPEVTCVVVDVSHEDPEVKFNWYVDGVEV | |
| | | HNAKTKPREEQYGSTYRVVSVLTVLHQDWL | |
| | | NGKEYKCKVSNKALPAPIEKTISKAKGQPR | |
| | | EPQVYTLPPSRDELTKNQVSLWCLVKGFYP | |
| | | SDIAVEWESNGQPENNYKTTPPVLDSDGSF | |
| | | FLYSKLTVDKSRWQQGNVFSCSVMHEALHN | |
| | | HYTQKSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| IL-32a | Knob: | MCFPKVLSDDMKKLKARMHQAIERFYDKMQ | |
| | | NAESGRGQVMSSLAELEDDFKEGYLETVAA | |
| | | YYEEQHPELTPLLEKERDGLRCRGNRSPVP | |
| | | DVEDPATEEPGESFCDKSYGAPRGDKEELT | |
| | | PQKCSEPQSSKEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLWCLVKGFYPSDIAVEWESN | |
| | | GQPENNYKTTPPVLDSDGSFFLYSKLTVDK | |
| | | SRWQQGNVFSCSVMHEALHNHYTQKSLSLS | |
| | | PGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVC | |
| | | LLNNFYPREAKVQWKVDNALQSGNSQESVT | |
| | | EQDSKDSTYSLSSTLTLSKADYEKHKVYAC | |
| | | EVTHQGLSSPVTKSFNRGEC | |
| IL-33 | Knob: | SITGISPITEYLASLSTYNDQSITFALEDE | |
| | | SYEIYVEDLKKDEKKDKVLLSYYESQHPSN | |
| | | ESGDGVDGKMLMVTLSPTKDFWLHANNKEH | |
| | | SVELHKCEKPLPDQAFFVLHNMHSNCVSFE | |
| | | CKTDPGVFIGVKDNHLALIKVDSSENLCTE | |
| | | NILFKLSETEPKSCDKTHTCPPCPAPELLG | |
| | | GPSVFLFPPKPKDTLMISRTPEVTCVVVDV | |
| | | SHEDPEVKFNWYVDGVEVHNAKTKPREEQY | |
| | | GSTYRVVSVLTVLHQDWLNGKEYKCKVSNK | |
| | | ALPAPIEKTISKAKGQPREPQVYTLPPSRD | |
| | | ELTKNQVSLWCLVKGFYPSDIAVEWESNGQ | |
| | | PENNYKTTPPVLDSDGSFFLYSKLTVDKSR | |
| | | WQQGNVFSCSVMHEALHNHYTQKSLSLSPG | |
| | | KGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| IL-34 | Knob: | NEPLEMWPLTQNEECTVTGFLRDKLQYRSR | |
| | | LQYMKHYFPINYKISVPYEGVFRIANVTRL | |
| | | QRAQVSERELRYLWVLVSLSATESVQDVLL | |
| | | EGHPSWKYLQEVETLLLNVQQGLTDVEVSP | |
| | | KVESVLSLLNAPGPNLKLVRPKALLDNCFR | |
| | | VMELLYCSCCKQSSVLNWQDCEVPSPQSCS | |
| | | PEPSLQYAATQLYPPPPWSPSSPPHSTGSV | |
| | | RPVRAQGEGLLPEPKSCDKTHTCPPCPAPE | |
| | | LLGGPSVFLFPPKPKDTLMISRTPEVTCVV | |
| | | VDVSHEDPEVKFNWYVDGVEVHNAKTKPRE | |
| | | EQYGSTYRVVSVLTVLHQDWLNGKEYKCKV | |
| | | SNKALPAPIEKTISKAKGQPREPQVYTLPP | |
| | | SRDELTKNQVSLWCLVKGFYPSDIAVEWES | |
| | | NGQPENNYKTTPPVLDSDGSFFLYSKLTVD | |
| | | KSRWQQGNVFSCSVMHEALHNHYTQKSLSL | |
| | | SPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | Q | |
| | | YGSTYRVVSVLTVLHQDWLNGKEYKCKVSN | |
| | | KALPAPIEKTISKAKGQPREPQVYTLPPSR | |
| | | DELTKNQVSLSCAVKGFYPSDIAVEWESNG | |
| | | QPENNYKTTPPVLDSDGSFFLVSKLTVDKS | |
| | | RWQQGNVFSCSVMHEALHNHYTQKSLSLSP | |
| | | GKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| IL-36A | Knob: | KIDTPQQGSIQDINHRVWVLQDQTLIAVPR | |
| | | KDRMSPVTIALISCRHVETLEKDRGNPIYL | |
| | | GLNGLNLCLMCAKVGDQPTLQLKEKDIMDL | |
| | | YNQPEPVKSFLFYHSQSGRNSTFESVAFPG | |
| | | WFIAVSSEGGCPLILTQELGKANTTDFGLT | |
| | | MLFEPKSCDKTHTCPPCPAPELLGGPSVFL | |
| | | FPPKPKDTLMISRTPEVTCVVVDVSHEDPE | |
| | | VKFNWYVDGVEVHNAKTKPREEQYGSTYRV | |
| | | VSVLTVLHQDWLNGKEYKCKVSNKALPAPI | |
| | | EKTISKAKGQPREPQVYTLPPSRDELTKNQ | |
| | | VSLWCLVKGFYPSDIAVEWESNGQPENNYK | |
| | | TTPPVLDSDGSFFLYSKLTVDKSRWQQGNV | |
| | | FSCSVMHEALHNHYTQKSLSLSPGKGGSHH | |
| | | HHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| IL-36RA | Knob: | VLSGALCFRMKDSALKVLYLHNNQLLAGGL | |
| | | HAGKVIKGEEISVVPNRWLDASLSPVILGV | |
| | | QGGSQCLSCGVGQEPTLTLEPVNIMELYLG | |
| | | AKESKSFTFYRRDMGLTSSFESAAYPGWFL | |
| | | CTVPEADQPVRLTQLPENGGWNAPITDFYF | |
| | | QQCDEPKSCDKTHTCPPCPAPELLGGPSVF | |
| | | LFPPKPKDTLMISRTPEVTCVVVDVSHEDP | |
| | | EVKFNWYVDGVEVHNAKTKPREEQYGSTYR | |
| | | VVSVLTVLHQDWLNGKEYKCKVSNKALPAP | |
| | | IEKTISKAKGQPREPQVYTLPPSRDELTKN | |
| | | QVSLWCLVKGFYPSDIAVEWESNGQPENNY | |
| | | KTTPPVLDSDGSFFLYSKLTVDKSRWQQGN | |
| | | VFSCSVMHEALHNHYTQKSLSLSPGKGGSH | |
| | | HHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| IL-37 | Knob: | VHTSPKVKNLNPKKFSIHDQDHKVLVLDSG | |
| | | NLIAVPDKNYIRPEIFFALASSLSSASAEK | |
| | | GSPILLGVSKGEFCLYCDKDKGQSHPSLQL | |
| | | KKEKLMKLAAQKESARRPFIFYRAQVGSWN | |
| | | MLESAAHPGWFICTSCNCNEPVGVTDKFEN | |
| | | RKHIEFSFQPVCKAEMSPSEVSDEPKSCDK | |
| | | THTCPPCPAPELLGGPSVFLFPPKPKDTLM | |
| | | ISRTPEVTCVVVDVSHEDPEVKFNWYVDGV | |
| | | EVHNAKTKPREEQYGSTYRVVSVLTVLHQD | |
| | | WLNGKEYKCKVSNKALPAPIEKTISKAKGQ | |
| | | PREPQVYTLPPSRDELTKNQVSLWCLVKGF | |
| | | YPSDIAVEWESNGQPENNYKTTPPVLDSDG | |
| | | SFFLYSKLTVDKSRWQQGNVFSCSVMHEAL | |
| | | HNHYTQKSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| IL-38 | Knob: | CSLPMARYYIIKYADQKALYTRDGQLLVGD | |
| | | PVADNCCAEKICILPNRGLARTKVPIFLGI | |
| | | QGGSRCLACVETEEGPSLQLEDVNIEELYK | |
| | | GGEEATRFTFFQSSSGSAFRLEAAAWPGWF | |
| | | LCGPAEPQQPVQLTKESEPSARTKFYFEQS | |
| | | WEPKSCDKTHTCPPCPAPELLGGPS | |
| | | VFLFPPKPKDTLMISRTPEVTCVVVDVSHE | |
| | | DPEVKFNWYVDGVEVHNAKTKPREEQYGST | |
| | | YRVVSVLTVLHQDWLNGKEYKCKVSNKALP | |
| | | APIEKTISKAKGQPREPQVYTLPPSRDELT | |
| | | KNQVSLWCLVKGFYPSDIAVEWESNGQPEN | |
| | | NYKTTPPVLDSDGSFFLYSKLTVDKSRWQQ | |
| | | GNVFSCSVMHEALHNHYTQKSLSLSPGKGG | |
| | | SHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| IFN-α | Knob: | CDLPETHSLDNRRTLMLLAQMSRISPSSCL | |
| | | MDRHDFGFPQEEFDGNQFQKAPAISVLHEL | |
| | | IQQIFNLFTTKDSSAAWDEDLLDKFCTELY | |
| | | QQLNDLEACVMQEERVGETPLMNADSILAV | |
| | | KKYFRRITLYLTEKKYSPCAWEVVRAEIMR | |
| | | SLSLSTNLQERLRRKEEPKSCDKTHTCPPC | |
| | | PAPELLGGPSVFLFPPKPKDTLMISRTPEV | |
| | | TCVVVDVSHEDPEVKFNWYVDGVEVHNAKT | |
| | | KPREEQYGSTYRVVSVLTVLHQDWLNGKEY | |
| | | KCKVSNKALPAPIEKTISKAKGQPREPQVY | |
| | | TLPPSRDELTKNQVSLWCLVKGFYPSDIAV | |
| | | EWESNGQPENNYKTTPPVLDSDGSFFLYSK | |
| | | LTVDKSRWQQGNVFSCSVMHEALHNHYTQK | |
| | | SLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVC | |
| | | LLNNFYPREAKVQWKVDNALQSGNSQESVT | |
| | | EQDSKDSTYSLSSTLTLSKADYEKHKVYAC | |
| | | EVTHQGLSSPVTKSFNRGEC | |
| IFN-β | Knob: | MSYNLLGFLQRSSNFQCQKLLWQLNGRLEY | |
| | | CLKDRMNFDIPEEIKQLQQFQKEDAALTIY | |
| | | EMLQNIFAIFRQDSSSTGWNETIVENLLAN | |
| | | VYHQINHLKTVLEEKLEKEDFTRGKLMSSL | |
| | | HLKRYYGRILHYLKAKEYSHCAWTIVRVEI | |
| | | LRNFYFINRLTGYLRNEPKSCDKTHTCPPC | |
| | | PAPELLGGPSVFLFPPKPKDTLMISRTPEV | |
| | | TCVVVDVSHEDPEVKFNWYVDGVEVHNAKT | |
| | | KPREEQYGSTYRVVSVLTVLHQDWLNGKEY | |
| | | KCKVSNKALPAPIEKTISKAKGQPREPQVY | |
| | | TLPPSRDELTKNQVSLWCLVKGFYPSDIAV | |
| | | EWESNGQPENNYKTTPPVLDSDGSFFLYSK | |
| | | LTVDKSRWQQGNVFSCSVMHEALHNHYTQK | |
| | | SLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| IFN-γ | Knob: | QDPYVKEAENLKKYFNAGHSDVADNGTLFL | |
| | | GILKNWKEESDRKIMQSQIVSFYFKLFKNF | |
| | | KDDQSIQKSVETIKEDMNVKFFNSNKKKRD | |
| | | DFEKLTNYSVTDLNVQRKAIHELIQVMAEL | |
| | | SPAAKTGKRKRSQMLFRGEPKSCDKTHTCP | |
| | | PCPAPELLGGPSVFLFPPKPKDTLMISRTP | |
| | | EVTCVVVDVSHEDPEVKFNWYVDGVEVHNA | |
| | | KTKPREEQYGSTYRVVSVLTVLHQDWLNGK | |
| | | EYKCKVSNKALPAPIEKTISKAKGQPREPQ | |
| | | VYTLPPSRDELTKNQVSLWCLVKGFYPSDI | |
| | | AVEWESNGQPENNYKTTPPVLDSDGSFFLY | |
| | | SKLTVDKSRWQQGNVFSCSVMHEALHNHYT | |
| | | QKSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEW | |
| | | ESNGQPENNYKTTPPVLDSDGSFFLVSKLT | |
| | | VDKSRWQQGNVFSCSVMHEALHNHYTQKSL | |
| | | SLSPGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RESGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| TGF-β | Knob: | ALDTNYCFSSTEKNCCVRQLYIDFRKDLGW | |
| | | KWIHEPKGYHANFCLGPCPYIWSLDTQYSK | |
| | | VLALYNQHNPGASAAPCCVPQALEPLPIVY | |
| | | YVGRKPKVEQLSNMIVRSCKCSEPKSCDKT | |
| | | HTCPPCPAPELLGGPSVFLFPPKPKDTLMI | |
| | | SRTPEVTCVVVDVSHEDPEVKFNWYVDGVE | |
| | | VHNAKTKPREEQYGSTYRVVSVLTVLHQDW | |
| | | LNGKEYKCKVSNKALPAPIEKTISKAKGQP | |
| | | REPQVYTLPPSRDELTKNQVSLWCLVKGFY | |
| | | PSDIAVEWESNGQPENNYKTTPPVLDSDGS | |
| | | FFLYSKLTVDKSRWQQGNVFSCSVMHEALH | |
| | | NHYTQKSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| TNF-α | Knob: | GPQREEFPRDLSLISPLAQAVRSSSRTPSD | |
| | | KPVAHVVANPQAEGQLQWLNRRANALLANG | |
| | | VELRDNQLVVPSEGLYLIYSQVLFKGQGCP | |
| | | STHVLLTHTISRIAVSYQTKVNLLSAIKSP | |
| | | CQRETPEGAEAKPWYEPIYLGGVFQLEKGD | |
| | | RLSAEINRPDYLDFAESGQVYFGIIALEPK | |
| | | SCDKTHTCPPCPAPELLGGPSVFLFPPKPK | |
| | | DTLMISRTPEVTCVVVDVSHEDPEVKFNWY | |
| | | VDGVEVHNAKTKPREEQYGSTYRVVSVLTV | |
| | | LHQDWLNGKEYKCKVSNKALPAPIEKTISK | |
| | | AKGQPREPQVYTLPPSRDELTKNQVSLWCL | |
| | | VKGFYPSDIAVEWESNGQPENNYKTTPPVL | |
| | | DSDGSFFLYSKLTVDKSRWQQGNVFSCSVM | |
| | | HEALHNHYTQKSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTS | |
| | | GVHTFPAVLQSSGLYSLSSVVTVPSSSLGT | |
| | | QTYICNVNHKPSNTKVDKKVEPKSCDKTHT | |
| | | CPPCPAPELLGGPSVFLFPPKPKDTLMISR | |
| | | TPEVTCVVVDVSHEDPEVKFNWYVDGVEVH | |
| | | NAKTKPREEQYGSTYRVVSVLTVLHQDWLN | |
| | | GKEYKCKVSNKALPAPIEKTISKAKGQPRE | |
| | | PQVYTLPPSRDELTKNQVSLSCAVKGFYPS | |
| | | DIAVEWESNGQPENNYKTTPPVLDSDGSFF | |
| | | LVSKLTVDKSRWQQGNVFSCSVMHEALHNH | |
| | | YTQKSLSLSPGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RESGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CXCL1 | Knob: | ASVATELRCQCLQTLQGIHPKNIQSVNVKS | |
| | | PGPHCAQTEVIATLKNGRKACLNPASPIVK | |
| | | KIIEKMLNSDKSNEPKSCDKTHTCPPCPAP | |
| | | ELLGGPSVFLFPPKPKDTLMISRTPEVTCV | |
| | | VVDVSHEDPEVKFNWYVDGVEVHNAKTKPR | |
| | | EEQYGSTYRVVSVLTVLHQDWLNGKEYKCK | |
| | | VSNKALPAPIEKTISKAKGQPREPQVYTLP | |
| | | PSRDELTKNQVSLWCLVKGFYPSDIAVEWE | |
| | | SNGQPENNYKTTPPVLDSDGSFFLYSKLTV | |
| | | DKSRWQQGNVFSCSVMHEALHNHYTQKSLS | |
| | | LSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CXCL2 | Knob: | APLATELRCQCLQTLQGIHLKNIQSVKVKS | |
| | | PGPHCAQTEVIATLKNGQKACLNPASPMVK | |
| | | KIIEKMLKNGKSNEPKSCDKTHTCPPCPAP | |
| | | ELLGGPSVFLFPPKPKDTLMISRTPEVTCV | |
| | | VVDVSHEDPEVKFNWYVDGVEVHNAKTKPR | |
| | | EEQYGSTYRVVSVLTVLHQDWLNGKEYKCK | |
| | | VSNKALPAPIEKTISKAKGQPREPQVYTLP | |
| | | PSRDELTKNQVSLWCLVKGFYPSDIAVEWE | |
| | | SNGQPENNYKTTPPVLDSDGSFFLYSKLTV | |
| | | DKSRWQQGNVFSCSVMHEALHNHYTQKSLS | |
| | | LSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNS | |
| | | LRAEDTAVYYCAKYPHYYGSSHWYFDVWGQ | |
| | | GTLVTVSSASTKGPSVFPLAPSSKSTSGGT | |
| | | AALGCLVKDYFPEPVTVSWNSGALTSGVHT | |
| | | FPAVLQSSGLYSLSSVVTVPSSSLGTQTYI | |
| | | CNVNHKPSNTKVDKKVEPKSCDKTHTCPPC | |
| | | PAPELLGGPSVFLFPPKPKDTLMISRTPEV | |
| | | TCVVVDVSHEDPEVKFNWYVDGVEVHNAKT | |
| | | KPREEQYGSTYRVVSVLTVLHQDWLNGKEY | |
| | | KCKVSNKALPAPIEKTISKAKGQPREPQVY | |
| | | TLPPSRDELTKNQVSLSCAVKGFYPSDIAV | |
| | | EWESNGQPENNYKTTPPVLDSDGSFFLVSK | |
| | | LTVDKSRWQQGNVFSCSVMHEALHNHYTQK | |
| | | SLSLSPGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CXCL3 | Knob: | ASVVTELRCQCLQTLQGIHLKNIQSVNVRS | |
| | | PGPHCAQTEVIATLKNGKKACLNPASPMVQ | |
| | | KIIEKILNKGSTNEPKSCDKTHTCPPCPAP | |
| | | ELLGGPSVFLFPPKPKDTLMISRTPEVTCV | |
| | | VVDVSHEDPEVKFNWYVDGVEVHNAKTKPR | |
| | | EEQYGSTYRVVSVLTVLHQDWLNGKEYKCK | |
| | | VSNKALPAPIEKTISKAKGQPREPQVYTLP | |
| | | PSRDELTKNQVSLWCLVKGFYPSDIAVEWE | |
| | | SNGQPENNYKTTPPVLDSDGSFFLYSKLTV | |
| | | DKSRWQQGNVFSCSVMHEALHNHYTQKSLS | |
| | | LSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CXCL5 | Knob: | AAVLRELRCVCLQTTQGVHPKMISNLQVFA | |
| | | IGPQCSKVEVVASLKNGKEICLDPEAPFLK | |
| | | KVIQKILDGGNKENEPKSCDKTHTCPPCPA | |
| | | PELLGGPSVFLFPPKPKDTLMISRTPEVTC | |
| | | VVVDVSHEDPEVKFNWYVDGVEVHNAKTKP | |
| | | REEQYGSTYRVVSVLTVLHQDWLNGKEYKC | |
| | | KVSNKALPAPIEKTISKAKGQPREPQVYTL | |
| | | PPSRDELTKNQVSLWCLVKGFYPSDIAVEW | |
| | | ESNGQPENNYKTTPPVLDSDGSFFLYSKLT | |
| | | VDKSRWQQGNVFSCSVMHEALHNHYTQKSL | |
| | | SLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CXCL6 | Knob: | VLTELRCTCLRVTLRVNPKTIGKLQVFPAG | |
| | | PQCSKVEVVASLKNGKQVCLDPEAPFLKKV | |
| | | IQKILDSGNKKNEPKSCDKTHTCPPCPAPE | |
| | | LLGGPSVFLFPPKPKDTLMISRTPEVTCVV | |
| | | VDVSHEDPEVKFNWYVDGVEVHNAKTKPRE | |
| | | EQYGSTYRVVSVLTVLHQDWLNGKEYKCKV | |
| | | SNKALPAPIEKTISKAKGQPREPQVYTLPP | |
| | | SRDELTKNQVSLWCLVKGFYPSDIAVEWES | |
| | | NGQPENNYKTTPPVLDSDGSFFLYSKLTVD | |
| | | KSRWQQGNVFSCSVMHEALHNHYTQKSLSL | |
| | | SPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CXCL7 | Knob: | AELRCMCIKTTSGIHPKNIQSLEVIGKGTH | |
| | | CNQVEVIATLKDGRKICLDPDAPRIKKIVQ | |
| | | KKLAGDESADEPKSCDKTHTCPPCPAPELL | |
| | | GGPSVFLFPPKPKDTLMISRTPEVTCVVVD | |
| | | VSHEDPEVKFNWYVDGVEVHNAKTKPREEQ | |
| | | YGSTYRVVSVLTVLHQDWLNGKEYKCKVSN | |
| | | KALPAPIEKTISKAKGQPREPQVYTLPPSR | |
| | | DELTKNQVSLWCLVKGFYPSDIAVEWESNG | |
| | | QPENNYKTTPPVLDSDGSFFL | |
| | | YSKLTVDKSRWQQGNVFSCSVMHEALHNHY | |
| | | TQKSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CXCL8 | Knob: | AVLPRSAKELRCQCIKTYSKPFHPKFIKEL | |
| | | RVIESGPHCANTEIIVKLSDGRELCLDPKE | |
| | | NWVQRVVEKFLKRAENSEPKSCDKTHTCPP | |
| | | CPAPELLGGPSVFLFPPKPKDTLMISRTPE | |
| | | VTCVVVDVSHEDPEVKFNWYVDGVEVHNAK | |
| | | TKPREEQYGSTYRVVSVLTVLHQDWLNGKE | |
| | | YKCKVSNKALPAPIEKTISKAKGQPREPQV | |
| | | YTLPPSRDELTKNQVSLWCLVKGFYPSDIA | |
| | | VEWESNGQPENNYKTTPPVLDSDGSFFLYS | |
| | | KLTVDKSRWQQGNVFSCSVMHEALHNHYTQ | |
| | | KSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CXCL9 | Knob: | TPVVRKGRCSCISTNQGTIHLQSLKDLKQF | |
| | | APSPSCEKIEIIATLKNGVQTCLNPDSADV | |
| | | KELIKKWEKQVSQKKKQKNGKKHQKKKVLK | |
| | | VRKSQRSRQKKTTEPKSCDKTHTCPPCPAP | |
| | | ELLGGPSVFLFPPKPKDTLMISRTPEVTCV | |
| | | VVDVSHEDPEVKFNWYVDGVEVHN | |
| | | AKTKPREEQYGSTYRVVSVLTVLHQDWLNG | |
| | | KEYKCKVSNKALPAPIEKTISKAKGQPREP | |
| | | QVYTLPPSRDELTKNQVSLWCLVKGFYPSD | |
| | | IAVEWESNGQPENNYKTTPPVLDSDGSFFL | |
| | | YSKLTVDKSRWQQGNVFSCSVMHEALHNHY | |
| | | TQKSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CXCL13 | Knob: | VLEVYYTSLRCRCVQESSVFIPRRFIDRIQ | |
| | | ILPRGNGCPRKEIIVWKKNKSIVCVDPQAE | |
| | | WIQRMMEVLRKRSSSTLPVPVFKRKIPEPK | |
| | | SCDKTHTCPPCPAPELLGGPSVFLFPPKPK | |
| | | DTLMISRTPEVTCVVVDVSHEDPEVKFNWY | |
| | | VDGVEVHNAKTKPREEQYGSTYRVVSVLTV | |
| | | LHQDWLNGKEYKCKVSNKALPAPIEKTISK | |
| | | AKGQPREPQVYTLPPSRDELTKNQVSLWCL | |
| | | VKGFYPSDIAVEWESNGQPENNYKTTPPVL | |
| | | DSDGSFFLYSKLTVDKSRWQQGNVFSCSVM | |
| | | HEALHNHYTQKSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CXCL16 | Knob: | NEGSVTGSCYCGKRISSDSPPSVQFMNRLR | |
| | | KHLRAYHRCLYYTRFQLLSWSVCGGNKDPW | |
| | | VQELMSCLDLKECGHAYSGIVAHQK | |
| | | HLLPEPKSCDKTHTCPPCPAPELLGGPSVF | |
| | | LFPPKPKDTLMISRTPEVTCVVVDVSHEDP | |
| | | EVKFNWYVDGVEVHNAKTKPREEQYGSTYR | |
| | | VVSVLTVLHQDWLNGKEYKCKVSNKALPAP | |
| | | IEKTISKAKGQPREPQVYTLPPSRDELTKN | |
| | | QVSLWCLVKGFYPSDIAVEWESNGQPENNY | |
| | | KTTPPVLDSDGSFFLYSKLTVDKSRWQQGN | |
| | | VFSCSVMHEALHNHYTQKSLSLSPGKGGSH | |
| | | HHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CXCL17 | Knob: | SSLNPGVARGHRDRGQASRRWLQEGGQECE | |
| | | CKDWFLRAPRRKFMTVSGLPKKQCPCDHFK | |
| | | GNVKKTRHQRHHRKPNKHSRACQQFLKQCQ | |
| | | LRSFALPLEPKSCDKTHTCPPCPAPELLGG | |
| | | PSVFLFPPKPKDTLMISRTPEVTCVVVDVS | |
| | | HEDPEVKFNWYVDGVEVHNAKTKPREEQYG | |
| | | STYRVVSVLTVLHQDWLNGKEYKCKVSNKA | |
| | | LPAPIEKTISKAKGQPREPQVYTLPPSRDE | |
| | | LTKNQVSLWCLVKGFYPSDIAVEWESNGQP | |
| | | ENNYKTTPPVLDSDGSFFLYSKLTVDKSRW | |
| | | QQGNVFSCSVMHEALHNHYTQKSLSLSPGK | |
| | | GGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CCL1 | Knob: | SKSMQVPFSRCCFSFAEQEIPLRAILCYRN | |
| | | TSSICSNEGLIFKLKRGKEACALDTVGWVQ | |
| | | RHRKMLRHCPSKRKEPKSCDKTHTCPPCPA | |
| | | PELLGGPSVFLFPPKPKDTLMISRTPEVTC | |
| | | VVVDVSHEDPEVKFNWYVDGVEVHNAKTKP | |
| | | REEQYGSTYRVVSVLTVLHQDWLNGKEYKC | |
| | | KVSNKALPAPIEKTISKAKGQPREPQVYTL | |
| | | PPSRDELTKNQVSLWCLVKGFYPSDIAVEW | |
| | | ESNGQPENNYKTTPPVLDSDGSFFLYSKLT | |
| | | VDKSRWQQGNVFSCSVMHEALHNHYTQKSL | |
| | | SLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CCL2 | Knob: | QPDAINAPVTCCYNFTNRKISVQRLASYRR | |
| | | ITSSKCPKEAVIFKTIVAKEICADPKQKWV | |
| | | QDSMDHLDKQTQTPKTEPKSCDKTHTCPPC | |
| | | PAPELLGGPSVFLFPPKPKDTLMISRTPEV | |
| | | TCVVVDVSHEDPEVKFNWYVDGVEVHNAKT | |
| | | KPREEQYGSTYRVVSVLTVLHQDWLNGKEY | |
| | | KCKVSNKALPAPIEKTISKAKGQPREPQVY | |
| | | TLPPSRDELTKNQVSLWCLVKGFYPSDIAV | |
| | | EWESNGQPENNYKTTPPVLDSDGSFFLYSK | |
| | | LTVDKSRWQQGNVFSCSVMHEALHNHYTQK | |
| | | SLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RESGSGSGTDFTLTISSLQPEDFATYYCQQ | |
| | | YSTVPWTFGQGTKVEIKRTVAAPSVFIFPP | |
| | | SDEQLKSGTASVVC | |
| | | LLNNFYPREAKVQWKVDNALQSGNSQESVT | |
| | | EQDSKDSTYSLSSTLTLSKADYEKHKVYAC | |
| | | EVTHQGLSSPVTKSFNRGEC | |
| CCL3 | Knob: | ASLAADTPTACCFSYTSRQIPQNFIADYFE | |
| | | TSSQCSKPGVIFLTKRSRQVCADPSEEWVQ | |
| | | KYVSDLELSAEPKSCDKTHTCPPCPAPELL | |
| | | GGPSVFLFPPKPKDTLMISRTPEVTCVVVD | |
| | | VSHEDPEVKENWYVDGVEVHNAKTKPREEQ | |
| | | YGSTYRVVSVLTVLHQDWLNGKEYKCKVSN | |
| | | KALPAPIEKTISKAKGQPREPQVYTLPPSR | |
| | | DELTKNQVSLWCLVKGFYPSDIAVEWESNG | |
| | | QPENNYKTTPPVLDSDGSFFLYSKLTVDKS | |
| | | RWQQGNVFSCSVMHEALHNHYTQKSLSLSP | |
| | | GKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CCL3L1 | Knob: | APLAADTPTACCFSYTSRQIPQNFIADYFE | |
| | | TSSQCSKPSVIFLTKRGRQVCADPSEEWVQ | |
| | | KYVSDLELSAEPKSCDKTHTCPPCPAPELL | |
| | | GGPSVFLFPPKPKDTLMISRTPEVTCVVVD | |
| | | VSHEDPEVKFNWYVDGVEVHNAKTKPREEQ | |
| | | YGSTYRVVSVLTVLHQDWLNGKEYKCKVSN | |
| | | KALPAPIEKTISKAKGQPREPQVYTLPPSR | |
| | | DELTKNQVSLWCLVKGFYPSDIAVEWESNG | |
| | | QPENNYKTTPPVLDSDGSFFLYSKLTVDKS | |
| | | RWQQGNVFSCSVMHEALHNHYTQKSLSLSP | |
| | | GKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CCL4 | Knob: | APMGSDPPTACCFSYTARKLPRNFVVDYYE | |
| | | TSSLCSQPAVVFQTKRSKQVCADPSESWVQ | |
| | | EYVYDLELNEPKSCDKTHTCPPCPAPELLG | |
| | | GPSVFLFPPKPKDTLMISRTPEVTCVVVDV | |
| | | SHEDPEVKFNWYVDGVEVHNAKTKPREEQY | |
| | | GSTYRVVSVLTVLHQDWLNGKEYKCKVSNK | |
| | | ALPAPIEKTISKAKGQPREPQVYTLPPSRD | |
| | | ELTKNQVSLWCLVKGFYPSDIAVEWESNGQ | |
| | | PENNYKTTPPVLDSDGSFFLYSKLTVDKSR | |
| | | WQQGNVFSCSVMHEALHNHYTQKSLSLSPG | |
| | | KGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CC4L1 | Knob: | APMGSDPPTACCFSYTARKLPRNFVVDYYE | |
| | | TSSLCSQPAVVFQTKRGKQVCADPSESWVQ | |
| | | EYVYDLELNEPKSCDKTHTCPPCPAPELLG | |
| | | GPSVFLFPPKPKDTLMISRTPEVTCVVVDV | |
| | | SHEDPEVKFNWYVDGVEVHNAKTKPREEQY | |
| | | GSTYRVVSVLTVLHQDWLNGKEYKCKVSNK | |
| | | ALPAPIEKTISKAKGQPREPQVYTLPPSRD | |
| | | ELTKNQVSLWCLVKGFYPSDIAVEWESNGQ | |
| | | PENNYKTTPPVLDSDGSFFLYSKLTVDKSR | |
| | | WQQGNVFSCSVMHEALHNHYTQKSLSLSPG | |
| | | KGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEW | |
| | | ESNGQPENNYKTTPPVLDSDGSFFLVSKLT | |
| | | VDKSRWQQGNVFSCSVMHEALHNHYTQKSL | |
| | | SLSPGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CCL5 | Knob: | SPYSSDTTPCCFAYIARPLPRAHIKEYFYT | |
| | | SGKCSNPAVVFVTRKNRQVCANPEKKWVRE | |
| | | YINSLEMSEPKSCDKTHTCPPCPAPELLGG | |
| | | PSVFLFPPKPKDTLMISRTPEVTCVVVDVS | |
| | | HEDPEVKENWYVDGVEVHNAKTKPREEQYG | |
| | | STYRVVSVLTVLHQDWLNGKEYKCKVSNKA | |
| | | LPAPIEKTISKAKGQPREPQVYTLPPSRDE | |
| | | LTKNQVSLWCLVKGFYPSDIAVEWESNGQP | |
| | | ENNYKTTPPVLDSDGSFFLYSKLTVDKSRW | |
| | | QQGNVFSCSVMHEALHNHYTQKSLSLSPGK | |
| | | GGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CCL7 | Knob: | QPVGINTSTTCCYRFINKKIPKQRLESYRR | |
| | | TTSSHCPREAVIFKTKLDKEICADPTQKWV | |
| | | QDFMKHLDKKTQTPKLEPKSCDKTHTCPPC | |
| | | PAPELLGGPSVFLFPPKPKDTLMISRTPEV | |
| | | TCVVVDVSHEDPEVKFNWYVDGVEVHNAKT | |
| | | KPREEQYGSTYRVVSVLTVLHQDWLNGKEY | |
| | | KCKVSNKALPAPIEKTISKAKGQPREPQVY | |
| | | TLPPSRDELTKNQVSLWCLVKGFYPSDIAV | |
| | | EWESNGQPENNYKTTPPVLDSDGSFFLYSK | |
| | | LTVDKSRWQQGNVFSCSVMHEALHNHYTQK | |
| | | SLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | Q | |
| | | YGSTYRVVSVLTVLHQDWLNGKEYKCKVSN | |
| | | KALPAPIEKTISKAKGQPREPQVYTLPPSR | |
| | | DELTKNQVSLSCAVKGFYPSDIAVEWESNG | |
| | | QPENNYKTTPPVLDSDGSFFLVSKLTVDKS | |
| | | RWQQGNVFSCSVMHEALHNHYTQKSLSLSP | |
| | | GKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CCL8 | Knob: | QPDSVSIPITCCFNVINRKIPIQRLESYTR | |
| | | ITNIQCPKEAVIFKTKRGKEVCADPKERWV | |
| | | RDSMKHLDQIFQNLKPEPKSCDKTHTCPPC | |
| | | PAPELLGGPSVFLFPPKPKDTLMISRTPEV | |
| | | TCVVVDVSHEDPEVKFNWYVDGVEVHNAKT | |
| | | KPREEQYGSTYRVVSVLTVLHQDWLNGKEY | |
| | | KCKVSNKALPAPIEKTISKAKGQPREPQVY | |
| | | TLPPSRDELTKNQVSLWCLVKGFYPSDIAV | |
| | | EWESNGQPENNYKTTPPVLDSDGSFFLYSK | |
| | | LTVDKSRWQQGNVFSCSVMHEALHNHYTQK | |
| | | SLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CCL11 | Knob: | GPASVPTTCCFNLANRKIPLQRLESYRRIT | |
| | | SGKCPQKAVIFKTKLAKDICADPKKKWVQD | |
| | | SMKYLDQKSPTPKPEPKSCDKTHTCPPCPA | |
| | | PELLGGPSVFLFPPKPKDTLMISRTPEVTC | |
| | | VVVDVSHEDPEVKFNWYVDGVEVHNAKTKP | |
| | | REEQYGSTYRVVSVLTVLHQDWLNGKEYKC | |
| | | KVSNKALPAPIEKTISKAKGQPREPQVYTL | |
| | | PPSRDELTKNQVSLWCLVKGFYPSDIAVEW | |
| | | ESNGQPENNYKTTPPVLDSDGSFFLYSKLT | |
| | | VDKSRWQQGNVFSCSVMHEALHNHYTQKSL | |
| | | SLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNT | |
| | | KVDKKVEPKSCDKTHTCPPCPAPELLGGPS | |
| | | VFLFPPKPKDTLMISRTPEVTCVVVDVSHE | |
| | | DPEVKFNWYVDGVEVHNAKTKPREEQYGST | |
| | | YRVVSVLTVLHQDWLNGKEYKCKVSNKALP | |
| | | APIEKTISKAKGQPREPQVYTLPPSRDELT | |
| | | KNQVSLSCAVKGFYPSDIAVEWESNGQPEN | |
| | | NYKTTPPVLDSDGSFFLVSKLTVDKSRWQQ | |
| | | GNVFSCSVMHEALHNHYTQKSLSLSPGKGG | |
| | | SGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CCL13 | Knob: | QPDALNVPSTCCFTFSSKKISLQRLKSYVI | |
| | | TTSRCPQKAVIFRTKLGKEICADPKEKWVQ | |
| | | NYMKHLGRKAHTLKTEPKSCDKTHTCPPCP | |
| | | APELLGGPSVFLFPPKPKDTLMISRTPEVT | |
| | | CVVVDVSHEDPEVKFNWYVDGVEVHNAKTK | |
| | | PREEQYGSTYRVVSVLTVLHQDWLNGKEYK | |
| | | CKVSNKALPAPIEKTISKAKGQPREPQVYT | |
| | | LPPSRDELTKNQVSLWCLVKGFYPSDIAVE | |
| | | WESNGQPENNYKTTPPVLDSDGSFFLYSKL | |
| | | TVDKSRWQQGNVFSCSVMHEALHNHYTQKS | |
| | | LSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CCL14 | Knob: | TESSSRGPYHPSECCFTYTTYKIPRQRIMD | |
| | | YYETNSQCSKPGIVFITKRGHSVCTNPSDK | |
| | | WVQDYIKDMKENEPKSCDKTHTCPPCPAPE | |
| | | LLGGPSVFLFPPKPKDTLMISRTPEVTCVV | |
| | | VDVSHEDPEVKFNWYVDGVEVHNAKTKPRE | |
| | | EQYGSTYRVVSVLTVLHQDWLNGKEYKCKV | |
| | | SNKALPAPIEKTISKAKGQPREPQVYTLPP | |
| | | SRDELTKNQVSLWCLVKGFYPSDIAVEWES | |
| | | NGQPENNYKTTPPVLDSDGSFFLYSKLTVD | |
| | | KSRWQQGNVFSCSVMHEALHNHYTQKSLSL | |
| | | SPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTK | |
| | | GPSVFPLAPSSKSTSGGTAALGCLVKDYFP | |
| | | EPVTVSWNSGALTSGVHTFPAVLQSSGLYS | |
| | | LSSVVTVPSSSLGTQTYICNVNHKPSNTKV | |
| | | DKKVEPKSCDKTHTCPPCPAPELLGGPSVF | |
| | | LFPPKPKDTLMISRTPEVTCVVVDVSHEDP | |
| | | EVKFNWYVDGVEVHNAKTKPREEQYGSTYR | |
| | | VVSVLTVLHQDWLNGKEYKCKVSNKALPAP | |
| | | IEKTISKAKGQPREPQVYTLPPSRDELTKN | |
| | | QVSLSCAVKGFYPSDIAVEWESNGQPENNY | |
| | | KTTPPVLDSDGSFFLVSKLTVDKSRWQQGN | |
| | | VFSCSVMHEALHNHYTQKSLSLSPGKGGSG | |
| | | AWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CCL15 | Knob: | QFINDAETELMMSKLPLENPVVLNSFHFAA | |
| | | DCCTSYISQSIPCSLMKSYFETSSECSKPG | |
| | | VIFLTKKGRQVCAKPSGPGVQDCMKKLKPY | |
| | | SIEPKSCDKTHTCPPCPAPELLGGPSVFLF | |
| | | PPKPKDTLMISRTPEVTCVVVDVSHEDPEV | |
| | | KFNWYVDGVEVHNAKTKPREEQYGSTYRVV | |
| | | SVLTVLHQDWLNGKEYKCKVSNKALPAPIE | |
| | | KTISKAKGQPREPQVYTLPPSRDELTKNQV | |
| | | SLWCLVKGFYPSDIAVEWESNGQPENNYKT | |
| | | TPPVLDSDGSFFLYSKLTVDKSRWQQGNVF | |
| | | SCSVMHEALHNHYTQKSLSLSPGKGGSHHH | |
| | | HHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RESGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CCL16 | Knob: | QPKVPEWVNTPSTCCLKYYEKVLPRRLVVG | |
| | | YRKALNCHLPAIIFVTKRNREVCTNPNDDW | |
| | | VQEYIKDPNLPLLPTRNLSTVKIITAKNGQ | |
| | | PQLLNSQEPKSCDKTHTCPPCPAPELLGGP | |
| | | SVFLFPPKPKDTLMISRTPEVTCVVVDVSH | |
| | | EDPEVKFNWYVDGVEVHNAKTKPREEQYGS | |
| | | TYRVVSVLTVLHQDWLNGKEYKCKVSNKAL | |
| | | PAPIEKTISKAKGQPREPQVYTLPPSRDEL | |
| | | TKNQVSLWCLVKGFYPSDIAVEWESNGQPE | |
| | | NNYKTTPPVLDSDGSFFLYSKLTVDKSRWQ | |
| | | QGNVFSCSVMHEALHNHYTQKSLSLSPGKG | |
| | | GSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CCL17 | Knob: | ARGTNVGRECCLEYFKGAIPLRKLKTWYQT | |
| | | SEDCSRDAIVFVTVQGRAICSDPNNKRVKN | |
| | | AVKYLQSLERSEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLWCLVKGFYPSDIAVEWESN | |
| | | GQPENNYKTTPPVLDSDGSFFLYSKLTVDK | |
| | | SRWQQGNVFSCSVMHEALHNHYTQKSLSLS | |
| | | PGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CCL18 | Knob: | AQVGTNKELCCLVYTSWQIPQKFIVDYSET | |
| | | SPQCPKPGVILLTKRGRQICADPNKKWVQK | |
| | | YISDLKLNAEPKSCDKTHTCPPCPAPELLG | |
| | | GPSVFLFPPKPKDTLMISRTPEVTCVVVDV | |
| | | SHEDPEVKENWYVDGVEVHNAKTKPREEQY | |
| | | GSTYRVVSVLTVLHQDWLNGKEYKCKVSNK | |
| | | ALPAPIEKTISKAKGQPREPQVYTLPPSRD | |
| | | ELTKNQVSLWCLVKGFYPSDIAVEWESNGQ | |
| | | PENNYKTTPPVLDSDGSFF | |
| | | LYSKLTVDKSRWQQGNVFSCSVMHEALHNH | |
| | | YTQKSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CCL19 | Knob: | GTNDAEDCCLSVTQKPIPGYIVRNFHYLLI | |
| | | KDGCRVPAVVFTTLRGRQLCAPPDQPWVER | |
| | | IIQRLQRTSAKMKRRSSEPKSCDKTHTCPP | |
| | | CPAPELLGGPSVFLFPPKPKDTLMISRTPE | |
| | | VTCVVVDVSHEDPEVKFNWYVDGVEVHNAK | |
| | | TKPREEQYGSTYRVVSVLTVLHQDWLNGKE | |
| | | YKCKVSNKALPAPIEKTISKAKGQPREPQV | |
| | | YTLPPSRDELTKNQVSLWCLVKGFYPSDIA | |
| | | VEWESNGQPENNYKTTPPVLDSDGSFFLYS | |
| | | KLTVDKSRWQQGNVFSCSVMHEALHNHYTQ | |
| | | KSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CCL20 | Knob: | ASNFDCCLGYTDRILHPKFIVGFTRQLANE | |
| | | GCDINAIIFHTKKKLSVCANPKQTWVKYIV | |
| | | RLLSKKVKNMEPKSCDKTHTCPPCPAPELL | |
| | | GGPSVFLFPPKPKDTLMISRTPEVTCVVVD | |
| | | VSHEDPEVKENWYVDGVEVHNAKTKPREEQ | |
| | | YGSTYRVVSVLTVLHQDWLNGKE | |
| | | YKCKVSNKALPAPIEKTISKAKGQPREPQV | |
| | | YTLPPSRDELTKNQVSLWCLVKGFYPSDIA | |
| | | VEWESNGQPENNYKTTPPVLDSDGSFFLYS | |
| | | KLTVDKSRWQQGNVFSCSVMHEALHNHYTQ | |
| | | KSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CCL21 | Knob: | SDGGAQDCCLKYSQRKIPAKVVRSYRKQEP | |
| | | SLGCSIPAILFLPRKRSQAELCADPKELWV | |
| | | QQLMQHLDKTPSPQKPAQGCRKDRGASKTG | |
| | | KKGKGSKGCKRTERSQTPKGPEPKSCDKTH | |
| | | TCPPCPAPELLGGPSVFLFPPKPKDTLMIS | |
| | | RTPEVTCVVVDVSHEDPEVKFNWYVDGVEV | |
| | | HNAKTKPREEQYGSTYRVVSVLTVLHQDWL | |
| | | NGKEYKCKVSNKALPAPIEKTISKAKGQPR | |
| | | EPQVYTLPPSRDELTKNQVSLWCLVKGFYP | |
| | | SDIAVEWESNGQPENNYKTTPPVLDSDGSF | |
| | | FLYSKLTVDKSRWQQGNVFSCSVMHEALHN | |
| | | HYTQKSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CCL22 | Knob: | GPYGANMEDSVCCRDYVRYRLPLRVVKHFY | |
| | | WTSDSCPRPGVVLLTFRDKEICADPRVPWV | |
| | | KMILNKLSQEPKSCDKTHTCPPCPAPELLG | |
| | | GPSVFLFPPKPKDTLMISRTPEVTCVVVDV | |
| | | SHEDPEVKENWYVDGVEVHNAKTKPREEQY | |
| | | GSTYRVVSVLTVLHQDWLNGKEYKCKVSNK | |
| | | ALPAPIEKTISKAKGQPREPQVYTLPPSRD | |
| | | ELTKNQVSLWCLVKGFYPSDIAVEWESNGQ | |
| | | PENNYKTTPPVLDSDGSFFLYSKLTVDKSR | |
| | | WQQGNVFSCSVMHEALHNHYTQKSLSLSPG | |
| | | KGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CCL23 | Knob: | RVTKDAETEFMMSKLPLENPVLLDRFHATS | |
| | | ADCCISYTPRSIPCSLLESYFETNSECSKP | |
| | | GVIFLTKKGRRFCANPSDKQVQVCVRMLKL | |
| | | DTRIKTRKNEPKSCDKTHTCPPCPAPELLG | |
| | | GPSVFLFPPKPKDTLMISRTPEVTCVVVDV | |
| | | SHEDPEVKFNWYVDGVEVHNAKTKPREEQY | |
| | | GSTYRVVSVLTVLHQDWLNGKEYKCKVSNK | |
| | | ALPAPIEKTISKAKGQPREPQVYTLPPSRD | |
| | | ELTKNQVSLWCLVKGFYPSDIAVEWESNGQ | |
| | | PENNYKTTPPVLDSDGSFFLYSKLTVDKSR | |
| | | WQQGNVFSCSVMHEALHNHYTQKSLSLSPG | |
| | | KGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVC | |
| | | LLNNFYPREAKVQWKVDNALQSGNSQESVT | |
| | | EQDSKDSTYSLSSTLTLSKADYEKHKVYAC | |
| | | EVTHQGLSSPVTKSFNRGEC | |
| CCL24 | Knob: | VVIPSPCCMFFVSKRIPENRVVSYQLSSRS | |
| | | TCLKAGVIFTTKKGQQFCGDPKQEWVQRYM | |
| | | KNLDAKQKKASPRARAVAEPKSCDKTHTCP | |
| | | PCPAPELLGGPSVFLFPPKPKDTLMISRTP | |
| | | EVTCVVVDVSHEDPEVKFNWYVDGVEVHNA | |
| | | KTKPREEQYGSTYRVVSVLTVLHQDWLNGK | |
| | | EYKCKVSNKALPAPIEKTISKAKGQPREPQ | |
| | | VYTLPPSRDELTKNQVSLWCLVKGFYPSDI | |
| | | AVEWESNGQPENNYKTTPPVLDSDGSFFLY | |
| | | SKLTVDKSRWQQGNVFSCSVMHEALHNHYT | |
| | | QKSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CCL25 | Knob: | QGVFEDCCLAYHYPIGWAVLRRAWTYRIQE | |
| | | VSGSCNLPAAIFYLPKRHRKVCGNPKSREV | |
| | | QRAMKLLDARNKVFAKLHHNTQTFQAGPHA | |
| | | VKKLSSGNSKLSSSKFSNPISSSKRNVSLL | |
| | | ISANSGLEPKSCDKTHTCPPCPAPELLGGP | |
| | | SVFLFPPKPKDTLMISRTPEVTCVVVDVSH | |
| | | EDPEVKFNWYVDGVEVHNAKTKPREEQYGS | |
| | | TYRVVSVLTVLHQDWLNGKEYKCKVSNKAL | |
| | | PAPIEKTISKAKGQPREPQVYTLPPSRDEL | |
| | | TKNQVSLWCLVKGFYPSDIAVEWESNGQPE | |
| | | NNYKTTPPVLDSDGSFFLYSKLTVDKSRWQ | |
| | | QGNVFSCSVMHEALHNHYTQKSLSLSPGKG | |
| | | GSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CCL26 | Knob: | TRGSDISKTCCFQYSHKPLPWTWVRSYEFT | |
| | | SNSCSQRAVIFTTKRGKKVCTHPRKKWVQK | |
| | | YISLLKTPKQLEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLWCLVKGFYPSDIAVEWESN | |
| | | GQPENNYKTTPPVLDSDGSFFLYSKLTVDK | |
| | | SRWQQGNVFSCSVMHEALHNHYTQKSLSLS | |
| | | PGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CCL27 | Knob: | FLLPPSTACCTQLYRKPLSDKLLRKVIQVE | |
| | | LQEADGDCHLQAFVLHLAQRSICIHPQNPS | |
| | | LSQWFEHQERKLHGTLPKLNFGMLRKMGEP | |
| | | KSCDKTHTCPPCPAPELLGGPSVFLFPPKP | |
| | | KDTLMISRTPEVTCVVVDVSHEDPEVKFNW | |
| | | YVDGVEVHNAKTKPREEQYGSTYRVVSVLT | |
| | | VLHQDWLNGKEYKCKVSNKALPAPIEKTIS | |
| | | KAKGQPREPQVYTLPPSRDELTKNQVSLWC | |
| | | LVKGFYPSDIAVEWESNGQPENNYKTTPPV | |
| | | LDSDGSFFLYSKLTVDKSRWQQGNVFSCSV | |
| | | MHEALHNHYTQKSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CCL28 | Knob: | SEAILPIASSCCTEVSHHISRRLLERVNMC | |
| | | RIQRADGDCDLAAVILHVKRRRICVSPHNH | |
| | | TVKQWMKVQAAKKNGKGNVCHRKKHHGKRN | |
| | | SNRAHQGKHETYGHKTPYEPKSCDKTHTCP | |
| | | PCPAPELLGGPSVFLFPPKPKDTLMISRTP | |
| | | EVTCVVVDVSHEDPEVKFNWYVDGVEVHNA | |
| | | KTKPREEQYGSTYRVVSVLTVLHQDWLNGK | |
| | | EYKCKVSNKALPAPIEKTISKAKGQPREPQ | |
| | | VYTLPPSRDELTKNQVSLWCLVKGFYPSDI | |
| | | AVEWESNGQPENNYKTTPPVLDSDGSFFLY | |
| | | SKLTVDKSRWQQGNVFSCSVMHEALHNHYT | |
| | | QKSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| CX3CL1 | Knob: | QHHGVTKCNITCSKMTSKIPVALLIHYQQN | |
| | | QASCGKRAIILETRQHRLFCADPKEQWVKD | |
| | | AMQHLDRQAAALTRNGEPKSCDKTHTCPPC | |
| | | PAPELLGGPSVFLFPPKPKDTLMISRTPEV | |
| | | TCVVVDVSHEDPEVKFNWYVDGVEVHNAKT | |
| | | KPREEQYGSTYRVVSVLTVLHQDWLNGKEY | |
| | | KCKVSNKALPAPIEKTISKAKGQPREPQVY | |
| | | TLPPSRDELTKNQVSLWCLVKGFYPSDIAV | |
| | | EWESNGQPENNYKTTPPVLDSDGSFFLYSK | |
| | | LTVDKSRWQQGNVFSCSVMHEALHNHYTQK | |
| | | SLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMI | |
| | | SRTPEVTCVVVDVSHEDPEVKFNWYVDGVE | |
| | | VHNAKTKPREEQYGSTYRVVSVLTVLHQDW | |
| | | LNGKEYKCKVSNKALPAPIEKTISKAKGQP | |
| | | REPQVYTLPPSRDELTKNQVSLSCAVKGFY | |
| | | PSDIAVEWESNGQPENNYKTTPPVLDSDGS | |
| | | FFLVSKLTVDKSRWQQGNVFSCSVMHEALH | |
| | | NHYTQKSLSLSPGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| XCL1,2 | Knob: | VGSEVSDKRTCVSLTTQRLPVSRIKTYTIT | |
| | | EGSLRAVIFITKRGLKVCADPQATWVRDVV | |
| | | RSMDRKSNTRNNMIQTKPTGTQQSTNTAVT | |
| | | LTGEPKSCDKTHTCPPCPAPELLGGPSVFL | |
| | | FPPKPKDTLMISRTPEVTCVVVDVSHEDPE | |
| | | VKFNWYVDGVEVHNAKTKPREEQYGSTYRV | |
| | | VSVLTVLHQDWLNGKEYKCKVSNKALPAPI | |
| | | EKTISKAKGQPREPQVYTLPPSRDELTKNQ | |
| | | VSLWCLVKGFYPSDIAVEWESNGQPENNYK | |
| | | TTPPVLDSDGSFFLYSKLTVDKSRWQQGNV | |
| | | FSCSVMHEALHNHYTQKSLSLSPGKGGSHH | |
| | | HHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| FGF-1 | Knob: | FNLPPGNYKKPKLLYCSNGGHFLRILPDGT | |
| | | VDGTRDRSDQHIQLQLSAESVGEVYIKSTE | |
| | | TGQYLAMDTDGLLYGSQTPNEECLFLERLE | |
| | | ENHYNTYISKKHAEKNWFVGLKKNGSCKRG | |
| | | PRTHYGQKAILFLPLPVSSDEPKSCDKTHT | |
| | | CPPCPAPELLGGPSVFLFPPKPKDTLMISR | |
| | | TPEVTCVVVDVSHEDPEVKFNWYVDGVEVH | |
| | | NAKTKPREEQYGSTYRVVSVLTVLHQDWLN | |
| | | GKEYKCKVSNKALPAPIEKTISKAKGQPRE | |
| | | PQVYTLPPSRDELTKNQVSLWCLVKGFYPS | |
| | | DIAVEWESNGQPENNYKTTPPVLDSDGSFF | |
| | | LYSKLTVDKSRWQQGNVFSCSVMHEALHNH | |
| | | YTQKSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTK | |
| | | GPSVFPLAPSSKSTSGGTAALGCLVKDYFP | |
| | | EPVTVSWNSGALTSGVHTFPAVLQSSGLYS | |
| | | LSSVVTVPSSSLGTQTYICNVNHKPSNTKV | |
| | | DKKVEPKSCDKTHTCPPCPAPELLGGPSVF | |
| | | LFPPKPKDTLMISRTPEVTCVVVDVSHEDP | |
| | | EVKFNWYVDGVEVHNAKTKPREEQYGSTYR | |
| | | VVSVLTVLHQDWLNGKEYKCKVSNKALPAP | |
| | | IEKTISKAKGQPREPQVYTLPPSRDELTKN | |
| | | QVSLSCAVKGFYPSDIAVEWESNGQPENNY | |
| | | KTTPPVLDSDGSFFLVSKLTVDKSRWQQGN | |
| | | VFSCSVMHEALHNHYTQKSLSLSPGKGGSG | |
| | | AWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| FGF-2 | Knob: | PALPEDGGSGAFPPGHFKDPKRLYCKNGGF | |
| | | FLRIHPDGRVDGVREKSDPHIKLQLQAEER | |
| | | GVVSIKGVCANRYLAMKEDGRLLASKCVTD | |
| | | ECFFFERLESNNYNTYRSRKYTSWYVALKR | |
| | | TGQYKLGSKTGPGQKAILFLPMSAKSEPKS | |
| | | CDKTHTCPPCPAPELLGGPSVFLFPPKPKD | |
| | | TLMISRTPEVTCVVVDVSHEDPEVKFNWYV | |
| | | DGVEVHNAKTKPREEQYGSTYRVVSVLTVL | |
| | | HQDWLNGKEYKCKVSNKALPAPIEKTISKA | |
| | | KGQPREPQVYTLPPSRDELTKNQVSLWCLV | |
| | | KGFYPSDIAVEWESNGQPENNYKTTPPVLD | |
| | | SDGSFFLYSKLTVDKSRWQQGNVFSCSVMH | |
| | | EALHNHYTQKSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| FGF-3 | Knob: | AAGPGARLRRDAGGRGGVYEHLGGAPRRRK | |
| | | LYCATKYHLQLHPSGRVNGSLENSAYSILE | |
| | | ITAVEVGIVAIRGLFSGRYLAMNKRGRLYA | |
| | | SEHYSAECEFVERIHELGYNTYASRLYRTV | |
| | | SSTPGARRQPSAERLWYVSVNGKGRPRRGF | |
| | | KTRRTQKSSLFLPRVLDHRDHEMVRQLQSG | |
| | | LPRPPGKGVQPRRRRQKQSPDNLEPSHVQA | |
| | | SRLGSQLEASAHEPKSCDKTHTCPPCPAPE | |
| | | LLGGPSVFLFPPKPKDTLMISRTPEVTCVV | |
| | | VDVSHEDPEVKFNWYVDGVEVHNAKTKPRE | |
| | | EQYGSTYRVVSVLTVLHQDWLNGKEYKCKV | |
| | | SNKALPAPIEKTISKAKGQPREPQVYTLPP | |
| | | SRDELTKNQVSLWCLVKGFYPSDIAVEWES | |
| | | NGQPENNYKTTPPVLDSDGSFFLYSKLTVD | |
| | | KSRWQQGNVFSCSVMHEALHNHYTQKSLSL | |
| | | SPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| FGF-4 | Knob: | APTAPNGTLEAELERRWESLVALSLARLPV | |
| | | AAQPKEAAVQSGAGDYLLGIKRLRRLYCNV | |
| | | GIGFHLQALPDGRIGGAHADTRDSLLELSP | |
| | | VERGVVSIFGVASRFFVAMSSKGKLYGSPF | |
| | | FTDECTFKEILLPNNYNAYESYKYPGMFIA | |
| | | LSKNGKTKKGNRVSPTMKVTHFLPRLEPKS | |
| | | CDKTHTCPPCPAPELLGGPSVFLFPPKPKD | |
| | | TLMISRTPEVTCVVVDVSHEDPEVKFNWYV | |
| | | DGVEVHNAKTKPREEQYGSTYRVVSVLTVL | |
| | | HQDWLNGKEYKCKVSNKALPAPIEKTISKA | |
| | | KGQPREPQVYTLPPSRDELTKNQVSLWCLV | |
| | | KGFYPSDIAVEWESNGQPENNYKTTPPVLD | |
| | | SDGSFFLYSKLTVDKSRWQQGNVFSCSVMH | |
| | | EALHNHYTQKSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| FGF-5 | Knob: | HGEKRLAPKGQPGPAATDRNPRGSSSRQSS | |
| | | SSAMSSSSASSSPAASLGSQGSGLEQSSFQ | |
| | | WSPSGRRTGSLYCRVGIGFHLQIYPDGKVN | |
| | | GSHEANMLSVLEIFAVSQGIVGIRGVFSNK | |
| | | FLAMSKKGKLHASAKFTDDCKFRERFQENS | |
| | | YNTYASAIHRTEKTGREWYVALNKRGKAKR | |
| | | GCSPRVKPQHISTHFLPRFKQSEQPELSFT | |
| | | VTVPEKKKPPSPIKPKIPLSAPRKNTNSVK | |
| | | YRLKFRFGEPKSCDKTHTCPPCPAPELLGG | |
| | | PSVFLFPPKPKDTLMISRTPEVTCVVVDVS | |
| | | HEDPEVKFNWYVDGVEVHNAKTKPREEQYG | |
| | | STYRVVSVLTVLHQDWLNGKEYKCKVSNKA | |
| | | LPAPIEKTISKAKGQPREPQVYTLPPSRDE | |
| | | LTKNQVSLWCLVKGFYPSDIAVEWESNGQP | |
| | | ENNYKTTPPVLDSDGSFFLYSKLTVDKSRW | |
| | | QQGNVFSCSVMHEALHNHYTQKSLSLSPGK | |
| | | GGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| FGF-19 | Knob: | LAFSDAGPHVHYGWGDPIRLRHLYTSGPHG | |
| | | LSSCFLRIRADGVVDCARGQSAHSLLEIKA | |
| | | VALRTVAIKGVHSVRYLCMGADGKMQGLLQ | |
| | | YSEEDCAFEEEIRPDGYNVYRSEKHRLPVS | |
| | | LSSAKQRQLYKNRGFLPLSHELPMLPMVPE | |
| | | EPEDLRGHLESDMFSSPLETDSMDPFGLVT | |
| | | GLEAVRSPSFEKEPKSCDKTHTCPPCPAPE | |
| | | LLGGPSVFLFPPKPKDTLMISRTPEVTCVV | |
| | | VDVSHEDPEVKFNWYVDGVEVHNAKTKPRE | |
| | | EQYGSTYRVVSVLTVLHQDWLNGKEYKCKV | |
| | | SNKALPAPIEKTISKAKGQPREPQVYTLPP | |
| | | SRDELTKNQVSLWCLVKGFYPSDIAVEWES | |
| | | NGQPENNYKTTPPVLDSDGSFFLYSKLTVD | |
| | | KSRWQQGNVFSCSVMHEALHNHYTQKSLSL | |
| | | SPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| FGF-21 | Knob: | HPIPDSSPLLQFGGQVRQRYLYTDDAQQTE | |
| | | AHLEIREDGTVGGAADQSPESLLQLKALKP | |
| | | GVIQILGVKTSRFLCQRPDGALYGSLHFDP | |
| | | EACSFRELLLEDGYNVYQSEAHGLPLHLPG | |
| | | NKSPHRDPAPRGPARFLPLPGLPPALPEPP | |
| | | GILAPQPPDVGSSDPLSMVGPSQGRSPSYA | |
| | | SEPKSCDKTHTCPPCPAPELLGGPSVFLFP | |
| | | PKPKDTLMISRTPEVTCVVVDVSHEDPEVK | |
| | | FNWYVDGVEVHNAKTKPREEQYGSTYRVVS | |
| | | VLTVLHQDWLNGKEYKCKVSNKALPAPIEK | |
| | | TISKAKGQPREPQVYTLPPSRDELTKNQVS | |
| | | LWCLVKGFYPSDIAVEWESNGQPENNYKTT | |
| | | PPVLDSDGSFFLYSKLTVDKSRWQQGNVFS | |
| | | CSVMHEALHNHYTQKSLSLSPGKGGSHHHH | |
| | | HH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| FGF-21 | Knob: | CGEGLFTCRSTNICISHAWVCDGVDDCEDN | |
| Avimer | | SDENNCSAPASEPPGSLCGADQFRCGNGSC | |
| Mimic | | VPRAWRCDGVDDCGDGSDEAPEICETPTCQ | |
| | | SNEFRCRSGRCIPQHWLCDGLNDCGDGSDE | |
| | | SQQCSAPASEPPGSLEPKSCDKTHTCPPCP | |
| | | APELLGGPSVFLFPPKPKDTLMISRTPEVT | |
| | | CVVVDVSHEDPEVKFNWYVDGVEVHNAKTK | |
| | | PREEQYGSTYRVVSVLTVLHQDWLNGKEYK | |
| | | CKVSNKALPAPIEKTISKAKGQPREPQVYT | |
| | | LPPSRDELTKNQVSLWCLVKGFYPSDIAVE | |
| | | WESNGQPENNYKTTPPVLDSDGSFFLYSKL | |
| | | TVDKSRWQQGNVFSCSVMHEALHNHYTQKS | |
| | | LSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTK | |
| | | GPSVFPLAPSSKSTSGGTAALGCLVKDYFP | |
| | | EPVTVSWNSGALTSGVHTFPAVLQSSGLYS | |
| | | LSSVVTVPSSSLGTQTYICNVNHKPSNTKV | |
| | | DKKVEPKSCDKTHTCPPCPAPELLGGPSVF | |
| | | LFPPKPKDTLMISRTPEVTCVVVDVSHEDP | |
| | | EVKFNWYVDGVEVHNAKTKPREEQYGSTYR | |
| | | VVSVLTVLHQDWLNGKEYKCKVSNKALPAP | |
| | | IEKTISKAKGQPREPQVYTLPPSRDELTKN | |
| | | QVSLSCAVKGFYPSDIAVEWESNGQPENNY | |
| | | KTTPPVLDSDGSFFLVSKLTVDKSRWQQGN | |
| | | VFSCSVMHEALHNHYTQKSLSLSPGKGGSG | |
| | | AWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| FGF-23 | Knob: | YPNASPLLGSSWGGLIHLYTATARNSYHLQ | |
| | | IHKNGHVDGAPHQTIYSALMIRSEDAGFVV | |
| | | ITGVMSRRYLCMDFRGNIFGSHYFDPENCR | |
| | | FQHQTLENGYDVYHSPQYHFLVSLGRAKRA | |
| | | FLPGMNPPPYSQFLSRRNEIPLIHFNTPIP | |
| | | RRHTRSAEDDSERDPLNVLKPRARMTPAPA | |
| | | SCSQELPSAEDNSPMASDPLGVVRGGRVNT | |
| | | HAGGTGPEGCRPFAKFIEPKSCDKTHTCPP | |
| | | CPAPELLGGPSVFLFPPKPKDTLMISRTPE | |
| | | VTCVVVDVSHEDPEVKFNWYVDGVEVHNAK | |
| | | TKPREEQYGSTYRVVSVLTVLHQDWLNGKE | |
| | | YKCKVSNKALPAPIEKTISKAKGQPREPQV | |
| | | YTLPPSRDELTKNQVSLWCLVKGFYPSDIA | |
| | | VEWESNGQPENNYKTTPPVLDSDGSFFLYS | |
| | | KLTVDKSRWQQGNVFSCSVMHEALHNHYTQ | |
| | | KSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| KGF | Knob: | CNDMTPEQMATNVNCSSPERHTRSYDYMEG | |
| | | GDIRVRRLFCRTQWYLRIDKRGKVKGTQEM | |
| | | KNNYNIMEIRTVAVGIVAIKGVESEFYLAM | |
| | | NKEGKLYAKKECNEDCNFKELILENHYNTY | |
| | | ASAKWTHNGGEMFVALNQKGIPVRGKKTKK | |
| | | EQKTAHFLPMAITEPKSCDKTHTCPPCPAP | |
| | | ELLGGPSVFLFPPKPKDTLMISRTPEVTCV | |
| | | VVDVSHEDPEVKFNWYVDGVEVHNAKTKPR | |
| | | EEQYGSTYRVVSVLTVL | |
| | | HQDWLNGKEYKCKVSNKALPAPIEKTISKA | |
| | | KGQPREPQVYTLPPSRDELTKNQVSLWCLV | |
| | | KGFYPSDIAVEWESNGQPENNYKTTPPVLD | |
| | | SDGSFFLYSKLTVDKSRWQQGNVFSCSVMH | |
| | | EALHNHYTQKSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| VEGF | Knob: | APMAEGGGQNHHEVVKFMDVYQRSYCHPIE | |
| | | TLVDIFQEYPDEIEYIFKPSCVPLMRCGGC | |
| | | CNDEGLECVPTEESNITMQIMRIKPHQGQH | |
| | | IGEMSFLQHNKCECRPKKDRARQEKKSVRG | |
| | | KGKGQKRKRKKSRYKSWSVYVGARCCLMPW | |
| | | SLPGPHPCGPCSERRKHLFVQDPQTCKCSC | |
| | | KNTDSRCKARQLELNERTCRCDKPRREPKS | |
| | | CDKTHTCPPCPAPELLGGPSVFLFPPKPKD | |
| | | TLMISRTPEVTCVVVDVSHEDPEVKFNWYV | |
| | | DGVEVHNAKTKPREEQYGSTYRVVSVLTVL | |
| | | HQDWLNGKEYKCKVSNKALPAPIEKTISKA | |
| | | KGQPREPQVYTLPPSRDELTKNQVSLWCLV | |
| | | KGFYPSDIAVEWESNGQPENNYKTTPPVLD | |
| | | SDGSFFLYSKLTVDKSRWQQGNVFSCSVMH | |
| | | EALHNHYTQKSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVC | |
| | | LLNNFYPREAKVQWKVDNALQSGNSQESVT | |
| | | EQDSKDSTYSLSSTLTLSKADYEKHKVYAC | |
| | | EVTHQGLSSPVTKSFNRGEC | |
| PDGFαα | Knob: | SIEEAVPAVCKTRTVIYEIPRSQVDPTSAN | |
| | | FLIWPPCVEVKRCTGCCNTSSVKCQPSRVH | |
| | | HRSVKVAKVEYVRKKPKLKEVQVRLEEHLE | |
| | | CACATTSLNPDYREEDTGRPRESGKKRKRK | |
| | | RLKPTEPKSCDKTHTCPPCPAPELLGGPSV | |
| | | FLFPPKPKDTLMISRTPEVTCVVVDVSHED | |
| | | PEVKFNWYVDGVEVHNAKTKPREEQYGSTY | |
| | | RVVSVLTVLHQDWLNGKEYKCKVSNKALPA | |
| | | PIEKTISKAKGQPREPQVYTLPPSRDELTK | |
| | | NQVSLWCLVKGFYPSDIAVEWESNGQPENN | |
| | | YKTTPPVLDSDGSFFLYSKLTVDKSRWQQG | |
| | | NVFSCSVMHEALHNHYTQKSLSLSPGKGGS | |
| | | HHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| PDGFββ | Knob: | SLGSLTIAEPAMIAECKTRTEVFEISRRLI | |
| | | DRTNANFLVWPPCVEVQRCSGCCNNRNVQC | |
| | | RPTQVQLRPVQVRKIEIVRKKPIFKKATVT | |
| | | LEDHLACKCETVAAARPVTEPKSCDKTHTC | |
| | | PPCPAPELLGGPSVFLFPPKPKDTLMISRT | |
| | | PEVTCVVVDVSHEDPEVKFNWYVDGVEVHN | |
| | | AKTKPREEQYGSTYRVVSVLTVLHQDWLNG | |
| | | KEYKCKVSNKALPAPIEKTISKAKGQPREP | |
| | | QVYTLPPSRDELTKNQVSLWCLVKGFYPSD | |
| | | IAVEWESNGQPENNYKTTPPVLDSDGSFFL | |
| | | YSKLTVDKSRWQQGNVFSCSVMHEALHNHY | |
| | | TQKSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEW | |
| | | ESNGQPENNYKTTPPVLDSDGSFFLVSKLT | |
| | | VDKSRWQQGNVFSCSVMHEALHNHYTQKSL | |
| | | SLSPGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| NGF | Knob: | SSSHPIFHRGEFSVCDSVSVWVGDKTTATD | |
| | | IKGKEVMVLGEVNINNSVFKQYFFETKCRD | |
| | | PNPVDSGCRGIDSKHWNSYCTTTHTFVKAL | |
| | | TMDGKQAAWRFIRIDTACVCVLSRKAVRRA | |
| | | EPKSCDKTHTCPPCPAPELLGGPSVFLFPP | |
| | | KPKDTLMISRTPEVTCVVVDVSHEDPEVKF | |
| | | NWYVDGVEVHNAKTKPREEQYGSTYRVVSV | |
| | | LTVLHQDWLNGKEYKCKVSNKALPAPIEKT | |
| | | ISKAKGQPREPQVYTLPPSRDELTKNQVSL | |
| | | WCLVKGFYPSDIAVEWESNGQPENNYKTTP | |
| | | PVLDSDGSFFLYSKLTVDKSRWQQGNVFSC | |
| | | SVMHEALHNHYTQKSLSLSPGKGGSHHHHH | |
| | | H | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| NTF3 | Knob: | YAEHKSHRGEYSVCDSESLWVTDKSSAIDI | |
| | | RGHQVTVLGEIKTGNSPVKQYFYETRCKEA | |
| | | RPVKNGCRGIDDKHWNSQCKTSQTYVRALT | |
| | | SENNKLVGWRWIRIDTSCVCALSRKIGRTE | |
| | | PKSCDKTHTCPPCPAPELLGGPSVFLFPPK | |
| | | PKDTLMISRTPEVTCVVVDVSHEDPEVKFN | |
| | | WYVDGVEVHNAKTKPREEQYGSTYRVVSVL | |
| | | TVLHQDWLNGKEYKCKVSNKALPAPIEKTI | |
| | | SKAKGQPREPQVYTLPPSRDELTKNQVSLW | |
| | | CLVKGFYPSDIAVEWESNGQPENNYKTTPP | |
| | | VLDSDGSFFLYSKLTVDKSRWQQGNVFSCS | |
| | | VMHEALHNHYTQKSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNT | |
| | | KVDKKVEPKSCDKTHTCPPCPAPELLGGPS | |
| | | VFLFPPKPKDTLMISRTPEVTCVVVDVSHE | |
| | | DPEVKFNWYVDGVEVHNAKTKPREEQYGST | |
| | | YRVVSVLTVLHQDWLNGKEYKCKVSNKALP | |
| | | APIEKTISKAKGQPREPQVYTLPPSRDELT | |
| | | KNQVSLSCAVKGFYPSDIAVEWESNGQPEN | |
| | | NYKTTPPVLDSDGSFFLVSKLTVDKSRWQQ | |
| | | GNVFSCSVMHEALHNHYTQKSLSLSPGKGG | |
| | | SGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| NTF4 | Knob: | GVSETAPASRRGELAVCDAVSGWVTDRRTA | |
| | | VDLRGREVEVLGEVPAAGGSPLRQYFFETR | |
| | | CKADNAEEGGPGAGGGGCRGVDRRHWVSEC | |
| | | KAKQSYVRALTADAQGRVGWRWIRIDTACV | |
| | | CTLLSRTGRAEPKSCDKTHTCPPCPAPELL | |
| | | GGPSVFLFPPKPKDTLMISRTPEVTCVVVD | |
| | | VSHEDPEVKFNWYVDGVEVHNAKTKPREEQ | |
| | | YGSTYRVVSVLTVLHQDWLNGKEYKCKVSN | |
| | | KALPAPIEKTISKAKGQPREPQVYTLPPSR | |
| | | DELTKNQVSLWCLVKGFYPSDIAVEWESNG | |
| | | QPENNYKTTPPVLDSDGSFFLYSKLTVDKS | |
| | | RWQQGNVFSCSVMHEALHNHYTQKSLSLSP | |
| | | GKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| BDNF | Knob: | HSDPARRGELSVCDSISEWVTAADKKTAVD | |
| | | MSGGTVTVLEKVPVSKGQLKQYFYETKCNP | |
| | | MGYTKEGCRGIDKRHWNSQCRTTQSYVRAL | |
| | | TMDSKKRIGWRFIRIDTSCVCTLTIKRGRE | |
| | | PKSCDKTHTCPPCPAPELLGGPSVFLFPPK | |
| | | PKDTLMISRTPEVTCVVVDVSHEDPEVKFN | |
| | | WYVDGVEVHNAKTKPREEQYGSTYRVVSVL | |
| | | TVLHQDWLNGKEYKCKVSNKALPAPIEKTI | |
| | | SKAKGQPREPQVYTLPPSRDELTKNQVSLW | |
| | | CLVKGFYPSDIAVEWESNGQPENNYKTTPP | |
| | | VLDSDGSFFLYSKLTVDKSRWQQGNVFSCS | |
| | | VMHEALHNHYTQKSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| TGFβ1 | Knob: | ALDTNYCFSSTEKNCCVRQLYIDFRKDLGW | |
| | | KWIHEPKGYHANFCLGPCPYIWSLDTQYSK | |
| | | VLALYNQHNPGASAAPCCVPQALEPLPIVY | |
| | | YVGRKPKVEQLSNMIVRSCKCSEPKSCDKT | |
| | | HTCPPCPAPELLGGPSVFLFPPKPKDTLMI | |
| | | SRTPEVTCVVVDVSHEDPEVKFNWYVDGVE | |
| | | VHNAKTKPREEQYGSTYRVVSVLTVLHQDW | |
| | | LNGKEYKCKVSNKALPAPIEKTISKAKGQP | |
| | | REPQVYTLPPSRDELTKNQVSLWCLVKGFY | |
| | | PSDIAVEWESNGQPENNYKTTPPVLDSDGS | |
| | | FFLYSKLTVDKSRWQQGNVFSCSVMHEALH | |
| | | NHYTQKSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| TGFβ2 | Knob: | ALDAAYCFRNVQDNCCLRPLYIDFKRDLGW | |
| | | KWIHEPKGYNANFCAGACPYLWSSDTQHSR | |
| | | VLSLYNTINPEASASPCCVSQDLEPLTILY | |
| | | YIGKTPKIEQLSNMIVKSCKCSEPKSCDKT | |
| | | HTCPPCPAPELLGGPSVFLFPPKPKDTLMI | |
| | | SRTPEVTCVVVDVSHEDPEVKFNWYVDGVE | |
| | | VHNAKTKPREEQYGSTYRVVSVLTVLHQDW | |
| | | LNGKEY | |
| | | KCKVSNKALPAPIEKTISKAKGQPREPQVY | |
| | | TLPPSRDELTKNQVSLWCLVKGFYPSDIAV | |
| | | EWESNGQPENNYKTTPPVLDSDGSFFLYSK | |
| | | LTVDKSRWQQGNVFSCSVMHEALHNHYTQK | |
| | | SLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |
| HGF β | Knob: | VVNGIPTRTNIGWMVSLRYRNKHICGGSLI | |
| chain | | KESWVLTARQCFPSRDLKDYEAWLGIHDVH | |
| | | GRGDEKCKQVLNVSQLVYGPEGSDLVLMKL | |
| | | ARPAVLDDFVSTIDLPNYGCTIPEKTSCSV | |
| | | YGWGYTGLINYDGLLRVAHLYIMGNEKCSQ | |
| | | HHRGKVTLNESEICAGAEKIGSGPCEGDYG | |
| | | GPLVCEQHKMRMVLGVIVPGRGCAIPNRPG | |
| | | IFVRVAYYAKWIHKIILTYKVPQSEPKSCD | |
| | | KTHTCPPCPAPELLGGPSVFLFPPKPKDTL | |
| | | MISRTPEVTCVVVDVSHEDPEVKFNWYVDG | |
| | | VEVHNAKTKPREEQYGSTYRVVSVLTVLHQ | |
| | | DWLNGKEYKCKVSNKALPAPIEKTISKAKG | |
| | | QPREPQVYTLPPSRDELTKNQVSLWCLVKG | |
| | | FYPSDIAVEWESNGQPENNYKTTPPVLDSD | |
| | | GSFFLYSKLTVDKSRWQQGNVFSCSVMHEA | |
| | | LHNHYTQKSLSLSPGKGGSHHHHHH | |
| | Hole: | EVQLVESGGGLVQPGGSLRLSCAASGYTFT | |
| | | NYGMNWVRQAPGKGLEWVGWINTYTGEPTY | |
| | | AADFKRRFTFSLDTSKSTAYLQMNSLRAED | |
| | | TAVYYCAKYPHYYGSSHWYFDVWGQGTLVT | |
| | | VSSASTKGPSVFPLAPSSKSTSGGTAALGC | |
| | | LVKDYFPEPVTVSWNSGALTSGVHTFPAVL | |
| | | QSSGLYSLSSVVTVPSSSLGTQTYICNVNH | |
| | | KPSNTKVDKKVEPKSCDKTHTCPPCPAPEL | |
| | | LGGPSVFLFPPKPKDTLMISRTPEVTCVVV | |
| | | DVSHEDPEVKFNWYVDGVEVHNAKTKPREE | |
| | | QYGSTYRVVSVLTVLHQDWLNGKEYKCKVS | |
| | | NKALPAPIEKTISKAKGQPREPQVYTLPPS | |
| | | RDELTKNQVSLSCAVKGFYPSDIAVEWESN | |
| | | GQPENNYKTTPPVLDSDGSFFLVSKLTVDK | |
| | | SRWQQGNVFSCSVMHEALHNHYTQKSLSLS | |
| | | PGKGGSGAWSHPQFEK | |
| | L: | DIQMTQSPSSLSASVGDRVTITCSASQDIS | |
| | | NYLNWYQQKPGKAPKVLIYFTSSLHSGVPS | |
| | | RFSGSGSGTDFTLTISSLQPEDFATYYCQQ | |
| | | YSTVPWTFGQGTKVEIKRTVAAPSVFIFPP | |
| | | SDEQLKSGTASVVCLLNNFYPREAKVQWKV | |
| | | DNALQSGNSQESVTEQDSKDSTYSLSSTLT | |
| | | LSKADYEKHKVYACEVTHQGLSSPVTKSFN | |
| | | RGEC | |

### Example 12: Methods for Example 11

Cell culture of THP-1s: THP-1 cells are cultured between 0.3×10⁶-1×10⁶ cells/mL in RPMI-1640 supplemented with 10% FBS and 1% Pen/Strep at 37 C and 5% CO2 according to supplier recommendations (ATCC).

Flow cytometry: Cells are either harvested directly (if in suspension) or by PBS wash followed by trypsinization for 5 mins with 0.25% trypsin-EDTA. Samples are spun at 1000×g for 5 mins, then washed with PBS 3× before quantifying fluorescence on a CytoFLEX cytomer (Beckman Coulter).

KineTAC expression: 30 mL Expi293 cells are transfected with 10 μg of each plasmid using an Expifectamine 293 Transfection kit (ThermoScientific) according to manufacturer specifications. Plasmids are as follows: 1) knob-kine pFUSE, 2) protein of interest (POI) binding heavy chain-hole pFUSE, 3) POI binding LC pFuse. 3 days post transfection, cells are harvested by centrifugation at 4000×g for 20 mins before filtration through a 0.22 m PES filter. 5 mM imidazole is then added to the media along with 500 μL slurry Hi-Bind Ni QR Agarose Beads (BioVision). The resin is incubated at 4° C. in the media for 1 hr, then collected by gravity column. 3×4 column volume (CV) washes with 20 mM imidazole in PBS are conducted before eluting in 2 CV 300 mM imidazole in PBS twice. Bispecifics are then concentrated and resuspended in 20% glycerol before being analyzed, aliquoted and flash frozen at −80° C. for dosing experiments.

VEGF chemical conjugation with pHrodo: 5 μM biotinylated VEGF165 (Acro Biosystems) is resuspended in PBS 0.1M sodium bicarbonate. pHrodo NHS-ester (ThermoScientific) is resuspended in PBS with 10% DMSO, then added at 25 μM. After reacting in the dark at 25° C. for 1 hr, the reaction is quenched with 500× molar excess glycine pH 8 for an additional 1 hr at room temperature in the dark. Zeba Spin Desalting Columns (ThermoScientific) with a 7 KDa MWCO is used according to manufacturer specifications to remove excess dye and glycine. Final protein is resuspended in PBS and analyzed for labeling efficiency by spectrophotometry or MS.

General flow soluble ligand uptake protocol: 50,000-5,000,000 relevant cells (THP-1s, HeLa, MDA-MB-231, Jurkat, Daudi, etc) are incubated at 4-37° C. in 100-1000 μL conditioned media containing control compounds or bispecifics and protein of interest. Biotin-streptavidin dye linked VEGF or chemically conjugated VEGF-dye was diluted at 0.05-200 nM in RPMI-1640. Bispecifics are then added to conjugated VEGF for a final concentration of 0.05-500 nM bispecific and 0.05-500 nM VEGF conjugate. The mixture is then immediately dosed onto cells and allowed to incubate for 0.5-72 hrs before harvesting by centrifugation at 1000×g, washing 3× with PBS, and performing flow cytometry. Plots are gated for live and singlet cells and MFI and the relevant fluorescence channel was used to quantify efficiency of the KineTAC.

### Example 13: Additional Constructs

This example described additional constructs to be made and tested as described in Example 14.

| | | Seq |
|---|---|---|
| Construct | | ID |
| Name | Construct Sequence | No |
| IL12A | Knob: | |
| | RNLPVATPDPGMFPCLHHSQNLLRAVSNMLQKARQTLEFYPCTSE | |
| | EIDHEDITKDKTSTVEACLPLELTKNESCLNSRETSFITNGSCLASR | |
| | KTSFMMALCLSSIYEDLKMYQVEFKTMNAKLLMDPKRQIFLDQN | |
| | MLAVIDELMQALNFNSETVPQKSSLEEPDFYKTKIKLCILLHAFRIR | |
| | AVTIDRVMSYLNASEPKSCDKTHTCPPCPAPELLGGPSVFLFPPKP | |
| | KDTLMISRTPEVTCVVVDVSHEDPEVKFNWYVDGVEVHNAKTKP | |
| | REEQYGSTYRVVSVLTVLHQDWLNGKEYKCKVSNKALPAPIEKTI | |
| | SKAKGQPREPQVYTLPPSRDELTKNQVSLWCLVKGFYPSDIAVEW | |
| | ESNGQPENNYKTTPPVLDSDGSFFLYSKLTVDKSRWQQGNVFSCS | |
| | VMHEALHNHYTQKSLSLSPGKGGSHHHHHH | |
| IL12B | Knob: | |
| | IWELKKDVYVVELDWYPDAPGEMVVLTCDTPEEDGITWTLDQSS | |
| | EVLGSGKTLTIQVKEFGDAGQYTCHKGGEVLSHSLLLLHKKEDGI | |
| | WSTDILKDQKEPKNKTFLRCEAKNYSGRFTCWWLTTISTDLTFSV | |
| | KSSRGSSDPQGVTCGAATLSAERVRGDNKEYEYSVECQEDSACPA | |
| | AEESLPIEVMVDAVHKLKYENYTSSFFIRDIIKPDPPKNLQLKPLKN | |
| | SRQVEVSWEYPDTWSTPHSYFSLTFCVQVQGKSKREKKDRVFTD | |
| | KTSATVICRKNASISVRAQDRYYSSSWSEWASVPCSEPKSCDKTH | |
| | TCPPCPAPELLGGPSVFLFPPKPKDTLMISRTPEVTCVVVDVSHEDP | |
| | EVKFNWYVDGVEVHNAKTKPREEQYGSTYRVVSVLTVLHQDWL | |
| | NGKEYKCKVSNKALPAPIEKTISKAKGQPREPQVYTLPPSRDELTK | |
| | NQVSLWCLVKGFYPSDIAVEWESNGQPENNYKTTPPVLDSDGSFF | |
| | LYSKLTVDKSRWQQGNVFSCSVMHEALHNHYTQKSLSLSPGKGG | |
| | SHHHHHH | |
| IL28B | Knob: | |
| | VPVARLRGALPDARGCHIAQFKSLSPQELQAFKRAKDALEESLLL | |
| | KDCKCRSRLFPRTWDLRQLQVRERPVALEAELALTLKVLEATADT | |
| | DPALGDVLDQPLHTLHHILSQLRACIQPQPTAGPRTRGRLHHWLH | |
| | RLQEAPKKESPGCLEASVTFNLFRLLTRDLNCVASGDLCVEPKSC | |
| | DKTHTCPPCPAPELLGGPSVFLFPPKPKDTLMISRTPEVTCVVVDV | |
| | SHEDPEVKFNWYVDGVEVHNAKTKPREEQYGSTYRVVSVLTVLH | |
| | QDWLNGKEYKCKVSNKALPAPIEKTISKAKGQPREPQVYTLPPSR | |
| | DELTKNQVSLWCLVKGFYPSDIAVEWESNGQPENNYKTTPPVLDS | |
| | DGSFFLYSKLTVDKSRWQQGNVFSCSVMHEALHNHYTQKSLSLS | |
| | PGKGGSHHHHHH | |
| IL29 | Knob: | |
| | GPVPTSKPTTTGKGCHIGRFKSLSPQELASFKKARDALEESLKLKN | |
| | WSCSSPVFPGNWDLRLLQVRERPVALEAELALTLKVLEAAAGPAL | |
| | EDVLDQPLHTLHHILSQLQACIQPQPTAGPRPRGRLHHWLHRLQE | |
| | APKKESAGCLEASVTFNLFRLLTRDLKYVADGNLCLRTSTHPESTE | |
| | PKSCDKTHTCPPCPAPELLGGPSVFLFPPKPKDTLMISRTPEVTCVV | |
| | VDVSHEDPEVKFNWYVDGVEVHNAKTKPREEQYGSTYRVVSVL | |
| | TVLHQDWLNGKEYKCKVSNKALPAPIEKTISKAKGQPREPQVYTL | |
| | PPSRDELTKNQVSLWCLVKGFYPSDIAVEWESNGQPENNYKTTPP | |
| | VLDSDGSFFLYSKLTVDKSRWQQGNVFSCSVMHEALHNHYTQKS | |
| | LSLSPGKGGSHHHHHH | |
| IL1A | Knob: | |
| | SAPFSFLSNVKYNFMRIIKYEFILNDALNQSIIRANDQYLTAAALHN | |
| | LDEAVKFDMGAYKSSKDDAKITVILRISKTQLYVTAQDEDQPVLL | |
| | KEMPEIPKTITGSETNLLFFWETHGTKNYFTSVAHPNLFIATKQDY | |
| | WVCLAGGPPSITDFQILENQAEPKSCDKTHTCPPCPAPELLGGPSV | |
| | FLFPPKPKDTLMISRTPEVTCVVVDVSHEDPEVKFNWYVDGVEVH | |
| | NAKTKPREEQYGSTYRVVSVLTVLHQDWLNGKEYKCKVSNKAL | |
| | PAPIEKTISKAKGQPREPQVYTLPPSRDELTKNQVSLWCLVKGFYP | |
| | SDIAVEWESNGQPENNYKTTPPVLDSDGSFFLYSKLTVDKSRWQQ | |
| | GNVFSCSVMHEALHNHYTQKSLSLSPGKGGSHHHHHH | |
| IL1B | Knob: | |
| | APVRSLNCTLRDSQQKSLVMSGPYELKALHLQGQDMEQQVVFSM | |
| | SFVQGEESNDKIPVALGLKEKNLYLSCVLKDDKPTLQLESVDPKN | |
| | YPKKKMEKRFVFNKIEINNKLEFESAQFPNWYISTSQAENMPVFLG | |
| | GTKGGQDITDFTMQFVSSEPKSCDKTHTCPPCPAPELLGGPSVFLF | |
| | PPKPKDTLMISRTPEVTCVVVDVSHEDPEVKFNWYVDGVEVHNA | |
| | KTKPREEQYGSTYRVVSVLTVLHQDWLNGKEYKCKVSNKALPAP | |
| | IEKTISKAKGQPREPQVYTLPPSRDELTKNQVSLWCLVKGFYPSDI | |
| | AVEWESNGQPENNYKTTPPVLDSDGSFFLYSKLTVDKSRWQQGN | |
| | VFSCSVMHEALHNHYTQKSLSLSPGKGGSHHHHHH | |
| IL1RN | Knob: | |
| | RPSGRKSSKMQAFRIWDVNQKTFYLRNNQLVAGYLQGPNVNLEE | |
| | KIDVVPIEPHALFLGIHGGKMCLSCVKSGDETRLQLEAVNITDLSE | |
| | NRKQDKRFAFIRSDSGPTTSFESAACPGWFLCTAMEADQPVSLTN | |
| | MPDEGVMVTKFYFQEDEEPKSCDKTHTCPPCPAPELLGGPSVFLF | |
| | PPKPKDTLMISRTPEVTCVVVDVSHEDPEVKFNWYVDGVEVHNA | |
| | KTKPREEQYGSTYRVVSVLTVLHQDWLNGKEYKCKVSNKALPAP | |
| | IEKTISKAKGQPREPQVYTLPPSRDELTKNQVSLWCLVKGFYPSDI | |
| | AVEWESNGQPENNYKTTPPVLDSDGSFFLYSKLTVDKSRWQQGN | |
| | VFSCSVMHEALHNHYTQKSLSLSPGKGGSHHHHHH | |
| IL36B | Knob: | |
| | REAAPKSYAIRDSRQMVWVLSGNSLIAAPLSRSIKPVTLHLIACRD | |
| | TEFSDKEKGNMVYLGIKGKDLCLFCAEIQGKPTLQLKLQGSQDNI | |
| | GKDTCWKLVGIHTCINLDVRESCFMGTLDQWGIGVGRKKWKSSF | |
| | QHHHLRKKDKDFSSMRTNIGMPGRMEPKSCDKTHTCPPCPAPELL | |
| | GGPSVFLFPPKPKDTLMISRTPEVTCVVVDVSHEDPEVKFNWYVD | |
| | GVEVHNAKTKPREEQYGSTYRVVSVLTVLHQDWLNGKEYKCKV | |
| | SNKALPAPIEKTISKAKGQPREPQVYTLPPSRDELTKNQVSLWCLV | |
| | KGFYPSDIAVEWESNGQPENNYKTTPPVLDSDGSFFLYSKLTVDK | |
| | SRWQQGNVFSCSVMHEALHNHYTQKSLSLSPGKGGSHHHHHH | |
| IL36G | Knob: | |
| | SMCKPITGTINDLNQQVWTLQGQNLVAVPRSDSVTPVTVAVITCK | |
| | YPEALEQGRGDPIYLGIQNPEMCLYCEKVGEQPTLQLKEQKIMDL | |
| | YGQPEPVKPFLFYRAKTGRTSTLESVAFPDWFIASSKRDQPIILTSE | |
| | LGKSYNTAFELNINDEPKSCDKTHTCPPCPAPELLGGPSVFLFPPKP | |
| | KDTLMISRTPEVTCVVVDVSHEDPEVKFNWYVDGVEVHNAKTKP | |
| | REEQYGSTYRVVSVLTVLHQDWLNGKEYKCKVSNKALPAPIEKTI | |
| | SKAKGQPREPQVYTLPPSRDELTKNQVSLWCLVKGFYPSDIAVEW | |
| | ESNGQPENNYKTTPPVLDSDGSFFLYSKLTVDKSRWQQGNVFSCS | |
| | VMHEALHNHYTQKSLSLSPGKGGSHHHHHH | |
| CXCL4 | Knob: | |
| | EAEEDGDLQCLCVKTTSQVRPRHITSLEVIKAGPHCPTAQLIATLK | |
| | NGRKICLDLQAPLYKKIIKKLLESEPKSCDKTHTCPPCPAPELLGGP | |
| | SVFLFPPKPKDTLMISRTPEVTCVVVDVSHEDPEVKFNWYVDGVE | |
| | VHNAKTKPREEQYGSTYRVVSVLTVLHQDWLNGKEYKCKVSNK | |
| | ALPAPIEKTISKAKGQPREPQVYTLPPSRDELTKNQVSLWCLVKGF | |
| | YPSDIAVEWESNGQPENNYKTTPPVLDSDGSFFLYSKLTVDKSRW | |
| | QQGNVFSCSVMHEALHNHYTQKSLSLSPGKGGSHHHHHH | |
| CXCL10 | Knob: | |
| | VPLSRTVRCTCISISNQPVNPRSLEKLEIIPASQFCPRVEIIATMKKK | |
| | GEKRCLNPESKAIKNLLKAVSKERSKRSPEPKSCDKTHTCPPCPAP | |
| | ELLGGPSVFLFPPKPKDTLMISRTPEVTCVVVDVSHEDPEVKFNW | |
| | YVDGVEVHNAKTKPREEQYGSTYRVVSVLTVLHQDWLNGKEYK | |
| | CKVSNKALPAPIEKTISKAKGQPREPQVYTLPPSRDELTKNQVSLW | |
| | CLVKGFYPSDIAVEWESNGQPENNYKTTPPVLDSDGSFFLYSKLT | |
| | VDKSRWQQGNVFSCSVMHEALHNHYTQKSLSLSPGKGGSHHHH | |
| | HH | |
| CXCL14 | Knob: | |
| | SKCKCSRKGPKIRYSDVKKLEMKPKYPHCEEKMVIITTKSVSRYR | |
| | GQEHCLHPKLQSTKRFIKWYNAWNEKRRVYEEEPKSCDKTHTCP | |
| | PCPAPELLGGPSVFLFPPKPKDTLMISRTPEVTCVVVDVSHEDPEV | |
| | KFNWYVDGVEVHNAKTKPREEQYGSTYRVVSVLTVLHQDWLNG | |
| | KEYKCKVSNKALPAPIEKTISKAKGQPREPQVYTLPPSRDELTKNQ | |
| | VSLWCLVKGFYPSDIAVEWESNGQPENNYKTTPPVLDSDGSFFLY | |
| | SKLTVDKSRWQQGNVFSCSVMHEALHNHYTQKSLSLSPGKGGSH | |
| | HHHHH | |
| XCL2 | Knob: | |
| | VGSEVSHRRTCVSLTTQRLPVSRIKTYTITEGSLRAVIFITKRGLKV | |
| | CADPQATWVRDVVRSMDRKSNTRNNMIQTKPTGTQQSTNTAVTL | |
| | TGEPKSCDKTHTCPPCPAPELLGGPSVFLFPPKPKDTLMISRTPEVT | |
| | CVVVDVSHEDPEVKFNWYVDGVEVHNAKTKPREEQYGSTYRVV | |
| | SVLTVLHQDWLNGKEYKCKVSNKALPAPIEKTISKAKGQPREPQV | |
| | YTLPPSRDELTKNQVSLWCLVKGFYPSDIAVEWESNGQPENNYKT | |
| | TPPVLDSDGSFFLYSKLTVDKSRWQQGNVFSCSVMHEALHNHYT | |
| | QKSLSLSPGKGGSHHHHHH | |
| IFNA1 | Knob: | |
| | CDLPETHSLDNRRTLMLLAQMSRISPSSCLMDRHDFGFPQEEFDG | |
| | NQFQKAPAISVLHELIQQIFNLFTTKDSSAAWDEDLLDKFCTELYQ | |
| | QLNDLEACVMQEERVGETPLMNADSILAVKKYFRRITLYLTEKKY | |
| | SPCAWEVVRAEIMRSLSLSTNLQERLRRKEEPKSCDKTHTCPPCPA | |
| | PELLGGPSVFLFPPKPKDTLMISRTPEVTCVVVDVSHEDPEVKFNW | |
| | YVDGVEVHNAKTKPREEQYGSTYRVVSVLTVLHQDWLNGKEYK | |
| | CKVSNKALPAPIEKTISKAKGQPREPQVYTLPPSRDELTKNQVSLW | |
| | CLVKGFYPSDIAVEWESNGQPENNYKTTPPVLDSDGSFFLYSKLT | |
| | VDKSRWQQGNVFSCSVMHEALHNHYTQKSLSLSPGKGGSHHHH | |
| | HH | |
| IFNA2 | Knob: | |
| | CDLPQTHSLGSRRTLMLLAQMRKISLFSCLKDRHDFGFPQEEFGN | |
| | QFQKAETIPVLHEMIQQIFNLFSTKDSSAAWDETLLDKFYTELYQQ | |
| | LNDLEACVIQGVGVTETPLMKEDSILAVRKYFQRITLYLKEKKYSP | |
| | CAWEVVRAEIMRSFSLSTNLQESLRSKEEPKSCDKTHTCPPCPAPE | |
| | LLGGPSVFLFPPKPKDTLMISRTPEVTCVVVDVSHEDPEVKFNWY | |
| | VDGVEVHNAKTKPREEQYGSTYRVVSVLTVLHQDWLNGKEYKC | |
| | KVSNKALPAPIEKTISKAKGQPREPQVYTLPPSRDELTKNQVSLWC | |
| | LVKGFYPSDIAVEWESNGQPENNYKTTPPVLDSDGSFFLYSKLTV | |
| | DKSRWQQGNVFSCSVMHEALHNHYTQKSLSLSPGKGGSHHHHH | |
| | H | |
| IFNA4 | Knob: | |
| | CDLPQTHSLGNRRALILLAQMGRISHFSCLKDRHDFGFPEEEFDGH | |
| | QFQKAQAISVLHEMIQQTFNLFSTEDSSAAWEQSLLEKFSTELYQQ | |
| | LNDLEACVIQEVGVEETPLMNEDSILAVRKYFQRITLYLTEKKYSP | |
| | CAWEVVRAEIMRSLSFSTNLQKRLRRKDEPKSCDKTHTCPPCPAP | |
| | ELLGGPSVFLFPPKPKDTLMISRTPEVTCVVVDVSHEDPEVKFNW | |
| | YVDGVEVHNAKTKPREEQYGSTYRVVSVLTVLHQDWLNGKEYK | |
| | CKVSNKALPAPIEKTISKAKGQPREPQVYTLPPSRDELTKNQVSLW | |
| | CLVKGFYPSDIAVEWESNGQPENNYKTTPPVLDSDGSFFLYSKLT | |
| | VDKSRWQQGNVFSCSVMHEALHNHYTQKSLSLSPGKGGSHHHH | |
| | HH | |
| IFNA14 | Knob: | |
| | CNLSQTHSLNNRRTLMLMAQMRRISPFSCLKDRHDFEFPQEEFDG | |
| | NQFQKAQAISVLHEMMQQTFNLFSTKNSSAAWDETLLEKFYIELF | |
| | QQMNDLEACVIQEVGVEETPLMNEDSILAVKKYFQRITLYLMEKK | |
| | YSPCAWEVVRAEIMRSLSFSTNLQKRLRRKDEPKSCDKTHTCPPC | |
| | PAPELLGGPSVFLFPPKPKDTLMISRTPEVTCVVVDVSHEDPEVKF | |
| | NWYVDGVEVHNAKTKPREEQYGSTYRVVSVLTVLHQDWLNGKE | |
| | YKCKVSNKALPAPIEKTISKAKGQPREPQVYTLPPSRDELTKNQVS | |
| | LWCLVKGFYPSDIAVEWESNGQPENNYKTTPPVLDSDGSFFLYSK | |
| | LTVDKSRWQQGNVFSCSVMHEALHNHYTQKSLSLSPGKGGSHHH | |
| | HHH | |
| IFNA8 | Knob: | |
| | CDLPQTHSLGNRRALILLAQMRRISPFSCLKDRHDFEFPQEEFDDK | |
| | QFQKAQAISVLHEMIQQTFNLFSTKDSSAALDETLLDEFYIELDQQ | |
| | LNDLESCVMQEVGVIESPLMYEDSILAVRKYFQRITLYLTEKKYSS | |
| | CAWEVVRAEIMRSFSLSINLQKRLKSKEEPKSCDKTHTCPPCPAPE | |
| | LLGGPSVFLFPPKPKDTLMISRTPEVTCVVVDVSHEDPEVKENWY | |
| | VDGVEVHNAKTKPREEQYGSTYRVVSVLTVLHQDWLNGKEYKC | |
| | KVSNKALPAPIEKTISKAKGQPREPQVYTLPPSRDELTKNQVSLWC | |
| | LVKGFYPSDIAVEWESNGQPENNYKTTPPVLDSDGSFFLYSKLTV | |
| | DKSRWQQGNVFSCSVMHEALHNHYTQKSLSLSPGKGGSHHHHH | |
| | H | |
| IFNA7 | Knob: | |
| | CDLPQTHSLRNRRALILLAQMGRISPFSCLKDRHEFRFPEEEFDGH | |
| | QFQKTQAISVLHEMIQQTFNLFSTEDSSAAWEQSLLEKFSTELYQQ | |
| | LNDLEACVIQEVGVEETPLMNEDFILAVRKYFQRITLYLMEKKYSP | |
| | CAWEVVRAEIMRSFSFSTNLKKGLRRKDEPKSCDKTHTCPPCPAP | |
| | ELLGGPSVFLFPPKPKDTLMISRTPEVTCVVVDVSHEDPEVKFNW | |
| | YVDGVEVHNAKTKPREEQYGSTYRVVSVLTVLHQDWLNGKEYK | |
| | CKVSNKALPAPIEKTISKAKGQPREPQVYTLPPSRDELTKNQVSLW | |
| | CLVKGFYPSDIAVEWESNGQPENNYKTTPPVLDSDGSFFLYSKLT | |
| | VDKSRWQQGNVFSCSVMHEALHNHYTQKSLSLSPGKGGSHHHH | |
| | HH | |
| IFNA21 | Knob: | |
| | CDLPQTHSLGNRRALILLAQMGRISPFSCLKDRHDFGFPQEEFDGN | |
| | QFQKAQAISVLHEMIQQTFNLFSTKDSSATWEQSLLEKFSTELNQQ | |
| | LNDLEACVIQEVGVEETPLMNVDSILAVKKYFQRITLYLTEKKYSP | |
| | CAWEVVRAEIMRSFSLSKIFQERLRRKEEPKSCDKTHTCPPCPAPE | |
| | LLGGPSVFLFPPKPKDTLMISRTPEVTCVVVDVSHEDPEVKENWY | |
| | VDGVEVHNAKTKPREEQYGSTYRVVSVLTVLHQDWLNGKEYKC | |
| | KVSNKALPAPIEKTISKAKGQPREPQVYTLPPSRDELTKNQVSLWC | |
| | LVKGFYPSDIAVEWESNGQPENNYKTTPPVLDSDGSFFLYSKLTV | |
| | DKSRWQQGNVFSCSVMHEALHNHYTQKSLSLSPGKGGSHHHHH | |
| | H | |
| IFNA17 | Knob: | |
| | CDLPQTHSLGNRRALILLAQMGRISPFSCLKDRHDFGLPQEEFDGN | |
| | QFQKTQAISVLHEMIQQTFNLFSTEDSSAAWEQSLLEKFSTELYQQ | |
| | LNNLEACVIQEVGMEETPLMNEDSILAVRKYFQRITLYLTEKKYSP | |
| | CAWEVVRAEIMRSLSFSTNLQKILRRKDEPKSCDKTHTCPPCPAPE | |
| | LLGGPSVFLFPPKPKDTLMISRTPEVTCVVVDVSHEDPEVKENWY | |
| | VDGVEVHNAKTKPREEQYGSTYRVVSVLTVLHQDWLNGKEYKC | |
| | KVSNKALPAPIEKTISKAKGQPREPQVYTLPPSRDELTKNQVSLWC | |
| | LVKGFYPSDIAVEWESNGQPENNYKTTPPVLDSDGSFFLYSKLTV | |
| | DKSRWQQGNVFSCSVMHEALHNHYTQKSLSLSPGKGGSHHHHH | |
| | H | |
| IFNA5 | Knob: | |
| | LGCDLPQTHSLSNRRTLMIMAQMGRISPFSCLKDRHDFGFPQEEFD | |
| | GNQFQKAQAISVLHEMIQQTFNLFSTKDSSATWDETLLDKFYTEL | |
| | YQQLNDLEACMMQEVGVEDTPLMNVDSILTVRKYFQRITLYLTE | |
| | KKYSPCAWEVVRAEIMRSFSLSANLQERLRRKEEPKSCDKTHTCP | |
| | PCPAPELLGGPSVFLFPPKPKDTLMISRTPEVTCVVVDVSHEDPEV | |
| | KFNWYVDGVEVHNAKTKPREEQYGSTYRVVSVLTVLHQDWLNG | |
| | KEYKCKVSNKALPAPIEKTISKAKGQPREPQVYTLPPSRDELTKNQ | |
| | VSLWCLVKGFYPSDIAVEWESNGQPENNYKTTPPVLDSDGSFFLY | |
| | SKLTVDKSRWQQGNVFSCSVMHEALHNHYTQKSLSLSPGKGGSH | |
| | HHHHH | |
| IFNA6 | Knob: | |
| | SLDCDLPQTHSLGHRRTMMLLAQMRRISLFSCLKDRHDFRFPQEE | |
| | FDGNQFQKAEAISVLHEVIQQTFNLFSTKDSSVAWDERLLDKLYT | |
| | ELYQQLNDLEACVMQEVWVGGTPLMNEDSILAVRKYFQRITLYL | |
| | TEKKYSPCAWEVVRAEIMRSFSSSRNLQERLRRKEEPKSCDKTHT | |
| | CPPCPAPELLGGPSVFLFPPKPKDTLMISRTPEVTCVVVDVSHEDPE | |
| | VKFNWYVDGVEVHNAKTKPREEQYGSTYRVVSVLTVLHQDWLN | |
| | GKEYKCKVSNKALPAPIEKTISKAKGQPREPQVYTLPPSRDELTKN | |
| | QVSLWCLVKGFYPSDIAVEWESNGQPENNYKTTPPVLDSDGSFFL | |
| | YSKLTVDKSRWQQGNVFSCSVMHEALHNHYTQKSLSLSPGKGGS | |
| | HHHHHH | |
| IFNA10 | Knob: | |
| | CDLPQTHSLGNRRALILLGQMGRISPFSCLKDRHDFRIPQEEFDGN | |
| | QFQKAQAISVLHEMIQQTFNLFSTEDSSAAWEQSLLEKFSTELYQQ | |
| | LNDLEACVIQEVGVEETPLMNEDSILAVRKYFQRITLYLIERKYSP | |
| | CAWEVVRAEIMRSLSFSTNLQKRLRRKDEPKSCDKTHTCPPCPAP | |
| | ELLGGPSVFLFPPKPKDTLMISRTPEVTCVVVDVSHEDPEVKENW | |
| | YVDGVEVHNAKTKPREEQYGSTYRVVSVLTVLHQDWLNGKEYK | |
| | CKVSNKALPAPIEKTISKAKGQPREPQVYTLPPSRDELTKNQVSLW | |
| | CLVKGFYPSDIAVEWESNGQPENNYKTTPPVLDSDGSFFLYSKLT | |
| | VDKSRWQQGNVFSCSVMHEALHNHYTQKSLSLSPGKGGSHHHH | |
| | HH | |
| IFNA16 | Knob: | |
| | CDLPQTHSLGNRRALILLAQMGRISHFSCLKDRYDFGFPQEVEDG | |
| | NQFQKAQAISAFHEMIQQTFNLFSTKDSSAAWDETLLDKFYIELFQ | |
| | QLNDLEACVTQEVGVEEIALMNEDSILAVRKYFQRITLYLMGKKY | |
| | SPCAWEVVRAEIMRSFSFSTNLQKGLRRKDEPKSCDKTHTCPPCP | |
| | APELLGGPSVFLFPPKPKDTLMISRTPEVTCVVVDVSHEDPEVKEN | |
| | WYVDGVEVHNAKTKPREEQYGSTYRVVSVLTVLHQDWLNGKEY | |
| | KCKVSNKALPAPIEKTISKAKGQPREPQVYTLPPSRDELTKNQVSL | |
| | WCLVKGFYPSDIAVEWESNGQPENNYKTTPPVLDSDGSFFLYSKL | |
| | TVDKSRWQQGNVFSCSVMHEALHNHYTQKSLSLSPGKGGSHHH | |
| | HHH | |
| GDF15 | Knob: | |
| | ARARNGDHCPLGPGRCCRLHTVRASLEDLGWADWVLSPREVQV | |
| | TMCIGACPSQFRAANMHAQIKTSLHRLKPDTVPAPCCVPASYNPM | |
| | VLIQKTDTGVSLQTYDDLLAKDCHCIEPKSCDKTHTCPPCPAPELL | |
| | GGPSVFLFPPKPKDTLMISRTPEVTCVVVDVSHEDPEVKFNWYVD | |
| | GVEVHNAKTKPREEQYGSTYRVVSVLTVLHQDWLNGKEYKCKV | |
| | SNKALPAPIEKTISKAKGQPREPQVYTLPPSRDELTKNQVSLWCLV | |
| | KGFYPSDIAVEWESNGQPENNYKTTPPVLDSDGSFFLYSKLTVDK | |
| | SRWQQGNVFSCSVMHEALHNHYTQKSLSLSPGKGGSHHHHHH | |
| GDF2 | Knob: | |
| | SAGAGSHCQKTSLRVNFEDIGWDSWIIAPKEYEAYECKGGCFFPL | |
| | ADDVTPTKHAIVQTLVHLKFPTKVGKACCVPTKLSPISVLYKDDM | |
| | GVPTLKYHYEGMSVAECGCREPKSCDKTHTCPPCPAPELLGGPSV | |
| | FLFPPKPKDTLMISRTPEVTCVVVDVSHEDPEVKFNWYVDGVEVH | |
| | NAKTKPREEQYGSTYRVVSVLTVLHQDWLNGKEYKCKVSNKAL | |
| | PAPIEKTISKAKGQPREPQVYTLPPSRDELTKNQVSLWCLVKGFYP | |
| | SDIAVEWESNGQPENNYKTTPPVLDSDGSFFLYSKLTVDKSRWQQ | |
| | GNVFSCSVMHEALHNHYTQKSLSLSPGKGGSHHHHHH | |
| BMP10 | Knob: | |
| | NAKGNYCKRTPLYIDFKEIGWDSWIIAPPGYEAYECRGVCNYPLA | |
| | EHLTPTKHAIIQALVHLKNSQKASKACCVPTKLEPISILYLDKGVV | |
| | TYKFKYEGMAVSECGCREPKSCDKTHTCPPCPAPELLGGPSVFLFP | |
| | PKPKDTLMISRTPEVTCVVVDVSHEDPEVKFNWYVDGVEVHNAK | |
| | TKPREEQYGSTYRVVSVLTVLHQDWLNGKEYKCKVSNKALPAPI | |
| | EKTISKAKGQPREPQVYTLPPSRDELTKNQVSLWCLVKGFYPSDIA | |
| | VEWESNGQPENNYKTTPPVLDSDGSFFLYSKLTVDKSRWQQGNV | |
| | FSCSVMHEALHNHYTQKSLSLSPGKGGSHHHHHH | |
| INHA | Knob: | |
| | STPLMSWPWSPSALRLLQRPPEEPAAHANCHRVALNISFQELGWE | |
| | RWIVYPPSFIFHYCHGGCGLHIPPNLSLPVPGAPPTPAQPYSLLPGA | |
| | QPCCAALPGTMRPLHVRTTSDGGYSFKYETVPNLLTQHCACI | |
| | EPKSCDKTHTCPPCPAPELLGGPSVFLFPPKPKDTLMISRTPEVTCV | |
| | VVDVSHEDPEVKFNWYVDGVEVHNAKTKPREEQYGSTYRVVSV | |
| | LTVLHQDWLNGKEYKCKVSNKALPAPIEKTISKAKGQPREPQVYT | |
| | LPPSRDELTKNQVSLWCLVKGFYPSDIAVEWESNGQPENNYKTTP | |
| | PVLDSDGSFFLYSKLTVDKSRWQQGNVFSCSVMHEALHNHYTQK | |
| | SLSLSPGKGGSHHHHHH | |
| BMP3 | Knob: | |
| | QWIEPRNCARRYLKVDFADIGWSEWIISPKSFDAYYCSGACQFPM | |
| | PKSLKPSNHATIQSIVRAVGVVPGIPEPCCVPEKMSSLSILFFDENK | |
| | NVVLKVYPNMTVESCACREPKSCDKTHTCPPCPAPELLGGPSVFL | |
| | FPPKPKDTLMISRTPEVTCVVVDVSHEDPEVKFNWYVDGVEVHN | |
| | AKTKPREEQYGSTYRVVSVLTVLHQDWLNGKEYKCKVSNKALP | |
| | APIEKTISKAKGQPREPQVYTLPPSRDELTKNQVSLWCLVKGFYPS | |
| | DIAVEWESNGQPENNYKTTPPVLDSDGSFFLYSKLTVDKSRWQQG | |
| | NVFSCSVMHEALHNHYTQKSLSLSPGKGGSHHHHHH | |
| TRAIL (APO2L) | Knob: | |
| | TNELKQMQDKYSKSGIACFLKEDDSYWDPNDEESMNSPCWQVK | |
| | WQLRQLVRKMILRTSEETISTVQEKQQNISPLVRERGPQRVAAHIT | |
| | GTRGRSNTLSSPNSKNEKALGRKINSWESSRSGHSFLSNLHLRNGE | |
| | LVIHEKGFYYIYSQTYFRFQEEIKENTKNDKQMVQYIYKYTSYPDP | |
| | ILLMKSARNSCWSKDAEYGLYSIYQGGIFELKENDRIFVSVTNEHL | |
| | IDMDHEASFFGAFLVGEPKSCDKTHTCPPCPAPELLGGPSVFLFPP | |
| | KPKDTLMISRTPEVTCVVVDVSHEDPEVKFNWYVDGVEVHNAKT | |
| | KPREEQYGSTYRVVSVLTVLHQDWLNGKEYKCKVSNKALPAPIE | |
| | KTISKAKGQPREPQVYTLPPSRDELTKNQVSLWCLVKGFYPSDIAV | |
| | EWESNGQPENNYKTTPPVLDSDGSFFLYSKLTVDKSRWQQGNVF | |
| | SCSVMHEALHNHYTQKSLSLSPGKGGSHHHHHH | |
| TL1 (VEGI) | Knob: | |
| | LKGQEFAPSHQQVYAPLRADGDKPRAHLTVVRQTPTQHFKNQFP | |
| | ALHWEHELGLAFTKNRMNYTNKFLLIPESGDYFIYSQVTFRGMTS | |
| | ECSEIRQAGRPNKPDSITVVITKVTDSYPEPTQLLMGTKSVCEVGS | |
| | NWFQPIYLGAMESLQEGDKLMVNVSDISLVDYTKEDKTFFGAFLL | |
| | EPKSCDKTHTCPPCPAPELLGGPSVFLFPPKPKDTLMISRTPEVTCV | |
| | VVDVSHEDPEVKFNWYVDGVEVHNAKTKPREEQYGSTYRVVSV | |
| | LTVLHQDWLNGKEYKCKVSNKALPAPIEKTISKAKGQPREPQVYT | |
| | LPPSRDELTKNQVSLWCLVKGFYPSDIAVEWESNGQPENNYKTTP | |
| | PVLDSDGSFFLYSKLTVDKSRWQQGNVFSCSVMHEALHNHYTQK | |
| | SLSLSPGKGGSHHHHHH | |
| LTA (TNFB) | Knob: | |
| | LPGVGLTPSAAQTARQHPKMHLAHSTLKPAAHLIGDPSKQNSLLW | |
| | RANTDRAFLQDGFSLSNNSLLVPTSGIYFVYSQVVFSGKAYSPKAT | |
| | SSPLYLAHEVQLFSSQYPFHVPLLSSQKMVYPGLQEPWLHSMYHG | |
| | AAFQLTQGDQLSTHTDGIPHLVLSPSTVFFGAFALEPKSCDKTHTC | |
| | PPCPAPELLGGPSVFLFPPKPKDTLMISRTPEVTCVVVDVSHEDPE | |
| | VKFNWYVDGVEVHNAKTKPREEQYGSTYRVVSVLTVLHQDWLN | |
| | GKEYKCKVSNKALPAPIEKTISKAKGQPREPQVYTLPPSRDELTKN | |
| | QVSLWCLVKGFYPSDIAVEWESNGQPENNYKTTPPVLDSDGSFFL | |
| | YSKLTVDKSRWQQGNVFSCSVMHEALHNHYTQKSLSLSPGKGGS | |
| | HHHHHH | |
| BLYS (BAFF) | Knob: | |
| | AVQGPEETVTQDCLQLIADSETPTIQKGSYTFVPWLLSFKRGSALE | |
| | EKENKILVKETGYFFIYGQVLYTDKTYAMGHLIQRKKVHVFGDEL | |
| | SLVTLFRCIQNMPETLPNNSCYSAGIAKLEEGDELQLAIPRENAQIS | |
| | LDGDVTFFGALKLLEPKSCDKTHTCPPCPAPELLGGPSVFLFPPKP | |
| | KDTLMISRTPEVTCVVVDVSHEDPEVKFNWYVDGVEVHNAKTKP | |
| | REEQYGSTYRVVSVLTVLHQDWLNGKEYKCKVSNKALPAPIEKTI | |
| | SKAKGQPREPQVYTLPPSRDELTKNQVSLWCLVKGFYPSDIAVEW | |
| | ESNGQPENNYKTTPPVLDSDGSFFLYSKLTVDKSRWQQGNVFSCS | |
| | VMHEALHNHYTQKSLSLSPGKGGSHHHHHH | |
| APRIL | Knob: | |
| | AVLTQKQKKQHSVLHLVPINATSKDDSDVTEVMWQPALRRGRGL | |
| | QAQGYGVRIQDAGVYLLYSQVLFQDVTFEPKSCDKTHTCPPCPAP | |
| | ELLGGPSVFLFPPKPKDTLMISRTPEVTCVVVDVSHEDPEVKENW | |
| | YVDGVEVHNAKTKPREEQYGSTYRVVSVLTVLHQDWLNGKEYK | |
| | CKVSNKALPAPIEKTISKAKGQPREPQVYTLPPSRDELTKNQVSLW | |
| | CLVKGFYPSDIAVEWESNGQPENNYKTTPPVLDSDGSFFLYSKLT | |
| | VDKSRWQQGNVFSCSVMHEALHNHYTQKSLSLSPGKGGSHHHH | |
| | HH | |
| RANKL | Knob: | |
| | IRAEKAMVDGSWLDLAKRSKLEAQPFAHLTINATDIPSGSHKVSLS | |
| | SWYHDRGWAKISNMTFSNGKLIVNQDGFYYLYANICFRHHETSG | |
| | DLATEYLQLMVYVTKTSIKIPSSHTLMKGGSTKYWSGNSEFHFYSI | |
| | NVGGFFKLRSGEEISIEVSNPSLLDPDQDATYFGAFKVRDIDEPKSC | |
| | DKTHTCPPCPAPELLGGPSVFLFPPKPKDTLMISRTPEVTCVVVDV | |
| | SHEDPEVKFNWYVDGVEVHNAKTKPREEQYGSTYRVVSVLTVLH | |
| | QDWLNGKEYKCKVSNKALPAPIEKTISKAKGQPREPQVYTLPPSR | |
| | DELTKNQVSLWCLVKGFYPSDIAVEWESNGQPENNYKTTPPVLDS | |
| | DGSFFLYSKLTVDKSRWQQGNVFSCSVMHEALHNHYTQKSLSLS | |
| | PGKGGSHHHHHH | |
| CD40LG | Knob: | |
| | MQKGDQNPQIAAHVISEASSKTTSVLQWAEKGYYTMSNNLVTLE | |
| | NGKQLTVKRQGLYYIYAQVTFCSNREASSQAPFIASLCLKSPGRFE | |
| | RILLRAANTHSSAKPCGQQSIHLGGVFELQPGASVFVNVTDPSQVS | |
| | HGTGFTSFGLLKLEPKSCDKTHTCPPCPAPELLGGPSVFLFPPKPKD | |
| | TLMISRTPEVTCVVVDVSHEDPEVKFNWYVDGVEVHNAKTKPRE | |
| | EQYGSTYRVVSVLTVLHQDWLNGKEYKCKVSNKALPAPIEKTISK | |
| | AKGQPREPQVYTLPPSRDELTKNQVSLWCLVKGFYPSDIAVEWES | |
| | NGQPENNYKTTPPVLDSDGSFFLYSKLTVDKSRWQQGNVFSCSV | |
| | MHEALHNHYTQKSLSLSPGKGGSHHHHHH | |
| EDA | Knob: | |
| | SKSNEGADGPVKNKKKGKKAGPPGPNGPPGPPGPPGPQGPPGIPGI | |
| | PGIPGTTVMGPPGPPGPPGPQGPPGLQGPSGAADKAGTRENQPAV | |
| | VHLQGQGSAIQVKNDLSGGVLNDWSRITMNPKVFKLHPRSGELE | |
| | VLVDGTYFIYSQVEVYYINFTDFASYEVVVDEKPFLQCTRSIETGK | |
| | TNYNTCYTAGVCLLKARQKIAVKMVHADISINMSKHTTFFGAIRL | |
| | GEAPASEPKSCDKTHTCPPCPAPELLGGPSVFLFPPKPKDTLMISRT | |
| | PEVTCVVVDVSHEDPEVKFNWYVDGVEVHNAKTKPREEQYGST | |
| | YRVVSVLTVLHQDWLNGKEYKCKVSNKALPAPIEKTISKAKGQP | |
| | REPQVYTLPPSRDELTKNQVSLWCLVKGFYPSDIAVEWESNGQPE | |
| | NNYKTTPPVLDSDGSFFLYSKLTVDKSRWQQGNVFSCSVMHEAL | |
| | HNHYTQKSLSLSPGKGGSHHHHHH | |
| FASLG | Knob: | |
| | QIGHPSPPPEKKELRKVAHLTGKSNSRSMPLEWEDTYGIVLLSGVK | |
| | YKKGGLVINETGLYFVYSKVYFRGQSCNNLPLSHKVYMRNSKYP | |
| | QDLVMMEGKMMSYCTTGQMWARSSYLGAVENLTSADHLYVNV | |
| | SELSLVNFEESQTFFGLYKLEPKSCDKTHTCPPCPAPELLGGPSVFL | |
| | FPPKPKDTLMISRTPEVTCVVVDVSHEDPEVKFNWYVDGVEVHN | |
| | AKTKPREEQYGSTYRVVSVLTVLHQDWLNGKEYKCKVSNKALP | |
| | APIEKTISKAKGQPREPQVYTLPPSRDELTKNQVSLWCLVKGFYPS | |
| | DIAVEWESNGQPENNYKTTPPVLDSDGSFFLYSKLTVDKSRWQQG | |
| | NVFSCSVMHEALHNHYTQKSLSLSPGKGGSHHHHHH | |
| CD70 | Knob: | |
| | QRFAQAQQQLPLESLGWDVAELQLNHTGPQQDPRLYWQGGPAL | |
| | GRSFLHGPELDKGQLRIHRDGIYMVHIQVTLAICSSTTASRHHPTT | |
| | LAVGICSPASRSISLLRLSFHQGCTIASQRLTPLARGDTLCTNLTGT | |
| | LLPSRNTDETFFGVQWVRPEPKSCDKTHTCPPCPAPELLGGPSVFL | |
| | FPPKPKDTLMISRTPEVTCVVVDVSHEDPEVKFNWYVDGVEVHN | |
| | AKTKPREEQYGSTYRVVSVLTVLHQDWLNGKEYKCKVSNKALP | |
| | APIEKTISKAKGQPREPQVYTLPPSRDELTKNQVSLWCLVKGFYPS | |
| | DIAVEWESNGQPENNYKTTPPVLDSDGSFFLYSKLTVDKSRWQQG | |
| | NVFSCSVMHEALHNHYTQKSLSLSPGKGGSHHHHHH | |
| EPO | Knob: | |
| | APPRLICDSRVLERYLLEAKEAENITTGCAEHCSLNENITVPDTKV | |
| | NFYAWKRMEVGQQAVEVWQGLALLSEAVLRGQALLVNSSQPWE | |
| | PLQLHVDKAVSGLRSLTTLLRALGAQKEAISPPDAASAAPLRTITA | |
| | DTFRKLFRVYSNFLRGKLKLYTGEACRTGDREPKSCDKTHTCPPC | |
| | PAPELLGGPSVFLFPPKPKDTLMISRTPEVTCVVVDVSHEDPEVKF | |
| | NWYVDGVEVHNAKTKPREEQYGSTYRVVSVLTVLHQDWLNGKE | |
| | YKCKVSNKALPAPIEKTISKAKGQPREPQVYTLPPSRDELTKNQVS | |
| | LWCLVKGFYPSDIAVEWESNGQPENNYKTTPPVLDSDGSFFLYSK | |
| | LTVDKSRWQQGNVFSCSVMHEALHNHYTQKSLSLSPGKGGSHHH | |
| | HHH | |
| GH1 | Knob: | |
| | FPTIPLSRLFDNAMLRAHRLHQLAFDTYQEFEEAYIPKEQKYSFLQ | |
| | NPQTSLCFSESIPTPSNREETQQKSNLELLRISLLLIQSWLEPVQFLR | |
| | SVFANSLVYGASDSNVYDLLKDLEEGIQTLMGRLEDGSPRTGQIF | |
| | KQTYSKFDTNSHNDDALLKNYGLLYCFRKDMDKVETFLRIVQCR | |
| | SVEGSCGFEPKSCDKTHTCPPCPAPELLGGPSVFLFPPKPKDTLMIS | |
| | RTPEVTCVVVDVSHEDPEVKFNWYVDGVEVHNAKTKPREEQYGS | |
| | TYRVVSVLTVLHQDWLNGKEYKCKVSNKALPAPIEKTISKAKGQP | |
| | REPQVYTLPPSRDELTKNQVSLWCLVKGFYPSDIAVEWESNGQPE | |
| | NNYKTTPPVLDSDGSFFLYSKLTVDKSRWQQGNVFSCSVMHEAL | |
| | HNHYTQKSLSLSPGKGGSHHHHHH | |
| GH2 | Knob: | |
| | FPTIPLSRLFDNAMLRARRLYQLAYDTYQEFEEAYILKEQKYSFLQ | |
| | NPQTSLCFSESIPTPSNRVKTQQKSNLELLRISLLLIQSWLEPVQLLR | |
| | SVFANSLVYGASDSNVYRHLKDLEEGIQTLMWRLEDGSPRTGQIF | |
| | NQSYSKFDTKSHNDDALLKNYGLLYCFRKDMDKVETFLRIVQCR | |
| | SVEGSCGFEPKSCDKTHTCPPCPAPELLGGPSVFLFPPKPKDTLMIS | |
| | RTPEVTCVVVDVSHEDPEVKFNWYVDGVEVHNAKTKPREEQYGS | |
| | TYRVVSVLTVLHQDWLNGKEYKCKVSNKALPAPIEKTISKAKGQP | |
| | REPQVYTLPPSRDELTKNQVSLWCLVKGFYPSDIAVEWESNGQPE | |
| | NNYKTTPPVLDSDGSFFLYSKLTVDKSRWQQGNVFSCSVMHEAL | |
| | HNHYTQKSLSLSPGKGGSHHHHHH | |
| PRL | Knob: | |
| | LPICPGGAARCQVTLRDLFDRAVVLSHYIHNLSSEMFSEFDKRYTH | |
| | GRGFITKAINSCHTSSLATPEDKEQAQQMNQKDFLSLIVSILRSWN | |
| | EPLYHLVTEVRGMQEAPEAILSKAVEIEEQTKRLLEGMELIVSQVH | |
| | PETKENEIYPVWSGLPSLQMADEESRLSAYYNLLHCLRRDSHKID | |
| | NYLKLLKCRIIHNNNCEPKSCDKTHTCPPCPAPELLGGPSVFLFPPK | |
| | PKDTLMISRTPEVTCVVVDVSHEDPEVKFNWYVDGVEVHNAKTK | |
| | PREEQYGSTYRVVSVLTVLHQDWLNGKEYKCKVSNKALPAPIEK | |
| | TISKAKGQPREPQVYTLPPSRDELTKNQVSLWCLVKGFYPSDIAVE | |
| | WESNGQPENNYKTTPPVLDSDGSFFLYSKLTVDKSRWQQGNVFS | |
| | CSVMHEALHNHYTQKSLSLSPGKGGSHHHHHH | |
| CSF3 | Knob: | |
| | TPLGPASSLPQSFLLKCLEQVRKIQGDGAALQEKLVSECATYKLCH | |
| | PEELVLLGHSLGIPWAPLSSCPSQALQLAGCLSQLHSGLFLYQGLL | |
| | QALEGISPELGPTLDTLQLDVADFATTIWQQMEELGMAPALQPTQ | |
| | GAMPAFASAFQRRAGGVLVASHLQSFLEVSYRVLRHLAQPEPKSC | |
| | DKTHTCPPCPAPELLGGPSVFLFPPKPKDTLMISRTPEVTCVVVDV | |
| | SHEDPEVKFNWYVDGVEVHNAKTKPREEQYGSTYRVVSVLTVLH | |
| | QDWLNGKEYKCKVSNKALPAPIEKTISKAKGQPREPQVYTLPPSR | |
| | DELTKNQVSLWCLVKGFYPSDIAVEWESNGQPENNYKTTPPVLDS | |
| | DGSFFLYSKLTVDKSRWQQGNVFSCSVMHEALHNHYTQKSLSLS | |
| | PGKGGSHHHHHH | |
| LEP | Knob: | |
| | VPIQKVQDDTKTLIKTIVTRINDISHTQSVSSKQKVTGLDFIPGLHPI | |
| | LTLSKMDQTLAVYQQILTSMPSRNVIQISNDLENLRDLLHVLAFSK | |
| | SCHLPWASGLETLDSLGGVLEASGYSTEVVALSRLQGSLQDMLW | |
| | QLDLSPGCEPKSCDKTHTCPPCPAPELLGGPSVFLFPPKPKDTLMIS | |
| | RTPEVTCVVVDVSHEDPEVKFNWYVDGVEVHNAKTKPREEQYGS | |
| | TYRVVSVLTVLHQDWLNGKEYKCKVSNKALPAPIEKTISKAKGQP | |
| | REPQVYTLPPSRDELTKNQVSLWCLVKGFYPSDIAVEWESNGQPE | |
| | NNYKTTPPVLDSDGSFFLYSKLTVDKSRWQQGNVFSCSVMHEAL | |
| | HNHYTQKSLSLSPGKGGSHHHHHH | |
| CSF1 | Knob: | |
| | EEVSEYCSHMIGSGHLQSLQRLIDSQMETSCQITFEFVDQEQLKDP | |
| | VCYLKKAFLLVQDIMEDTMRFRDNTPNAIAIVQLQELSLRLKSCFT | |
| | KDYEEHDKACVRTFYETPLQLLEKVKNVFNETKNLLDKDWNIFS | |
| | KNCNNSFAECSSQDVVTKPDCNCLYPKAIPSSDPASVSPHQPLAPS | |
| | MAPVAGLTWEDSEGTEGSSLLPGEQPLHTVDPGSAKQRPPRSTCQ | |
| | SFEPPETPVVKDSTIGGSPQPRPSVGAFNPGMEDILDSAMGTNWVP | |
| | EEASGEASEIPVPQGTELSPSRPGGGSMQTEPARPSNFLSASSPLPA | |
| | SAKGQQPADVTGTALPRVGPVRPTGQDWNHTPQKTDHPSALLRD | |
| | PPEPGSPRISSLRPQGLSNPSTLSAQPQLSRSHSSGSVLPLGELEGRR | |
| | STRDRREPKSCDKTHTCPPCPAPELLGGPSVFLFPPKPKDTLMISRT | |
| | PEVTCVVVDVSHEDPEVKFNWYVDGVEVHNAKTKPREEQYGST | |
| | YRVVSVLTVLHQDWLNGKEYKCKVSNKALPAPIEKTISKAKGQP | |
| | REPQVYTLPPSRDELTKNQVSLWCLVKGFYPSDIAVEWESNGQPE | |
| | NNYKTTPPVLDSDGSFFLYSKLTVDKSRWQQGNVFSCSVMHEAL | |
| | HNHYTQKSLSLSPGKGGSHHHHHH | |
| IGF1 | Knob: | |
| | GPETLCGAELVDALQFVCGDRGFYFNKPTGYGSSSRRAPQTGIVD | |
| | ECCFRSCDLRRLEMYCAPLKPAKSAEPKSCDKTHTCPPCPAPELLG | |
| | GPSVFLFPPKPKDTLMISRTPEVTCVVVDVSHEDPEVKFNWYVDG | |
| | VEVHNAKTKPREEQYGSTYRVVSVLTVLHQDWLNGKEYKCKVS | |
| | NKALPAPIEKTISKAKGQPREPQVYTLPPSRDELTKNQVSLWCLVK | |
| | GFYPSDIAVEWESNGQPENNYKTTPPVLDSDGSFFLYSKLTVDKSR | |
| | WQQGNVFSCSVMHEALHNHYTQKSLSLSPGKGGSHHHHHH | |
| IGF2 | Knob: | |
| | AYRPSETLCGGELVDTLQFVCGDRGFYFSRPASRVSRRSRGIVEEC | |
| | CFRSCDLALLETYCATPAKSEEPKSCDKTHTCPPCPAPELLGGPSV | |
| | FLFPPKPKDTLMISRTPEVTCVVVDVSHEDPEVKFNWYVDGVEVH | |
| | NAKTKPREEQYGSTYRVVSVLTVLHQDWLNGKEYKCKVSNKAL | |
| | PAPIEKTISKAKGQPREPQVYTLPPSRDELTKNQVSLWCLVKGFYP | |
| | SDIAVEWESNGQPENNYKTTPPVLDSDGSFFLYSKLTVDKSRWQQ | |
| | GNVFSCSVMHEALHNHYTQKSLSLSPGKGGSHHHHHH | |

### Example 14: Methods for Example 13

Extracellular and membrane protein degradation protocol: Cells (e.g., human cancer cell lines (MDA-MB-231, HeLa, A431, etc.) are plated in 6- or 12-well plates and grown to ˜70% confluency before treatment. Media is aspirated and cells are treated with bispecifics or control antibodies in complete growth medium at a concentration range of about 0.01 nM to 1 uM. For soluble ligand uptake experiments, conjugated (e.g, biotinylated) soluble ligand (e.g., VEGF, TNFa) is pre-incubated with a fluorescent dye (e.g., Streptavidin 647, pHrodo red. Concentration range of ligand-dye to add to bispecific also from about 0.01 nM-1 μM) at 37° C. for about 10, 20, 30, 40, 50, 60, 70, 80, 90, 100, 110, or 120 minutes, then mixed with bispecific (e.g., knob-in-hole, cytokine-IgG fusion, cytokine-Fab fusion, cytokine-scFv fusion) or control antibodies and added to cells. After incubation at about 4, 8, 12, 16, 20, 24, 28, 32, or 37° C. for the about 0, 1, 2, 3, 4, 5, 6, 7, days, cells are washed with phosphate-buffered saline (PBS), lifted with versene, and harvested by centrifugation at 300×g for 5 min at 4° C. Samples are then tested by western blotting or flow cytometry to quantify protein levels.

Extracellular target uptake readout protocol: Cell pellets are washed three times with cold PBS and centrifuged at 300×g for 5 minutes. Cells are then resuspended in cold PBS. Flow cytometry is performed on a CytoFLEX cytometer (Beckman Coulter) and gating is performed on single cells and live cells before acquisition of 10,000 cells. Analysis is performed using the FlowJo software package.

Membrane protein degradation readout by western blotting protocol: Cell pellets are lysed with 1× RIPA buffer containing cOmplete mini protease inhibitor cocktail (Sigma-Aldrich) at 4° C. for 40 minutes. Lysates are centrifuged at 16,000×g for 10 min at 4° C. and protein concentrations are normalized using BCA assay (Pierce). 4× NuPAGE LDS sample buffer (Invitrogen) and 2-mercaptoethanol (BME) is added to the lysates and boiled for 10 min. Equal amounts of lysates are loaded onto a 4-12% Bis-Tris gel and ran at 200V for 37 min. The gel is incubated in 20% ethanol for 10 min and transferred onto a polyvinylidene difluoride (PVDF) membrane. The membrane is blocked in PBS with 0.1% Tween-20+5% bovine serum albumin (BSA) for 30 min at room temperature with gentle shaking. Membranes are incubated overnight with primary antibodies at respective dilutions at 4° C. with gentle shaking in PBS+0.2% Tween-20+5% BSA. Membranes are washed four times with tris-buffered saline (TBS)+0.1% Tween-20 and then co-incubated with secondary antibodies for 1 hr at room temperature. Membranes are washed four times with TBS+0.1% Tween-20, then washed with PBS. Membranes are imaged using an OdysseyCLxImager (L1-COR). SuperSignal West Pico PLUS Chemiluminescent Substrate (Thermo Fisher Scientific) is then added and imaged using a ChemiDoc Imager (BioRad). Band intensities are quantified using Image Studio Software (L1-COR).

Membrane protein degradation readout by flow cytometry protocol: Cell pellets are washed with cold PBS and centrifuged at 300×g for 5 min. Cells are blocked with cold PBS+3% BSA and centrifuged (300×g for 5 min). Cells are incubated with primary antibodies diluted in PBS+3% BSA for 30 min at 4° C. Cells are washed three times with cold PBS+3% BSA and secondary antibodies (if applicable) diluted in PBS+3% BSA added and incubated for 30 min at 4° C. Cells are washed three times with cold PBS+3% BSA and resuspended in cold PBS. Flow cytometry is performed on a CytoFLEX cytometer (Beckman Coulter) and gating is performed on single cells and live cells before acquisition of 10,000 cells. Analysis is performed using the FlowJo software package.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

Throughout this specification, various patents, patent applications and other types of publications (e.g., journal articles, electronic database entries, etc.) are referenced. The disclosure of all patents, patent applications, and other publications cited herein are hereby incorporated by reference in their entirety for all purpose.

What is claimed is:

1. A bispecific binding agent comprising:
a) a first binding domain comprising a cytokine selected from the group consisting of CXCL12, CCL1, CCL2, CCL3, CCL3L1, CCL4, CC4L1, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, CCL28, CXCL11, CXCL1, CXCL2, CXCL3, CXCL4, CXCL5, CXCL6, CXCL7, CXCL8, CXCL9, CXCL10, CXCL13, CXCL14, CXCL16, CXCL17, CX3CL1, XCL1, XCL2, vMIPII, vCXC1 that specifically binds to at least one endogenous cell surface receptor, and
b) a second binding domain that specifically binds to a target protein, wherein the endogenous cell surface receptor is membrane associated, and wherein the binding of the first binding domain to the at least one endogenous cell surface receptor results in the internalization of the target protein bound to the bispecific binding agent.

2. A bispecific binding agent comprising:
a) a first binding domain that specifically binds to at least one endogenous cell surface receptor, and
b) a second binding domain that specifically binds to a target protein,
wherein the endogenous cell surface receptor is membrane associated, and wherein the binding of the first binding domain to the at least one endogenous cell surface receptor results in the internalization of the target protein bound to the bispecific binding agent.

3. The bispecific binding agent of any preceding claim, wherein the first binding domain specifically binds to one endogenous cell surface receptor.

4. The bispecific binding agent of any preceding claim wherein the first binding domain specifically binds to no more than two endogenous cell surface receptors.

5. The bispecific binding agent of any preceding claim, wherein the at least one endogenous cell surface receptor comprises targeting receptors and recycling receptors.

6. The bispecific binding agent of any one of the preceding claims, wherein the at least one endogenous cell surface receptor comprises single-pass and multi-pass membrane proteins.

7. The bispecific binding agent of any one of the preceding claims, wherein the at least one endogenous cell surface receptor comprises at least one cytokine receptor.

8. The bispecific binding agent of claim 7, wherein the at least one cytokine receptor comprises at least one chemokine receptor.

9. The bispecific binding agent of claim 8, wherein the at least one chemokine receptors are selected from the group consisting of CXCR1, CXCR2, CXCR3, CXCR4, CXCR5, CXCR6, CXCR7 (or ACKR3), XCR1, XCR2, CCR1, CCR2, CCR3, CCR4, CCR5, CCR6, CCR7, CCR8, CCR9, CCR10, CCR11, CX3CR1, ACKR1, ACKR2, ACKR4, and ACKR5.

10. The bispecific binding agent of claim 8, wherein the at least one chemokine receptors are selected from the group consisting of CCR1, CCR2, CCR3, CCR4, CCR5, CCR6, CCR7, CCR8, CCR9, CCR10, CCR11,

11. The bispecific binding agent of claim 8, wherein the at least one chemokine receptors are selected from the group consisting of CXCR7, CXCR4, CXCR3, CXCR1, CXCR2, CXCR5, CXCR6, CX3CR1, XCR1, XCR2.

12. The bispecific binding agent of claim 8, wherein the at least one chemokine receptors are selected from the group consisting of ACKR1, ACKR2, CXCR7, ACKR4.

13. The bispecific binding agent of claim 7, wherein the at least one cytokine receptor comprises at least one interleukin receptor.

14. The bispecific binding agent of claim 13 wherein the at least one interleukin receptors are selected from the group consisting of CD25, IL2RB, IL2RG, TL3RA, IL4R, IL13RA1, IL13RA2, IL5RA, IL6R, TL7R, IL9R, IL10RA, IL10RB, IL11RA, IL12RB1, IL12RB2, IL15RA, CD4, IL17RA, IL17RC, IL17RB, IL17RE, IL27RA, IL18R1, IL20RA, IL20RB, IL22RA1, IL21R, IL28RA, IL31RA, ST2, IL1RAP, CSF1R, IL1R1, IL1RL2, IL1R2.

15. The bispecific binding agent of claim 7, wherein the at least one cytokine receptor comprises at least one interferon receptor.

16. The bispecific binding agent of claim 15, wherein the at least one interferon receptors are selected from the group consisting of IFNAR1, IFNAR2, IFNGR1, IFNGR2.

17. The bispecific binding agent of any claim 7, wherein the at least one cytokine receptor comprises at least one prolactin receptor.

18. The bispecific binding agent of claim 17, wherein the at least one prolactin receptors are selected from the group consisting of EPOR, GHR, PRLR, CSF3R, LEPR, CSF1R.

19. The bispecific binding agent of claim 7, wherein the at least one cytokine receptor comprises at least one TNF receptor.

20. The bispecific binding agent of claim 19, wherein the at least one TNF receptors are selected from the group consisting of TNFR1, TNFR2, DR4, DR5, DCR1, DCR2, DR3, LTBR, BAFFR, TACI, OPG, RANK, CD40, EDAR, DCR3, FAS, CD27.

21. The bispecific binding agent of claim 3, wherein the at least one endogenous cell surface receptor comprises at least one growth factor receptor.

22. The bispecific binding agent of claim 21, wherein the at least one growth factor receptors are selected from the group consisting of FGFR2B, VEGFR2, PDGFRA, PDGFRB, NGFR, TRKC, TRKB, M6PR, IGF1R.

23. The bispecific binding agent of any one of the preceding claims, wherein the binding of the first binding domain to the at least one endogenous cell surface receptor results in the degradation of the target protein bound to the bispecific binding agent.

24. The bispecific binding agent of any one of claims 2-23, wherein the first binding domain comprises a cytokine, a chemokine, a growth factor or an isoform or a derivative capable of binding thereof.

25. The bispecific binding agent of claim 24, wherein the chemokine comprises a CXC chemokine, CCL chemokine, viral chemokine, or an isoform or a derivative capable of binding thereof.

26. The bispecific binding agent of claim 25, wherein the chemokine is selected from the group consisting of CCL1, CCL2, CCL3, CCL3L1, CCL4, CC4L1, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, CCL28.

27. The bispecific binding agent of claim 25, wherein the chemokine is selected from the group consisting of CXCL12, CXCL11, CXCL1, CXCL2, CXCL3, CXCL4, CXCL5, CXCL6, CXCL7, CXCL8, CXCL9, CXCL10, CXCL13, CXCL14, CXCL16, CXCL17, CX3CL1, XCL1, XCL2,

28. The bispecific binding agent of claim 25, wherein the chemokine is selected from the group consisting of vMIPII, vCXC1.

29. The bispecific binding agent of claim 24, wherein the cytokine is selected from the group consisting of interleukins, interferons, prolactins, tumor necrosis factors, and TGF-betas.

30. The bispecific binding agent of claim 29, wherein the cytokine is an interleukin.

31. The bispecific binding agent of claim 30, wherein the interleukin is selected from the group consisting of IL2, TL3, IL4, IL5, IL6, IL7, IL9, IL10, IL11, IL12A, IL12B, IL13, IL15, IL16, IL17A, IL17B, IL17C, IL17F, IL18, IL19, IL20, IL21, IL22, IL24, IL25, IL26, IL27, IL28A, IL28B, IL29, IL31, IL32, IL33, IL34, IL36A, IL36B, IL36G, IL36RA, IL37, IL38, IL1A, IL1B, IL1RN.

32. The bispecific binding agent of claim 29, wherein the cytokine is an interferon.

33. The bispecific binding agent of claim 32, wherein the interferon is selected from the group consisting of IFNA, IFNA1, IFNA2, IFNA4, IFNA5, IFNA6, IFNA7, IFNA8, IFNA10, IFNA14, IFNA16, IFNB, IFNG.

34. The bispecific binding agent of claim 29, wherein the cytokine is a prolactin,

35. The bispecific binding agent of claim 34, wherein the prolactin is selected from the group consisting of EPO, GH1, GH2, PRL, CSF3, LEP, CSF1.

36. The bispecific binding agent of claim 29, wherein the cytokine is an tumor necrosis factor.

37. The bispecific binding agent of claim 36, wherein the tumor necrosis factor is selected from the group consisting of TNFA. TNFB, TRAIL, TL1, BAFF, APRIL, RANKL, CD40LG, EDA, FASLG, and CD70.

38. The bispecific binding agent of claim 29, wherein the cytokine is a TGF-beta.

39. The bispecific binding agent of claim 38, wherein the TGF-beta is selected from the group consisting of TGFB1, TGFB2, TGFB3, GDF15, GDF2, BMP10, INHA, BMP3.

40. The bispecific binding agent of claim 24, wherein the first binding domain comprises a growth factor.

41. The bispecific binding agent of claim 10, wherein the growth factor is selected from the group consisting of FGF1, FGF2, FGF3, FGF4, FGF5, FGF19, FGF21, FGF23, KGF, VEGF, PDGFA, PDGFB, NGF, NTF3, NTF4, BDNF, IGF1, IGF2.

42. The bispecific binding agent of any one of the preceding claims, wherein the target protein comprises a soluble target protein and a membrane-associated target protein.

43. The bispecific binding agent of any one of the preceding claims, wherein the target protein is a membrane-associated target protein, and wherein the second binding domain binds to an extracellular epitope of a membrane-associated target protein.

44. The bispecific binding agent of any one of the preceding claims, wherein the target cell comprises a neoplastic cell.

45. The bispecific binding agent of any one of the preceding claims, wherein the target cell is a cancer cell selected from the group consisting of breast cancer, B cell lymphoma, pancreatic cancer, Hodgkin's lymphoma, ovarian cancer, prostate cancer, mesothelioma, lung cancer, non-Hodgkin's B-cell (B-NHL), melanoma, chronic lymphocytic leukemia, acute lymphocytic leukemia, neuroblastoma, glioma, glioblastoma, bladder cancer, and colorectal cancer.

46. The bispecific binding agent of any one of the preceding claims, wherein the target cell comprises an immune cell.

47. The bispecific binding agent of any one of the preceding claims, wherein the target protein is an immune checkpoint protein.

48. The bispecific binding agent of any one of the preceding claims, wherein the target protein comprises a cancer antigen.

49. The bispecific binding agent of any one of the preceding claims, wherein the cancer antigen comprises HER2, EGFR, CDCP1, CD38, IGF-1R, MMP14, and TROP2.

50. The bispecific binding agent of any one of the preceding claims, wherein the target protein comprises an immunomodulatory protein.

51. The bispecific binding agent of any one of the preceding claims, wherein the immunomodulatory protein comprises PD-L1, PD-1, CTLA-4, B7-H3, B7-H4, LAG3, NKG2D, TIM-3, VISTA, CD39, CD73 (NT5E), A2AR, SIGLEC7, and SIGLEC15.

52. The bispecific binding agent of any one of the preceding claims, wherein the target protein comprises a B cell antigen.

53. The bispecific binding agent of any one of the preceding claims, wherein the B cell antigen comprises CD19 and CD20.

54. The bispecific binding agent of any one of the preceding claims, wherein the target protein comprises a soluble target protein.

55. The bispecific binding agent of any one of the preceding claims, wherein the soluble target protein comprises an inflammatory cytokine, a growth factor (GF), a toxic enzyme, an autoantibody, a target associated with metabolic diseases, or a neuronal aggregate.

56. The bispecific binding agent of any one of the preceding claims, wherein the inflammatory cytokine comprises lymphotoxin, interleukin-1 (IL-1), IL-2, IL-5, IL-6, IL-12, IL-13, IL-17, IL-18, IL-23, tumor necrosis factor alpha (TNF-α), interferon gamma (IFNγ), and granulocyte-macrophage colony stimulating factor (GM-CSF).

57. The bispecific binding agent of any one of the preceding claims, wherein the growth factor comprises EGF, FGF, NGF, PDGF, VEGF, IGF, GMCSF, GCSF, TGF, RANK-L, erythropieitn, TPO, BMP, HGF, GDF, neurotrophins, MSF, SGF, GDF, and an isoform thereof.

58. The bispecific binding agent of any one of the preceding claims, wherein the toxic enzyme comprises a protein arginine deiminase 1 (PAD1), PAD2, PAD3, PAD4, and PAD6, leucocidin, hemolysin, coagulase, treptokinase, hyaluronidase.

59. The bispecific binding agent of claim 58, wherein the toxic enzyme comprises PAD2 or PAD4.

60. The bispecific binding agent of any one of the preceding claims, wherein the neuronal aggregate comprises Aβ, TTR, α-synuclein, TAO, and prion.

61. The bispecific binding agent of any one of the preceding claims, wherein the first binding domain and the second binding domain are each independently selected from the group consisting of natural ligands or a fragment, derivative, or small molecule mimetic thereof, IgG, half antibodies, single-domain antibodies, nanobodies, Fabs, monospecific Fab2, Fc, scFv, minibodies, IgNAR, V-NAR, hcIgG, VHH domains, camelid antibodies, and peptibodies.

62. The bispecific binding agent of any one of the preceding claims, wherein the first binding domain and the second binding domain together form a bispecific antibody, a bispecific diabody, a bispecific Fab2, a bispecific camelid antibody, a bispecific peptibody, scFv-Fc, a bispecific IgG, and a knob and hole bispecific IgG, a Fc-Fab, a knob and hole bispecific Fc-Fab, a cytokine-IgG fusion, a cytokine-Fab fusion, a cytokine-Fc-scFc fusion.

63. The bispecific binding agent of any one of the preceding claims, wherein the first binding domain comprises an Fc-fusion, and the second binding domain comprises a Fc-Fab.

64. The bispecific binding agent of any one of the preceding claims, comprising one or more sequences selected from Table 2.

65. A nucleic acid that encodes the bispecific binding agent of any one of the preceding claims.

66. The nucleic acid of claim 65, wherein the nucleic acid is operably connected to a promoter.

67. An engineered cell capable of protein expression comprising the nucleic acid of claim 65 or 66.

68. The engineered cell of claim 67, wherein the cell is a B cell, a B memory cell, or a plasma cell.

69. A method for making a bispecific binding agent, the method comprising:
i) providing a cell capable of protein synthesis, comprising the nucleic acid of claim 65 or 66; and
ii) inducing expression of the bispecific binding agent.

70. A vector, comprising the nucleic acid of claim 65 or 66.

71. The vector of claim 70, further comprising a promoter, wherein the promoter is operably linked to the nucleic acid.

72. An immunoconjugate comprising:
i) a bispecific binding agent of any one of the preceding claims,
ii) a small molecule, and
iii) a linker.

73. A pharmaceutical composition, comprising the bispecific binding agent, the nucleic acid, the vector, the engineered cell, or the immunoconjugate of any one of the preceding claims, and a pharmaceutically acceptable excipient.

74. A method of treating a disorder in a subject, the method comprising administering to a subject in need thereof, a therapeutically effective amount of the bispecific binding agent, the nucleic acid, the vector, the engineered cell, the immunoconjugate, or the pharmaceutical composition of any one of the preceding claims.

75. The method of claim 74, wherein the disorder comprises a neoplastic disorder, an inflammatory disease, a metabolic disorder, an endocrine disorder, and a neurological disorder.

76. The method of claim 75, wherein the neoplastic disorder comprises breast cancer, B cell lymphoma, pancreatic cancer, Hodgkin's lymphoma, ovarian cancer, prostate cancer, mesothelioma, lung cancer, non-Hodgkin's B-cell (B-NHL), melanoma, chronic lymphocytic leukemia, acute lymphocytic leukemia, neuroblastoma, glioma, glioblastoma, bladder cancer, and colorectal cancer.

77. The method of claim 75, wherein the inflammatory disease comprises inflammatory intestinal disease, rheumatoid arthritis, lupus, Crohn's disease, and ulcerative colitis.

78. The method of claim 75, wherein the metabolic disorder comprises diabetes, Gaucher disease, Hunter syndrome, Krabbe disease, maple syrup urine disease, metachromatic leukodystrophy, mitochondrial encephalopathy, lactic acidosis, stroke-like episodes (MELAS), Niemann-Pick, phenylketonuria (PKU), porphyria, Tay-Sachs disease, and Wilson's disease.

79. The method of claim 75, wherein the neurological disorder comprises Parkinson's disease, Alzheimer's disease, and multiple sclerosis.
